(19) United States
(12) Patent Application Publication
(10) Pub. No.: US 2017/0325457 A1
(43) Pub. Date: Nov. 16, 2017

(54) FORMULATIONS FOR THE DELIVERY OF ACTIVE AGENTS TO INSECTS, PLANTS, AND PLANT PATHOGENS

(71) Applicant: Preceres Inc., Cambridge, MA (US)

(72) Inventors: Richard Wayne Heidebrecht, JR., Somerville, MA (US); Sonke Svenson, Arlington, MA (US); Roger Wiegand, Wayland, MA (US); Jungyeon Hwang, Lexington, MA (US); Jen Beaudoin, Holden, MA (US); Cheng Zhong, Belmont, MA (US)

(73) Assignee: Preceres Inc., Cambridge, MA (US)

(21) Appl. No.: 15/498,013

(22) Filed: Apr. 26, 2017

Related U.S. Application Data
(60) Provisional application No. 62/328,838, filed on Apr. 28, 2016.
(60) Provisional application No. 62/341,306, filed on May 25, 2016.

(51) Int. Cl. A01N 43/90; C07J 41/00; A01N 45/00; C07D 233/64; C07C 229/24; C07C 229/26

(52) U.S. Cl. CPC A01N 43/90; C07J 41/0033; C07J 41/0088; A01N 45/00; C07C 229/24; C07C 229/26; C07D 233/64

(57) ABSTRACT

The present disclosure is directed to formulations comprising (1) at least one formulation transport agent, (2) at least one complexing agent, and (3) at least one active agent that modulates one or more traits of a target insect, plant, or plant pathogen. The present disclosure is also directed to methods of delivering such formulations to the target organism, as well as to formulation transport agents used to prepare such formulations.

### RELATED APPLICATIONS

This application claims priority to U.S. Provisional Patent Application No. 62/328,838 filed on Apr. 28, 2016, and U.S. Provisional Patent Application No. 62/341,306 filed on May 25, 2016, the contents of each of which are incorporated herein in their entirety.

### FIELD OF THE INVENTION

The present disclosure relates generally to novel formulations for delivery of active agents that modulate one or more traits of target insects, plants, and plant pathogens. In addition to the active agent, the formulations comprise at least one formulation transport agent and at least one complexing agent. The present disclosure also relates generally to methods of delivering such formulations to the target organisms, as well as to novel formulation transport agents.

### BACKGROUND OF THE INVENTION

Numerous auxiliary compounds have been employed in lipid- and lipidoid-based formulations of poly- or oligonucleotides to assist in their delivery to mammals and mammalian cellular systems. Some of these compounds, such as cholesterol, facilitate delivery of the poly- or oligonucleotide into the target mammalian cell by packing inside the lipid/lipioid bilayer of the formulation, affording the formulation with improved metastability and phase/melting temperatures. Other compounds facilitate delivery of the poly- or oligonucleotide across the mammalian cell membrane by engaging its endogenous transport mechanisms. However, lipid- and lipidoid formulations have yet to be developed which contain auxiliary compounds that (1) facilitate delivery of nucleotides to non-mammalian cells, such as insect, plant, and plant pathogen cells, by engaging their unique endogenous transport mechanisms and (2) impart the robust metastability required for delivery of the formulation in an agricultural environment. Thus, there exists a continuing need for lipid- and lipidoid-based formulations of nucleotides that possess an improved ability to deliver nucleotides to non-mammalians cells in the agricultural setting.

### Embodiments of the Invention

One embodiment of the present invention is a formulation comprising (1) at least one formulation transport agent, (2) at least one complexing agent, and (3) a first active agent that modulates a trait of a target organism, wherein the target organism is an insect, a plant, or a plant pathogen.

Another embodiment of the present invention is the above formulation, wherein the at least one formulation transport agent is a cell targeting agent, a membrane penetration agent, an intracellular transport agent, a decomplexing agent, or any combination thereof.

Another embodiment of the present invention is the above formulation, wherein the at least one formulation transport agent is an insect-, plant-, or plant pathogen-derived steroid or derivative thereof.

Another embodiment of the present invention is the above formulation, wherein the at least one formulation transport agent is a phytol derivative.

Another embodiment of the present invention is the above formulation, wherein the at least one formulation transport agent is an insect-, plant-, or plant pathogen-derived hormone or hormone mimic.

Another embodiment of the present invention is the above formulation, wherein the at least one formulation transport agent is a surfactant.

Another embodiment of the present invention is the above formulation, wherein the at least one formulation transport agent is a compound of formula (I):

A-B-C (I)

wherein
A is a group that facilitates transport of the formulation to, into, and within a cell of the target organism and/or decomplexation of the formulation;
B is a linker; and
C is a group that is non-covalently associated to the at least one complexing agent;
wherein the linker B is at least in part formed from a moiety of A and a moiety of C.

Another embodiment of the present invention is the above formulation, wherein A is a cationic group, a group derived from an insect-, plant-, or plant pathogen-derived hormone, and/or a group derived from a carbohydrate.

Another embodiment of the present invention is the above formulation, wherein C is a group derived from an insect-, plant-, or plant pathogen-derived steroid or a group derived from a tocopherol.

Another embodiment of the present invention is the above formulation, wherein: A is a group derived from glucose, sucrose, maltose, kanamycin, arginine, lysine, or histidine, or a group selected from the group consisting of formulae (II), (III), (IV), (V), (VI), (VII), (VIII), (IX), and (X):

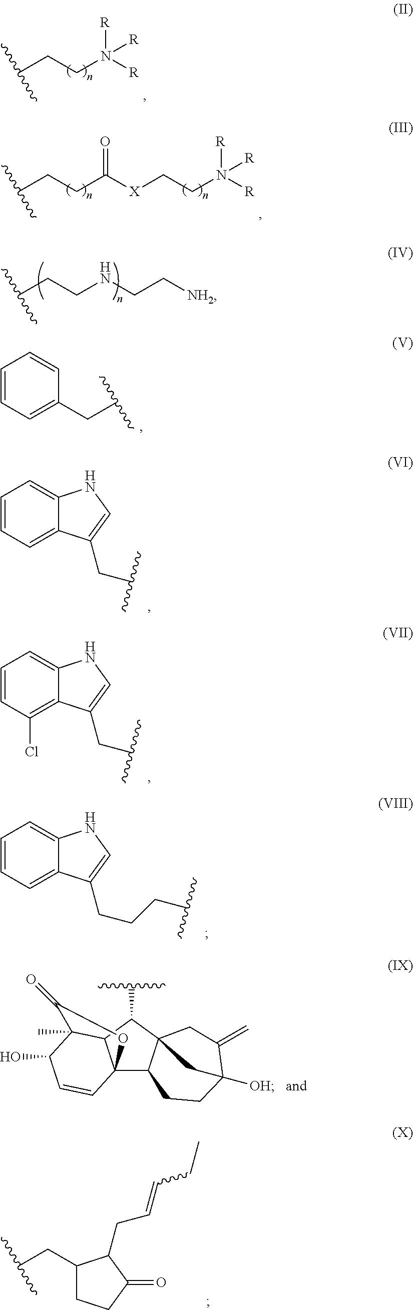

wherein

### X is O or NH;

R is —H, —CH₃, —CH₂CH₃, or —CH₂CH₂OH; and
n is 0, 1, or 2.

Another embodiment of the present invention is the above formulation, wherein:

B is a covalent bond or a group selected from the group consisting of formulae (XI), (XII), (XIII), (XIV), (XV), (XVI), (XVII), and (XVIII):

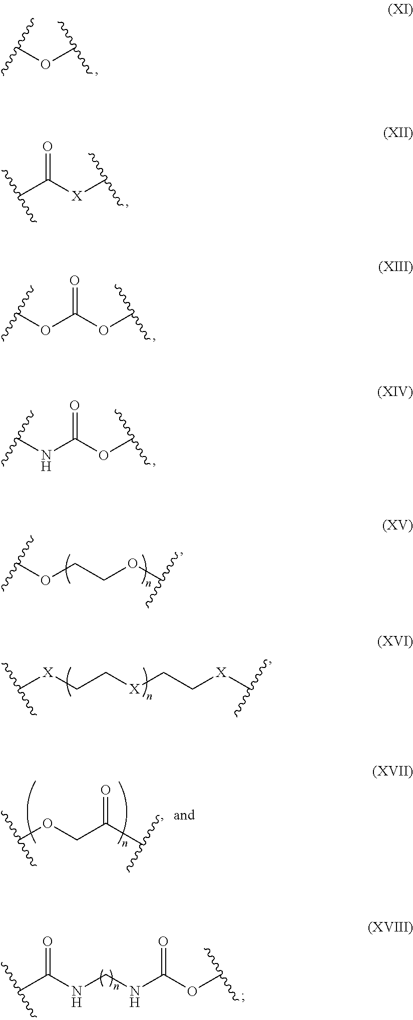

wherein
X is, independently, O or NH; and
n and integer in the range of from 1 to 10.

Another embodiment of the present invention is the above formulation, wherein:

C is a group selected from the group consisting of formulae (XIX), (XX), (XXI), (XXII), (XXIII), (XXIV), (XXV), and (XXVI):

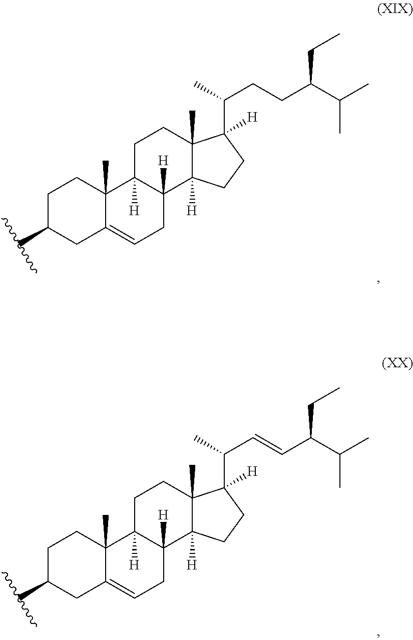

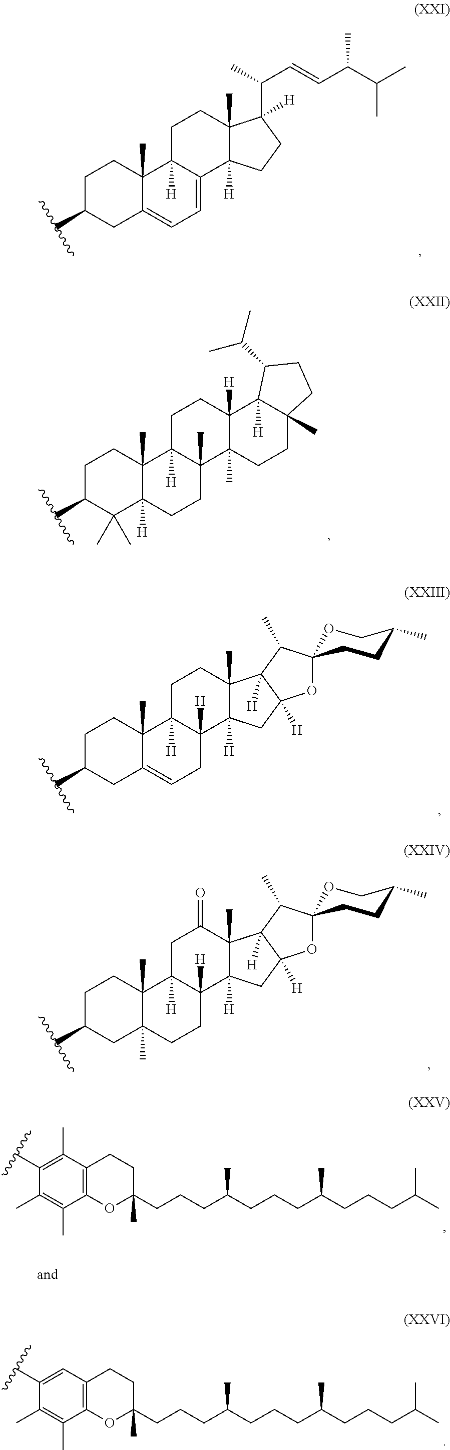

Another embodiment of the present invention is the above formulation, wherein:

A is a group derived from glucose, sucrose, maltose, kanamycin, arginine, lysine, or histidine, or a group selected from the group consisting of formulae (II), (III), (IV), (V), (VI), (VII), (VIII), (IX), and (X):

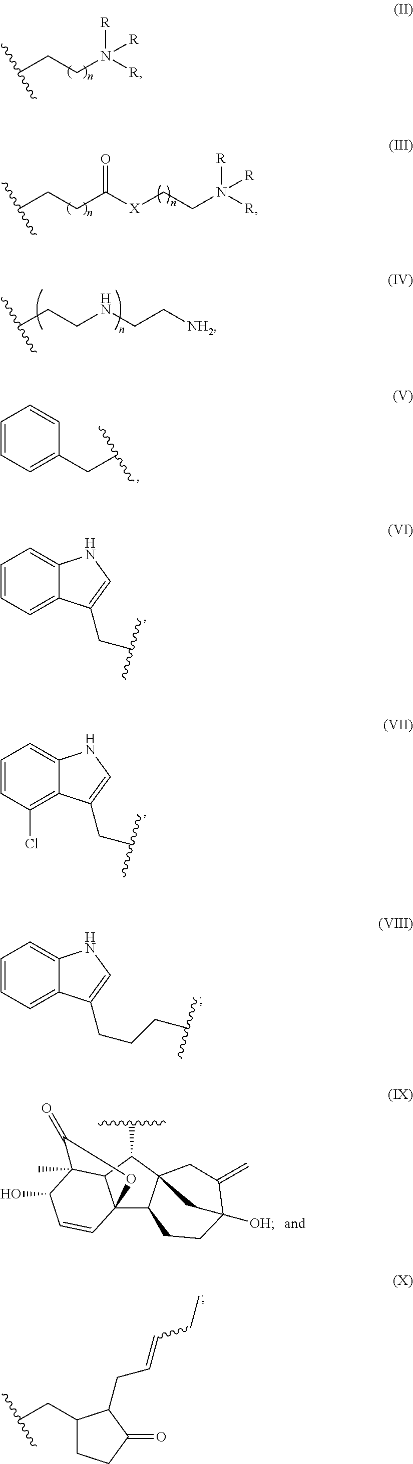

wherein
X is O or NH;
R is —H, —CH₃, —CH₂CH₃, or —CH₂CH₂OH; and
n is 0, 1, or 2; and
   B is a covalent bond or a group selected from the group consisting of formulae (XI), (XII), (XIII), (XIV), (XV), (XVI), (XVII), and (XVIII):

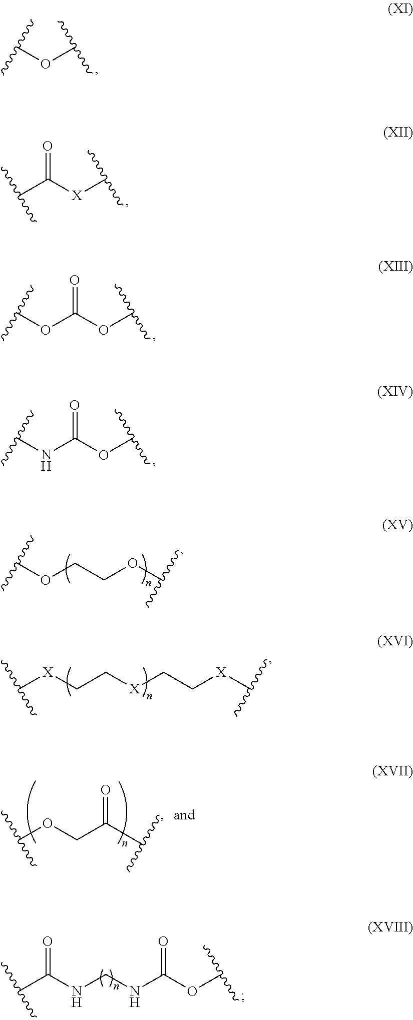

wherein
X is, independently, O or NH; and
n and integer in the range of from 1 to 10.

Another embodiment of the present invention is the above formulation, wherein:

A is a group derived from glucose, sucrose, maltose, kanamycin, arginine, lysine, or histidine, or a group selected from the group consisting of formulae (II), (III), (IV), (V), (VI), (VII), (VIII), (IX), and (X):

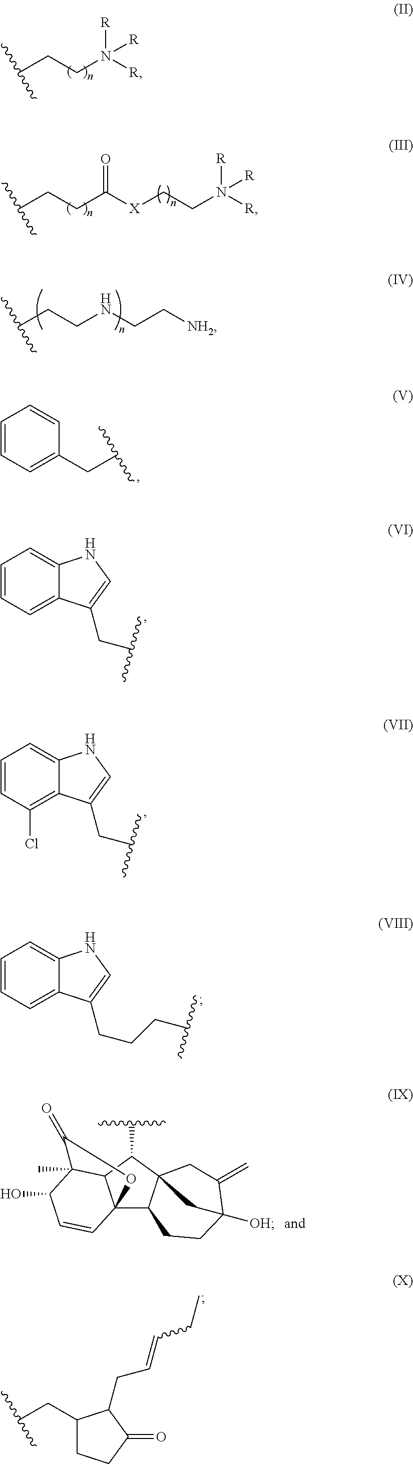

wherein
X is O or NH;
R is —H, —CH₃, —CH₂CH₃, or —CH₂CH₂OH; and
n is 0, 1, or 2;
   B is a covalent bond or a group selected from the group consisting of formulae (XI), (XII), (XIII), (XIV), (XV), (XVI), (XVII), and (XVIII):

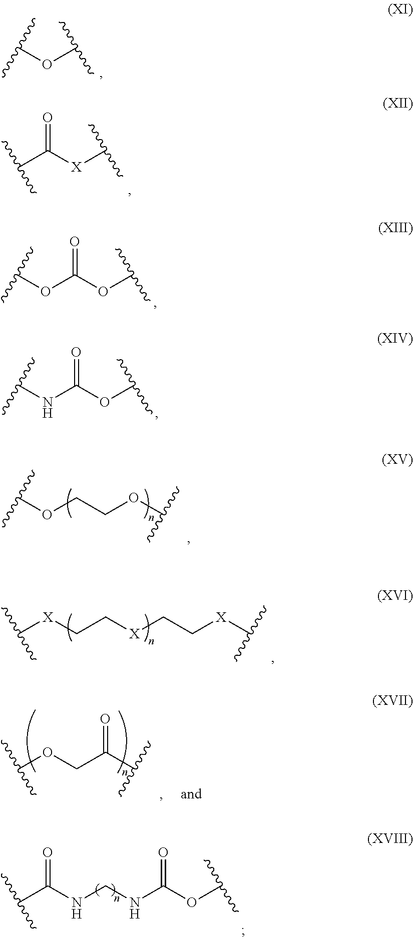

wherein
X is, independently, O or NH; and
n and integer in the range of from 1 to 10; and
   C is a group selected from the group consisting of formulae (XIX), (XX), (XXI), (XXII), (XXIII), (XXIV), (XXV), and (XXVI):

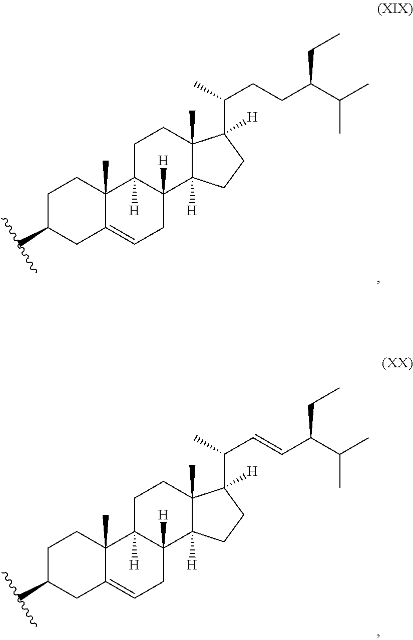

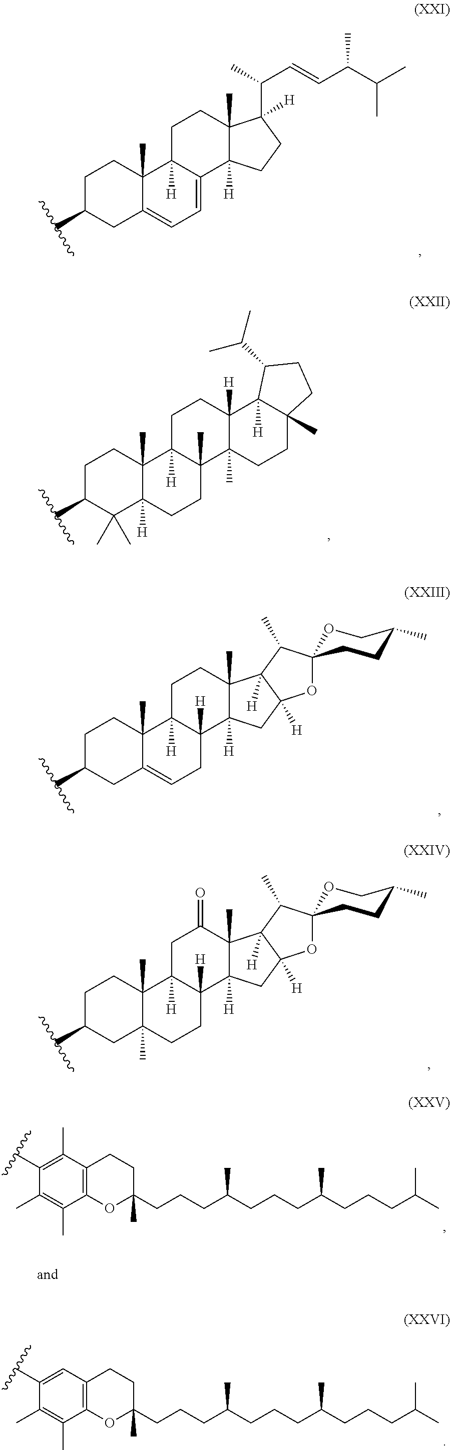

Another embodiment of the present invention is the above formulation, wherein the compound of formula (I) is a compound of structure (1) through (50):

| Compound | |
|---|---|
| Number | Structure |
| 1 | 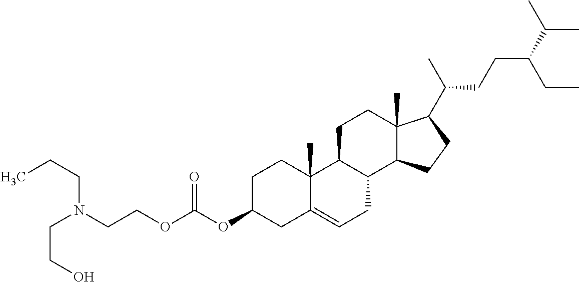 [SMILES: CCCN(CCO)CCOC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 2 | 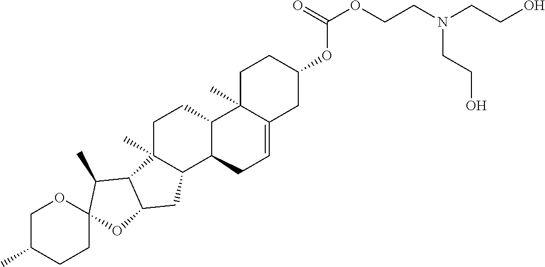 [SMILES: C[C@H]1CC[C@]2(OC1)O[C@H]1C[C@H]3[C@@H]4CC=C5C[C@@H](OC(=O)OCCN(CCO)CCO)CC[C@]5(C)[C@H]4CC[C@]3(C)[C@H]1[C@@H]2C] |
| 3 | 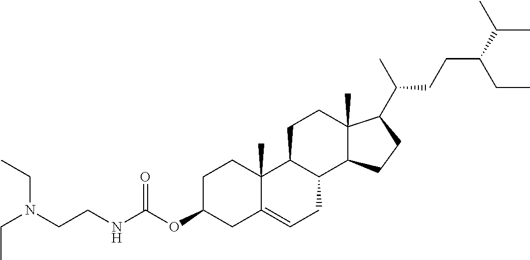 [SMILES: CC[C@H](CC[C@@H](C)[C@H]1CC[C@H]2[C@@H]3CC=C4C[C@@H](OC(=O)NCCN(CC)CC)CC[C@]4(C)[C@H]3CC[C@]12C)C(C)C] |
| 4 | 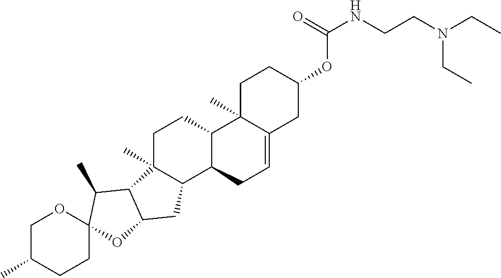 [SMILES: CCN(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1] |
| 5 | 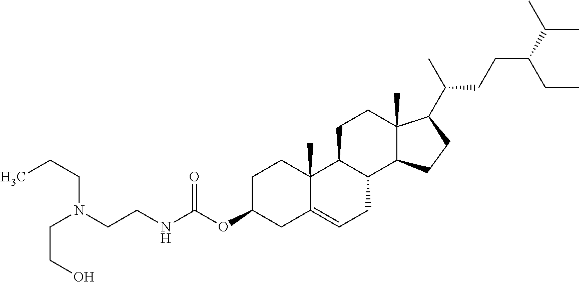 [SMILES: CCCN(CCO)CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 6 | 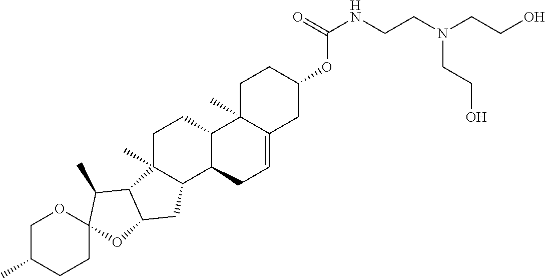 [SMILES: C[C@H]1CC[C@]2(OC1)O[C@H]1C[C@H]3[C@@H]4CC=C5C[C@@H](OC(=O)NCCN(CCO)CCO)CC[C@]5(C)[C@H]4CC[C@]3(C)[C@H]1[C@@H]2C] |
| 7 | 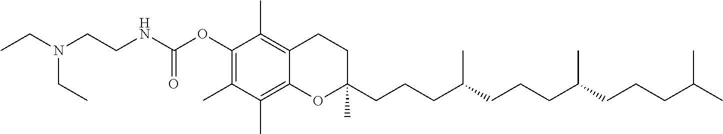 [SMILES: CCN(CC)CCNC(=O)Oc1c(C)c(C)c2c(c1C)CC[C@@](C)(CCC[C@H](C)CCC[C@H](C)CCCC(C)C)O2] |
| 8 | 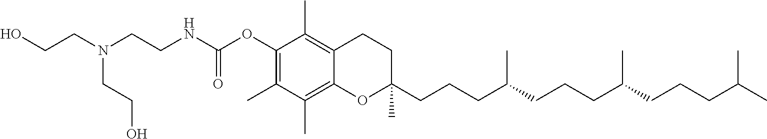 [SMILES: Cc1c(C)c2c(c(C)c1OC(=O)NCCN(CCO)CCO)CC[C@@](C)(CCC[C@H](C)CCC[C@H](C)CCCC(C)C)O2] |
| 9 | 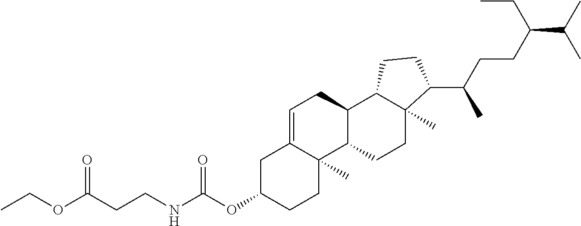 [SMILES: CCOC(=O)CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 10 | 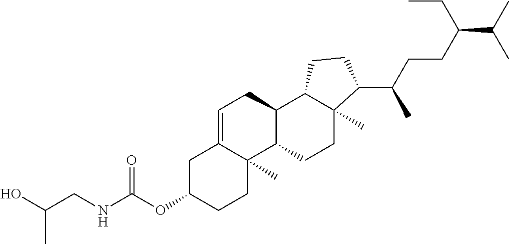 [SMILES: CC[C@H](CC[C@@H](C)[C@H]1CC[C@H]2[C@@H]3CC=C4C[C@@H](OC(=O)NCC(C)O)CC[C@]4(C)[C@H]3CC[C@]12C)C(C)C] |
| 11 | 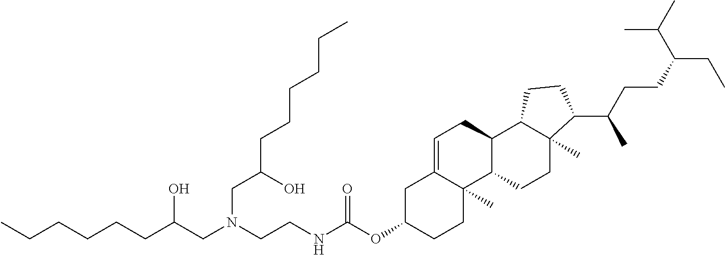 [SMILES: CCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCC] |
| 12 | 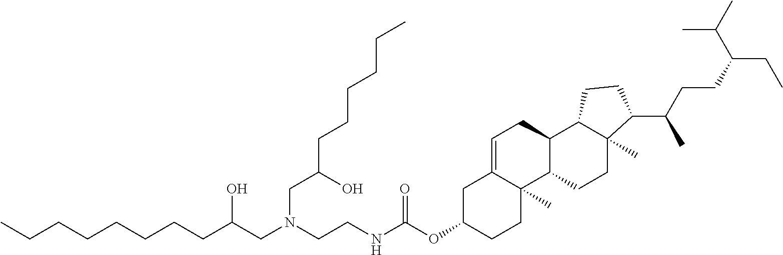 [SMILES: CCCCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCC] |
| 13 | 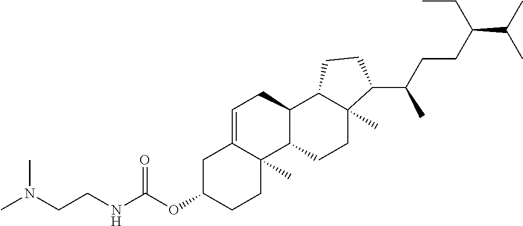 [SMILES: CC[C@H](CC[C@@H](C)[C@H]1CC[C@H]2[C@@H]3CC=C4C[C@@H](OC(=O)NCCN(C)C)CC[C@]4(C)[C@H]3CC[C@]12C)C(C)C] |
| 14 | 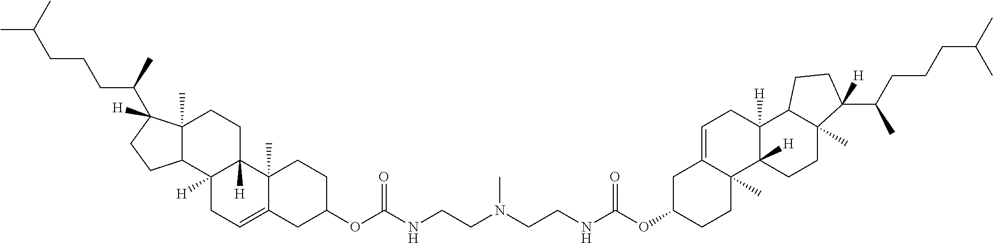 |
| 15 | 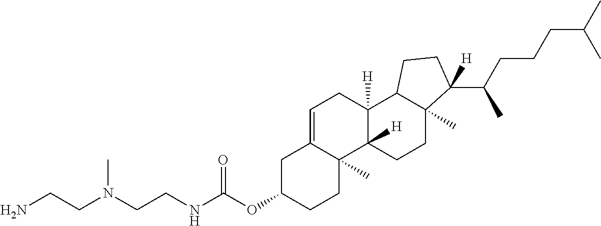 |
| 16 | 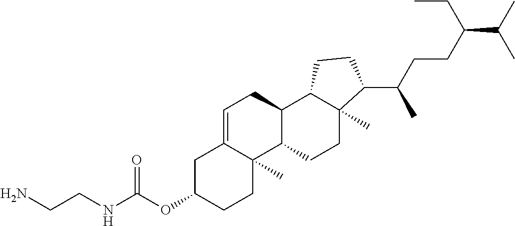 [SMILES: CC[C@H](CC[C@@H](C)[C@H]1CC[C@H]2[C@@H]3CC=C4C[C@@H](OC(=O)NCCN)CC[C@]4(C)[C@H]3CC[C@]12C)C(C)C] |
| 17 | 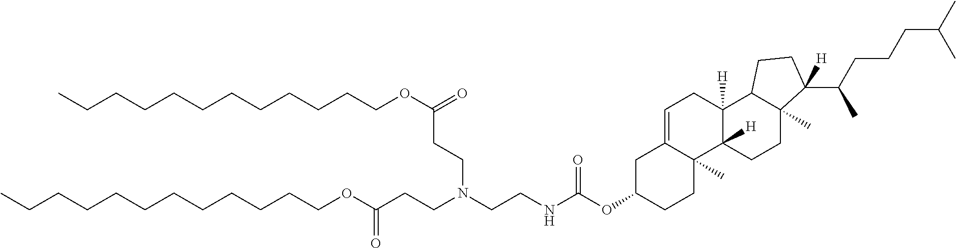 |
| 18 | 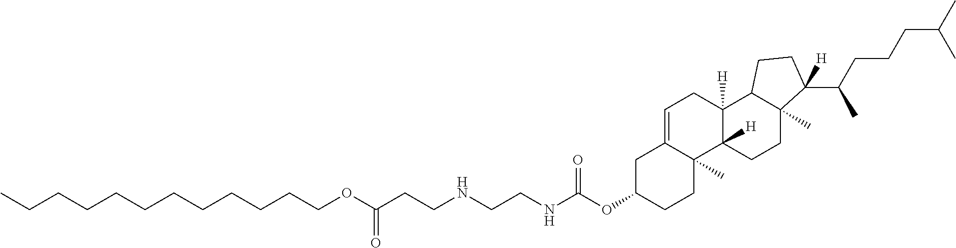 |
| 19 | 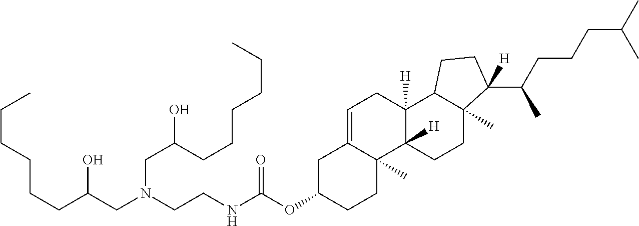 |
| 20 | 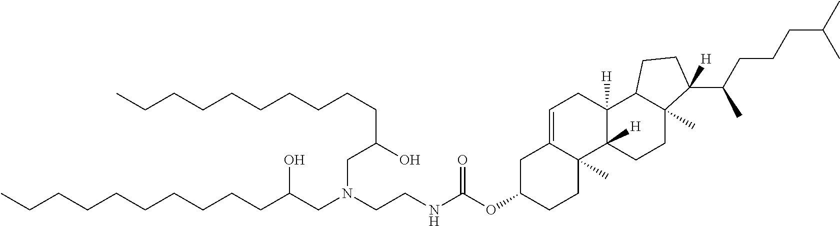 |
| 21 | 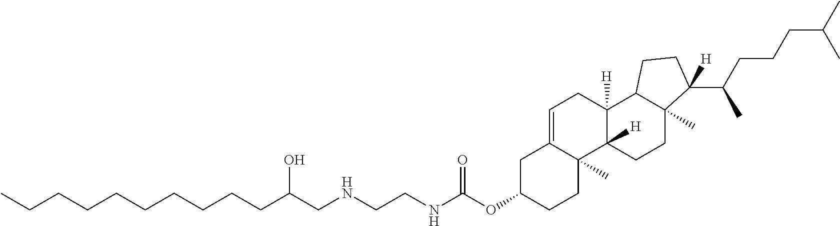 |
| 22 | 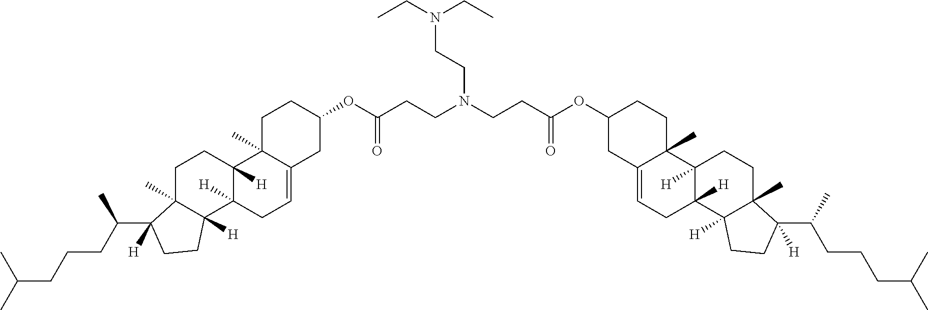 |
| 23 | 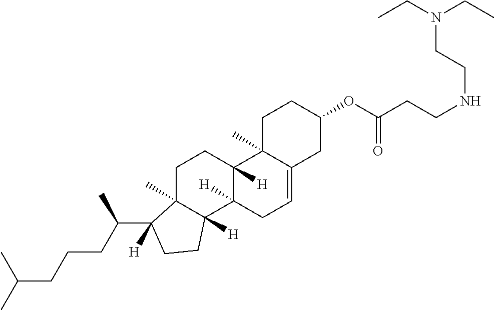 |
| 24 | 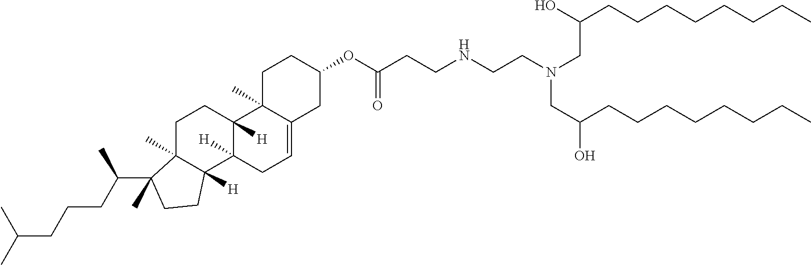 |
| 25 | 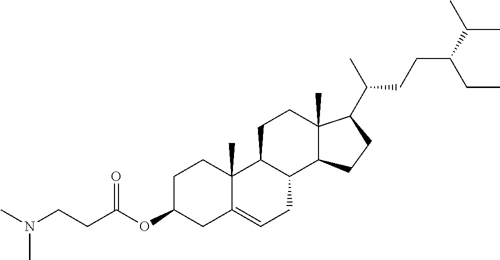 [SMILES: CC[C@H](CC[C@@H](C)[C@H]1CC[C@H]2[C@@H]3CC=C4C[C@@H](OC(=O)CCN(C)C)CC[C@]4(C)[C@H]3CC[C@]12C)C(C)C] |
| 26 | 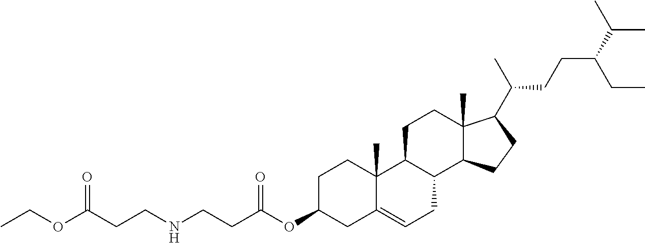 [SMILES: CCOC(=O)CCNCCC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 27 | 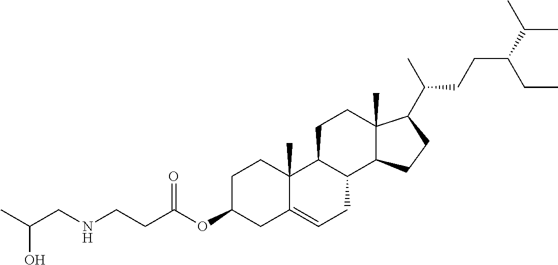 [SMILES: CC[C@H](CC[C@@H](C)[C@H]1CC[C@H]2[C@@H]3CC=C4C[C@@H](OC(=O)CCNCC(C)O)CC[C@]4(C)[C@H]3CC[C@]12C)C(C)C] |
| 28 | 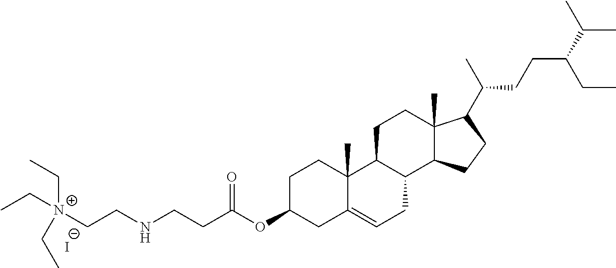 |
| 29 | 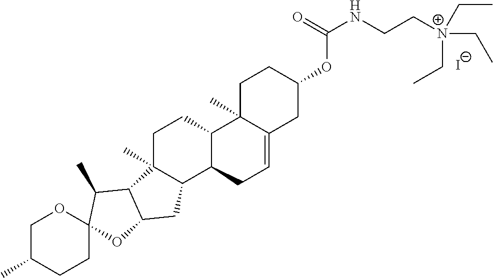 [SMILES: CC[N+](CC)(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1 ; [I-]] |
| 30 | 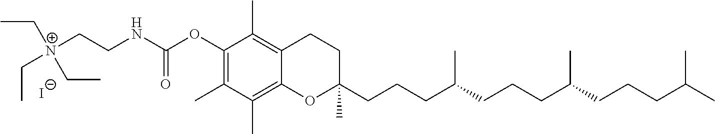 [SMILES: CC[N+](CC)(CC)CCNC(=O)Oc1c(C)c(C)c2c(c1C)CC[C@@](C)(CCC[C@H](C)CCC[C@H](C)CCCC(C)C)O2 ; [I-]] |
| 31 | 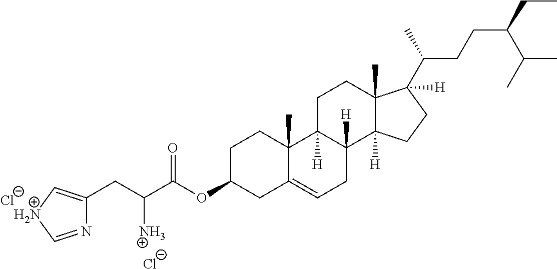 |
| 32 | 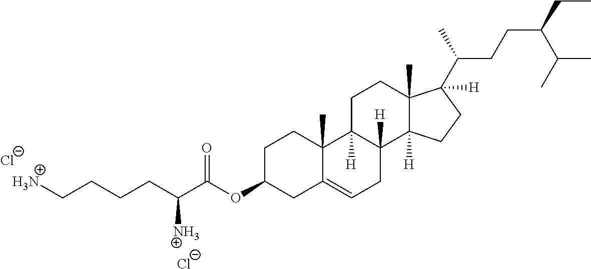 |
| 33 | 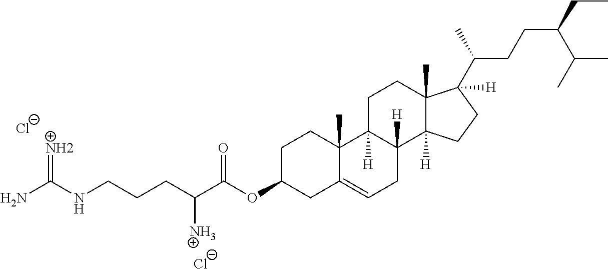 |
| 34 | 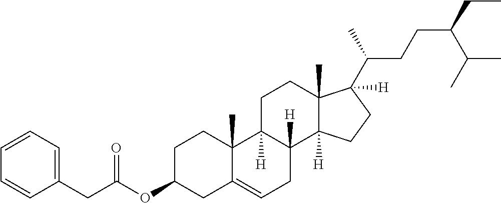 |
| 35 | 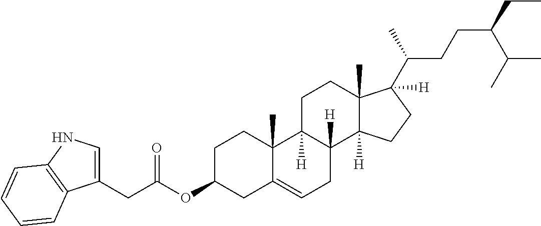 |
| 36 | 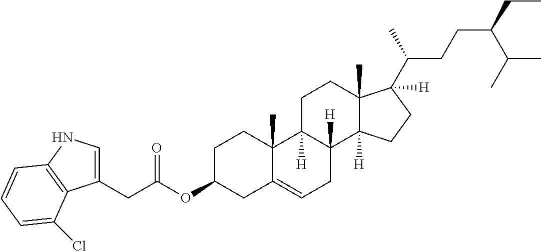 |
| 37 | 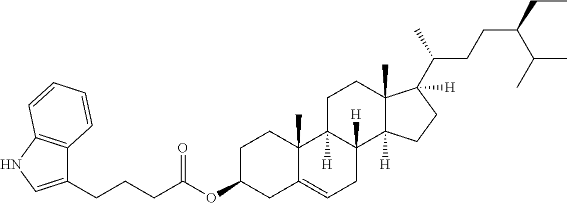 |
| 38 | 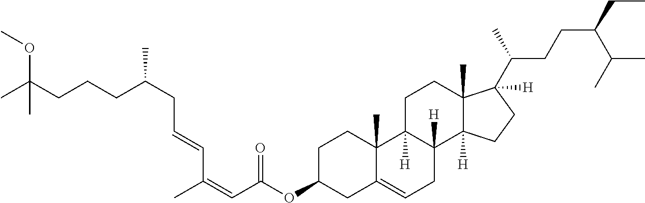 |
| 39 | 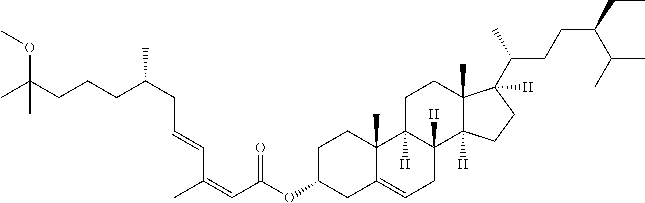 |
| 40 | 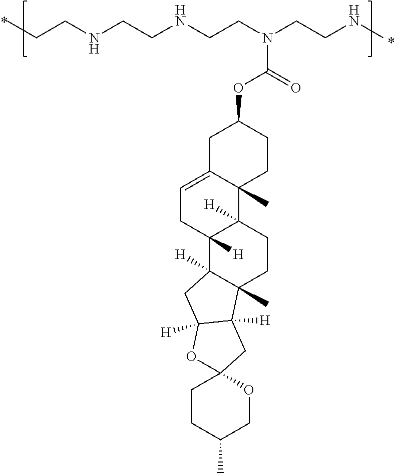 |
| 41 | 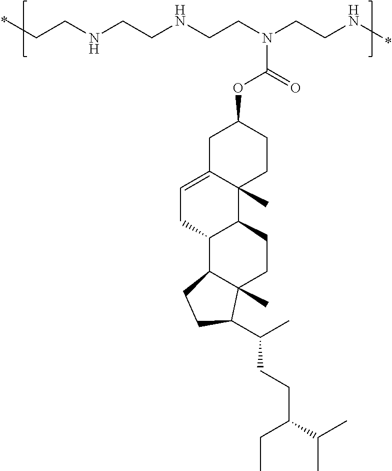 |
| 42 | 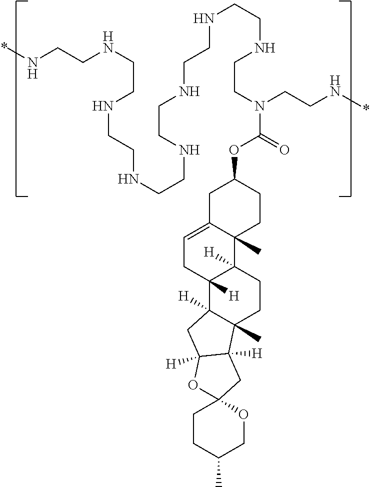 |
| 43 | 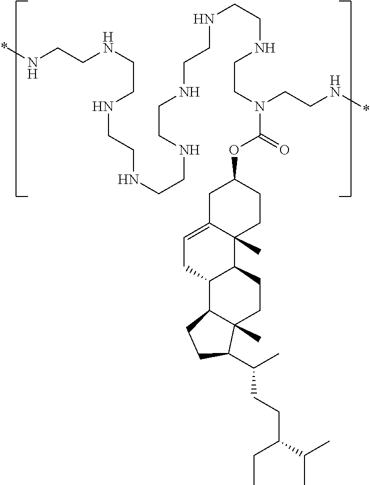 |
| 44 | 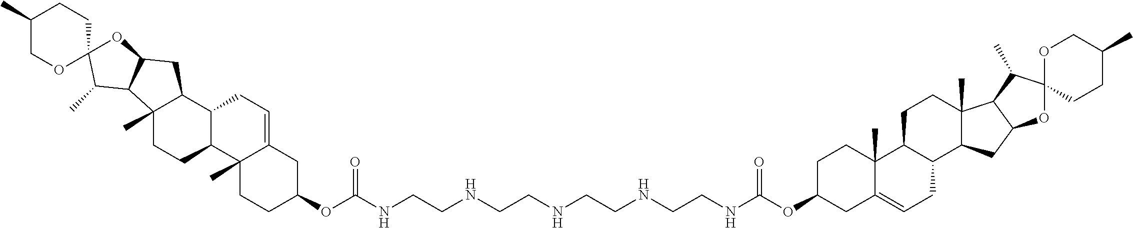 |
| 45 | 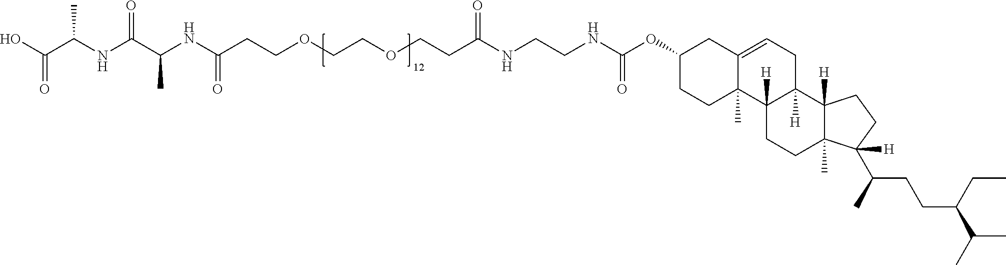 |
| 46 | 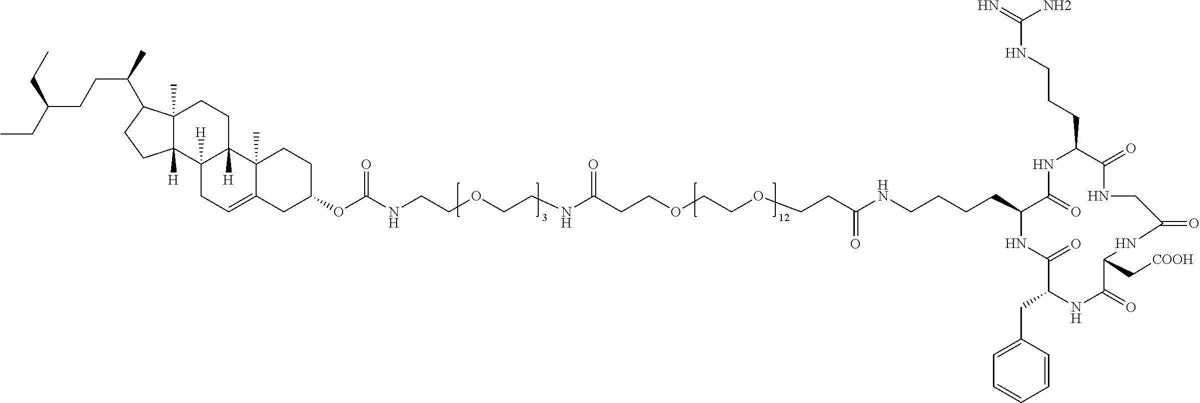 |
| 47 | 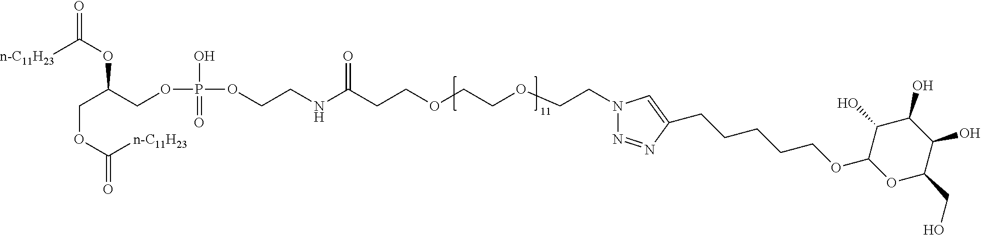 |
| 48 | 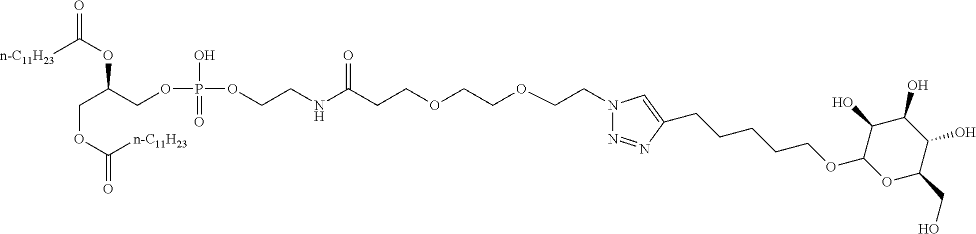 |
| 49 | 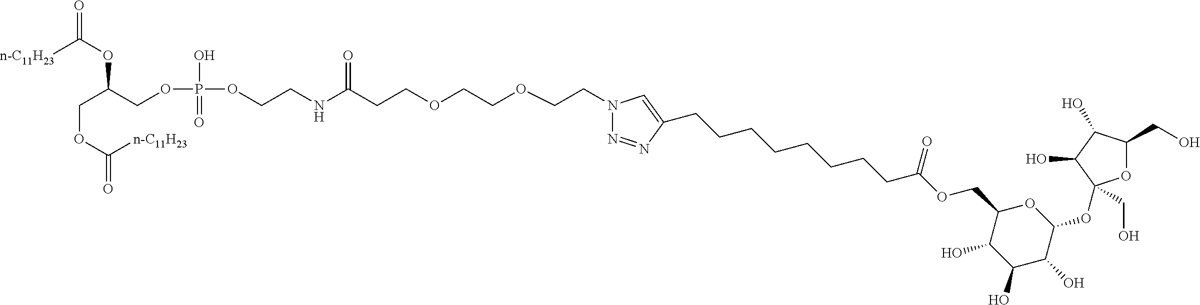 |
| 50 | 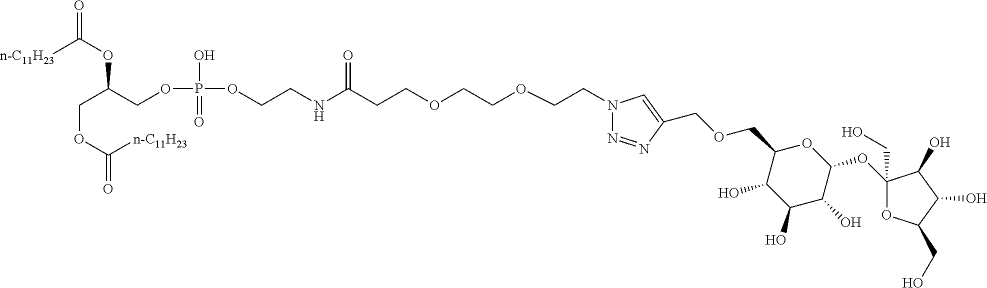 |

Another embodiment of the present invention is the above formulation, where the compound of formula (I) is a gibberellic acid derivative of formula (XXVII):

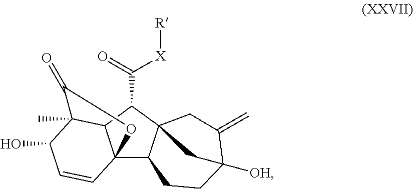

wherein

### X is O or NH; and

R′ is an alkyl group or the residue of any steroid, tocopherol, endogenous auxin, or carbohydrate.

Another embodiment of the present invention is the above formulation, wherein R′ is a C₁ to C₂₀ alkyl group.

Another embodiment of the present invention is the above formulation, wherein X is O and R′ is a C₁₂ alkyl group or X is O or NH and R′ is a group of formula (XXVIII):

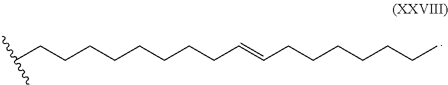 [SMILES: *CCCCCCCC/C=C/CCCCCCC]

Another embodiment of the present invention is the above formulation, wherein X is O and R′ is a group selected from the group consisting of formulae (V), (VI), (VII), (VIII), (XIX), (XX), (XXI), (XXII), (XXIII), (XXIV), (XXV), and (XXVI):

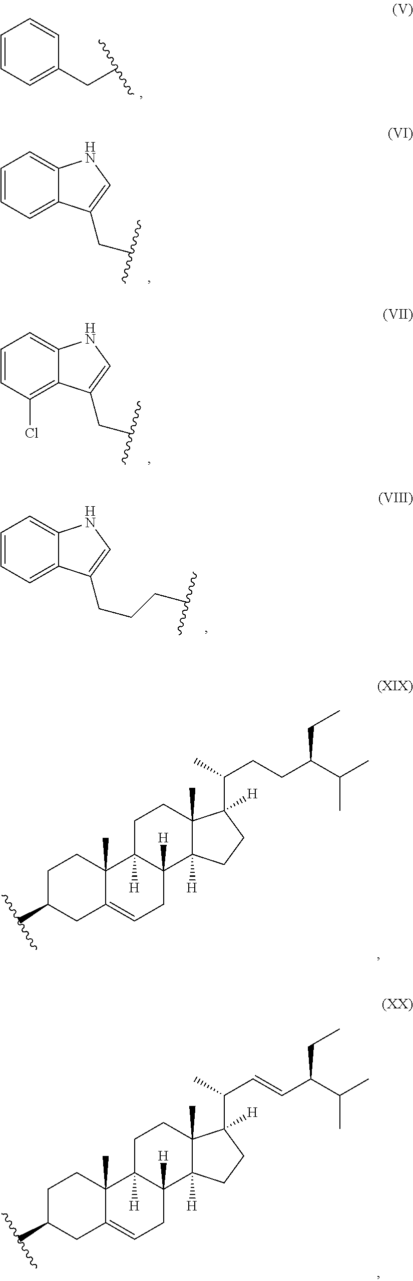

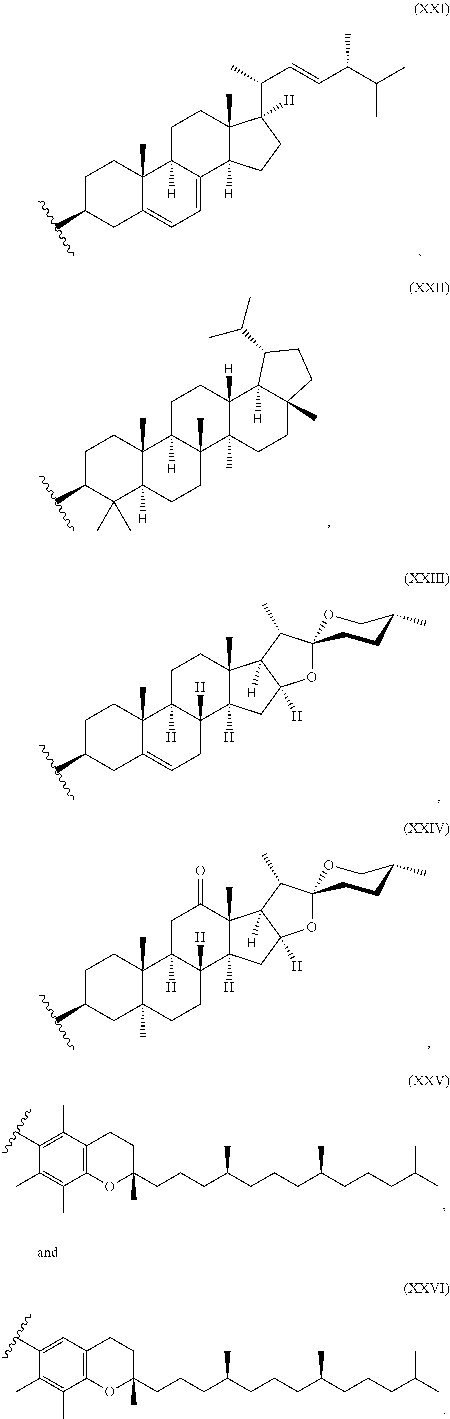

Another embodiment of the present invention is the above formulation, wherein B is O and C is a group derived from glucose, sucrose, maltose, or kanamycin.

Another embodiment of the present invention is the above formulation, further comprising an adjuvant selected from the group consisting of chloroquine, chlorpromazine, amodiaquine, perphenazine, coronatine, tolbutamide, glyburide, glybenclamide, arginine, lysine, and histidine.

Another embodiment of the present invention is the above formulation, further comprising at least one additional active agent to be delivered.

Another embodiment of the present invention is the above formulation, wherein the at least one additional active agent to be delivered is contained within or on the surface of the non-covalent complex.

Another embodiment of the present invention is the above formulation, wherein the at least one additional active agent to be delivered is not contained within or on the surface of the non-covalent complex.

Another embodiment of the present invention is the above formulation, further comprising one or more excipients.

Another embodiment of the present invention is the above formulation, wherein the one or more excipients is selected from the group consisting of fillers, extenders, binders, humectants, disintegrants, plasticizers, stabilizers, solution retarding agents, wetting agents, suspending agents, thickening agents, absorbents, lubricants, surfactants, buffering agents, diluents, solvents, emulsifying agents, suspending agents, sweetening agents, flavoring agents, perfuming agents, opacifying agents, separating agents, coating permeability adjusters, and combinations thereof.

Another embodiment of the present invention is the above formulation, wherein the one or more excipients is selected from the group consisting of carbohydrates, proteins, lipids, water-soluble polymers, and any combination thereof.

Another embodiment of the present invention is the above formulation, wherein the one or more water soluble polymers comprises a polyethylene glycol, a polypropylene oxide, a polyvinylpyrrolidone, a polyvinyl alcohol, polylactic acid, poly(lactic-co-glycolic acid), or any combination thereof.

Another embodiment of the present invention is the above formulation, wherein the first active agent to be delivered is an oligonucleotide or a polynucleotide.

Another embodiment of the present invention is the above formulation, further comprising an agriculturally acceptable carrier.

Another embodiment of the present invention is the above formulation, wherein the oligonucleotide or polynucleotide modulates the expression of a gene in a plant.

Another embodiment of the present invention is the above formulation, wherein the oligonucleotide or polynucleotide modulates the expression of a gene in an insect.

Another embodiment of the present invention is the above formulation, wherein the oligonucleotide or polynucleotide modulates the expression of a gene in a plant pathogen.

Another embodiment of the present invention is the above formulation, wherein the at least one additional active agent is selected from the group consisting of an herbicide, an insecticide, a fungicide, a nematicide, a bactericide, a viricide, and any combination thereof.

Yet another embodiment of the present invention is the above formulation, wherein the formulation is in the form of a microparticle or nanoparticle.

Another embodiment of the present invention is the above microparticle or nanoparticle formulation, wherein the active agent to be delivered is selected from the group consisting of polynucleotides, oligonucleotides, proteins, peptides, and small molecules.

Another embodiment of the present invention is the above microparticle or nanoparticle formulation, wherein the active agent to be delivered is an oligonucleotide or a polynucleotide.

Another embodiment of the present invention is the above microparticle or nanoparticle formulation, wherein the oligonucleotide or polynucleotide is modified.

Another embodiment of the present invention is the above microparticle or nanoparticle formulation, wherein the oligonucleotide or polynucleotide is unmodified.

Another embodiment of the present invention is the above microparticle or nanoparticle formulation, wherein the active agent to be delivered is an RNA.

Another embodiment of the present invention is the above microparticle or nanoparticle formulation, wherein the RNA is a single-stranded RNA.

Another embodiment of the present invention is the above microparticle or nanoparticle formulation, wherein the RNA is a double-stranded RNA.

Another embodiment of the present invention is the above microparticle or nanoparticle formulation, wherein the RNA is a siRNA.

Another embodiment of the present invention is the above microparticle or nanoparticle formulation, wherein the RNA is a mRNA.

Yet another embodiment of the present invention is a method of regulating expression of a gene in the target organism, comprising applying any of the above formulations to the target organism.

Yet another embodiment of the present invention is a method of modulating a trait of a plant, comprising delivering to the plant an effective amount of the above formulation comprising an oligonucleotide or a polynucleotide that modulates the expression of a gene in a plant.

Another embodiment of the present invention is the above method, wherein the trait is selected from the group consisting of total seed germination, rate of seed germination, disease tolerance, insect tolerance, drought tolerance, heat tolerance, cold tolerance, salinity tolerance, tolerance to heavy metals, total yield, seed yield, fruit yield, root growth, early vigor, plant growth, plant biomass, plant size, plant lifespan, total plant dry weight, above-ground dry weight, above-ground fresh weight, leaf area, stem volume, plant height, rosette diameter, leaf length, root length, root mass, tiller number, leaf number, fruit size, fruit freshness, fruit ripening time, fruit nutritional content, plant nutritional content, plant sensitivity to herbicide, and any combination thereof.

Another embodiment of the present invention is the above method, wherein one or more of the traits is improved relative to a plant not treated with the formulation.

Another embodiment of the present invention is the above method, wherein at least one trait selected from the group consisting of plant growth, plant lifespan, plant size, fruit size, fruit yield, total yield, fruit freshness, fruit ripening time, plant nutritional content, and fruit nutritional content, is improved relative to a plant not treated with the formulation.

Another embodiment of the present invention is the above method, wherein one or more of the traits is decreased relative to a plant not treated with the formulation.

Another embodiment of the present invention is the above method, wherein the plant growth and/or the plant lifespan is decreased relative to a plant not treated with the formulation.

Another embodiment of the present invention is the above method, wherein the fruit ripening time is decreased relative to a plant not treated with the formulation.

Another embodiment of the present invention is the above method, wherein the plant sensitivity to herbicide is increased relative to a plant not treated with the formulation.

Yet another embodiment of the present invention is a method of modulating a trait of an insect, comprising delivering to the insect, to a plant infested with the insect, or to a plant prior to infestation with the insect, an effective amount of the above formulation comprising an oligonucleotide or a polynucleotide that modulates the expression of a gene in an insect.

Another embodiment of the present invention is the above method, wherein the trait modulated is insect growth, development, and/or lifespan.

Yet another embodiment of the present invention is a method of modulating the pathogenicity of a plant pathogen, comprising applying to the plant pathogen, to a plant infected with the plant pathogen, or to a plant prior to infection with the plant pathogen, the above formulation comprising an oligonucleotide or a polynucleotide that modulates the expression of a gene in a plant pathogen.

Yet another embodiment of the present invention is a plant cell, an insect cell, a fungal cell, a nematodic cell, or a bacterial cell, comprising the above formulation.

Yet another embodiment of the present invention is a compound of formula (I):

A-B-C (I)

wherein
A is a group that can facilitate transport of a formulation to, into, and within a cell of a target organism and/or decomplexation of the formulation within the target organism;
B is a linker; and
C is a group that can non-covalently associate to at least one complexing agent of the formulation;
wherein
the linker B is at least in part formed from a moiety of A and a moiety of C;
the formulation comprises a first active agent that modulates a trait of a target organism and at least one complexing agent; and
the target organism is an insect, a plant, or a plant pathogen.

Another embodiment of the present invention is the above compound, wherein A is a cationic group, a group derived from an insect-, plant-, or plant pathogen-derived hormone, or a group derived from a carbohydrate.

Another embodiment of the present invention is the above compound, wherein C is a group derived from an insect-, plant-, or plant pathogen-derived steroid or a group derived from a tocopherol.

Another embodiment of the present invention is the above compound, wherein: A is a group derived from glucose, sucrose, maltose, kanamycin, arginine, lysine, or histidine or a group selected from the group consisting of formulae (II), (III), (IV), (V), (VI), (VII), (VIII), (IX), and (X):

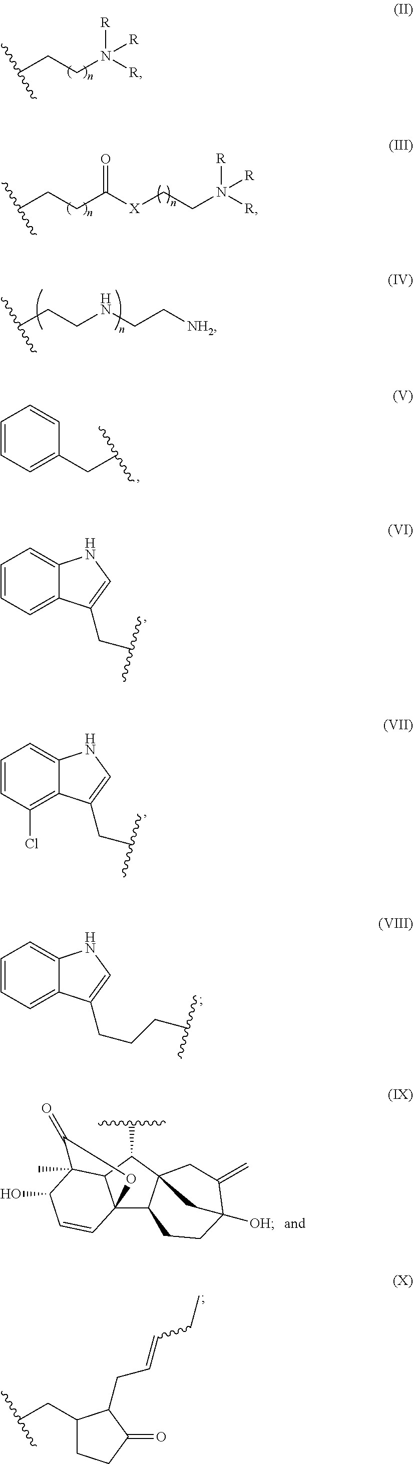

wherein

### X is O or NH;

R is —H, —CH₃, —CH₂CH₃, or —CH₂CH₂OH; and
n is 0, 1, or 2.

Another embodiment of the present invention is the above compound, wherein:

B is a covalent bond or a group selected from the group consisting of formulae (XI), (XII), (XIII), (XIV), (XV), (XVI), (XVII), and (XVIII):

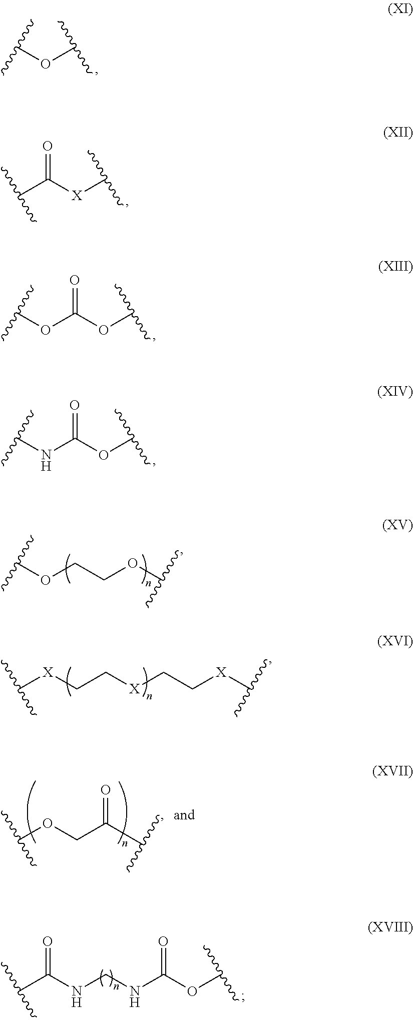

wherein
X is, independently, O or NH; and
n and integer in the range of from 1 to 10.

Another embodiment of the present invention is the above compound, wherein:

C is a group selected from the group consisting of formulae (XIX), (XX), (XXI), (XXII), (XXIII), (XXIV), (XXV), and (XXVI):

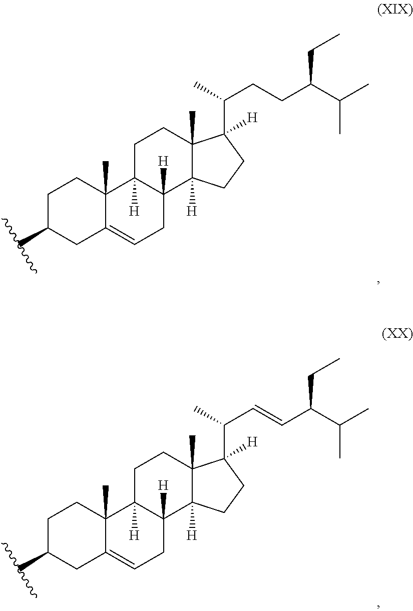

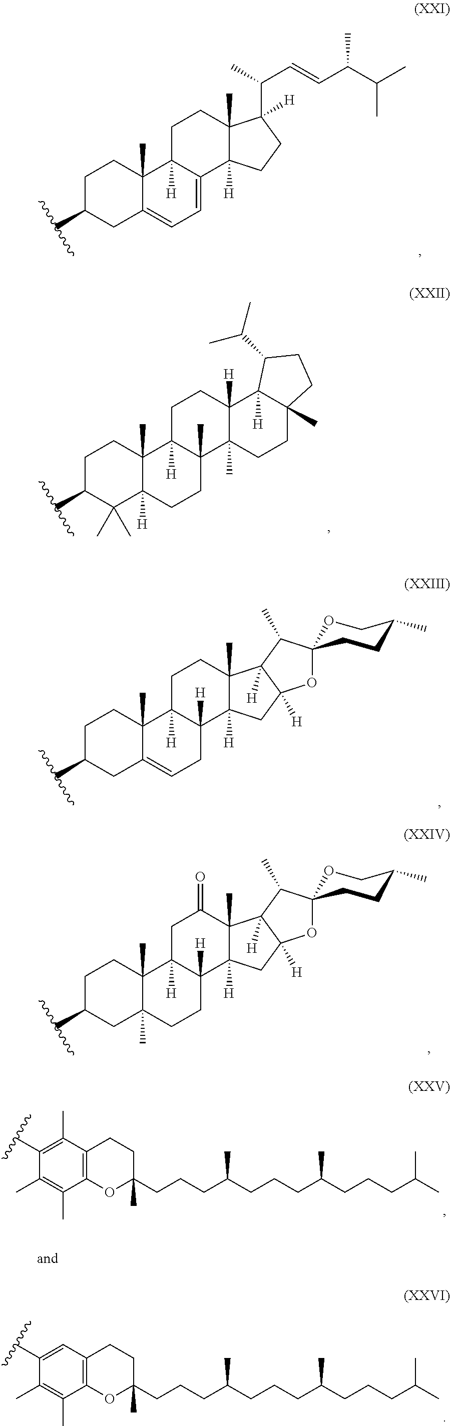

Another embodiment of the present invention is the above compound, wherein:

A is a group derived from glucose, sucrose, maltose, kanamycin, arginine, lysine, or histidine or a group selected from the group consisting of formulae (II), (III), (IV), (V), (VI), (VII), (VIII), (IX), and (X):

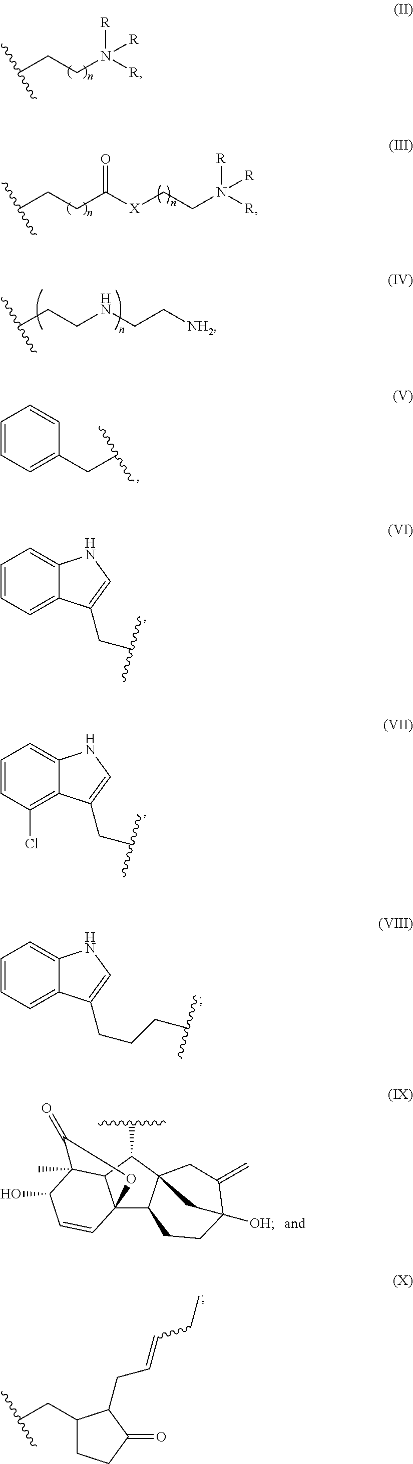

wherein
X is O or NH;
R is —H, —CH₃, —CH₂CH₃, or —CH₂CH₂OH; and
n is 0, 1, or 2; and
   B is a covalent bond or a group selected from the group consisting of formulae (XI), (XII), (XIII), (XIV), (XV), (XVI), (XVII), and (XVIII):

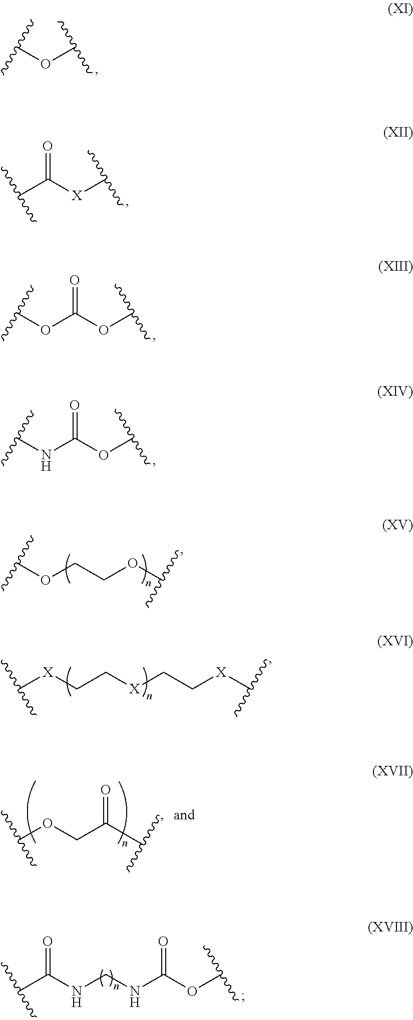

wherein
X is, independently, O or NH; and
n and integer in the range of from 1 to 10.

Another embodiment of the present invention is the above compound, wherein:

A is a group derived from glucose, sucrose, maltose, kanamycin, arginine, lysine, or histidine or a group selected from the group consisting of formulae (II), (III), (IV), (V), (VI), (VII), (VIII), (IX), and (X):

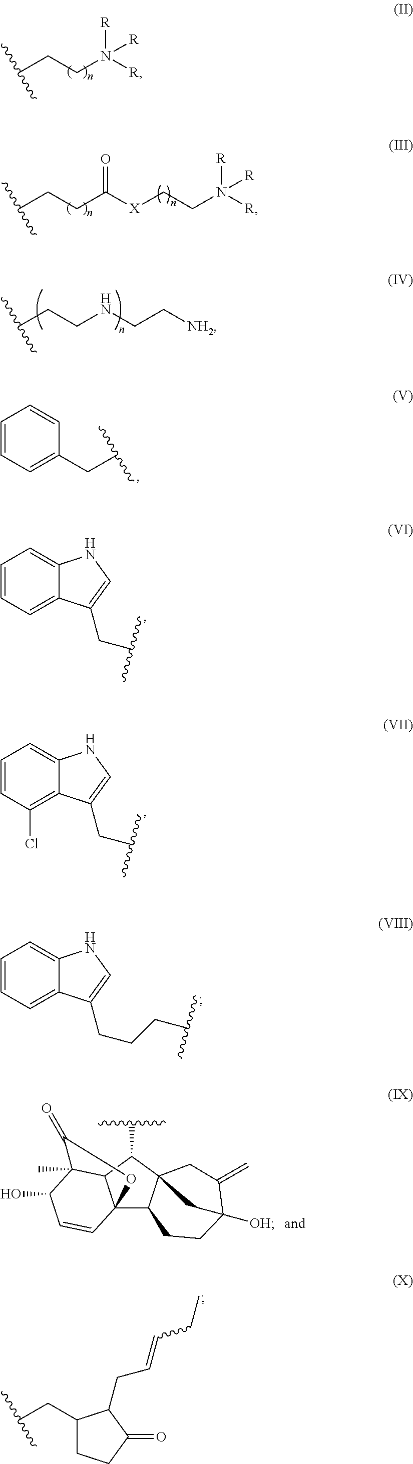

wherein
X is O or NH;
R is —H, —CH₃, —CH₂CH₃, or —CH₂CH₂OH; and
n is 0, 1, or 2;
   B is a covalent bond or a group selected from the group consisting of formulae (XI), (XII), (XIII), (XIV), (XV), (XVI), (XVII), and (XVIII):

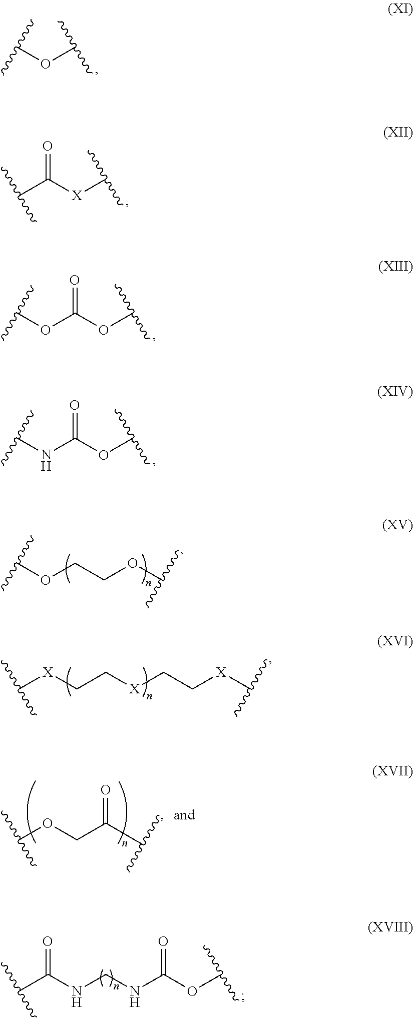

wherein
X is, independently, O or NH; and
n and integer in the range of from 1 to 10; and
   C is a group selected from the group consisting of formulae (XIX), (XX), (XXI), (XXII), (XXIII), (XXIV), (XXV), and (XXVI):

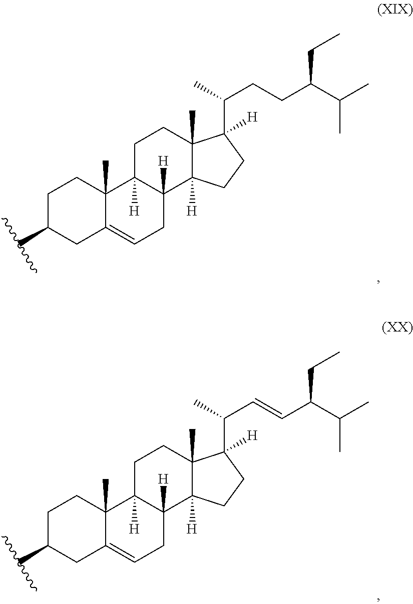

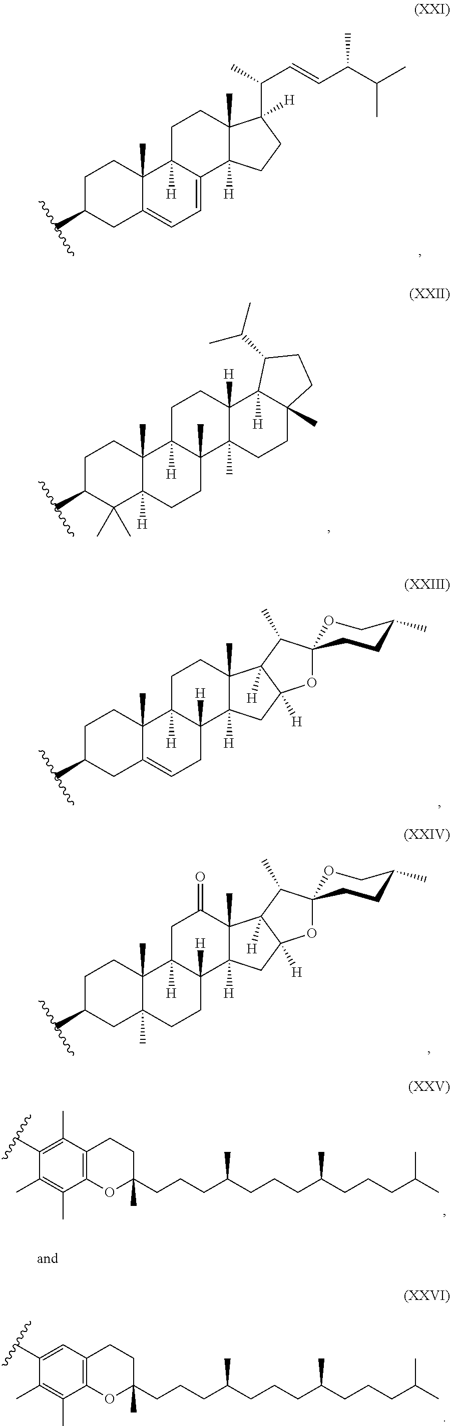

Another embodiment of the present invention is the above compound, wherein the compound is selected from the group consisting of structures (1) through (50):

| Compound | |
|---|---|
| Number | Structure |
| 1 | 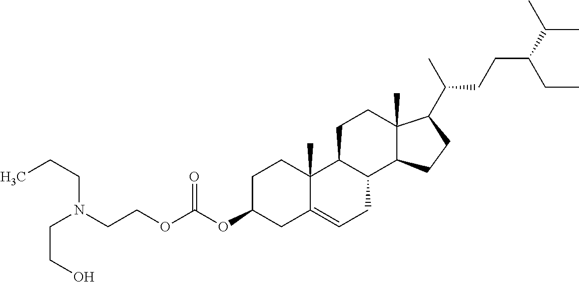 [SMILES: CCCN(CCO)CCOC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 2 | 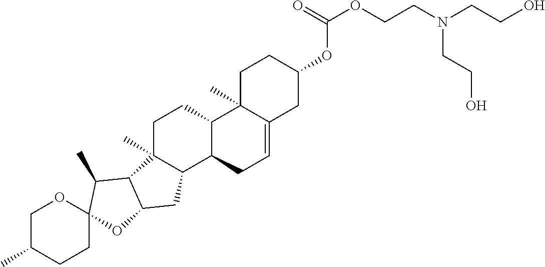 [SMILES: C[C@H]1CC[C@]2(OC1)O[C@H]1C[C@H]3[C@@H]4CC=C5C[C@@H](OC(=O)OCCN(CCO)CCO)CC[C@]5(C)[C@H]4CC[C@]3(C)[C@H]1[C@@H]2C] |
| 3 | 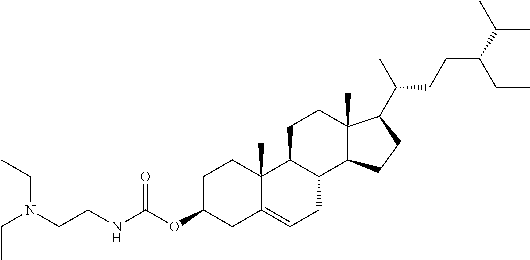 [SMILES: CC[C@H](CC[C@@H](C)[C@H]1CC[C@H]2[C@@H]3CC=C4C[C@@H](OC(=O)NCCN(CC)CC)CC[C@]4(C)[C@H]3CC[C@]12C)C(C)C] |
| 4 | 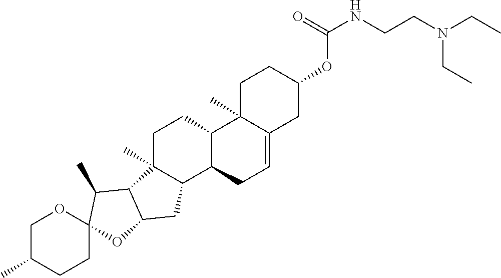 [SMILES: CCN(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1] |
| 5 | 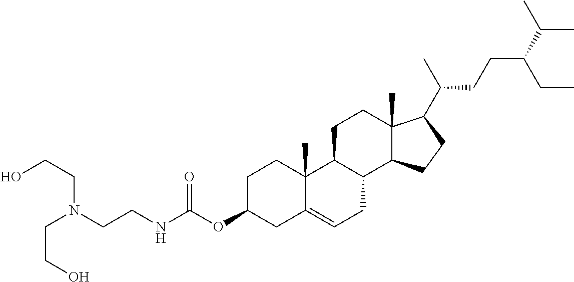 [SMILES: CC[C@H](CC[C@@H](C)[C@H]1CC[C@H]2[C@@H]3CC=C4C[C@@H](OC(=O)NCCN(CCO)CCO)CC[C@]4(C)[C@H]3CC[C@]12C)C(C)C] |
| 6 | 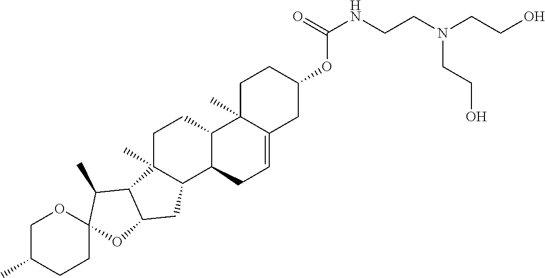 [SMILES: C[C@H]1CC[C@]2(OC1)O[C@H]1C[C@H]3[C@@H]4CC=C5C[C@@H](OC(=O)NCCN(CCO)CCO)CC[C@]5(C)[C@H]4CC[C@]3(C)[C@H]1[C@@H]2C] |
| 7 | 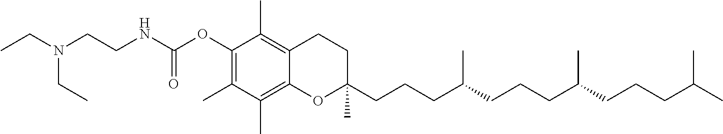 [SMILES: CCN(CC)CCNC(=O)Oc1c(C)c(C)c2c(c1C)CC[C@@](C)(CCC[C@H](C)CCC[C@H](C)CCCC(C)C)O2] |
| 8 | 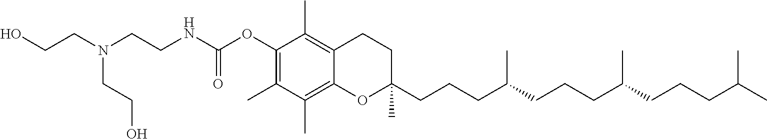 [SMILES: Cc1c(C)c2c(c(C)c1OC(=O)NCCN(CCO)CCO)CC[C@@](C)(CCC[C@H](C)CCC[C@H](C)CCCC(C)C)O2] |
| 9 | 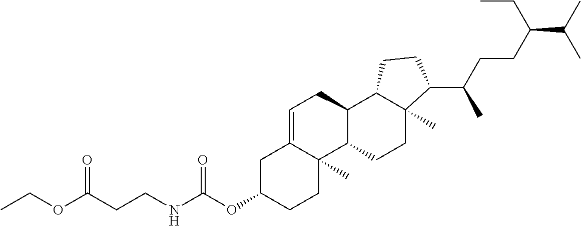 [SMILES: CCOC(=O)CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 10 | 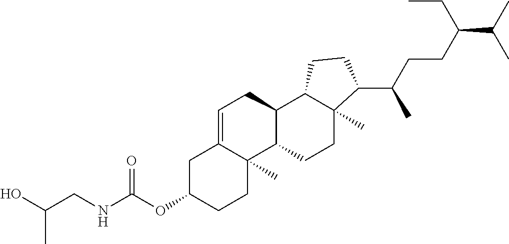 [SMILES: CC[C@H](CC[C@@H](C)[C@H]1CC[C@H]2[C@@H]3CC=C4C[C@@H](OC(=O)NCC(C)O)CC[C@]4(C)[C@H]3CC[C@]12C)C(C)C] |
| 11 | 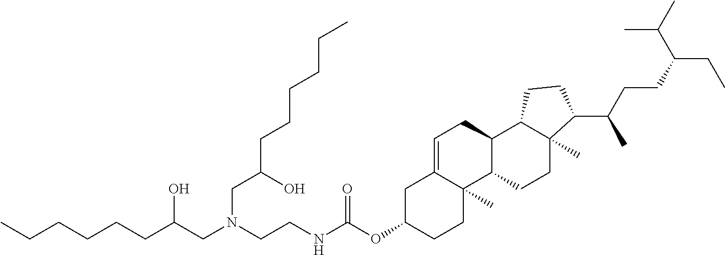 [SMILES: CCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCC] |
| 12 | 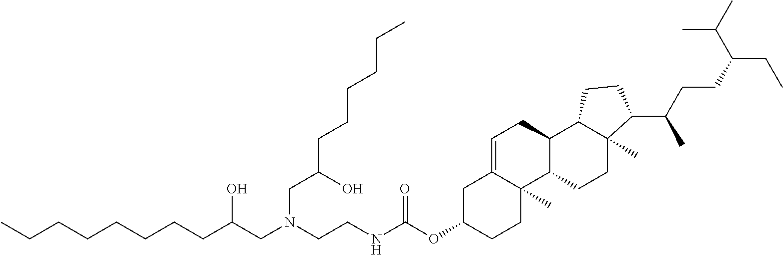 [SMILES: CCCCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCC] |
| 13 | 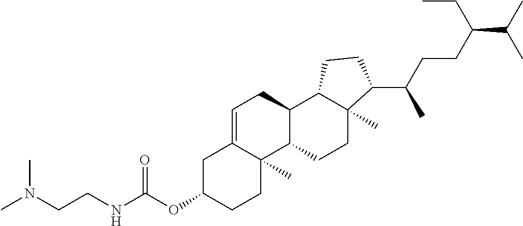 [SMILES: CC[C@H](CC[C@@H](C)[C@H]1CC[C@H]2[C@@H]3CC=C4C[C@@H](OC(=O)NCCN(C)C)CC[C@]4(C)[C@H]3CC[C@]12C)C(C)C] |
| 14 | 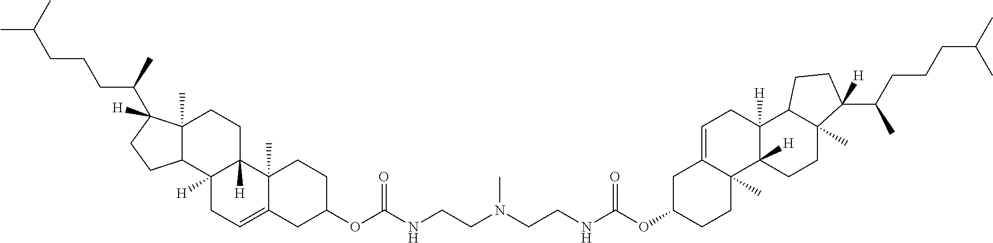 |
| 15 | 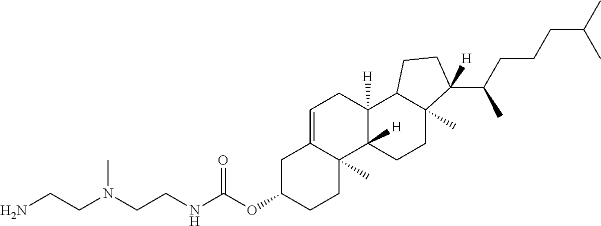 |
| 16 | 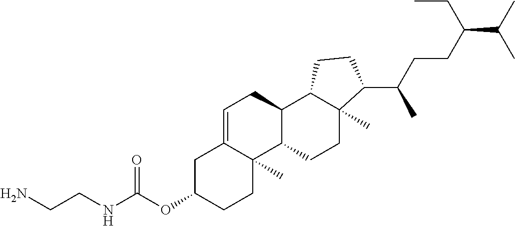 [SMILES: CC[C@H](CC[C@@H](C)[C@H]1CC[C@H]2[C@@H]3CC=C4C[C@@H](OC(=O)NCCN)CC[C@]4(C)[C@H]3CC[C@]12C)C(C)C] |
| 17 | 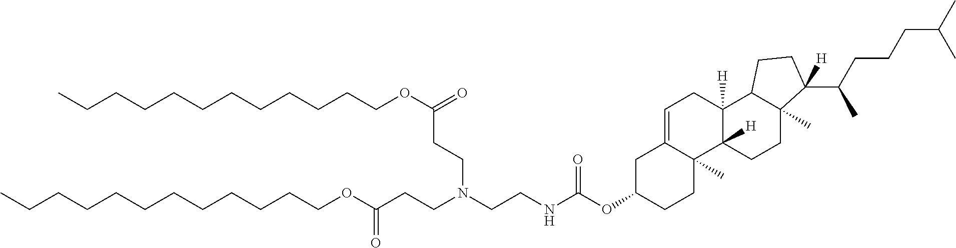 |
| 18 | 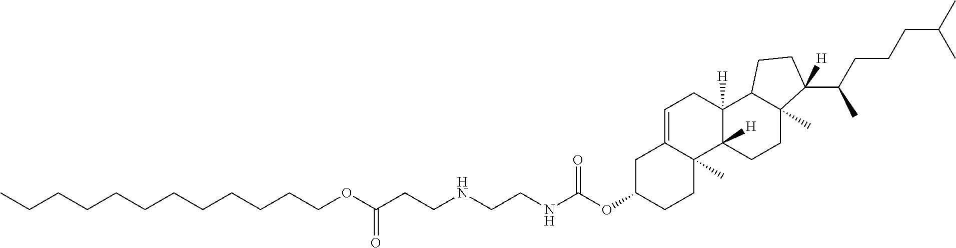 |
| 19 | 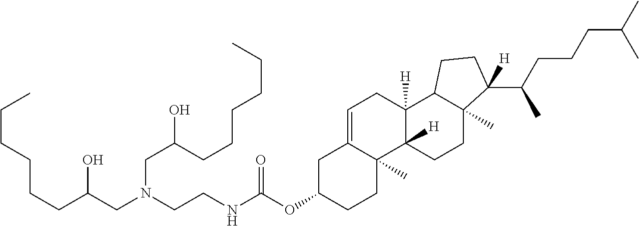 |
| 20 | 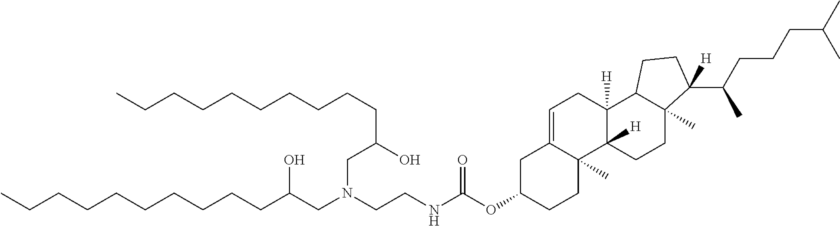 |
| 21 | 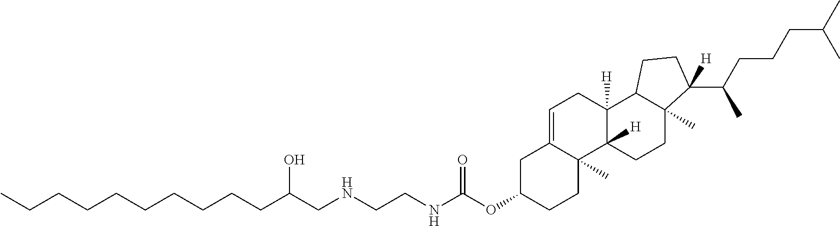 |
| 22 | 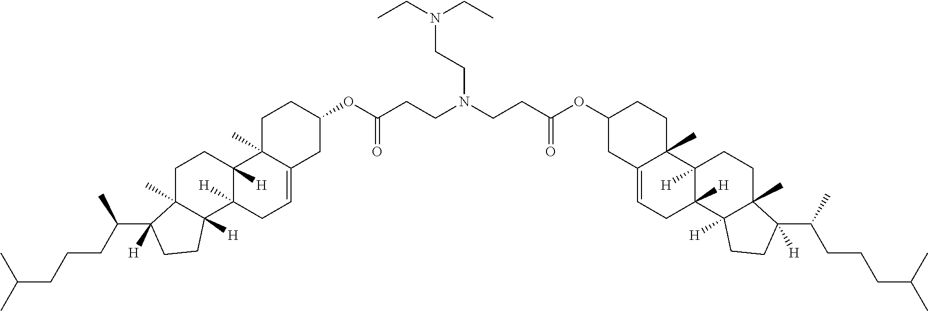 |
| 23 | 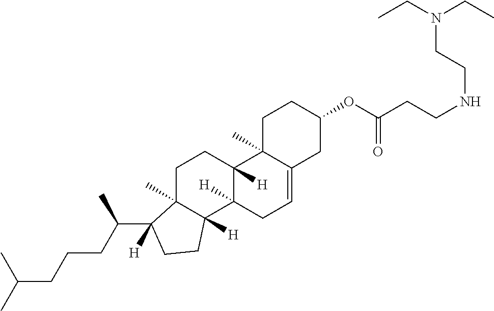 |
| 24 | 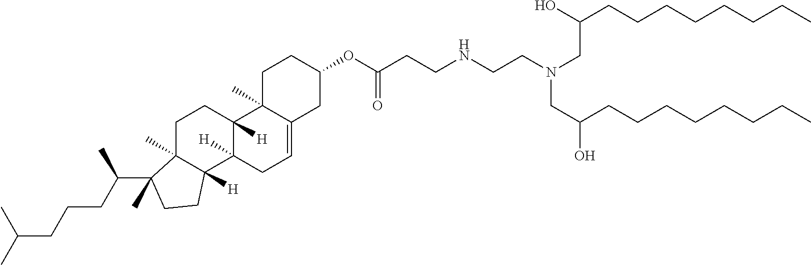 |
| 25 | 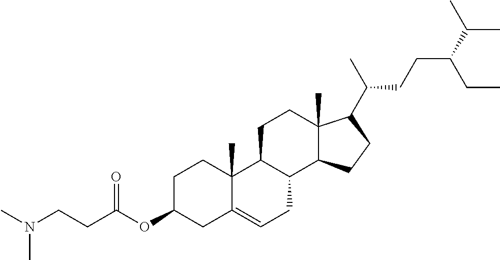 |
| 26 | 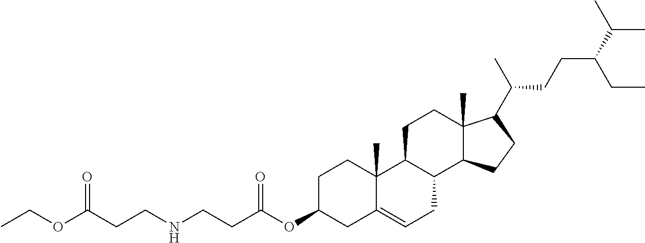 |
| 27 | 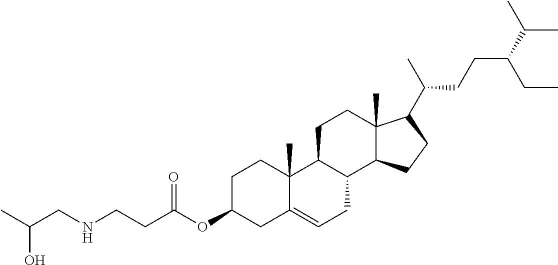 |
| 28 | 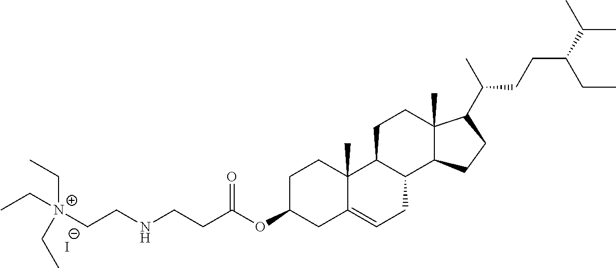 |
| 29 | 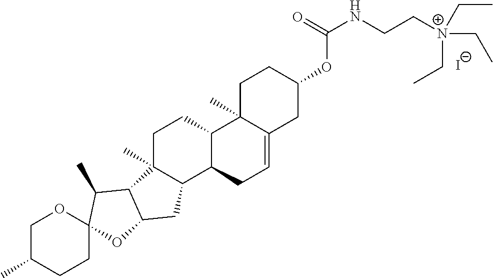 [SMILES: CC[N+](CC)(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1 ; [I-]] |
| 30 | 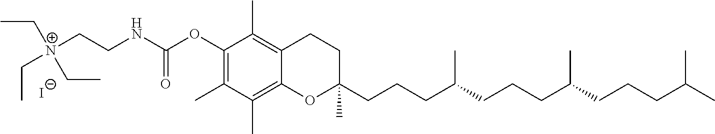 [SMILES: CC[N+](CC)(CC)CCNC(=O)Oc1c(C)c(C)c2c(c1C)CC[C@@](C)(CCC[C@H](C)CCC[C@H](C)CCCC(C)C)O2 ; [I-]] |
| 31 | 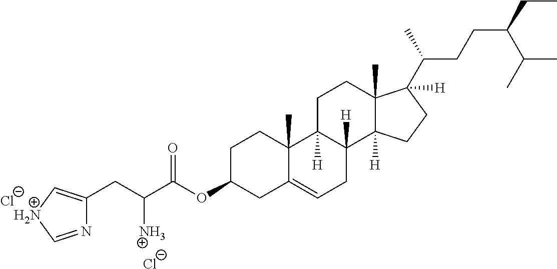 |
| 32 | 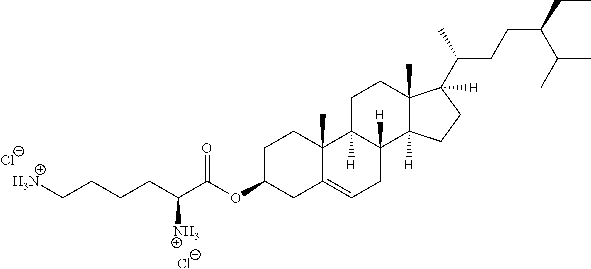 |
| 33 | 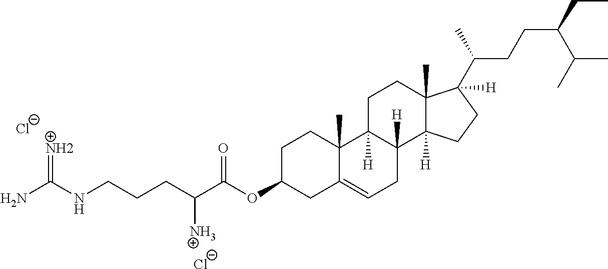 |
| 34 | 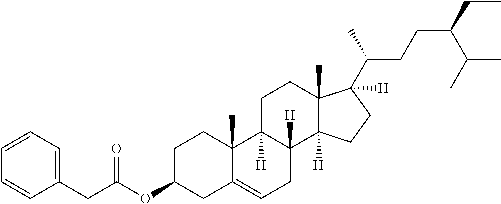 |
| 35 | 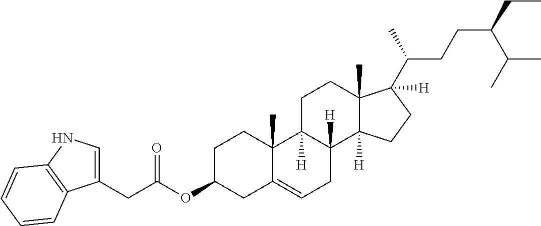 |
| 36 | 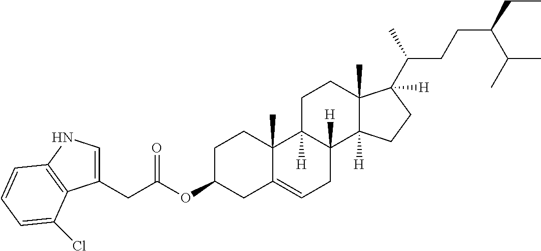 |
| 37 | 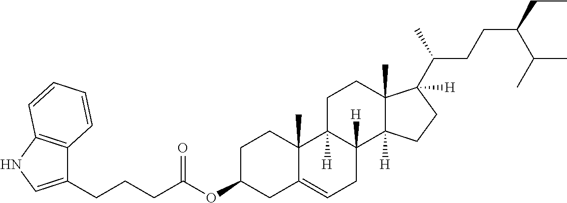 |
| 38 | 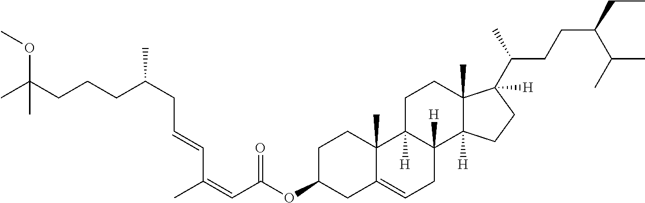 |
| 39 | 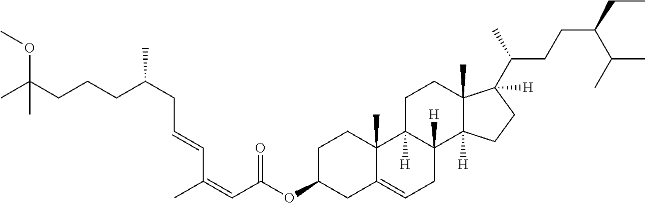 |
| 40 | 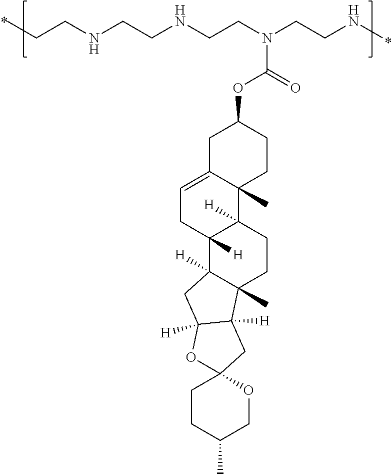 |
| 41 | 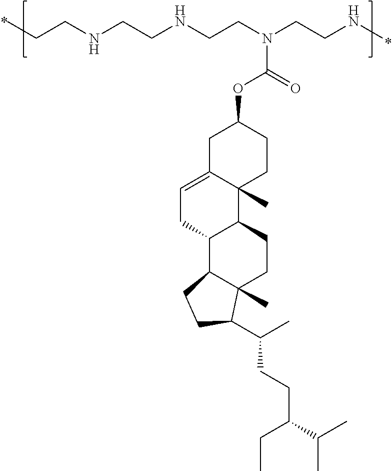 |
| 42 | 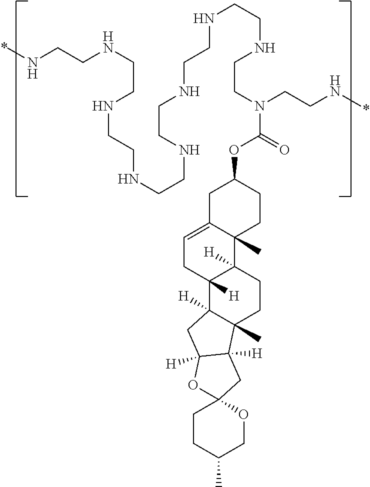 |
| 43 | 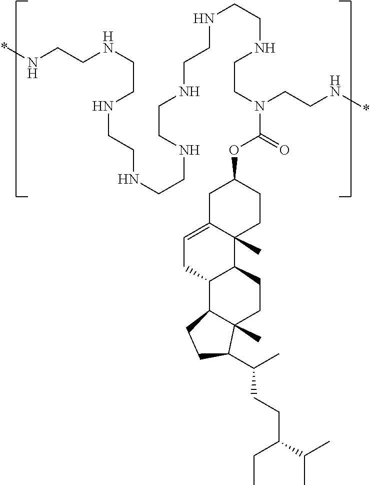 |
| 44 | 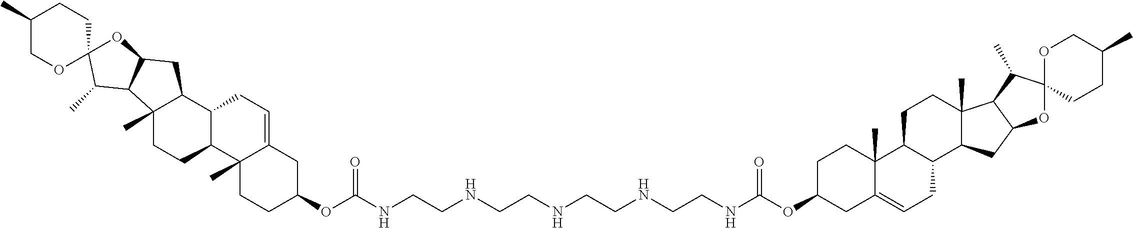 |
| 45 | 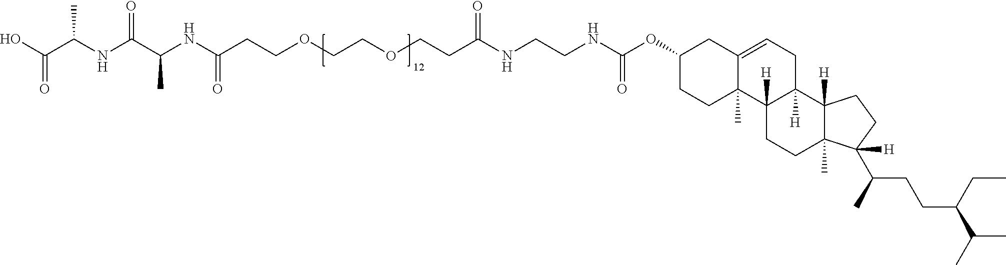 |
| 46 | 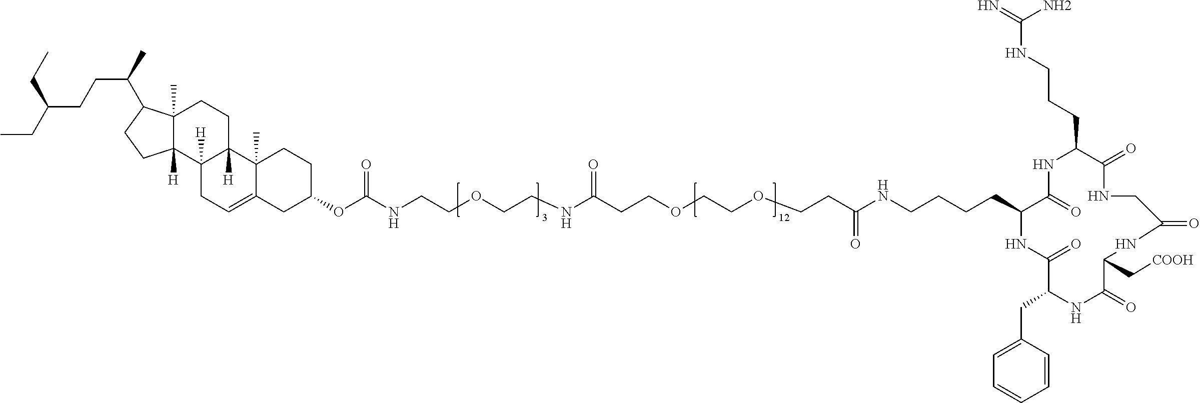 |
| 47 | 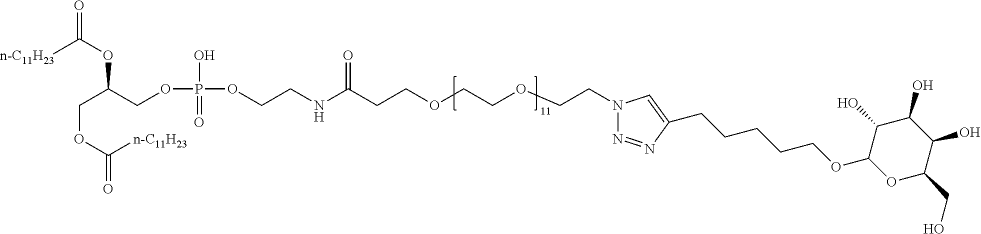 |
| 48 | 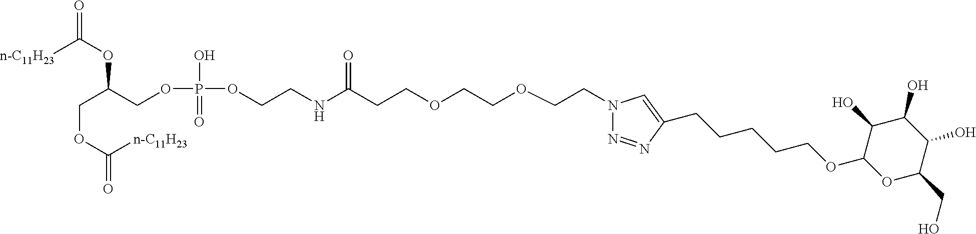 |
| 49 | 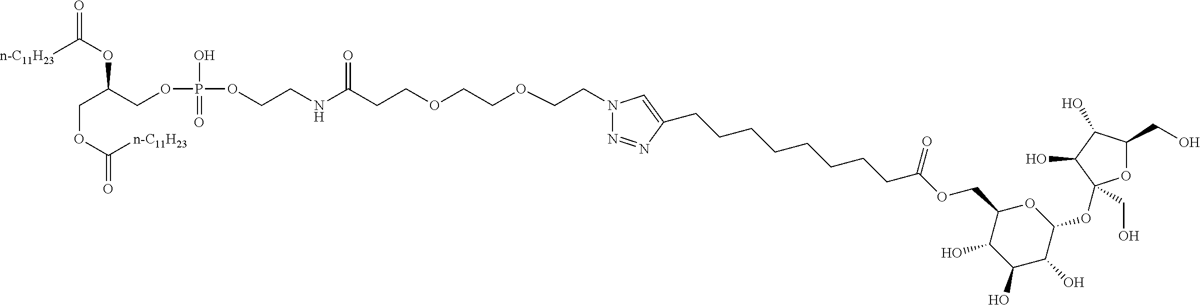 |
| 50 | 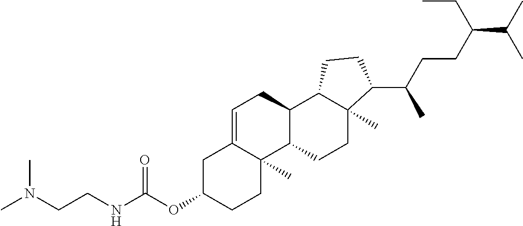 [SMILES: CC[C@H](CC[C@@H](C)[C@H]1CC[C@H]2[C@@H]3CC=C4C[C@@H](OC(=O)NCCN(C)C)CC[C@]4(C)[C@H]3CC[C@]12C)C(C)C] |

Yet another embodiment of the present invention is a compound of formula (XXVII):

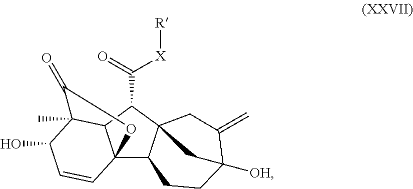

wherein

X is O or NH; and

R′ is an alkyl group or the residue of any steroid, tocopherol, or endogenous auxin, or carbohydrate.

Another embodiment of the present invention is the above compound, wherein R′ is a C₁ to C₂₀ alkyl group.

Another embodiment of the present invention is the above compound, wherein X is O and R′ is a C₁₂ alkyl group or X is O or NH and R′ is a group of formula (XXVIII):

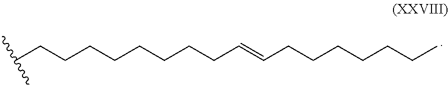 [SMILES: *CCCCCCCC/C=C/CCCCCCC]

Another embodiment of the present invention is the above compound, wherein X is O and R′ is a group selected from the group consisting of formulae (V), (VI), (VII), (VIII), (XIX), (XX), (XXI), (XXII), (XXIII), (XXIV), (XXV), and (XXVI):

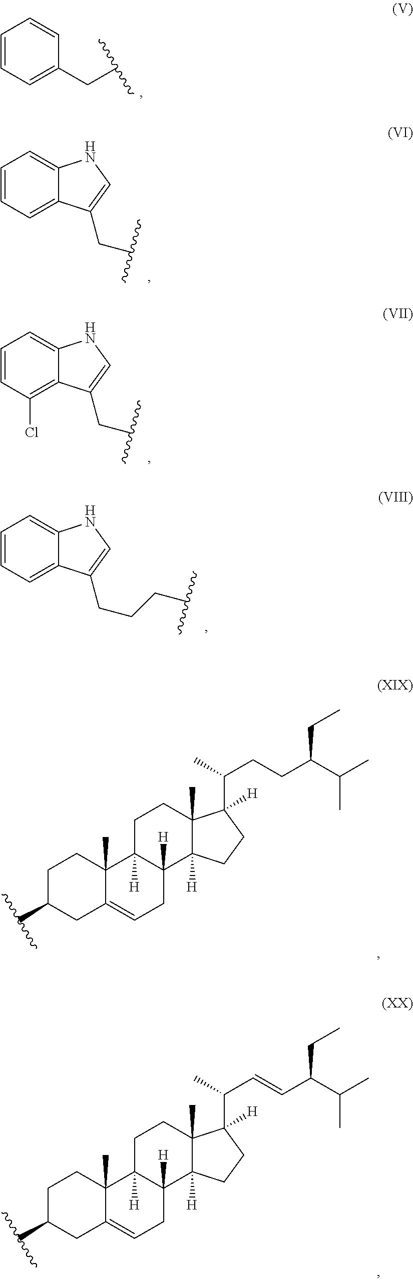

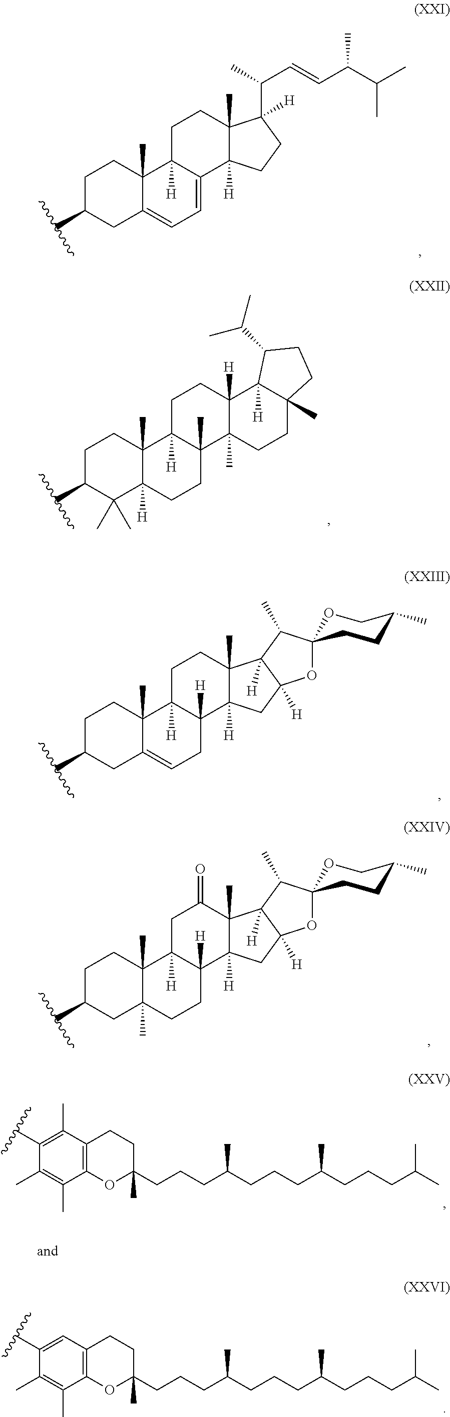

### DETAILED DESCRIPTION OF THE INVENTION

Any section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any ranges described herein will be understood to include the endpoints and all values between the endpoints.

In one aspect, the present disclosure provides for novel formulations comprising (1) at least one formulation transport agent, (2) at least one complexing agent, and (3) at least one active agent that modulates one or more traits of a target organism, wherein the target organism is selected from the group consisting of insects, plants, and plant pathogens.

Formulation Transport Agents

The presently disclosed formulations comprise at least one formulation transport agent. As used herein, the at least one “formulation transport agent” of the presently disclosed formulations is defined as any compound capable of facilitating the transport of the presently disclosed formulation (a) to the surface of a target cell in a target organism, (b) across the cell membrane of such target cells, and (c) through the cytosol of such target cells to the target DNA(s) and/or RNA(s) that govern the one or more traits of the target organism to be modulated, as well as any compound capable of facilitating the decomplexation of the active agent and the complexing agent once inside the target cell. Thus, in certain embodiments, such compounds include, but are not limited to, (1) compounds that can recognize and/or target specific cells, collections of cells, or tissues of the target organism (i.e., “cell targeting agents”), (2) compounds that can assist in the transport of the formulation across a cell membrane (i.e., “membrane penetration agents”); (3) compounds that can assist in the transport of the formulation within the cell (i.e., “intracellular transport agents”), and (4) compounds that can assist in the decomplexation of the formulation and release of the active agent once inside a cell (i.e., “decomplexing agents”), as well as any compounds that possess any combination of these capabilities.

As provided in Table 1, classes of compounds that, as part of the presently disclosed formulations, can act (1) as cell targeting agents include, but are not limited to, insect-, plant-, and plant pathogen-derived steroids and steroid derivatives; phytol derivatives; plant, insect, and plant pathogen hormones and hormone mimics; and carbohydrates; (2) as membrane penetration agents include, but are not limited to, insect-, plant-, and plant pathogen-derived steroids and steroid derivatives; and phytol derivatives; (3) as intracellular transport agents include, but are not limited to, insect-, plant-, and plant pathogen-derived steroids and steroid derivatives; phytol derivatives; and (4) as decomplexing agents include, but are not limited to, lipids and surfactants.

TABLE 1

| | |
|---|---|
| Cell | Insect-derived Steroids and Steroid Derivatives |
| Targeting | Plant-derived Steroids and Steroid Derivatives |
| Agents | Plant Pathogen-derived Steroids and Steroid Derivatives |
| | Phytol Derivatives |
| | Plant Hormones, Hormone Mimics, and Hormone |
| | Precursors |
| | Insect Hormones, Hormone Mimics, and Hormone |
| | Precursors |
| | Plant Pathogen Hormones, Hormone Mimics, and |
| | Hormone Precursors |
| | Sugars and Sugar Derivatives |
| | Naturally Occuring Plant- and Plant Pathogen Derived |
| | Compounds |
| Membrane | Insect-derived Steroids and Steroid Derivatives |
| Penetration | Plant-derived Steroids and Steroid Derivatives |
| Agents | Plant Pathogen-derived Steroids and Steroid Derivatives |
| | Phytol Derivatives |
| | Plant Hormones, Hormone Mimics, and Hormone |
| | Precursors |
| | Surfactants |
| | Naturally Occuring Plant- and Plant Pathogen Derived |
| | Compounds |
| Intracellular | Insect-derived Steroids and Steroid Derivatives |
| Transport | Plant-derived Steroids and Steroid Derivatives |
| Agents | Plant Pathogen-derived Steroids and Steroid Derivatives |
| | Phytol Derivatives |
| Decomplexing | Surfactants |
| Agents | |

Examples of such insect-, plant-, or plant pathogen-derived steroids and steroid derivatives; phytol derivatives; insect-, plant-, or plant pathogen hormones, hormone mimics, hormone precursors; lipids and surfactants which can be suitable for use in the presently disclosed formulations include, but are not limited to, those provided in Table 2.

TABLE 2

| | |
|---|---|
| Plant-derived | 20-Hydroxyecdysone |
| Steroids and | 22-Dihydrobrassicasterol |
| Steroid Derivatives | 28-Isofucosterol |
| | Asiatic Acid |
| | Avenasterol |
| | beta-Sitosterol |
| | Brassicasterol |
| | Brassinosteroids, such as Brassinolide and |
| | Castasterone |
| | Campestanol |
| | Campesterol |
| | Cholesta-5,7-dien-3β-ol |
| | Cycloartanol |
| | Cycloartenol |
| | Daucosterol |
| | Desmosterol |
| | Digitonin |
| | Digoxin |
| | Dioscin |
| | Diosgenin |
| | Epibrassicasterol |
| | Ergostanol |
| | Ergosterol |
| | Fucosterol |
| | Furostan sapogenins, such as Nuatigenin |
| | Furostanol |
| | Glycyrrhizic Acid |
| | Gramisterol |
| | Hecogenin |
| | Lathosterol |
| | Lupeol |
| | Sitostanol |
| | Sitostanyl Ferulate |
| | Sitosterol |
| | Sitosteryl (6′-O-stearoyl) β-D-glucoside |
| | Sitosteryl β-D-glucoside |
| | Sitosteryl stearate |
| | Solanidane agylcones, such as Demissidine |
| | and Solanidine |
| | Spinasterol |
| | Spirosolane aglycones, such as Soladulcidine, |
| | Solasodine, Tomatidenol, and Tomatidine |
| | Spirostan sapogenins, such as Porrigenin A and |
| | Yamogenin |
| | Spirostanol |
| | Stigmastenol |
| | Stigmasterol |
| | Ursolic Acid |
| Insect-derived | Ecdysterone (i.e., 20-Hydroxyecdysone) |
| Steroids and | Ecdysone |
| Steroid Derivatives | |
| Plant Pathogen- | Ergosterol |
| derived Steroids | |
| and Steroid | |
| Derivatives | |
| Phytol Derivatives | Phylloquinone (vitamin K1) |
| | Menaquinone (vitamin K2) |
| | Tocopherols, such as gamma-Tocopherol and |
| | alpha-Tocopherol |
| Plant Hormones, | 1-Aminocyclopropanecarboxylic Acid |
| Hormone Mimics, | Abscisic Acid |
| and Hormone | Ancymidol |
| Precursors | Benzyladenine |
| | Berberine |
| | Brassinolide |
| | Caulines, such as Caulocaline, Phyllocaline, |
| | and Rhizolcaline |
| | Chlormequat chloride |
| | Cytokinins, such as Zeatin |
| | Daminozide |
| | Dikegulac Sodium |
| | Ethephon |
| | Ethylene |
| | Florigen |
| | Fluridone |
| | Flurprimidol |
| | Gibberellins, such as Gibberelin A1 (GA1), |
| | Gibberelic Acid (GA3), ent-Gibberellane, and |
| | ent-Kaurene |
| | Indole-3-butyric acid |
| | Jasmonates, such as Jasmonic Acid and Methyl |
| | Jasmonate |
| | Karrikins, such as KAR₁, KAR₂, KAR₃, and |
| | KAR₄ |
| | Native Auxins, such as Indole 3-acetic Acid, |
| | Indole-3-acetaldehyde, Indole-3-pyruvic Acid, |
| | Indole-3-acetonitrile, Indole-3-ethanol, |
| | Phenylacetic Acid, 2-Phenylacetic Acid, 4- |
| | Chloroindole-3-acetic Acid, and Indole-3- |
| | butyric Acid |
| | Norflurazon |
| | Paclobutrazol |
| | Peptide Hormones, such as CLV3, DEVIL1 |
| | (DVL1), ENOD40, Inflorescence Deficient in |
| | Abscission (IDA), Phytosulfonkine (PSK), |
| | POLARIS (PLS), Rapid Alklinization Factor |
| | (RALF), ROTUNDIFOLIA4 (ROT4), SCR, |
| | SP11, and Systemin |
| | Prohexadione |
| | Salicylic acid |
| | Strigolactones |
| | Synthetic Auxins, such as 2,4- |
| | Dichlorophenoxyacetic aicd, α-Naphthalene |
| | acetic Acid, 2-Methoxy-3,6-dichlorobenzoic |
| | Acid, 4-Amino-3,5,6-trichloropicolinic Acid, |
| | and 2,4,5-Trichlorophenoxyacetic Acid |
| | Tetcyclasis |
| | Uniconazole |
| | Vernalin |
| Insect Hormones, | Adipokinetic Hormone |
| Hormone Mimics, | Allotostatin |
| and Hormone | Bursicon |
| Precursors | Ecdysone |
| | Ecdysterone (i.e., 20-Hydroxyecdysone) |
| | Juvenile Hormone (JHs), such as Fenoxycarb, |
| | JHI, JHII, JHIII, JHB₃, JHSB₃, Methoprene, |
| | and Pyriproxifen |
| | Prothoracicotropic Hormone |
| Plant Pathogen | Gibberellins |
| Hormones, | |
| Hormone Mimics, | |
| and Hormone | |
| Precursors | |
| Sugars and | C₄ to C₈ monosaccharides, such as 2- |
| Sugar Derivatives | deoxyglucose, 2-deoxyribose, 3-O- |
| | methylglucose, 6-deoxyglucose, allose, altrose, |
| | arabinose, daunosamine, deoxyribose, fructose, |
| | fucopyranose, fucose, galactose, |
| | glacactopyranosuronic acid, |
| | glucopyranosuronic acid, glucose, glucuronic |
| | acid, gulose, iduronic acid, iodose, lyxose, |
| | mannopyranosuronic acid, mannose, |
| | neuraminic acid, rhamnopyranose, ribose, |
| | sialic acid, sulfoquinovose, tagatose, talose, |
| | and xylose. |
| | Disaccharides, such as cellobiose, chitobiose, |
| | gentiobiose, gentiobiulose, isomaltose, |
| | kojibiose, lactose, lactulose, laminaribiose, |
| | maltose, maltulose, mannobiose, melibiose, |
| | melibiulose, nigerose, palatinose (i.e., |
| | isomaltulose), rutinose, rutinulose, sophorose, |
| | sucrose, trehalose, trehalulose, turanose, and |
| | xylobiose. |
| | Trisaccharides, such as isomaltotriose, |
| | nigerotriose, maltotriose, melezitose, |
| | maltotriulose, raffinose, and kestose. |
| | Tetrasaccarides, such as lychnose, |
| | maltotetraose, nigerotetraose, nystose, |
| | sesamose, and stachyose. |
| | Aminoglycosides, such as N- |
| | acetylglucosamine, N-acetylgalactosamine, N- |
| | acetylmannosamine, N-acetylneuraminic acid, |
| | N-glycolylneuraminic acid, galactosamine, and |
| | glucosamine, |
| | Sugar Lipids, such as Alkyl Glucosides (e.g., |
| | Octyl Glucoside, Decyl Glucoside), |
| | Mannosides, Maltosides (e.g., Octyl |
| | Maltoside), and Galactosides |
| | Glycolipids, such as |
| | Digalactosyldiacylglycerol (DGDG), |
| | Glucuronosyldiacylgylcerol (GlcADG), |
| | Monogalactosyldiacylglycerol (MGDG), and |
| | Sulfoquinovosyldiacylglycerol (SQDG) |
| | Saccharolipids |
| Naturally | Alamethicin |
| Occuring Plant- | Limonin |
| and Plant | Vitamin K |
| Pathogen Derived | Thiamine |
| Compounds | Riboflavin |
| | Pyridoxine |
| | Niacinamide |
| | Pantothenic acid |
| | Biotin |
| | Folic acid |
| | Choline |
| | Carnitine |
| | Ascorbic acid |
| | Carotene |
| | Vitamin A |
| | Vitamin E |
| Lipids/Surfactants | Alkyl Alcohols, such as Cetyl Alcohol |
| | Alkyl Polyoxyethylene Ethers, such as LECI- |
| | TECH, LI700 (lecithin-based surfactant |
| | derived from soybeans), the Brij ® family (e.g., |
| | Brij 35), Hexaethylene Glycol Monododecyl |
| | Ether (C12PEG6), Octaethylene Glycol |
| | Monotetradecyl Ether (C14PEG8), and |
| | Octaethylene Glycol Monohexadecyl Ether |
| | (C16PEG8) |
| | Amphoteric Surfactants (e.g., Betaine |
| | Surfactants) such as Cocamidopropyl Betaine |
| | and Lauryl Betaine |
| | Arachidonic Acid |
| | Benzalkonium Chloride |
| | Betaine Lipids, such as 1,2-Dipalmitoyl-sn- |
| | glycero-3-O-4′-[N,N,N-trimethyl(d9)]- |
| | homoserine (DGTS-d9) and 1,2-Dipalmitoyl- |
| | sn-glycero-3-O-4′-(N,N,N-trimethyl)- |
| | homoserine (DGTS) |
| | Cetyltrimethyl Ammonium Bromide (CTAB) |
| | Dioctyl Sulfosuccinate Sodium Salt (AOT) |
| | Docosahexaenoic Acid |
| | Eicosapentaenoic Acid |
| | Eugenol |
| | Fatty acids, such as Crepenynic Acid, Erucic |
| | Acid, Lauric Acid, Linoleic Acid, Linolenic |
| | Acid, Oleic Acid, Parinaric Acid, Palmitic |
| | Acid, Ricinoleic Acid, Stearic Acid, Sterculic |
| | Acid, and Vernolic Acid |
| | Fluorinated and Perfluorinated Surfactants |
| | Hexadecylpyridinium Chloride (C16Pyr) |
| | Linalool |
| | Myristic acid |
| | Oxylipins, such as 12-oxo-cis-Dodecenoic |
| | acid, 12,13-Epoxyl-18:3, cis-3-Hexenal, 13- |
| | Hydroperoxy-18:3, 9,12-Ketol, 12,13-Ketol, |
| | 12-oxo-Phytodienoic acid, and Traumatin |
| | Palmitic acid |
| | Phosphatidic acid |
| | Phospholipids, such as Cardiolipin (CL), |
| | Phosphatidylcholine (PC), |
| | Phosphatidylethanolamine (PE), |
| | Phosphatidylglycerol (PG), |
| | Phosphatidylinositol (PI), and |
| | Phosphatidylserine (PS) |
| | Phytantriol, Phytantriol derivatives (e.g., alkyl |
| | and acyl ethers and esters), and Phytantriol |
| | mixtures, such as (1) Phytantriol and Glyceryl |
| | Monooeleate (GMO), (2) Phytantriol and |
| | GMO, F127 for particle stabilization, and a |
| | hydrotrope (ethanol or polyethylene glycol |
| | (PEG₂₀₀) or propylene glycol (PG)), (3) |
| | Poly(ethylene glycol)-grafted 1,2-Distearoyl- |
| | sn-glycero-3-phosphoethanolamines (DSPE- |
| | mPEGs), and (4) Phytantriol and Alkylated |
| | Spiropyran (e.g., Spiropyran Laurate) |
| | Phytosphingolipids, such as |
| | Glycosylinositolphosphoceramides (GIPC) |
| | Polyethoxylated Sorbitan Esters (e.g., the |
| | Tween ® family) such as Tween 20 |
| | Polymeric Surfactants, such as Organosilicone |
| | surfactants (e.g., Silwet L-77, Breakthru S240), |
| | Pluronic/Poloxamer families (e.g., F127), and |
| | Tallowamines |
| | Prenol lipids |
| | Sodium Dodecyl Sulfonate (SDS) |
| | Sorbitan Esters (e.g., the Span ® family), such |
| | as Span 80 |
| | Sphingolipids, such as Phytosphingosine, |
| | Sphingomyelin, and Sphingosine |
| | Sterol Lipids |
| | Tri-, di-, and mono-glyceride, cholesterol, |
| | cholesteryl ester, phospholipids |
| | Triacylglycerol |
| | Wax Esters, such as Jojoba Oil |

In certain embodiments, the at least one formulation transport agent can be a compound of formula (I):

A-B-C (I)

wherein A is a group that can facilitate transport of the formulation to, into, and within a cell of the target organism and/or decomplexation of the formulation, B is a linker, and C is a group that can non-covalently associate to the at least one complexing agent. As such, in another aspect, the present disclosure provides for compounds of formula (I). In the presently disclosed compounds of formula (I), A is a group that can facilitate transport of the formulations of the present disclosure to, into, and within a cell of the target organism and/or decomplexation of the formulation within the target organism, B is a linker, and C is a group that can non-covalently associate to at least one complexing agent of the presently disclosed formulation.

In certain embodiments, group A of the presently disclosed compounds of formula (I) can be a cationic group (or a group that can become cationic), a group derived from an insect-, plant-, or plant pathogen-derived hormone, or a group derived from a carbohydrate. Examples of such cationic groups (including groups that can become cationic) include, but are not limited to, arginine, lysine, histidine, and groups of formulae (II), (III), and (IV):

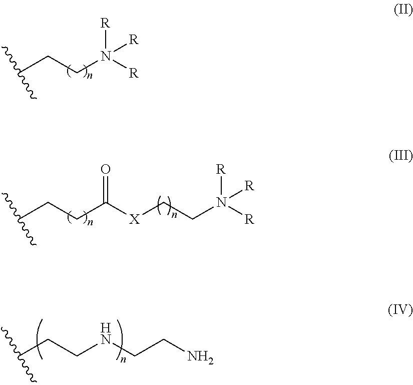

where X is O or NH, R is —H, —CH₃, —CH₂CH₃, or —CH₂CH₂OH, and n is 0, 1, or 2. Examples of such groups derived from insect-, plant-, or plant pathogen-derived hormones include groups of formulae (V) (i.e., the auxin 2-phenylacetic acid), (VI) (i.e., the auxin indole-3-acetic acid), (VII) (i.e., the auxin 4-chloroindole-3-acetic acid), (VIII) (i.e., the auxin indole-3-butyric acid), (IX) (i.e., gibberelic acid and esters thereof), and (X) (i.e., jasmonic acid/methyl jasmonate):

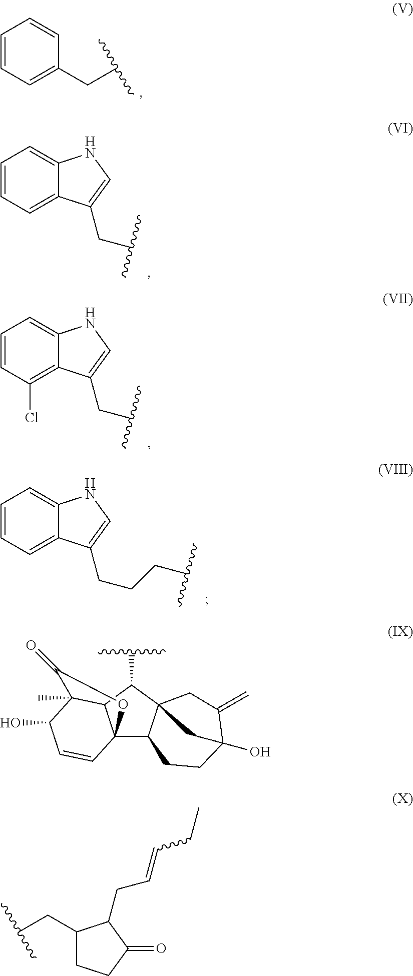

Examples of such groups derived from carbohydrates include, but are not limited to, groups derived from glucose, sucrose, maltose, and kanamycin.

In the presently disclosed compounds of formula (I), the linker B is at least in part formed from a moiety of A and a moiety of C. As such, the linker B can be a covalent bond or any divalent group. Examples of such divalent groups include, but are not limited to, groups of formulae (XI), (XII), (XIII), (XIV), (XV), (XVI), (XVII), and (XVIII), as provided in Table 3:

TABLE 3

| | |
|---|---|
| 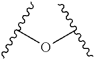 [SMILES: *O*] | (XI) |
| 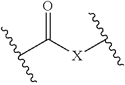 | (XII) |
| 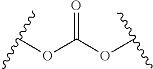 [SMILES: *OC(=O)O*] | (XIII) |
| 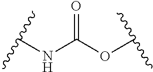 [SMILES: *NC(=O)O*] | (XIV) |
| 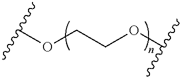 | (XV) |
| 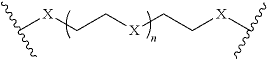 | (XVI) |
| 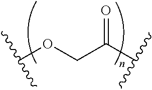 | (XVII) |
| 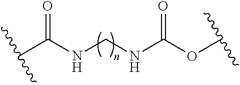 | (XVIII) |

wherein “X” in formulae (XII) and (XVI) is, independently, O or NH and “n” in formulae (XV), (XVI), (XVII), and (XVIII) is an integer in the range of from 1 to 10.

In certain embodiments, group C of the presently disclosed compounds of formula (I) can be derived from a steroid or a tocopherol. Examples of such groups derived from steroids include, but are not limited to, groups of formulae (XIX) (i.e., β-sitosterol), (XX) (i.e., stigmasterol), (XXI) (i.e., ergosterol), (XXII) (i.e., lupeol), (XXIII) (i.e., diosgenin), and (XXIV) (i.e., hecogenin):

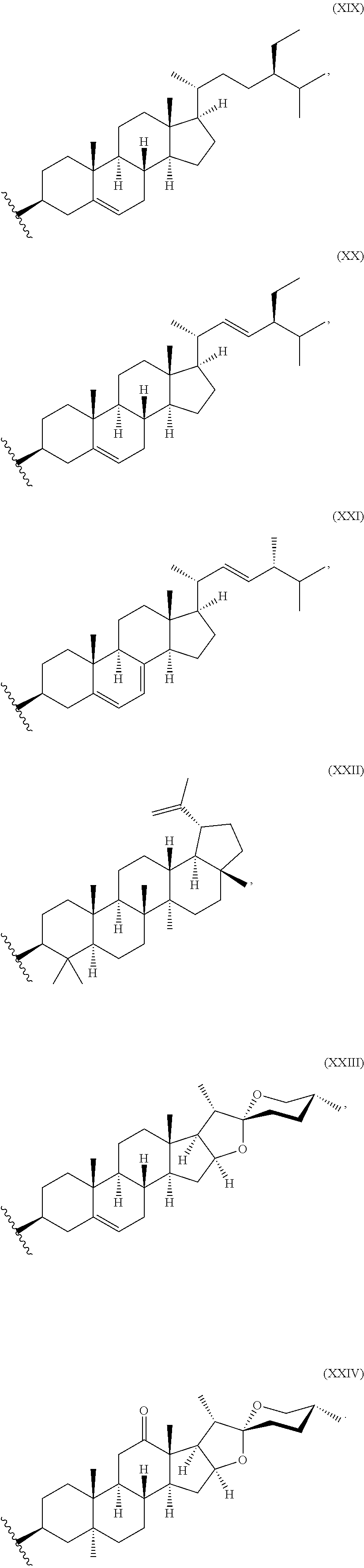

Examples of such groups derived from tocopherols include, but are not limited to, groups of formulae (XXV) (i.e., α-tocopherol), and (XXVI) (i.e., γ-tocopherol):

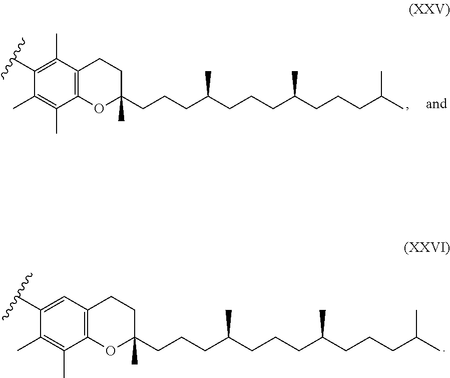

In certain embodiments, the presently disclosed compounds of formula (I) are gibberellic acid derivatives of formula (XXVII):

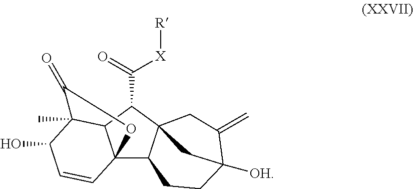

In the presently disclosed gibberellic acid derivatives of formula (XXVII), the group of formula (IX):

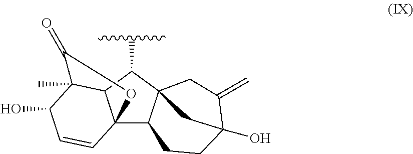 [SMILES: *[C@H]1C2[C@@]3(C=C[C@H](O)[C@@]2(C)C(=O)O3)[C@@H]2CCC3(O)C[C@@]21CC3=C]

corresponds to group A of the presently disclosed compounds of formula (I). The linker B is a an ester or amide group of formula (XII):

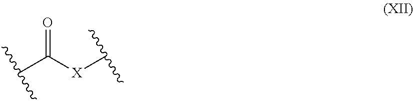

where X is O or NH. The group R′ of the presently disclosed gibberellic acid derivatives of formula (XXVII) corresponds to group C of the presently disclosed compounds of formula (I) and can be an alkyl or alkylene group or a group derived from any insect-, plant-, or plant pathogen-derived steroid or any tocopherol, endogenous auxin, or carbohydrate. In certain embodiments, R′ is a C₁ to C₂₀ alkyl group. In certain embodiments, X is O and R′ is a C₁₂ alkyl group or X is O or NH and R′ is a group of formula (XXVIII):

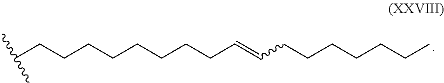 [SMILES: *CCCCCCCCC=CCCCCCCC]

In certain embodiments, X is O and R′ is a group derived from (1) an auxin, such as 2-phenylacetic acid (formula V), indole-3-acetic acid (formula VI), 4-chloroindole-3-acetic acid (formula VII), and indole-3-butyric acid (formula VIII), (2) a steroid, such as β-sitosterol (formula XIX), stigmasterol (formula XX), ergosterol (formula XXI), lupeol (formula XXII), diosgenin (formula XXIII), and hecogenin (formula XXIV), (3) a tocopherol, such as α-tocopherol (formula XXV) and γ-tocopherol (formula XXVI), or (4) a carbohydrate, such as glucose, sucrose, maltose, or kanamycin.

In certain embodiments, the presently disclosed compound of formula (I) is selected from the group consisting of compounds (1) through (50), as provided in Table 4:

TABLE 4

| Com- | |
|---|---|
| pound | |
| Number | Structure |
| 1 | 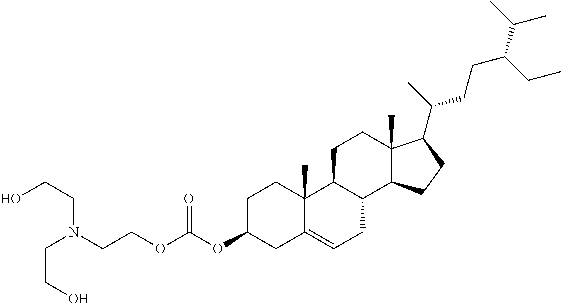 [SMILES: CC[C@H](CC[C@@H](C)[C@H]1CC[C@H]2[C@@H]3CC=C4C[C@@H](OC(=O)OCCN(CCO)CCO)CC[C@]4(C)[C@H]3CC[C@]12C)C(C)C] |
| 2 | 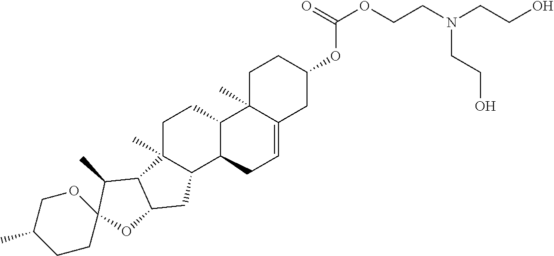 [SMILES: C[C@H]1CC[C@]2(OC1)O[C@H]1C[C@H]3[C@@H]4CC=C5C[C@@H](OC(=O)OCCN(CCO)CCO)CC[C@]5(C)[C@H]4CC[C@]3(C)[C@H]1[C@@H]2C] |
| 3 | 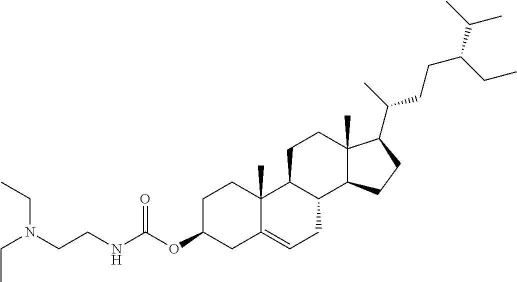 [SMILES: CC[C@H](CC[C@@H](C)[C@H]1CC[C@H]2[C@@H]3CC=C4C[C@@H](OC(=O)NCCN(CC)CC)CC[C@]4(C)[C@H]3CC[C@]12C)C(C)C] |
| 4 | 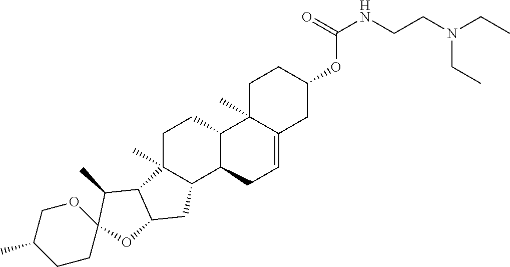 [SMILES: CCN(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1] |
| 5 | 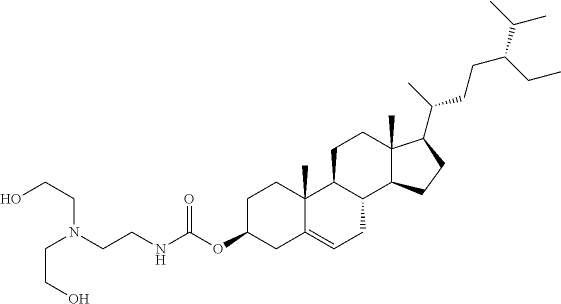 [SMILES: CC[C@H](CC[C@@H](C)[C@H]1CC[C@H]2[C@@H]3CC=C4C[C@@H](OC(=O)NCCN(CCO)CCO)CC[C@]4(C)[C@H]3CC[C@]12C)C(C)C] |
| 6 | 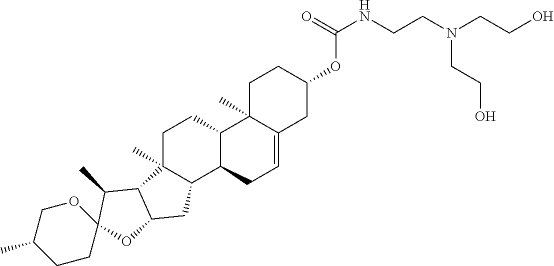 [SMILES: C[C@H]1CC[C@]2(OC1)O[C@H]1C[C@H]3[C@@H]4CC=C5C[C@@H](OC(=O)NCCN(CCO)CCO)CC[C@]5(C)[C@H]4CC[C@]3(C)[C@H]1[C@@H]2C] |
| 7 | 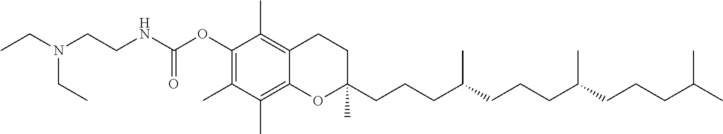 [SMILES: CCN(CC)CCNC(=O)Oc1c(C)c(C)c2c(c1C)CC[C@@](C)(CCC[C@H](C)CCC[C@H](C)CCCC(C)C)O2] |
| 8 | 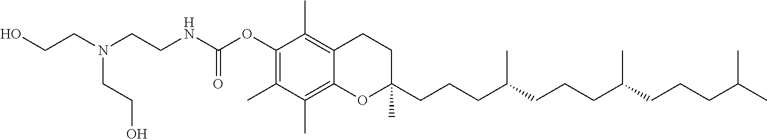 [SMILES: Cc1c(C)c2c(c(C)c1OC(=O)NCCN(CCO)CCO)CC[C@@](C)(CCC[C@H](C)CCC[C@H](C)CCCC(C)C)O2] |
| 9 | 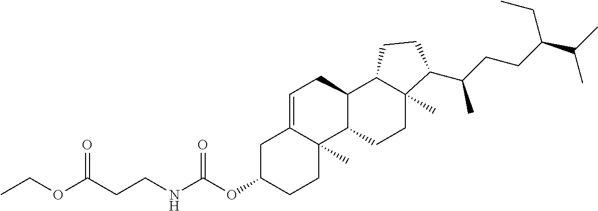 [SMILES: CCOC(=O)CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 10 | 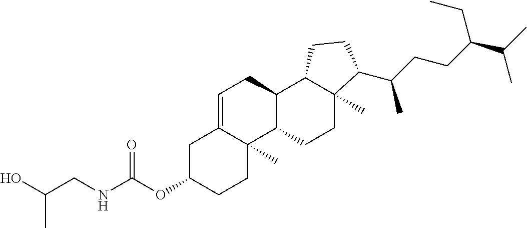 [SMILES: CC[C@H](CC[C@@H](C)[C@H]1CC[C@H]2[C@@H]3CC=C4C[C@@H](OC(=O)NCC(C)O)CC[C@]4(C)[C@H]3CC[C@]12C)C(C)C] |
| 11 | 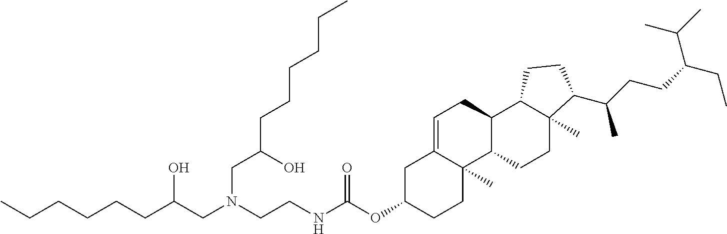 [SMILES: CCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCC] |
| 12 | 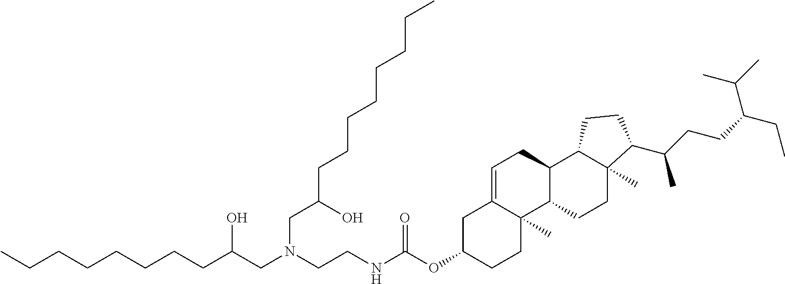 [SMILES: CCCCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCCCC] |
| 13 | 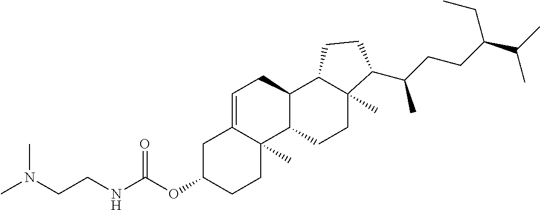 [SMILES: CC[C@H](CC[C@@H](C)[C@H]1CC[C@H]2[C@@H]3CC=C4C[C@@H](OC(=O)NCCN(C)C)CC[C@]4(C)[C@H]3CC[C@]12C)C(C)C] |
| 14 | 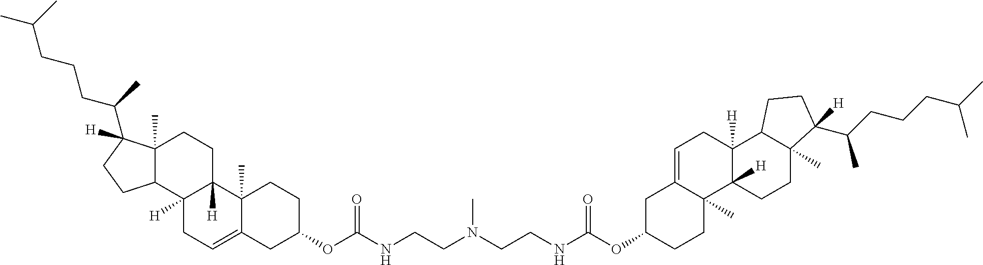 |
| 15 | 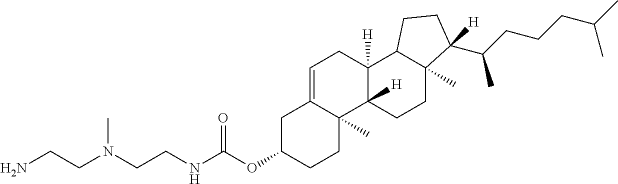 |
| 16 | 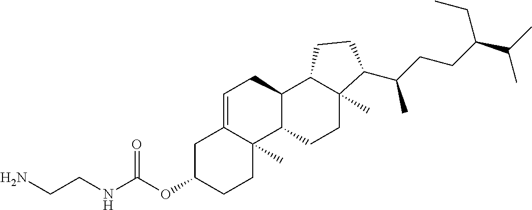 [SMILES: CC[C@H](CC[C@@H](C)[C@H]1CC[C@H]2[C@@H]3CC=C4C[C@@H](OC(=O)NCCN)CC[C@]4(C)[C@H]3CC[C@]12C)C(C)C] |
| 17 | 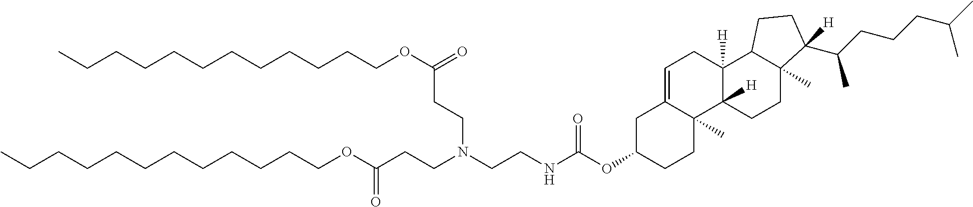 |
| 18 | 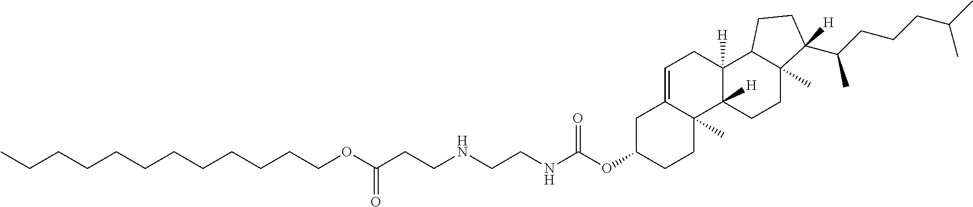 |
| 19 | 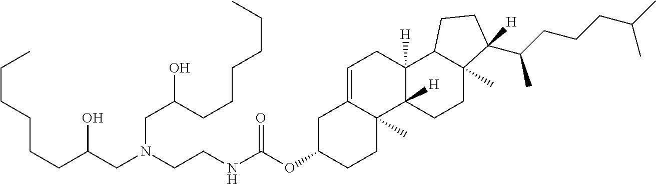 |
| 20 | 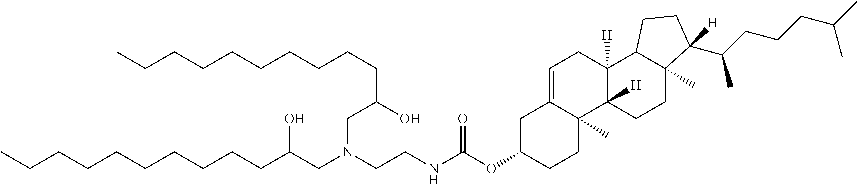 |
| 21 | 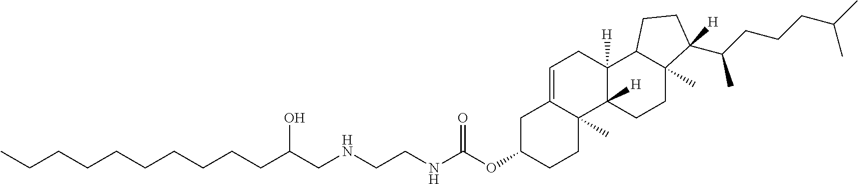 |
| 22 | 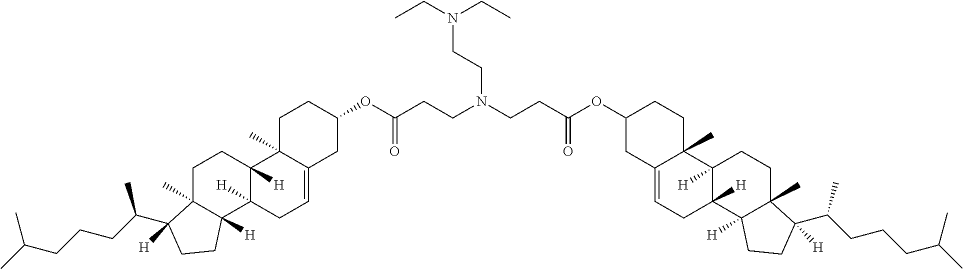 |
| 23 | 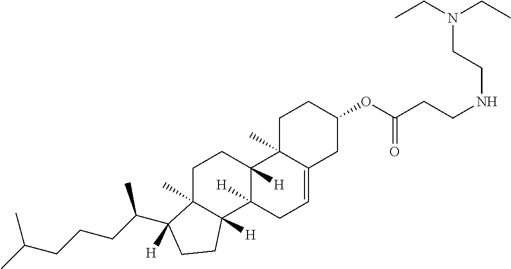 |
| 24 | 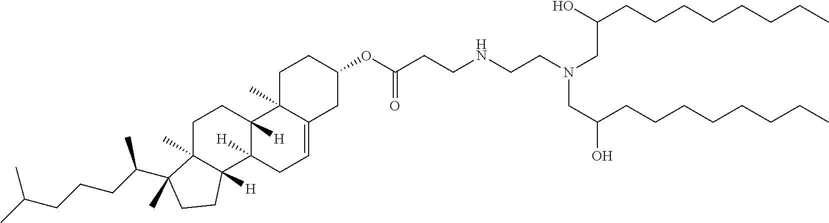 |
| 25 | 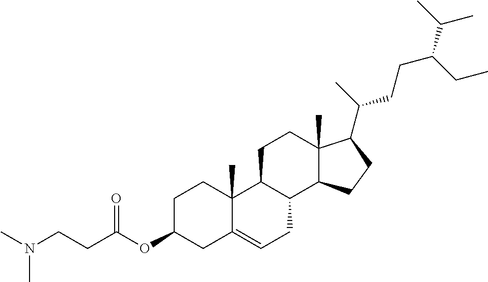 [SMILES: CC[C@H](CC[C@@H](C)[C@H]1CC[C@H]2[C@@H]3CC=C4C[C@@H](OC(=O)CCN(C)C)CC[C@]4(C)[C@H]3CC[C@]12C)C(C)C] |
| 26 | 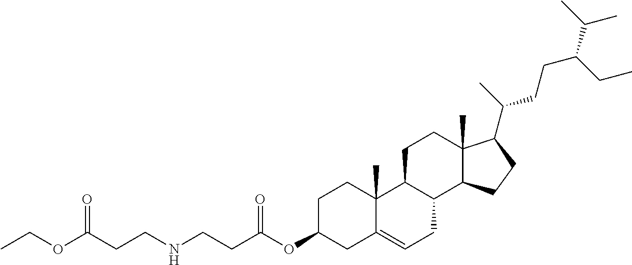 [SMILES: CCOC(=O)CCNCCC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 27 | 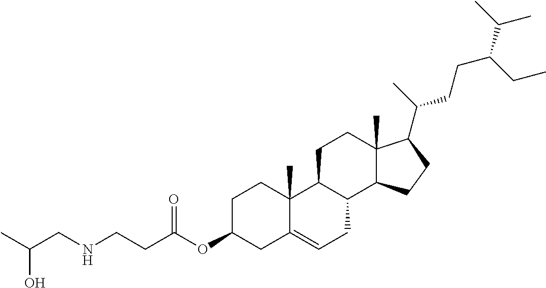 [SMILES: CC[C@H](CC[C@@H](C)[C@H]1CC[C@H]2[C@@H]3CC=C4C[C@@H](OC(=O)CCNCC(C)O)CC[C@]4(C)[C@H]3CC[C@]12C)C(C)C] |
| 28 | 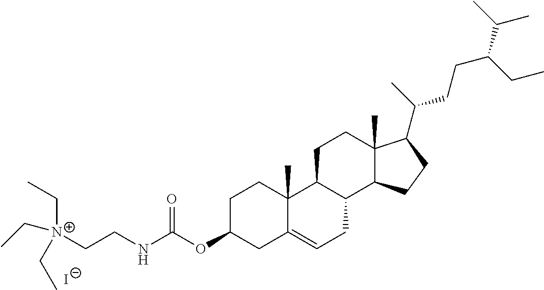 [SMILES: CC[C@H](CC[C@@H](C)[C@H]1CC[C@H]2[C@@H]3CC=C4C[C@@H](OC(=O)NCC[N+](CC)(CC)CC)CC[C@]4(C)[C@H]3CC[C@]12C)C(C)C ; [I-]] |
| 29 | 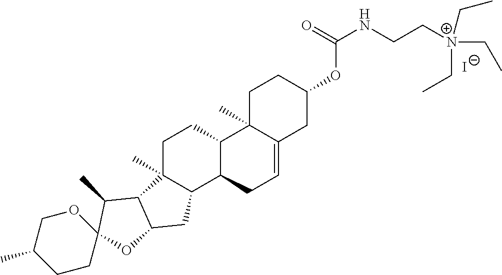 [SMILES: CC[N+](CC)(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1 ; [I-]] |
| 30 | 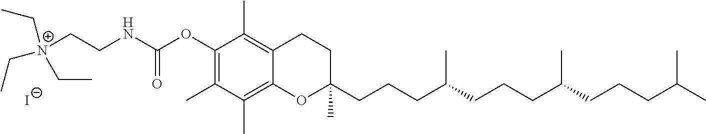 [SMILES: CC[N+](CC)(CC)CCNC(=O)Oc1c(C)c(C)c2c(c1C)CC[C@@](C)(CCC[C@H](C)CCC[C@H](C)CCCC(C)C)O2 ; [I-]] |
| 31 | 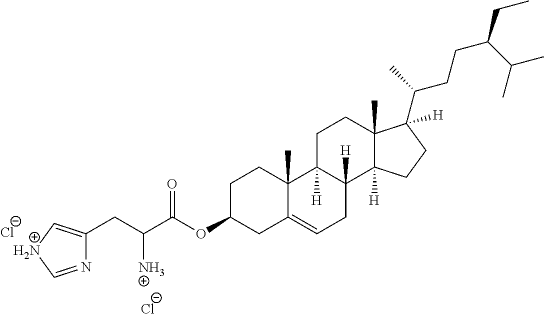 |
| 32 | 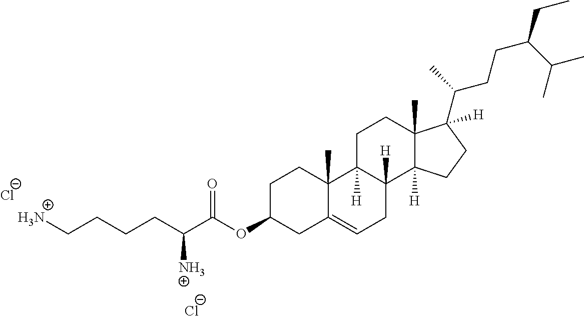 |
| 33 | 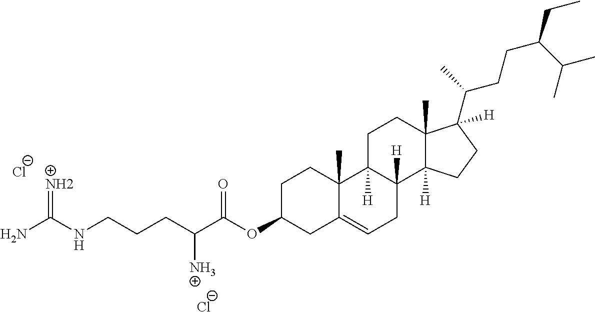 |
| 34 | 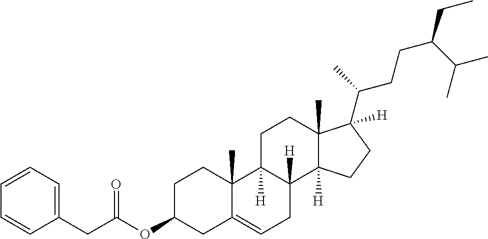 |
| 35 | 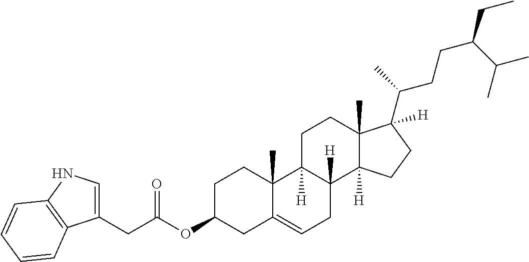 |
| 36 | 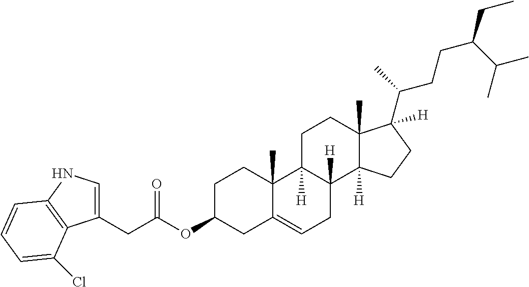 |
| 37 | 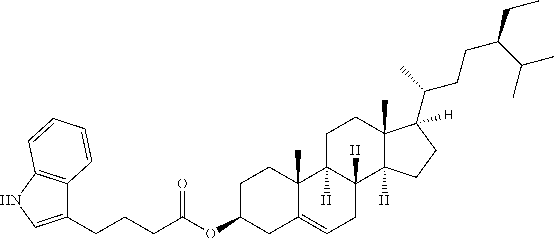 |
| 38 | 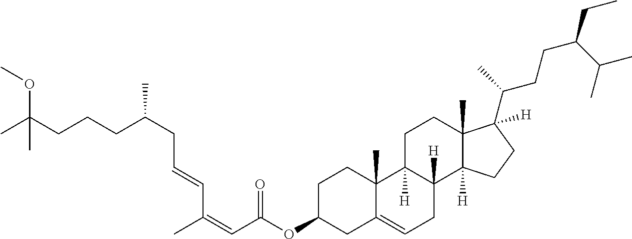 |
| 39 | 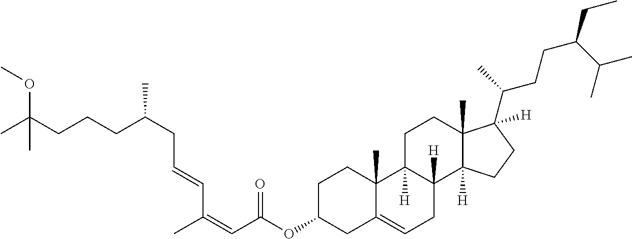 |
| 40 | 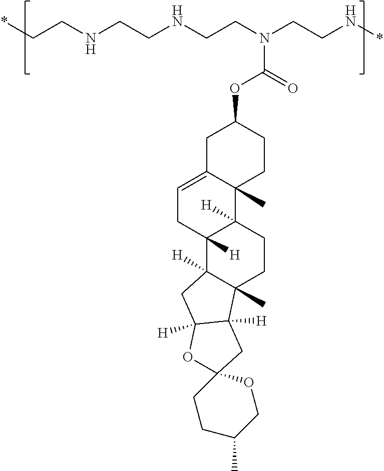 |
| 41 | 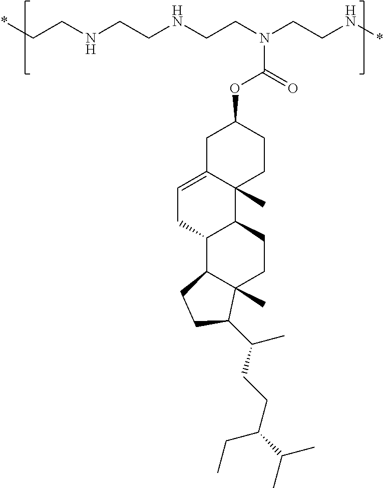 |
| 42 | 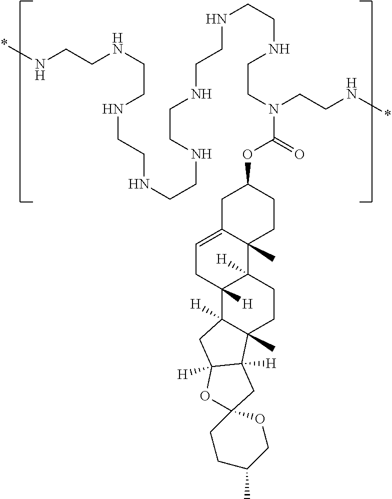 |
| 43 | 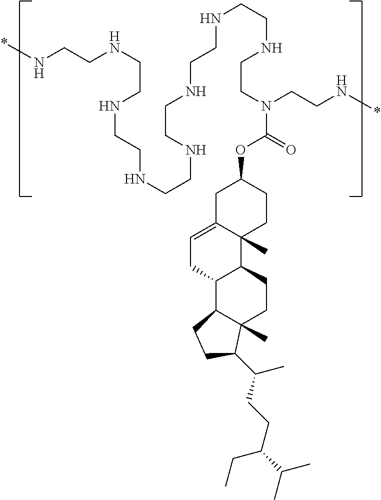 |
| 44 | 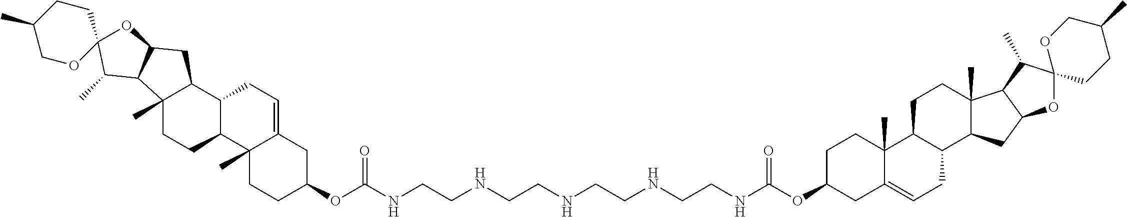 |
| 45 | 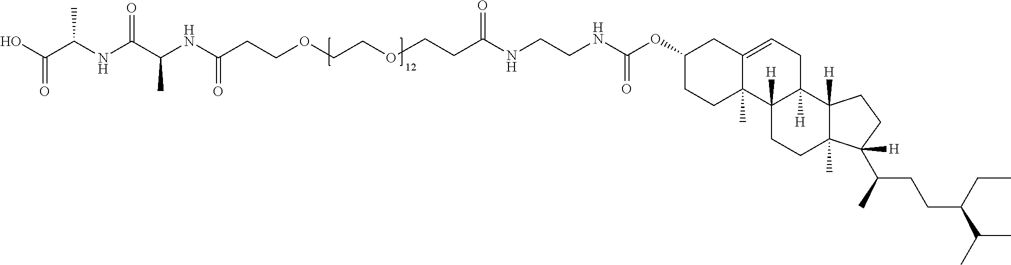 |
| 46 | 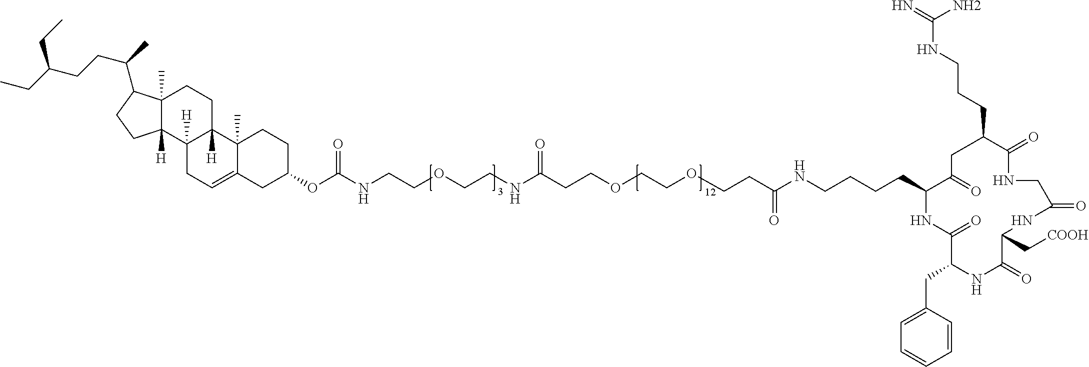 |
| 47 | 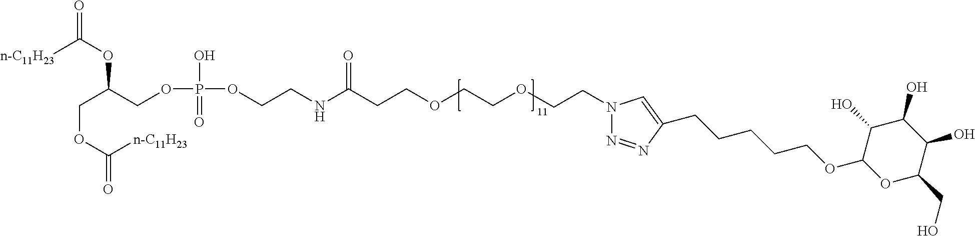 |
| 48 | 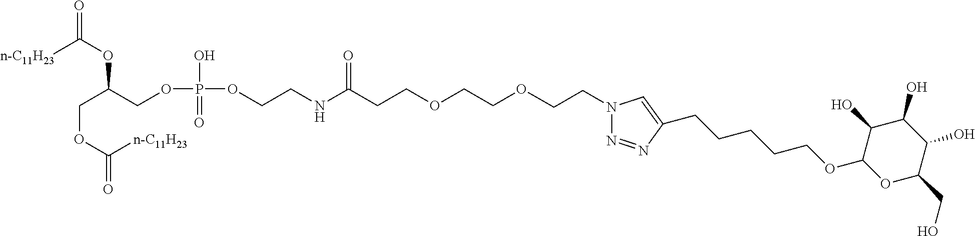 |
| 49 | 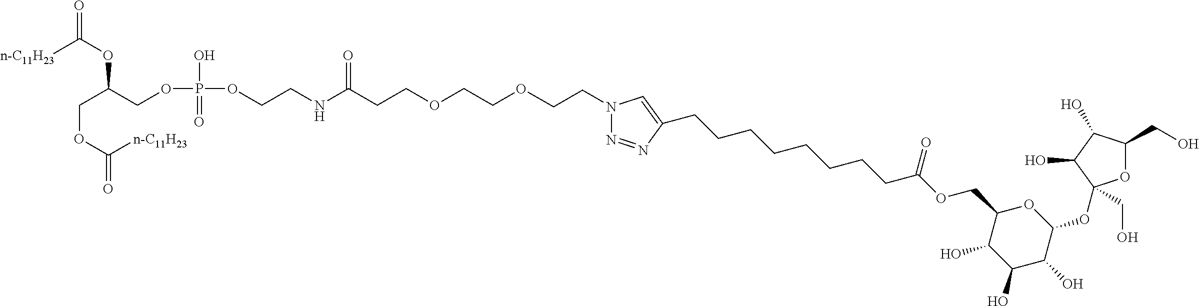 |
| 50 | 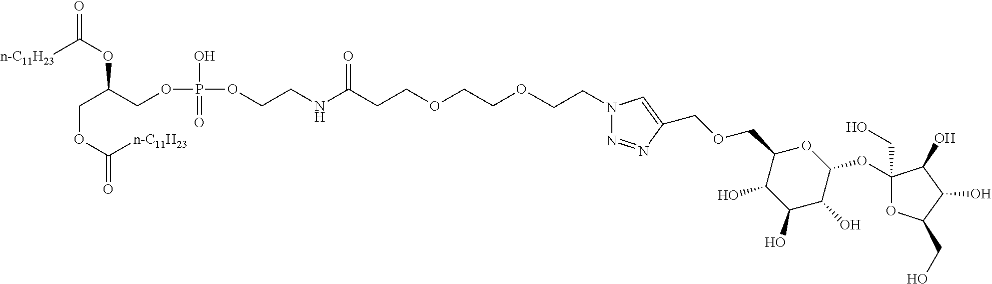 |

The presently disclosed compound of formula (I) may be prepared by any method known in the art. In certain embodiments, the presently disclosed compound of formula (I) are synthesized by directly reacting a compound from which group A of formula (I) will be derived with a compound from which group C of formula (I) will be derived, so as to form a covalent bond between the two compounds. As a result, the linker B of the compound of formula (I) is formed from the reaction of at least one moiety of the compound from which group A is derived with at least one moiety of the compound from which group B is derived. An example of such a direct reaction includes, but is not limited to, the formation of an ester group (esterification) between groups A and C of formula (I). Examples of esterification reactions that can be used to synthesize the compound of formula (I) include, but are not limited to, those depicted in reaction Scheme 1, as follows:

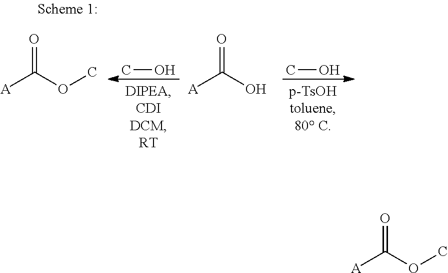

In certain embodiments, either of the carboxyl or hydroxyl moieties of groups A and C that ultimately form the ester group (linker B) between groups A and C can be converted into a more reactive group prior to esterification. Examples of such types of esterifications include, but are not limited to those depicted in reaction Schemes 2 and 3, as follows:

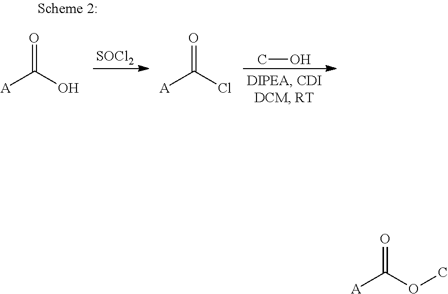

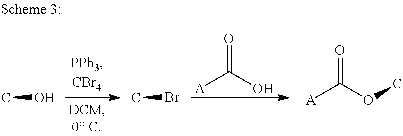

In certain other embodiments, the presently disclosed compounds of formula (I) are synthesized by first reacting (1) a compound from which group A of formula (I) will be derived and/or (2) a compound from which group C of formula (I) will be derived with one or more spacer molecules, followed by reacting the so-modified compound or compounds such that the two are tethered to each other via the spacer molecule (i.e., linker B). As a result, the linker B of the compounds of formula (I) can be formed from at least one moiety of the compound from which group A is derived, the linker molecule, and at least one moiety of the compound from which group B is derived. Examples of such a direct reaction includes, but is not limited to, the formation of an carbonate or carbamate between groups A and C of formula (I). Examples of such reactions that can be used to synthesize the compound of formula (I) include, but are not limited to, those depicted in reaction Schemes 4, 5, and 6, as follows:

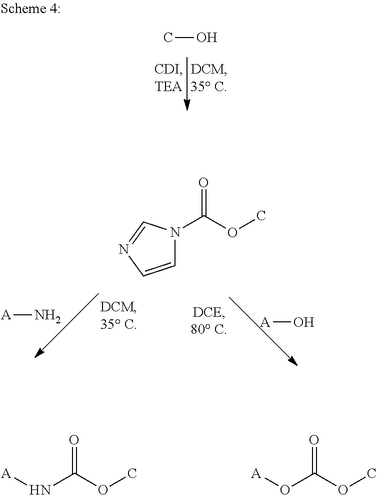

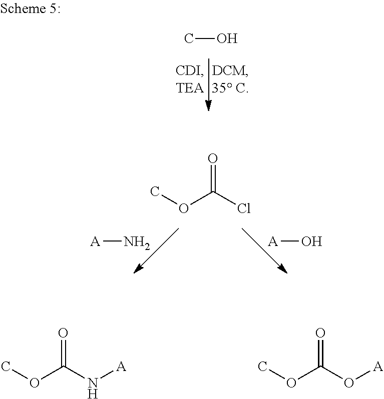

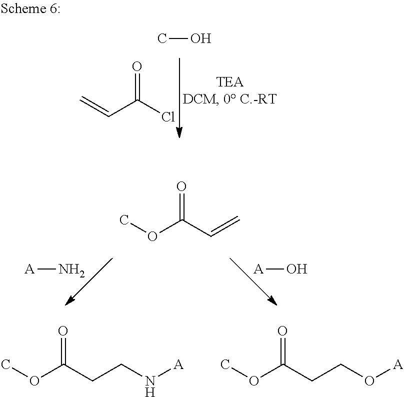

A further example of such a indirect reaction is the tethering of the auxin hormone indole-3-acetic acid with the phytosterol-sitosterol via a diaminoalkyl compound, as depicted in reaction Scheme 7, as follows:

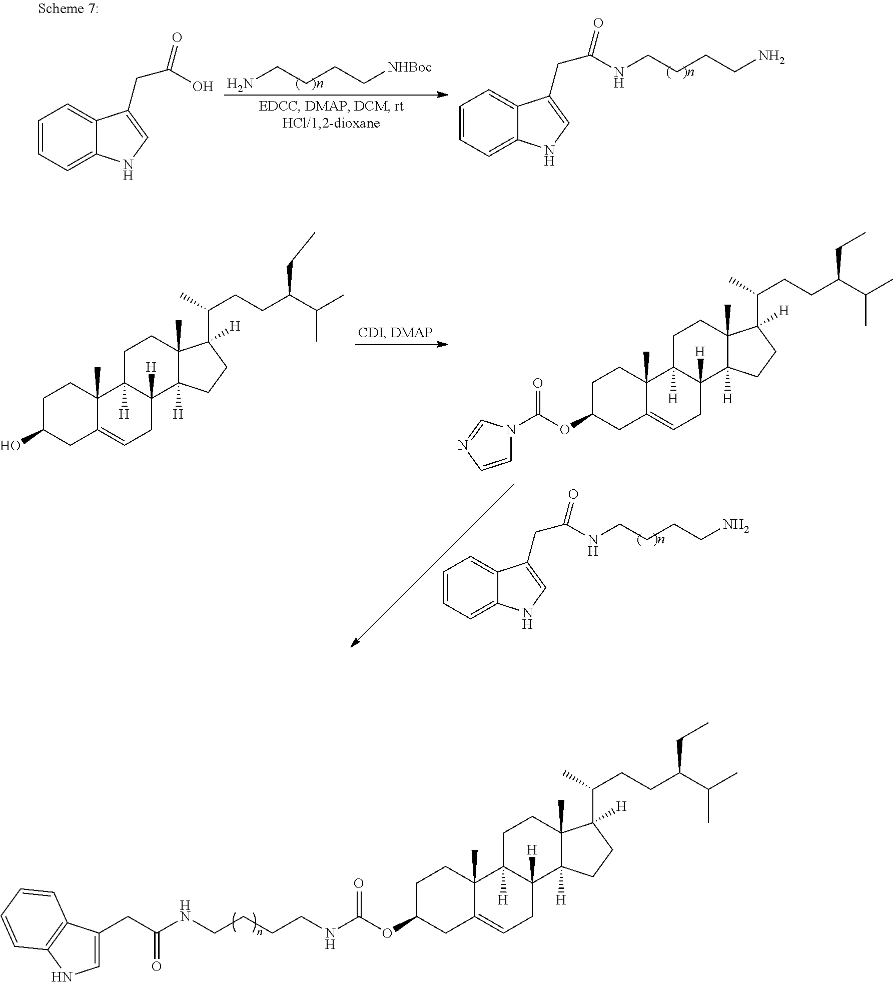

In certain embodiments, the starting materials used to prepare the compounds of formula (I) are commercially available and/or are easily and/or inexpensively prepared. In certain embodiments, the synthesis of the presently disclosed compounds of formula (I) is performed without solvent (i.e., neat). In certain other embodiments, the synthesis of the presently disclosed compounds of formula (I) is performed in a suitable solvent. In certain embodiments, these syntheses are performed at a temperature in the range of about ambient to about 120° C. for about 1 to about 96 hours. In certain embodiments, conventional heating sources can be employed. In certain other embodiments, non-conventional heating sources, such as microwave radiation, can be employed. In certain embodiments, after synthesis the crude product is purified or used in the next step “as is.” The synthesized compounds of formula (I) may be purified by any technique known in the art including, but not limited to, precipitation, crystallization, chromatography (e.g., silica gel chromatography, size exclusion chromatography, ion-exchange chromatography, and HPLC), and distillation. In certain embodiments, the crude product is purified by silica gel chromatography.

Complexing Agents

The presently disclosed formulations comprise at least one complexing agent. As used herein, the at least one “complexing agent” of the presently disclosed formulations encompasses any compound capable of non-covalently associating with the at least one active agent. Examples of such complexing agents that may be used in the preparation of the presently disclosed formulations include, but are not limited to, the compounds disclosed in U.S. Pat. No. 8,450,298 B2, U.S. Patent App. Pub. No. 2011/0293703 A1, WO 2010/053572 A1, U.S. Patent App. Pub. No. 2013/030240 A1, WO 2012/027675 A1, U.S. Patent App. Pub. No. 2011/0009641 A1, WO 2006/138380 A1, WO 2012/135025 A1, WO 2013/063468, A1, WO 2006/138380, WO 2010/053572 A1, WO 2002/31025 A1, WO 2008/011561 A1, WO 2013/090861 A1, WO 2012/027675 A1, U.S. Pat. No. 7,427,394, U.S. patent application Ser. No. 14/844,952, U.S. Provisional Patent App. Ser. No. 62/266,321, and U.S. Provisional Patent App. Ser. No. 62/387,296, the respective disclosures of which are each incorporated by reference herein in their entirety. Additional examples of complexing agents that may be used in the preparation of the presently disclosed formulations include, but are not limited to, compounds 1a-c, 2a-d, 3, 4a-n, 5a-b, 6a-b, and 7a-e, 8a-d, 9a-c, 10a-h, 11a-e, 12, 13, 14, 15, 17, 19a-f, 21, 22a-b, 23, 24a-b, 25-27, 30a-c, 31a-c, 32a-c, and 33-41, 42a-b, 43a-c, 44a-e, 45a-e, 46a-b, 47a-b, 48a-b, 49a-b, 50a-ad, 51a-ad, 52a-b, 53a-d, 54a-d, 55a-f, 56a-f, 57a-j, 58a-h, 59a-j, 60a-g, 61a-f, 62a-c, 63a-e, 64a-x, 65a-f, 66a-t, 67a-c, 68a-g, 69a-c, 70a-c, 71a-c, 72, 73a-g, 74, 75a-m, 76a-h, 77a-b, 78a-g, 79a-e, 83a-b, 86, 87a-b, 88, 91, 92, 93a-b, 94, 95a-ad, 96a, 96d, 96i, 96j, 96l, 96r, 96s-ad, 97a-ad, 98a-ad, 99-102, 103a-b, 104a-b, 105a-c, 106, 107, 108a-ab, 109a-ab, 110a-b, 111a-b, 112a-1, 113a-c, 114a-c, 115a-c, 116a-c, 117a-c, 118a-h, 119a-d, 120a-f, 121a-i, and 122a-e disclosed in “Synthetic Nucleic Acid Delivery Systems: Present and Perspectives” by Draghici, B. et al., J. Med. Chem., Vol. 58(10), pages 4091-4130 (2015), each of which are incorporated herein by reference. As used herein, the terms “non-covalently associating” and “non-covalently associated” encompass any kind of intermolecular interaction between the at least one complexing agent and the at least one active agent other than covalent interactions (i.e., interactions that involve the sharing of electrons). Examples of such non-covalent interactions include, but are not limited to, electrostatic interactions, such as ionic interactions, hydrogen bonding, and halogen bonding, Van der Waals forces, such as the Keesom force, the Debye force, and London dispersion forces, π-effects, such as π-n interactions, cation-π interactions, anion-π interactions, and polar π interactions, and hydrophobic interactions. Thus, in certain embodiments, the presently disclosed formulations are in the form of a non-covalent complex. As such, the term “non-covalent complex,” as used herein, encompasses a complex of at least one active agent that modulates one or more traits of a target insect, plant, or plant pathogen, (2) at least one complexing agent, and (3) at least one formulation transport agent, wherein the active agent and complexing agent are associated to each other via non-covalent interactions, as defined above, and the complexing agent and formulation transport agent may be associated to each other via non-covalent interactions, as defined above.

Active Agents

The presently disclosed formulations comprise at least one active agent that modulates one or more traits of the target organism (i.e., insects, plants, and plant pathogens). Such active agents include, but are not limited to, nucleic acids, peptides, polypeptides, small molecules, and mixtures thereof. In certain embodiments, the active agent comprises a nucleic acid. In certain embodiments, the nucleic acid comprises an interfering RNA molecule such as, e.g., an siRNA, aiRNA, miRNA, or mixtures thereof. In certain embodiments, the nucleic acid comprises a single-stranded or double-stranded DNA or RNA, or a DNA/RNA hybrid such as, e.g., an antisense oligonucleotide, a ribozyme, a plasmid, an immunostimulatory oligonucleotide, or mixtures thereof.

As used herein, the term “nucleic acid” includes any oligonucleotide or polynucleotide, with fragments containing up to 60 nucleotides generally termed oligonucleotides and longer fragments termed polynucleotides. In certain embodiments, oligonucleotides of the present disclosure are about 10, 15, 20, 25, 30, 35, 40, 45, 50, 55 or 60 nucleotides in length. Any of these values may be used to define a range for the size of the oligonucleotide. For example, the size of the oligonucleotide may range from 15-60, 20-60 or 25-60 nucleotides in length. In certain embodiments, the polynucleotide is 65, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, or more nucleotides in length. In certain embodiments, the polynucleotide is at least 65, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900 or 2000 nucleotides in length. Any of these values may be used to define a range for the size of the polynucleotide. For example, the polynucleotide may range from 100-150, 150-200, 200-250, 250-300, 300-350, 350-400, 400-450, 450-500, 500-550, 550-600, 600-650, 650-700, 700-750, 750-800, 800-850, 850-900, 900-950, or 950-1000 nucleotides in length. The nucleic acid may be administered alone in the particles of the present disclosure, or in combination (e.g., co-administered) with particles of the present disclosure comprising peptides, polypeptides, or small molecules, such as conventional drugs.

In the context of this invention, the terms “polynucleotide” and “oligonucleotide” refer to a polymer or oligomer of nucleotide or nucleoside monomers consisting of naturally-occurring bases, sugars, and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases.

Oligonucleotides are generally classified as deoxyribooligonucleotides or ribooligonucleotides. A deoxyribooligonucleotide consists of a 5-carbon sugar called deoxyribose joined covalently to phosphate at the 5′ and 3′ carbons of this sugar to form an alternating, unbranched polymer. A ribooligonucleotide consists of a similar repeating structure where the 5-carbon sugar is ribose.

Nucleic acids that can be used in the presently disclosed formulations includes any form of nucleic acid that is known. The nucleic acids used herein can be single-stranded DNA or RNA, or double-stranded DNA or RNA, or DNA-RNA hybrids. Examples of double-stranded DNA are described herein and include, e.g., structural genes, genes including control and termination regions, and self-replicating systems such as viral or plasmid DNA. Examples of double-stranded RNA are described herein and include, e.g., siRNA and other RNAi agents such as aiRNA and pre-miRNA. Single-stranded nucleic acids include, e.g., antisense oligonucleotides, ribozymes, mature miRNA, and triplex-forming oligonucleotides.

Nucleic acids that can be used in the formulations of the present disclosure may be of various lengths, which is generally dependent upon the particular form of nucleic acid. For example, in certain embodiments, plasmids or genes may be from about 1,000 to about 100,000 nucleotide residues in length. In certain embodiments, oligonucleotides may range from about 10 to about 100 nucleotides in length. In certain embodiments, oligonucleotides, both single-stranded, double-stranded, and triple-stranded, may range in length from about 10 to about 60 nucleotides, from about 15 to about 60 nucleotides, from about 20 to about 50 nucleotides, from about 15 to about 30 nucleotides, or from about 20 to about 30 nucleotides in length.

In certain embodiments, an oligonucleotide (or a strand thereof) that can be used in the presently disclosed formulations specifically hybridizes to or is complementary to a target polynucleotide sequence. The terms “specifically hybridizable” and “complementary” as used herein indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. In certain embodiments, an oligonucleotide is specifically hybridizable when binding of the oligonucleotide to the target sequence interferes with the normal function of the target sequence to cause a loss of utility or expression therefrom, and there is a sufficient degree of complementarity to avoid non-specific binding of the oligonucleotide to non-target sequences under conditions in which specific binding is desired. Thus, the oligonucleotide may include 1, 2, 3, or more base substitutions as compared to the region of a gene or mRNA sequence that it is targeting or to which it specifically hybridizes.

In certain embodiments, the oligo- or polynucleotide is optionally purified and substantially pure. In some embodiments, the polynucleotide is greater than 50% pure. In some embodiments, the oligo- or polynucleotide is greater than 75% pure. In some embodiments, the oligo- or polynucleotide is greater than 95% pure. The oligo- or polynucleotide may be provided by any means known in the art. In certain embodiments, the oligo- or polynucleotide has been engineered using recombinant techniques. The oligo- or polynucleotide may also be obtained from natural sources and purified from contaminating components found normally in nature. The oligo- or polynucleotide may also be chemically synthesized in a laboratory. In certain embodiments, the oligo- or polynucleotide is synthesized using standard solid phase chemistry.

The oligo- or polynucleotide may be modified by chemical or biological means. In certain embodiments, these modifications lead to increased stability of the oligo- or polynucleotide. Examples of such modifications include, but are not limited to, methylation, phosphorylation, and end-capping.

The oligo- or polynucleotide to be delivered may be in any form. Examples of such forms include, but are not limited to, a circular plasmid, a linearized plasmid, a cosmid, a viral genome, a modified viral genome, an artificial chromosome, dsRNA, ssRNA, dsDNA, ssDNA, RNA/DNA hybrids, dsRNA hairpins, siRNA, aiRNA, and miRNA.

The oligo- or polynucleotide may be of any sequence. In certain embodiments, the oligo- or polynucleotide encodes a protein or peptide. The encoded proteins may be enzymes, structural proteins, receptors, soluble receptors, ion channels, or cytokines. The oligo- or polynucleotide may also comprise regulatory regions to control the expression of a gene. These regulatory regions may include, but are not limited to, promoters, enhancer elements, repressor elements, TATA box, ribosomal binding sites, and stop site for transcription. In certain embodiments, the polynucleotide is not intended to encode a protein. For example, the polynucleotide may be used to fix an error in the genome of the cell being transfected.

In certain embodiments, the nucleic acid is modified. As used herein, the term “modified” in reference to a nucleic acid (e.g., an oligonucleotide or polynucleotide) is defined as a nucleic acid that contains variations of the standard bases, sugars and/or phosphate backbone chemical structures occurring in ribonucleic (i.e., A, C, G and U) and deoxyribonucleic (i.e., A, C, G and T) acids. Particular modifications of nucleic acids are further described below.

In certain embodiments, the oligo- or polynucleotide is an RNA that carries out RNA interference (RNAi). The term “interfering RNA” or “RNAi” or “interfering RNA sequence” refers to single-stranded RNA (e.g., mature miRNA) or double-stranded RNA (e.g., duplex RNA, such as siRNA, aiRNA, or pre-miRNA) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof.

siRNA

In certain embodiments, the active agent comprises an siRNA. The siRNA molecule can comprise a double-stranded region of about 15 to about 60 nucleotides in length (e.g., about 15 to 60, 15 to 50, 15 to 40, 15 to 30, 15 to 25, or 19 to 25 nucleotides in length, or 15, 16, 17, 18, 19, 20, 21, 22, 23, 24, or 25 nucleotides in length). The siRNA molecules used in the presently disclosed formulations are capable of silencing the expression of a target sequence in vitro and/or in vivo.

In certain embodiments, the siRNA molecule comprises modified nucleotides including, but not limited to, 2′-O-methyl (2′OMe) nucleotides, 2′-deoxy-2′-fluoro(2′F) nucleotides, 2′-deoxy nucleotides, 2′-O-(2-methoxyethyl) (MOE) nucleotides, locked nucleic acid (LNA) nucleotides, and mixtures thereof. In other embodiments, the siRNA comprises 2′OMe nucleotides (e.g., 2′OMe purine and/or pyrimidine nucleotides) such as, for example, 2′OMe-guanosine nucleotides, 2′OMe-uridine nucleotides, 2′OMe-adenosine nucleotides, 2′OMe-cytosine nucleotides, and mixtures thereof. In certain embodiments, the siRNA does not comprise 2′OMe-cytosine nucleotides. In certain embodiments, the siRNA comprises a hairpin loop structure.

In certain embodiments, the siRNA may comprise modified nucleotides in one strand (i.e., sense or antisense) or both strands of the double-stranded region of the siRNA molecule. In certain embodiments, uridine and/or guanosine nucleotides are modified at selective positions in the double-stranded region of the siRNA duplex. With regard to uridine nucleotide modifications, at least one, two, three, four, five, six, or more of the uridine nucleotides in the sense and/or antisense strand can be a modified uridine nucleotide such as a 2′OMe-uridine nucleotide. In certain embodiments, every uridine nucleotide in the sense and/or antisense strand is a 2′OMe-uridine nucleotide. With regard to guanosine nucleotide modifications, at least one, two, three, four, five, six, or more of the guanosine nucleotides in the sense and/or antisense strand can be a modified guanosine nucleotide such as a 2′OMe-guanosine nucleotide. In certain embodiments, every guanosine nucleotide in the sense and/or antisense strand is a 2′OMe-guanosine nucleotide.

In certain embodiments, at least one, two, three, four, five, six, seven, or more 5′-GU-3′ motifs in an siRNA sequence may be modified, e.g., by introducing mismatches to eliminate the 5′-GU-3′ motifs and/or by introducing modified nucleotides such as 2′OMe nucleotides. The 5′-GU-3′ motif can be in the sense strand, the antisense strand, or both strands of the siRNA sequence. The 5′-GU-3′ motifs may be adjacent to each other or, alternatively, they may be separated by 1, 2, 3, 4, 5, 6, 7, 8, 9, 10, 11, 12, or more nucleotides.

In certain embodiments, a modified siRNA molecule is capable of silencing at least about 5%, 10%, 15%, 20%, 25%, 30%, 35%, 40%, 45%, 50%, 55%, 60%, 65%, 70%, 75%, 80%, 85%, 90%, 95%, or 100% of the expression of the target sequence relative to the corresponding unmodified siRNA sequence.

In certain embodiments, the siRNA molecule does not comprise phosphate backbone modifications, e.g., in the sense and/or antisense strand of the double-stranded region. In certain embodiments, the siRNA comprises one, two, three, four, or more phosphate backbone modifications, e.g., in the sense and/or antisense strand of the double-stranded region. In certain embodiments, the siRNA does not comprise phosphate backbone modifications.

In certain embodiments, the siRNA does not comprise 2′-deoxy nucleotides, e.g., in the sense and/or antisense strand of the double-stranded region. In certain embodiments, the siRNA comprises one, two, three, four, or more 2′-deoxy nucleotides, e.g., in the sense and/or antisense strand of the double-stranded region. In certain embodiments, the siRNA does not comprise 2′-deoxy nucleotides.

In certain embodiments, the nucleotide at the 3′-end of the double-stranded region in the sense and/or antisense strand is not a modified nucleotide. In certain embodiments, the nucleotides near the 3′-end (e.g., within one, two, three, or four nucleotides of the 3′-end) of the double-stranded region in the sense and/or antisense strand are not modified nucleotides.

The siRNA molecules described herein may have 3′ overhangs of one, two, three, four, or more nucleotides on one or both sides of the double-stranded region, or may lack overhangs (i.e., have blunt ends) on one or both sides of the double-stranded region. In certain embodiments, the siRNA has 3′ overhangs of two nucleotides on each side of the double-stranded region. In certain embodiments, the 3′ overhang on the antisense strand has complementarity to the target sequence and the 3′ overhang on the sense strand has complementarity to a complementary strand of the target sequence. Alternatively, the 3′ overhangs do not have complementarity to the target sequence or the complementary strand thereof. In certain embodiments, the 3′ overhangs comprise one, two, three, four, or more nucleotides such as 2′-deoxy(2′H) nucleotides. In certain embodiments, the 3′ overhangs comprise deoxythymidine (dT) and/or uridine nucleotides. In certain embodiments, one or more of the nucleotides in the 3′ overhangs on one or both sides of the double-stranded region comprise modified nucleotides. Examples of modified nucleotides are described above and include, but are not limited to, 2′OMe nucleotides, 2′-deoxy-2′F nucleotides, 2′-deoxy nucleotides, 2′-O-2-MOE nucleotides, LNA nucleotides, and mixtures thereof. In certain embodiments, one, two, three, four, or more nucleotides in the 3′ overhangs present on the sense and/or antisense strand of the siRNA comprise 2′OMe nucleotides (e.g., 2′OMe purine and/or pyrimidine nucleotides) such as, for example, 2′OMe-guanosine nucleotides, 2′OMe-uridine nucleotides, 2′OMe-adenosine nucleotides, 2′OMe-cytosine nucleotides, and mixtures thereof.

The siRNA may comprise at least one or a cocktail (e.g., at least two, three, four, five, six, seven, eight, nine, ten, or more) of unmodified and/or modified siRNA sequences that silence target gene expression. The cocktail of siRNA may comprise sequences, which are directed to the same region or domain (e.g., a “hot spot”) and/or to different regions or domains of one or more target genes. In certain embodiments, one or more (e.g., at least two, three, four, five, six, seven, eight, nine, ten, or more) modified siRNA that silence target gene expression are present in a cocktail. In certain embodiments, one or more (e.g., at least two, three, four, five, six, seven, eight, nine, ten, or more) unmodified siRNA sequences that silence target gene expression are present in a cocktail.

In certain embodiments, the antisense strand of the siRNA molecule comprises or consists of a sequence that is at least about 80%, 85%, 90%, 95%, 96%, 97%, 98%, or 99% complementary to the target sequence or a portion thereof. In certain embodiments, the antisense strand of the siRNA molecule comprises or consists of a sequence that is 100% complementary to the target sequence or a portion thereof. In certain embodiments, the antisense strand of the siRNA molecule comprises or consists of a sequence that specifically hybridizes to the target sequence or a portion thereof.

In certain embodiments, the sense strand of the siRNA molecule comprises or consists of a sequence that is at least about 80%, 85%, 90%, 95%, 96%, 97%, 98%, or 99% identical to the target sequence or a portion thereof. In certain embodiments, the sense strand of the siRNA molecule comprises or consists of a sequence that is 100% identical to the target sequence or a portion thereof.

The siRNA that can be used in the presently disclosed formulations are capable of silencing the expression of a target gene of interest. Each strand of the siRNA duplex can be about 15 to about 60 nucleotides in length, or about 15 to about 30 nucleotides in length. In certain embodiments, the siRNA comprises at least one modified nucleotide. In some embodiments, the modified siRNA contains at least one 2′OMe purine or pyrimidine nucleotide such as a 2′OMe-guanosine, 2′OMe-uridine, 2′OMe-adenosine, and/or 2′OMe-cytosine nucleotide. In certain embodiments, one or more of the uridine and/or guanosine nucleotides are modified. The modified nucleotides can be present in one strand (i.e., sense or antisense) or both strands of the siRNA. The siRNA sequences may have overhangs or may lack overhangs (i.e., have blunt ends).

The modified siRNA generally comprises from about 1% to about 100% (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, or 100%) modified nucleotides in the double-stranded region of the siRNA duplex. In certain embodiments, one, two, three, four, five, six, seven, eight, nine, ten, or more of the nucleotides in the double-stranded region of the siRNA comprise modified nucleotides.

In certain embodiments, less than about 25% (e.g., less than about 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%) of the nucleotides in the double-stranded region of the siRNA comprise modified nucleotides.

In certain embodiments, from about 1% to about 25% (e.g., from about 1%-25%, 2%-25%, 3%-25%, 4%-25%, 5%-25%, 6%-25%, 7%-25%, 8%-25%, 9%-25%, 10%-25%, 11%-25%, 12%-25%, 13%-25%, 14%-25%, 15%-25%, 16%-25%, 17%-25%, 18%-25%, 19%-25%, 20%-25%, 21%-25%, 22%-25%, 23%-25%, 24%-25%, etc.) or from about 1% to about 20% (e.g., from about 1%-20%, 2%-20%, 3%-20%, 4%-20%, 5%-20%, 6%-20%, 7%-20%, 8%-20%, 9%-20%, 10%-20%, 11%-20%, 12%-20%, 13%-20%, 14%-20%, 15%-20%, 16%-20%, 17%-20%, 18%-20%, 19%-20%, 1%-19%, 2%-19%, 3%-19%, 4%-19%, 5%-19%, 6%-19%, 7%-19%, 8%-19%, 9%-19%, 10%-19%, 11%-19%, 12%-19%, 13%-19%, 14%-19%, 15%-19%, 16%-19%, 17%-19%, 18%-19%, 1%-18%, 2%-18%, 3%-18%, 4%-18%, 5%-18%, 6%-18%, 7%-18%, 8%-18%, 9%-18%, 10%-18%, 11%-18%, 12%-18%, 13%-18%, 14%-18%, 15%-18%, 16%-18%, 17%-18%, 1%-17%, 2%-17%, 3%-17%, 4%-17%, 5%-17%, 6%-17%, 7%-17%, 8%-17%, 9%-17%, 10%-17%, 11%-17%, 12%-17%, 13%-17%, 14%-17%, 15%-17%, 16%-17%, 1%-16%, 2%-16%, 3%-16%, 4%-16%, 5%-16%, 6%-16%, 7%-16%, 8%-16%, 9%-16%, 10%-16%, 11%-16%, 12%-16%, 13%-16%, 14%-16%, 15%-16%, 1%-15%, 2%-15%, 3%-15%, 4%-15%, 5%-15%, 6%-15%, 7%-15%, 8%-15%, 9%-15%, 10%-15%, 11%-15%, 12%-15%, 13%-15%, 14%-15%, etc.) of the nucleotides in the double-stranded region of the siRNA comprise modified nucleotides.

In certain embodiments, e.g., when one or both strands of the siRNA are selectively modified at uridine and/or guanosine nucleotides, the resulting modified siRNA can comprise less than about 30% modified nucleotides (e.g., less than about 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% modified nucleotides) or from about 1% to about 30% modified nucleotides (e.g., from about 1%-30%, 2%-30%, 3%-30%, 4%-30%, 5%-30%, 6%-30%, 7%-30%, 8%-30%, 9%-30%, 10%-30%, 11%-30%, 12%-30%, 13%-30%, 14%-30%, 15%-30%, 16%-30%, 17%-30%, 18%-30%, 19%-30%, 20%-30%, 21%-30%, 22%-30%, 23%-30%, 24%-30%, 25%-30%, 26%-30%, 27%-30%, 28%-30%, or 29%-30% modified nucleotides).

Examples of modified nucleotides suitable for use in the presently disclosed formulations include, but are not limited to, ribonucleotides having a 2′-O-methyl (2′OMe), 2′-deoxy-2′-fluoro(2′F), 2′-deoxy, 5-C-methyl, 2′-O-(2-methoxyethyl) (MOE), 4′-thio, 2′-amino, or 2′-C-allyl group. Modified nucleotides having a Northern conformation are also suitable for use in siRNA molecules. Such modified nucleotides include, without limitation, locked nucleic acid (LNA) nucleotides (e.g., 2′-O, 4′-C-methylene-(D-ribofuranosyl) nucleotides), 2′-O-(2-methoxyethyl) (MOE) nucleotides, 2′-methyl-thio-ethyl nucleotides, 2′-deoxy-2′-fluoro(2′F) nucleotides, 2′-deoxy-2′-chloro(2′Cl) nucleotides, and 2′-azido nucleotides. In certain instances, the siRNA molecules described herein include one or more G-clamp nucleotides. A G-clamp nucleotide refers to a modified cytosine analog wherein the modifications confer the ability to hydrogen bond both Watson-Crick and Hoogsteen faces of a complementary guanine nucleotide within a duplex. In addition, nucleotides having a nucleotide base analog such as, for example, C-phenyl, C-naphthyl, other aromatic derivatives, inosine, azole carboxamides, and nitroazole derivatives such as 3-nitropyrrole, 4-nitroindole, 5-nitroindole, and 6-nitroindole can be incorporated into siRNA molecules.

In certain embodiments, the siRNA molecules may further comprise one or more chemical modifications such as terminal cap moieties, phosphate backbone modifications, and the like. Examples of terminal cap moieties include, but are not limited to, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, c-nucleotides, modified base nucleotides, threo-pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3-aminopropyl phosphate, 6-aminohexyl phosphate, 1,2-aminododecyl phosphate, hydroxypropyl phosphate, 1,4-butanediol phosphate, 3′-phosphoramidate, 5′-phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties. Examples of phosphate backbone modifications (i.e., resulting in modified internucleotide linkages) include, but are not limited to, phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions. Such chemical modifications can occur at the 5′-end and/or 3′-end of the sense strand, antisense strand, or both strands of the siRNA.

In certain embodiments, the sense and/or antisense strand of the siRNA molecule can further comprise a 3′-terminal overhang having about 1 to about 4 (e.g., 1, 2, 3, or 4) 2′-deoxy ribonucleotides and/or any combination of modified and unmodified nucleotides.

The siRNA molecules can optionally comprise one or more non-nucleotides in one or both strands of the siRNA. As used herein, the term “non-nucleotide” refers to any group or compound that can be incorporated into a nucleic acid chain in the place of one or more nucleotide units, including sugar and/or phosphate substitutions, and allows the remaining bases to exhibit their activity. The group or compound is abasic in that it does not contain a commonly recognized nucleotide base such as adenosine, guanine, cytosine, uracil, or thymine and therefore lacks a base at the 1′-position.

In certain embodiments, chemical modification of the siRNA comprises attaching a conjugate to the siRNA molecule. The conjugate can be attached at the 5′ and/or 3′-end of the sense and/or antisense strand of the siRNA via a covalent attachment such as, e.g., a biodegradable linker. The conjugate can also be attached to the siRNA, e.g., through a carbamate group or other linking group. In certain instances, the conjugate is a molecule that facilitates the delivery of the siRNA into a cell.

aiRNA

In certain embodiments, the active agent comprises an asymmetrical interfering RNA (aiRNA). In certain embodiments, aiRNA duplexes of various lengths may be designed with overhangs at the 3′ and 5′ ends of the antisense strand to target an mRNA of interest. In certain embodiments, the sense strand of the aiRNA molecule is about 10-25, 12-20, 12-19, 12-18, 13-17, or 14-17 nucleotides in length, more typically 12, 13, 14, 15, 16, 17, 18, 19, or 20 nucleotides in length. In certain embodiments, the antisense strand of the aiRNA molecule is about 15-60, 15-50, or 15-40 nucleotides in length, or about 15-30, 15-25, or 19-25 nucleotides in length, or about 20-24, 21-22, or 21-23 nucleotides in length.

In certain embodiments, the 5′ antisense overhang contains one, two, three, four, or more nontargeting nucleotides (e.g., “AA”, “UU”, “dTdT”, etc.). In other embodiments, the 3′ antisense overhang contains one, two, three, four, or more nontargeting nucleotides (e.g., “AA”, “UU”, “dTdT”, etc.). In certain embodiments, the aiRNA molecules described herein may comprise one or more modified nucleotides, e.g., in the double-stranded (duplex) region and/or in the antisense overhangs. As a non-limiting example, aiRNA sequences may comprise one or more of the modified nucleotides described above for siRNA sequences. In certain embodiments, the aiRNA molecule comprises 2′OMe nucleotides such as, for example, 2′OMe-guanosine nucleotides, 2′OMe-uridine nucleotides, or mixtures thereof.

In certain embodiments, aiRNA molecules may comprise an antisense strand which corresponds to the antisense strand of an siRNA molecule, e.g., one of the siRNA molecules described herein. In certain embodiments, aiRNA molecules may be used to silence the expression of any of a target gene.

In certain embodiments, the aiRNA molecule comprises a double-stranded (duplex) region of about 10 to about 25 (base paired) nucleotides in length, wherein the aiRNA molecule comprises an antisense strand comprising 5′ and 3′ overhangs, and wherein the aiRNA molecule is capable of silencing target gene expression.

In certain embodiments, each of the 5′ and 3′ overhangs on the antisense strand comprises or consists of one, two, three, four, five, six, seven, or more nucleotides.

In certain embodiments, the aiRNA molecule comprises modified nucleotides selected from the group consisting of 2′OMe nucleotides, 2′F nucleotides, 2′-deoxy nucleotides, 2′-O-MOE nucleotides, LNA nucleotides, and mixtures thereof.

miRNA

In certain embodiments, the active agent comprises a microRNAs (miRNA). Generally, miRNA are single-stranded RNA molecules of about 21-23 nucleotides in length, which regulate gene expression. In certain embodiments, the miRNA molecules described herein are about 15-100, 15-90, 15-80, 15-75, 15-70, 15-60, 15-50, or 15-40 nucleotides in length, or about 15-30, 15-25, or 19-25 nucleotides in length, or about 20-24, 21-22, or 21-23 nucleotides in length. In certain embodiments, the miRNA molecule comprises about 15 to about 60 nucleotides in length, wherein the miRNA molecule is capable of silencing target gene expression.

In certain embodiments, miRNA molecules may comprise one or more modified nucleotides. As a non-limiting example, miRNA sequences may comprise one or more of the modified nucleotides described above for siRNA sequences. In certain embodiments, the miRNA molecule comprises 2′OMe nucleotides such as, for example, 2′OMe-guanosine nucleotides, 2′OMe-uridine nucleotides, or mixtures thereof. In certain embodiments, the miRNA molecule comprises modified nucleotides selected from the group consisting of 2′F nucleotides, 2′-deoxy nucleotides, 2′-O-MOE nucleotides, LNA nucleotides, and mixtures thereof.

dsRNA

In certain embodiments, the active agent is a dsRNA (double-stranded RNA). In certain embodiments, the active agent is an shRNA (short hairpin RNA).

Antisense Polynucleotide

In certain embodiments, the active agent is an antisense oligonucleotide. The terms “antisense polynucleotide” or “antisense” include polynucleotides that are complementary to a targeted polynucleotide sequence. Antisense polynucleotides are single strands of DNA or RNA that are complementary to a chosen sequence.

In certain embodiments, the polynucleotide is an antisense RNA. Antisense RNA polynucleotides prevent the translation of complementary RNA strands by binding to the RNA. Antisense DNA polynucleotides can be used to target a specific, complementary (coding or non-coding) RNA. If binding occurs, this DNA/RNA hybrid can be degraded by the enzyme RNase H. In certain embodiments, antisense polynucleotides comprise from about 10 to about 60 nucleotides, or from about 15 to about 30 nucleotides. The term also encompasses antisense polynucleotides that may not be exactly complementary to the desired target gene. Thus, the invention can be utilized in instances where non-target specific-activities are found with antisense, or where an antisense sequence containing one or more mismatches with the target sequence is the most preferred for a particular use.

Methods of producing antisense polynucleotides are known in the art and can be readily adapted to produce an antisense polynucleotides that targets any polynucleotide sequence. Selection of antisense polynucleotide sequences specific for a given target sequence is based upon analysis of the chosen target sequence and determination of secondary structure, Tm, binding energy, and relative stability. Antisense polynucleotides may be selected based upon their relative inability to form dimers, hairpins, or other secondary structures that would reduce or prohibit specific binding to the target mRNA in a host cell. Highly preferred target regions of the mRNA include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et al., Nucleic Acids Res., 25:3389-402 (1997)).

Ribozymes

In certain embodiments, the active agent is a ribozyme. Ribozymes are RNA-protein complexes having specific catalytic domains that possess endonuclease activity. For example, a large number of ribozymes accelerate phosphoester transfer reactions with a high degree of specificity, often cleaving only one of several phosphoesters in an oligonucleotide substrate. This specificity has been attributed to the requirement that the substrate bind via specific base-pairing interactions to the internal guide sequence (“IGS”) of the ribozyme prior to chemical reaction.

The enzymatic nucleic acid molecule may be formed in a hammerhead, hairpin, hepatitis δ virus, group I intron or RNaseP RNA (in association with an RNA guide sequence), or Neurospora VS RNA motif, for example. Important characteristics of enzymatic nucleic acid molecules used according to the invention are that they have a specific substrate binding site which is complementary to one or more of the target gene DNA or RNA regions, and that they have nucleotide sequences within or surrounding that substrate binding site which impart an RNA cleaving activity to the molecule.

Methods of producing a ribozyme targeted to any polynucleotide sequence are known in the art. Ribozyme activity can be optimized by altering the length of the ribozyme binding arms or chemically synthesizing ribozymes with modifications that prevent their degradation by serum ribonucleases, modifications which enhance their efficacy in cells, and removal of stem II bases to shorten RNA synthesis times and reduce chemical requirements.

Formulation Characteristics

The formulations of the present disclosure can take any form. Examples of such forms include, but are not limited to, complexes, particles (e.g., microparticles, nanoparticles, and picoparticles), micelles, liposomes, and lipoplexes. In certain embodiments, the presently disclosed the presently disclosed formulation transport agents and complexing agents are combined with an active agent to form microparticles, nanoparticles, liposomes, micelles, or lipoplexes. The active agent to be delivered by the particles, liposomes, micelles, or lipoplexes may be in the form of a gas, liquid, or solid, and the active agent may be a polynucleotide, protein, peptide, or small molecule. In certain embodiments, two or more active agents (e.g., two or more siRNA) can be formulated with the presently disclosed formulation transport agents and complexing agents to form a single complex, particle, micelle, or liposome containing the two or more active agents. Alternatively, in certain embodiments, the two or more active agents can each be separately formulated to form a single complex, particle, micelle, or liposome, each containing a single active agent, and are then combined to form a mixture prior to delivery to a target organism.

In certain embodiments, the diameter of the presently disclosed particles range from 1 to 1,000 micrometers. In certain embodiments, the diameter of the particles range from 1 to 100 micrometers. In certain embodiments, the diameter of the particles range from 1 to 10 micrometers. In certain embodiments, the diameter of the particles range from 10 to 100 micrometers. In certain embodiments, the diameter of the particles range from 100 to 1,000 micrometers. In certain embodiments, the diameter of the particles range from 1 to 5 micrometers. In certain embodiments, the diameter of the particles range from 1 to 1,000 nm. In certain embodiments, the diameter of the particles range from 1 to 100 nm. In certain embodiments, the diameter of the particles range from 1 to 10 nm. In certain embodiments, the diameter of the particles range from 10 nm to 100 nm. In certain embodiments, the diameter of the particles range from 100 nm to 1,000 nm. In certain embodiments, the diameters of the particles range from 1 to 5 nm. In certain embodiments, the diameter of the particles range from 1 to 1,000 pm. In certain embodiments, the diameter of the particles range from 1 to 100 pm. In certain embodiments, the diameter of the particles range from 1 to 10 pm. In certain embodiments, the diameter of the particles range from 10 to 100 pm. In certain embodiments, the diameter of the particles range from 100 to 1,000 pm. In certain embodiments, the diameter of the particles range from 1 to 5 pm.

The presently disclosed particles may be prepared using any method known in the art. These include, but are not limited to, spray drying, single and double emulsion solvent evaporation, solvent extraction, phase separation, simple and complex coacervation, and other methods well known to those of ordinary skill in the art. In certain embodiments, methods of preparing the particles are the double emulsion process and spray drying. In other embodiments, methods of preparing the particles are nanoprecipitation or flash precipitation, for example, as disclosed in U.S. Pat. Nos. 8,207,290, 8,404,799, 8,546,521, 8,618,240, and 8,809,492, each of which are incorporated herein in its entirety. The conditions used in preparing the particles may be altered to yield particles of a desired size or property (e.g., hydrophobicity, hydrophilicity, external morphology, “stickiness”, shape, etc.). The method of preparing the particle and the conditions (e.g., solvent, temperature, concentration, air flow rate, etc.) used may also depend on the agent being encapsulated and/or the composition of the matrix. Methods developed for making particles for delivery of encapsulated agents are described in the literature (e.g., Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference in their entirety). If the presently disclosed particles prepared by any of the above methods have a size range outside of the desired range, the particles can be sized, for example, using a sieve. The presently disclosed particles may also be coated. In certain embodiments, the particles are coated with a targeting agent. In other embodiments, the particles are coated to achieve desirable surface properties (e.g., a particular charge).

The presently disclosed micelles or liposomes may be prepared using any method known in the art. Micelles and liposomes are particularly useful in delivering hydrophobic agents, such as hydrophobic small molecules. In certain embodiments, the presently disclosed liposomes are formed through spontaneous assembly. In other embodiments, these liposomes are formed when thin lipid films or lipid cakes are hydrated and stacks of lipid crystalline bilayers become fluid and swell. The hydrated lipid sheets detach during agitation and self-close to form large, multilamellar vesicles (LMV). This prevents interaction of water with the hydrocarbon core of the bilayers at the edges. Once these particles have formed, reducing the size of the particle can be modified through input of sonic energy (sonication) or mechanical energy (extrusion). See Walde, P. “Preparation of Vesicles (Liposomes)” In Encyclopedia of Nanoscience and Nanotechnology; Nalwa, H. S. Ed. American Scientific Publishers: Los Angeles, 2004; Vol. 9, pp. 43-79; Szoka et al. “Comparative Properties and Methods of Preparation of Lipid Vesicles (Liposomes)” Ann. Rev. Biophys. Bioeng. 9:467-508, 1980; each of which is incorporated herein in its entirety.

In certain embodiments, the preparation of liposomes of the present disclosure can involve preparing the complexing agent for hydration, hydrating the complexing agent with agitation, and sizing the vesicles to achieve a homogenous distribution of liposomes. The complexing agent is first dissolved in an organic solvent to assure a homogeneous mixture. The solvent is then removed to form a lipidoid film/cake. This film is thoroughly dried to remove residual organic solvent by placing the vial or flask on a vacuum pump overnight. Hydration of the lipidoid film/cake is accomplished by adding an aqueous medium to the container of dry lipidoid and agitating the mixture. Disruption of LMV suspensions using sonic energy typically produces small unilamellar vesicles (SUV) with diameters in the range of from 15 to 50 nm. Lipid extrusion is a technique in which a lipid suspension is forced through a polycarbonate filter with a defined pore size to yield particles having a diameter near the pore size of the filter used. Extrusion through filters with 100 nm pores typically yields large, unilamellar vesicles (LUV) with a mean diameter of from 120 to 140 nm.

In certain embodiments, the presently disclosed formulations may further comprise at least one additional active agent to be delivered. In certain embodiments, this at least one additional active agent is part of the non-covalent complex of the presently disclosed formulation. In other words, the at least one additional active agent can be contained within the non-covalent complex or adhered to the surface of the non-covalent complex via non-covalent interactions, as defined above. In certain other embodiments, the at least one additional active agent is not contained within the non-covalent complex or adhered to the surface of the non-covalent complex, e.g., the at least one additional active agent is simply in a physical mixture with the non-covalent complex. In certain embodiments, the first active agent is an oligonucleotide or a polynucleotide, and the at least one additional active agent is an herbicide, an insecticide, a fungicide, a bactericide, and/or a viricide. In certain embodiments, the first active agent is used to increase the sensitivity of the target organism to the additional active agent, for example, to increase the sensitivity of a plant to an herbicide, or to increase the sensitivity of an insect to an insecticide.

In certain embodiments, the presently disclosed formulations may further comprise one or more adjuvants. As used herein, an “adjuvant” encompasses any compound that can assist the formulation transport agent in facilitating (1) the transport of the presently disclosed formulation (a) to the surface of a target cell in a target organism, (b) across the cell membrane of such target cells, and/or (c) through the cytosol of such target cells to the target DNA(s) and/or RNA(s) that govern the one or more traits of the target organism to be modulated, and/or (2) decomplexation of the active agent and the complexing agent once inside the target cell. In certain embodiments, the adjuvant is part of the non-covalent complex of the presently disclosed formulation. In other words, the adjuvant can be contained within the non-covalent complex or adhered to the surface of the non-covalent complex via non-covalent interactions, as defined above. In certain other embodiments, the adjuvant is not contained within the non-covalent complex or adhered to the surface of the non-covalent complex, e.g., the adjuvant is simply in a physical mixture with the non-covalent complex. Examples of such adjuvants include, but are not limited to, chloroquine, chlorpromazine, amodiaquine, perphenazine, coronatine, tolbutamide, glyburide, glybenclamide, arginine, lysine, and histidine.

In certain embodiments, the presently disclosed formulations may also comprise one or more excipients. Suitable excipients include, but are not limited to, fillers, extenders, binders, humectants, disintegrants, plasticizers, stabilizers, solution retarding agents, wetting agents, suspending agents, thickening agents, absorbents, lubricants, surfactants, buffering agents, diluents, solvents, emulsifying agents, suspending agents, sweetening agents, flavoring agents, perfuming agents, opacifying agents, separating agents, and coating permeability adjusters. In certain embodiments, the one or more excipients may be selected from the group consisting of carbohydrates, proteins, lipids, water-soluble polymers, and any combination thereof. In certain other embodiments, the one or more excipients comprises a water-soluble polymer such as polyethylene glycol (PEG), a polypropylene oxide (PPO), a polyvinylpyrrolidone (PVP), a polyvinyl alcohol (PVA), a polylactic acid (PLA), a poly(lactic-co-glycolic acid) (PLGA), or any combination thereof. In certain embodiments, the water-soluble polymer can be contained within or adhered to the surface of the non-covalent complexes of the present disclosure via non-covalent interactions, as defined above. In certain other embodiments, the water-soluble polymer can be tethered to the surface of the non-covalent complexes of the present disclosure via a lipid tail that is covalently bound on one end to the water-soluble polymer and which is entrained within the surface and/or interior of the non-covalent complex.

In certain embodiments, the presently disclosed formulations are combined with an agriculturally acceptable carrier. The agriculturally acceptable carrier can be solid or liquid and is a substance useful in formulation of agricultural products. Examples of such agricultural products include, but are not limited to, fertilizers, herbicides, insecticides, fungicides, bactericides, viricides, and nematicides. Examples of such agriculturally acceptable carriers for use in the presently disclosed formulations include, but are not limited to, surface active agents, stickers, spreader stickers, inert carriers, preservatives, humectants, dyes, UV (ultra-violet) protectants, buffers, flow agents, antifoams (e.g., polydimethylsiloxane), sodium aluminosilicate, or other components which facilitate product handling and application of the compositions. Examples of agriculturally acceptable inert carriers include inorganic minerals, such as kaolin, mica, gypsum, fertilizer, carbonates, sulfates, and phosphates, organic materials, such as sugar, starches, and cyclodextrins, and botanical materials, such as wood products, cork, powdered corn cobs, rice hulls, peanut hulls, and walnut shells. Agriculturally acceptable carriers are described, for example, in U.S. Pat. No. 6,984,609. In certain embodiments, the agriculturally acceptable carriers include, for example, natural or regenerated mineral substances, solvents, dispersants, wetting agents, tackifiers, thickeners, binders, or fertilizers. Such carriers are described, for example, in WO 97/33890. U.S. Pat. No. 6,984,609 and WO 97/33890 are incorporated by reference herein in their entireties.

In certain embodiments, the presently disclosed formulations may further comprise one or more additional compounds that can facilitate passage of the active agent(s) through the plant cell wall. Several technologies for facilitating passage of compounds through a plant cell wall are known in the art. For example, U.S. Pat. No. 8,609,420 describes conjugation of the active agent to a semi-conductor nanoparticle within the size range of 3-5 nm (e.g., a “quantum dot”) and one or more cell penetrating peptides to improve penetration of the plant cell and intracellular delivery of the active agent. U.S. Pat. No. 8,686,222 describes interacting a polyamidoamine dendrimer and one or more cell penetrating peptides with the active agent to improve cell penetration. U.S. Pat. No. 8,653,327 describes delivery of active agents through plant cell walls by coating a PEGylated semiconductor nanoparticle with the active agent. U.S. Pat. No. 8,722,410 describes transferring active agents into plant cells by applying the active agent to a nanoparticle coated with a subcellular compartment targeting protein. U.S. Pat. Nos. 8,609,420, 8,686,222, 8,653,327, and 8,722,410 are incorporated by reference herein in their entireties.

In certain embodiments, the complexes, microparticles, nanoparticles, picoparticles, liposomes, and micelles of the present disclosure may be modified to include targeting agents since it is often desirable to target a particular cell, collection of cells, or tissue. A variety of targeting agents that direct pharmaceutical compositions to particular cells are known in the art (e.g., Cotten et al. Methods Enzym. 217:618, 1993; which is incorporated herein by reference in its entirety). The targeting agents may be included throughout the particle or may be only on the surface. The targeting agent may be a protein, peptide, carbohydrate, glycoprotein, lipid, small molecule, and/or nucleic acid. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, and aptamers. If the targeting agent is included throughout the particle, the targeting agent may be included in the mixture that is used to form the particles. If the targeting agent is only on the surface of the particle, the targeting agent may be associated with (i.e., by covalent, hydrophobic, hydrogen bonding, van der Waals, or other interactions) the formed particles using standard chemical techniques.

In certain embodiments, the formulations of the present disclosure can be formulated as a bait, a food substance, or an attractant. For example, the formulations of the present disclosure can be incorporated into an insect bait suitable for oral administration of the formulation to the target insect. The bait may comprise the presently disclosed formulation dispersed in a carrier and an edible insect attractant. In certain embodiments, the bait comprises an edible insect attractant and a nanoparticle or microparticle formulation according to the present disclosure, wherein the nanoparticle or microparticle is dispersed in a carrier. The formulation of the present disclosure and attractant can be mixed together before being dispersed in the desired carrier. Suitable attractants include any type of insect food and/or attractant which will lure the insect to the bait to ingest the bait. Exemplary insect foods or attractants include, but are not limited to, any type of insect food, including various sugars, proteins, carbohydrates, yeast, fats, and/or oils. The bait can be in any form suitable for delivery and ingestion of the composition, depending on the habitat and target insect, but will typically be a liquid, gel, self-sustaining gel-matrix, or solid bait (e.g., tablets, granules, etc.). Exemplary carriers include, without limitation, agarose gel, gelatin gel, and/or pectin gel. In certain embodiments, the carrier is agarose gel, which is especially suited for aquatic habitats and breeding grounds. Insect baits are known in the art and are described, for example, in U.S. Pat. No. 8,841,272, which is incorporated herein by reference in its entirety.

The presently disclosed formulations can be present in the bait in an effective amount (i.e., concentration) for the activity of the active agent, such as gene silencing. The concentration of the active agent in the bait may be about 0.001, 0.005, 0.01, 0.05, 0.1, 0.5, 1, 2, 3, 4, 5, 6, 7, 8, 9, 10, 11, 12, 13, 14, 15, 16, 17, 18, 19 or 20% by weight of the bait. Any of these values may be used to define a range for the concentration of the active agent in the bait. For example, the concentration of the active agent in the bait may range from about 0.1 to about 1%, or from about 1 to about 5% by weight of the bait. The weight ratio of active agent to insect attractant (food) in the bait may be about 1:1, 1:5, 1:10, 1:20, 1:30, 1:40, 1:50, 1:60, 1:70, 1:80, 1:90, 1:100, 1:150 or 1:200. Any of these values may be used to define a range for the weight ratio of the active agent to the insect attractant in the bait. For example, the weight ratio of the active agent to the insect attractant in the bait may be from about 1:20 to about 1:200, or from about 1:50 to about 1:100.

In certain embodiments, the concentration of a microparticle or nanoparticle formulation according to the present disclosure in the bait may be about 0.001, 0.005, 0.01, 0.05, 0.1, 0.5, 1, 2, 3, 4, 5, 6, 7, 8, 9, 10, 11, 12, 13, 14, 15, 16, 17, 18, 19 or 20% by weight of the bait. Any of these values may be used to define a range for the concentration of the microparticle or nanoparticle in the bait. For example, the concentration of the microparticle or nanoparticle in the bait may range from about 0.1 to about 1%, or from about 1 to about 5% by weight of the bait. The weight ratio of the microparticle or nanoparticle to insect attractant (food) in the bait may be about 1:1, 1:5, 1:10, 1:20, 1:30, 1:40, 1:50, 1:60, 1:70, 1:80, 1:90, 1:100, 1:150 or 1:200. Any of these values may be used to define a range for the weight ratio of the microparticle or nanoparticle to the insect attractant in the bait. For example, the weight ratio of the microparticle or nanoparticle to the insect attractant in the bait may be from about 1:20 to about 1:200, or from about 1:50 to about 1:100.

Herbicidal and Pesticidal Applications

In another aspect, the presently disclosed formulations can be used to deliver an active agent to target organisms for the purpose of killing and/or controlling the proliferation of the target organisms, such as insects, weeds, and plant pathogens (e.g., fungi, bacteria, viruses, and nematodes). In certain embodiments, the presently disclosed formulations can comprise an insecticidal, nematicidal, fungicidal, bactericidal, viricidal, or herbicidal active agent, or combinations thereof. In certain embodiments, these formulations are combined with an agriculturally acceptable carrier to form a insecticidal, nematicidal, fungicidal, bactericidal, viricidal, or herbicidal formulation.

In certain embodiments, a target organism can be an organism in which the presently disclosed insecticidal, nematicidal, fungicidal, bactericidal, viricidal, or herbicidal formulations are intended to be functional, for example, to mediate gene silencing or suppression. In certain embodiments, a target organism is also a host organism, as described herein below. In certain other embodiments, a target organism is separate and distinct from a host organism that serves as a source of the active agent to be functional in the target organism.

In certain embodiments, the insecticidal, nematicidal, fungicidal, bactericidal, viricidal, or herbicidal formulation may further be combined with an agriculturally acceptable carrier. The agriculturally acceptale carrier can be solid or liquid and is a substance useful in formulation of agricultural products, for example, fertilizers, herbicides, insecticides, fungicides, bactericides, viricides, and nematicides. Agriculturally acceptable carriers include, for example, natural or regenerated mineral substances, solvents, dispersants, wetting agents, tackifiers, thickeners, binders or fertilizers. Such carriers are described, for example, in WO 97/33890, which is incorporated herein by reference.

The presently disclosed formulations can be applied to the crop area or plant to be treated, simultaneously or in succession with further compounds. These further compounds can be, for example, fertilizers or micronutrient donors or other preparations, which influence the growth of plants. They can also be selective herbicides or non-selective herbicides as well as insecticides, fungicides, bactericides, nematicides, viricides, molluscicides, or mixtures of several of these preparations, if desired together with further carriers, surfactants, or application promoting adjuvants customarily employed in the art of formulation.

Insecticides

In certain embodiments, one or more insecticides for killing or controlling the proliferation of an insect can be combined with one of the active agents described above or with the presently disclosed formulations. Examples of suitable insecticides include, but are not limited to, those provided in Table 2.

TABLE 5

| | |
|---|---|
| chloronicotinyls/ | acetamiprid, clothianidin, dinotefuran, imidacloprid, nitenpyram, |
| neonicotinoids | nithiazine, thiacloprid, thiamethoxam, imidaclothiz, (2E)-1-[(2-chloro-1,3- |
| | thiazol-5-yl)methyl]-3,5-dimethyl-N-nitro-1,3,5-tri-azinan-2-imine, |
| | acetylcholinesterase (AChE) inhibitors (such as carbamates and |
| | organophosphates) |
| carbamates | alanycarb, aldicarb, aldoxycarb, allyxycarb, aminocarb, bendiocarb, |
| | benfuracarb, bufencarb, butacarb, butocarboxim, butoxycarboxim, |
| | carbaryl, carbofuran, carbosulfan, chloethocarb, dimetilan, ethiofencarb, |
| | fenobucarb, fenothiocarb, formetanate, furathiocarb, isoprocarb, metam- |
| | sodium, methiocarb, methomyl, metolcarb, oxamyl, phosphocarb, |
| | pirimicarb, promecarb, propoxur, thiodicarb, thiofanox, triazamate, |
| | trimethacarb, XMC, xylylcarb |
| organophosphates | acephate, azamethiphos, azinphos (-methyl, -ethyl), bromophos-ethyl, |
| | bromfenvinfos (-methyl), butathiofos, cadusafos, carbophenothion, |
| | chlorethoxyfos, chlorfenvinphos, chlormephos, chlorpyrifos |
| | (-methyl/-ethyl), coumaphos, cyanofenphos, cyanophos, demeton-S- |
| | methyl, demeton-S-methylsulphon, dialifos, diazinon, dichlofenthion, |
| | dichlorvos/DDVP, dicrotophos, dimethoate, dimethylvinphos, |
| | dioxabenzofos, disulfoton, EPN, ethion, ethoprophos, etrimfos, famphur, |
| | fenamiphos, fenitrothion, fensulfothion, fenthion, flupyrazofos, fonofos, |
| | formothion, fosmethilan, fosthiazate, heptenophos, iodofenphos, |
| | iprobenfos, isazofos, isofenphos, isopropyl O-salicylate, isoxathion, |
| | malathion, mecarbam, methacrifos, methamidophos, methidathion, |
| | mevinphos, monocrotophos, naled, omethoate, oxydemeton-methyl, |
| | parathion (-methyl/-ethyl), phenthoate, phorate, phosalone, phosmet, |
| | phosphamidon, phosphocarb, phoxim, pirimiphos (-methyl/-ethyl), |
| | profenofos, propaphos, propetamphos, prothiofos, prothoate, pyraclofos, |
| | pyridaphenthion, pyridathion, quinalphos, sebufos, sulfotep, sulprofos, |
| | tebupirimfos, temephos, terbufos, tetrachlorvinphos, thiometon, |
| | triazophos, triclorfon, vamidothion |
| pyrethroids | acrinathrin, allethrin (d-cis-trans, d-trans), cypermethrin (alpha-, beta-, |
| | theta-, zeta-), permethrin (cis-, trans-), beta-cyfluthrin, bifenthrin, |
| | bioallethrin, bioallethrin-S-cyclopentyl-isomer, bioethanomethrin, |
| | biopermethrin, bioresmethrin, chlovaporthrin, cis-cypermethrin, cis- |
| | resmethrin, cis-permethrin, clocythrin, cycloprothrin, cyfluthrin, |
| | cyhalothrin, cyphenothrin, DDT, deltamethrin, empenthrin (1R-isomer), |
| | esfenvalerate, etofenprox, fenfluthrin, fenpropathrin, fenpyrithrin, |
| | fenvalerate, flubrocythrinate, flucythrinate, flufenprox, flumethrin, |
| | fluvalinate, fubfenprox, gamma-cyhalothrin, imiprothrin, kadethrin, |
| | lambda, cyhalothrin, metofluthrin, phenothrin (1R-trans isomer), |
| | prallethrin, profluthrin, protrifenbute, pyresmethrin, resmethrin, RU |
| | 15525, silafluofen, tau-fluvalinate, tefluthrin, terallethrin, tetramethrin |
| | (1R-isomer), tralocythrin, tralomethrin, transfluthrin, ZXI 8901, pyrethrins |
| | (pyrethrum) |
| oxadiazines | indoxacarb, acetylcholine receptor modulators (such as spinosyns) |
| spinosyns | spinosad |
| cyclodiene | camphechlor, chlordane, endosulfan, gamma-HCH, HCH, heptachlor, |
| organochlorines | lindane, methoxychlor |
| fiproles | acetoprole, ethiprole, vaniliprole, fipronil |
| mectins | abamectin, avermectin, emamectin, emamectin-benzoate, fenoxycarb, |
| | hydroprene, kinoprene, methoprene, ivermectin, lepimectin, epofenonane, |
| | pyriproxifen, milbemectin, milbemycin, triprene |
| diacylhydrazines | chromafenozide, halofenozide, methoxyfenozide, tebufenozide |
| benzoylureas | bistrifluoron, chlorfluazuron, diflubenzuron, fluazuron, flucycloxuron, |
| | flufenoxuron, hexaflumuron, lufenuron, novaluron, noviflumuron, |
| | penfluoron, teflubenzuron, triflumuron |
| organotins | azocyclotin, cyhexatin, fenbutatin oxide |
| pyrroles | chlorfenapyr |
| dinitrophenols | binapacyrl, dinobuton, dinocap, DNOC |
| METIs | fenazaquin, fenpyroximate, pyrimidifen, pyridaben, tebufenpyrad, |
| | tolfenpyrad, rotenone, acequinocyl, fluacrypyrim, microbial disrupters of |
| | the intestinal membrane of insects (such as Bacillus thuringiensis strains), |
| | inhibitors of lipid synthesis (such as tetronic acids and tetramic acids) |
| tetronic acids | spirodiclofen, spiromesifen, spirotetramat |
| tetramic acids | cis-3-(2,5-dimethylphenyl)-8-methoxy-2-oxo-1-azaspiro[4.5]dec-3-en-4-yl |
| | ethyl carbonate (alias: carbonic acid, 3-(2,5-dimethylphenyl)-8-methoxy- |
| | 2-oxo-1-azaspiro[4.5]dec-3-en-4-yl ethyl ester; CAS Reg. No.: 382608- |
| | 10-8), carboxamides (such as flonicamid), octopaminergic agonists (such |
| | as amitraz), inhibitors of the magnesium-stimulated ATPase (such as |
| | propargite), ryanodin receptor agonists (such as phthalamides or |
| | rynaxapyr) |
| phthalamides | N₂-[1,1-dimethyl-2-(methylsulphonyl)ethyl]-3-iodo-N₁-[2-methyl--4- |
| | [1,2,2,2-tetrafluoro-1-(trifluoromethyl)ethyl]phenyl]-1,2-benzenedi- |
| | carboxamide (i.e., flubendiamide; CAS reg. No.: 272451-65-7) |

Additional non-limiting examples of suitable insecticides include biologics, hormones or pheromones such as azadirachtin, Bacillus species, Beauveria species, codlemone, Metarrhizium species, Paecilomyces species, thuringiensis and Verticillium species, and active compounds having unknown or non-specified mechanisms of action such as fumigants (such as aluminium phosphide, methyl bromide and sulphuryl fluoride) and selective feeding inhibitors (such as cryolite, flonicamid and pymetrozine). In certain embodiments, the insecticide can be a mite growth inhibitor. Examples of such mite growth inhibitors include, but are not limited to, clofentezine, etoxazole and hexythiazox, amidoflumet, benclothiaz, benzoximate, bifenazate, bromopropylate, buprofezin, chinomethioat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyrafluprole, pyridalyl, pyriprole, sulfluramid, tetradifon, tetrasul, triarathene, verbutin, 3-methylphenyl propylcarbamate (Tsumacide Z), 3-(5-chloro-3-pyridinyl)-8-(2,2,2-trifluoroethyl)-8-azabicyclo[3.2.1]octane-3-carbonitrile (CAS reg. No. 185982-80-3) and the corresponding 3-endo isomer (CAS reg. No. 185984-60-5), and also preparations comprising insecticidally effective plant extracts, nematodes, fungi, or viruses.

Herbicides

In certain embodiments, one or more herbicides for killing or controlling the proliferation of weeds and other unwanted plants can be combined with one of the active agents described above or with the presently disclosed formulations. Examples of herbicides include, but are not limited to, benzoic acid herbicides, such as dicamba esters, phenoxyalkanoic acid herbicides, such as 2,4-D, MCPA and 2,4-DB esters, aryloxyphenoxypropionic acid herbicides, such as clodinafop, cyhalofop, fenoxaprop, fluazifop, haloxyfop, and quizalofop esters, pyridinecarboxylic acid herbicides, such as aminopyralid, picloram, and clopyralid esters, pyrimidinecarboxylic acid herbicides, such as aminocyclopyrachlor esters, pyridyloxyalkanoic acid herbicides, such as fluoroxypyr and triclopyr esters, and hydroxybenzonitrile herbicides, such as bromoxynil and ioxynil esters, esters of the arylpyridine carboxylic acids, and arylpyrimidine carboxylic acids of the generic structures disclosed in U.S. Pat. No. 7,314,849, U.S. Pat. No. 7,300,907, and U.S. Pat. No. 7,642,220, each of which is incorporated by reference herein in its entirety. In certain embodiments, the herbicide can be selected from the group consisting of 2,4-D, 2,4-DB, acetochlor, acifluorfen, alachlor, ametryn, amitrole, asulam, atrazine, azafenidin, benefin, bensulfuron, bensulide, bentazon, bromacil, bromoxynil, butylate, carfentrazone, chloramben, chlorimuron, chlorproham, chlorsulfuron, clethodim, clomazone, clopyralid, cloransulam, cyanazine, cycloate, DCPA, desmedipham, dichlobenil, diclofop, diclosulam, diethatyl, difenzoquat, diflufenzopyr, dimethenamid-p, diquat, diuron, DSMA, endothall, EPTC, ethalfluralin, ethametsulfuron, ethofumesate, fenoxaprop, fluazifop-P, flucarbazone, flufenacet, flumetsulam, flumiclorac, flumioxazin, fluometuron, fluroxypyr, fluthiacet, fomesafen, foramsulfuron, glufosinate, glyphosate, halosulfuron, haloxyfop, hexazinone, imazamethabenz, imazamox, imazapic, imazaquin, imazethapyr, isoxaben, isoxaflutole, lactofen, linuron, MCPA, MCPB, mesotrione, methazole, metolachlor-s, metribuzin, metsulfuron, molinate, MSMA, napropamide, naptalam, nicosulfuron, norflurazon, oryzalin, oxadiazon, oxasulfuron, oxyfluorfen, paraquat, pebulate, pelargonic acid, pendimethalin, phenmedipham, picloram, primisulfuron, prodiamine, prometryn, pronamide, propachlor, propanil, prosulfuron, pyrazon, pyridate, pyrithiobac, quinclorac, quizalofop, rimsulfuron, sethoxydim, siduron, simazine, sulfentrazone, sulfometuron, sulfosulfuron, tebuthiuron, terbacil, thiazopyr, thifensulfuron, thiobencarb, tralkoxydim, triallate, triasulfuron, tribenuron, triclopyr, trifluralin, triflusulfuron, vernolate.

Fungicides

In certain embodiments, one or more fungicides for killing or controlling the proliferation of a fungus can be combined with one of the active agents described above or with the presently disclosed formulations. Exemplary fungicides include, but are not limited to, strobilurins, azoxystrobin, dimoxystrobin, enestroburin, fluoxastrobin, kresoxim-methyl, metominostrobin, picoxystrobin, pyraclostrobin, trifloxystrobin, orysastrobin, carboxamides, carboxanilides, benalaxyl, benalaxyl-M, benodanil, carboxin, mebenil, mepronil, fenfuram, fenhexamid, flutolanil, furalaxyl, furcarbanil, furametpyr, metalaxyl, metalaxyl-M (mefenoxam), methfuroxam, metsulfovax, ofurace, oxadixyl, oxycarboxin, penthiopyrad, pyracarbolid, salicylanilide, tecloftalam, thifluzamide, tiadinil, N-biphenylamides, bixafen, boscalid, carboxylic acid morpholides, dimethomorph, flumorph, benzamides, flumetover, fluopicolid (picobenzamid), zoxamid, carboxamides, carpropamid, diclocymet, mandipropamid, silthiofam, azoles, triazoles, bitertanol, bromuconazole, cyproconazole, difenoconazole, diniconazole, enilconazole, epoxiconazole, fenbuconazole, flusilazol, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole, Imidazoles, cyazofamid, imazalil, pefurazoate, prochloraz, triflumizole, benzimidazoles, benomyl, carbendazim, fuberidazole, thiabendazole, ethaboxam, etridiazole, hymexazol, nitrogen-containing heterocyclyl compounds, pyridines, fuazinam, pyrifenox, pyrimidines, bupirimate, cyprodinil, ferimzone, fenarimol, mepanipyrim, nuarimol, pyrimethanil, piperazines, triforine, pyrroles, fludioxonil, fenpiclonil, morpholines, aldimorph, dodemorph, fenpropimorph, tridemorph, dicarboximides, iprodione, procymidone, vinclozolin, acibenzolar-S-methyl, anilazine, captan, captafol, dazomet, diclomezin, fenoxanil, folpet, fenpropidin, famoxadon, fenamidon, octhilinone, probenazole, proquinazid, pyroquilon, quinoxyfen, tricyclazole, carbamates, dithiocarbamates, ferbam, mancozeb, maneb, metiram, metam, propineb, thiram, zineb, ziram, diethofencarb, flubenthiavalicarb, iprovalicarb, propamocarb, guanidines, dodine, iminoctadine, guazatine, kasugamycin, polyoxins, streptomycin, validamycin A, organometallic compounds, fentin salts, sulfur-containing heterocyclyl compounds, isoprothiolane, dithianone, organophosphorous compounds, edifenphos, fosetyl, fosetyl-aluminum, iprobenfos, pyrazophos, tolclofos-methyl, Organochlorine compounds, thiophanate-methyl, chlorothalonil, dichlofluanid, tolylfluanid, flusulfamide, phthalide, hexachlorobenzene, pencycuron, quintozene, nitrophenyl derivatives, binapacryl, dinocap, dinobuton, spiroxamine, cyflufenamid, cymoxanil, metrafenon, N-2-cyanophenyl-3,4-dichloroisothiazol-5-carboxamide (isotianil), N-(3′,4′,5′-trifluorobiphenyl-2-yl)-3-difluoromethyl-1-methylpyrazole-4-carboxamide, 3-[5-(4-chlorophenyl)-2,3-dimethylisoxazolidin-3-yl]-pyridine, N-(3′,4′-dichloro-4-fluorobiphenyl-2-yl)-3-difluoromethyl-1-methylpyrazol-e-4-carboxamide, 5-chloro-7-(4-methylpiperidin-1-yl)-6-(2,4,6-trifluorophenyl)-[1,2,4]tria-zolo[1,5-a]pyrimidine, 2-butoxy-6-iodo-3-propylchromen-4-one, N,N-dimethyl-3-(3-bromo-6-fluoro-2-methylindole-1-sulfonyl)-[1,2,4]triazo-le-1-sulfonamide, methyl-(2-chloro-5-[1-(3-methylbenzyloxyimino)-ethyl]benzyl)carbamate, methyl-(2-chloro-5-[1-(6-methylpyridin-2-ylmethoxy-imino)ethyl]benzyl)carbamate, methyl 3-(4-chlorophenyl)-3-(2-isopropoxycarbonylamino-3-methylbutyryl-amino)propionate, 4-fluorophenyl N-(1-(1-(4-cyanophenyl)ethanesulfonyl)but-2-yl)carbamate, N-(2-(4-[3-(4-chlorophenyl)prop-2-ynyloxy]-3-methoxyphenyl)ethyl)-2-metha-nesulfonylamino-3-methylbutyramide, N-(2-(4-[3-(4-chlorophenyl)prop-2-ynyloxy]-3-methoxyphenyl)ethyl)-2-ethan-esulfonylamino-3-methylbutyramide, N-(4′-bromobiphenyl-2-yl)-4-difluoromethyl-2-methylthiazol-5-carboxamide, N-(4′-trifluoromethylbiphenyl-2-yl)-4-difluoromethyl-2-methylthiazol-5-carboxamide, N-(4′-chloro-3′-fluorobiphenyl-2-yl)-4-difluoromethyl-2-methylt-hiazol-5-carboxamide, and methyl 2-(ortho-((2,5-dimethylphenyloxy-methylene)phenyl)-3-methoxyacrylate.

Modulation of Traits in Plants, Insects, and Plant Pathogens

Plants

In another aspect, the present disclosure provides for methods of modulating a trait of a plant, comprising delivering to the plant an effective amount of the presently disclosed formulation comprising an oligonucleotide or polynucleotide that modulates the expression of a gene in the plant. Oligonucleotides or polynucleotides that modulate the expression of a gene in a plant include, but are not limited to, RNA molecules (e.g., siRNA, aiRNA, miRNA, dsRNA, and shRNA) and DNA molecules (e.g., antisense polynucleotides) that decrease expression of the gene in the plant, and RNA molecules (e.g., mRNA) and DNA molecules (e.g., expression cassettes and plasmids) that increase expression of the gene in the plant. In certain embodiments, the oligonucleotide or polynucleotide modulates the expression of a gene that is endogenous to the plant. In other embodiments, the oligonucleotide or polynucleotide modulates the expression of a gene that is heterologous to the plant, e.g., a transgene that does not naturally occur within the plant. In certain embodiments, the oligonucleotide or polynucleotide that modulates the expression of a gene in the plant hybridizes to a gene or gene product that is endogenous to the plant.

In certain embodiments, traits that may be modulated in a plant include, but are not limited to, total seed germination, rate of seed germination, disease tolerance, insect tolerance, drought tolerance, heat tolerance, cold tolerance, salinity tolerance, tolerance to heavy metals, total yield, seed yield, fruit yield, root growth, early vigor, plant growth, plant biomass, plant size, plant lifespan, total plant dry weight, above-ground dry weight, above-ground fresh weight, leaf area, stem volume, plant height, rosette diameter, leaf length, root length, root mass, tiller number, leaf number, fruit size, fruit freshness, fruit ripening time, fruit nutritional content, plant nutritional content, and any combination thereof. In certain embodiments, the presently disclosed formulations can be used to deliver an active agent to a plant (e.g., a weed), for the purpose of killing and/or controlling the proliferation of the plant.

In certain embodiments, one or more of the above-mentioned traits in a plant is increased or improved relative to a plant that is not treated with the presently disclosed formulation. The trait in the plant as described herein may be increased by at least about 5, 10, 15, 20, 25, 30, 35, 40, 45, 50, 55, 60, 65, 70, 75, 80, 85, 90, 95, 100, 150, 200, 300, 400, 500, 600, 700, 800, 900, or 1000% by delivery of the presently disclosed formulation to the plant relative to a plant that is not treated with the formulation. In other embodiments, one or more of the above mentioned traits is decreased relative to a plant that is not treated with the presently disclosed formulation. The trait in the plant as described herein may be decreased by at least about 5, 10, 15, 20, 25, 30, 35, 40, 45, 50, 55, 60, 65, 70, 75, 80, 85, 90, 95, or 100% by delivery of the presently disclosed formulation to the plant relative to a plant that is not treated with the formulation.

Insects

In another aspect, the present disclosure provides for a method of modulating a trait of an insect, comprising delivering to the insect, to a plant infested with the insect, or to a plant prior to infestation with the insect an effective amount of the presently disclosed formulation comprising an oligonucleotide or polynucleotide that modulates expression of a gene in the insect. Oligonucleotides or polynucleotides that modulate the expression of a gene in the insect include, but are not limited to, RNA molecules (e.g., siRNA, aiRNA, miRNA, dsRNA, and shRNA) and DNA molecules (e.g., antisense polynucleotides) that decrease expression of the gene in the insect, and RNA molecules (e.g., mRNA) and DNA molecules (e.g., expression cassettes and plasmids) that increase expression of the gene in the insect. In certain embodiments, the oligonucleotide or polynucleotide that modulates the expression of a gene in the insect hybridizes to a gene or gene product that is endogenous to the insect.

Traits that may be modulated in the insect include, but are not limited to, insect growth, development, activity, and/or lifespan. For example, in certain embodiments, delivery of the presently disclosed formulation to the insect kills the insect. In certain embodiments, delivery of the presently disclosed formulation to the insect reduces its growth and/or lifespan, thereby reducing the damage done by the insect to a plant. In certain embodiments, delivery of the presently disclosed formulation to the insect causes the insect to remain in a young or immature stage, thus preventing the insect from completing its lifecycle. For example, in certain embodiments, delivery of the presently disclosed formulation to the insect interferes with enzymes involved in the molting process that stimulate the synthesis and formation of chitin, which is an essential component of an insect's exoskeleton. As a result, the insect fails to reach adulthood because it dies in an immature stage. In certain embodiments, delivery of the presently disclosed formulation to the insect disrupts the feeding activity of the insect. As a result, insects starve to death because they are unable to obtain nutrients.

In certain embodiments, the delivery of the presently disclosed formulation to the insect decreases its growth, activity or lifespan by at least about 5, 10, 15, 20, 25, 30, 35, 40, 45, 50, 55, 60, 65, 70, 75, 80, 85, 90, 95, or 100% relative to an insect that is not treated with the formulation. In certain embodiments, the delivery of the presently disclosed formulation to the insect increases its growth, activity or lifespan by at least about 5, 10, 15, 20, 25, 30, 35, 40, 45, 50, 55, 60, 65, 70, 75, 80, 85, 90, 95, 100, 150, 200, 300, 400, 500, 600, 700, 800, 900 or 1000% relative to an insect that is not treated with the formulation.

Plant Pathogens

In another aspect, the present disclosure provides a method of modulating the pathogenicity of a plant pathogen, comprising applying to the plant pathogen, to a plant infected with the plant pathogen, or to a plant prior to infection with the plant pathogen the presently disclosed formulation comprising an oligonucleotide or polynucleotide that modulates expression of a gene in the plant pathogen. For example, in certain embodiments, the pathogenicity of the plant pathogen is decreased, for example by decreasing the growth, activity, or lifespan of the plant pathogen, or delaying the development of the plant pathogen. In a particular embodiment, the presently disclosed formulation is used to kill the plant pathogen and/or control its proliferation. In certain other embodiments, the pathogenicity of the plant pathogen is increased, for example, by increasing the growth, activity or lifespan of the plant pathogen, or accelerating its development. Increasing pathogenicity of a plant pathogen may be used, for example, to kill or reduce the growth of a plant such as a weed. In certain embodiments, the growth, activity, or lifespan of the plant pathogen may be decreased by about 5, 10, 15, 20, 25, 30, 35, 40, 45, 50, 55, 60, 65, 70, 75, 80, 85, 90, 95, or 100% relative to a plant pathogen that is not treated with the presently disclosed formulation. In certain embodiments, the growth, activity, or lifespan of the plant pathogen may be increased by about 5, 10, 15, 20, 25, 30, 35, 40, 45, 50, 55, 60, 65, 70, 75, 80, 85, 90, 95, 100, 150, 200, 300, 400, 500, 600, 700, 800, 900 or 1000% relative to a plant pathogen that is not treated with the presently disclosed formulation.

Target Organisms

In certain embodiments, the target organism is any organism in which one or more traits is modulated by the presently disclosed active agent. In certain embodiments, a target organism is also a host organism, as described herein below. For example, in certain embodiments, the target organism is an organism comprising one or more genes that is targeted by an oligonucleotide or polynucleotide active agent. In certain embodiments, the target organism is a plant in which one or more yield-related traits is improved by the active agent. In certain embodiments, the target organism is a beneficial insect whose growth, fecundity, or disease resistance is improved by the active agent. In certain embodiments, the target organisms are plant pests or pathogens whose damage to the plant can be reduced or eliminated by active agents according to the invention. Examples of plant pests and pathogens include, but are not limited to, insects, nematodes, fungi, bacteria, viruses, and parasitic plants such as striga, dodder, and mistletoe. Insect pests that may be targeted according to the invention include, but are not limited to, chewing, sucking, and boring insects that belong, for example, to the non-limiting Orders Coleoptera, Diptera, Hemiptera, Heteroptera, Homoptera, Hymenoptera, Lepidoptera, and Orthoptera.

Insects

In certain embodiments, the presently disclosed formulations may be taken up by an insect by direct contact with the formulation, for example, by topical adsorption or inhalation of the formulation or by direct feeding on a bait comprising the formulation, as described below. The formulations may also be taken up by the insect by direct feeding on a plant that has been treated with the formulation. Examples of insect pests that may be targeted with the presently disclosed formulations include, but are not limited to, those provided in Table 3.

TABLE 6

| Latin Name | Common Name |
|---|---|
| Ostrinia nubilalis | European corn borer |
| Helicoverpa zea | Corn earworm |
| Spodoptera exigua | Beet armyworm |
| Spodoptera frugiperda | Fall armyworm |
| Diatraea grandiosella | Southwestern corn borer |
| Elasmopalpus lignosellus | Lesser cornstalk borer |
| Papaipema nebris | Stalk borer |
| Pseudaletia unipuncta | Common armyworm |
| Agrotis ipsilon | Black cutworm |
| Striacosta albicosta | Western bean cutworm |
| Spodoptera ornithogalli | Yellowstriped armyworm |
| Spodoptera praefica | Western yellowstriped |
| | armyworm |
| Spodoptera eridania | Southern armyworm |
| Spodoptera eridania | Southern armyworm |
| Peridroma saucia | Variegated cutworm |
| Papaipema nebris | Stalk borer |
| Trichoplusia ni | Cabbage looper |
| Keiferia lycopersicella | Tomato pinworm |
| Manduca sexta | Tobacco hornworm |
| Manduca quinquemaculata | Tomato hornworm |
| Artogeia rapae | Imported cabbageworm |
| Pieris brassicae | Cabbage butterfly |
| Trichoplusia ni | Cabbage looper |
| Plutella xylostella | Diamondback moth |
| Spodoptera exigua | Beet armyworm |
| Agrotis segetum | Common cutworm |
| Phthorimaea operculella | Potato tuberworm |
| Plutella xylostella | Diamondback moth |
| Diatraea saccharalis | Sugarcane borer |
| Crymodes devastator | Glassy cutworm |
| Feltia ducens | Dingy cutworm |
| Agrotis gladiaria | Claybacked cutworm |
| Plathypena scabra | Green cloverworm |
| Pseudoplusia includes | Soybean looper |
| Anticarsia gemmatalis | Velvetbean caterpillar |
| Coleoptera Diabrotica barberi | Northern corn rootworm |
| Diabrotica undecimpunctata | Southern corn rootworm |
| Diabrotica virgifera | Western corn rootworm |
| Sitophilus zeamais | Maize weevil |
| Leptinotarsa decemlineata | Colorado potato beetle |
| Epitrix hirtipennis | Tobacco flea beetle |
| Phyllotreta cruciferae | Crucifer flea beetle |
| Phyllotreta pusilla | Western black flea beetle |
| Anthonomus eugenii | Pepper weevil |
| Leptinotarsa decemlineata | Colorado potato beetle |
| Epitrix cucumeris | Potato flea beetle |
| Hemicrepidus memnonius | Wireworms Melanpotus spp. |
| Ceutorhychus assimilis | Wireworms |
| Phyllotreta cruciferae | Cabbage seedpod weevil |
| Melanolus spp. | Crucifer flea beetle |
| Aeolus mellillus | Wireworm |
| Aeolus mancus | Wheat wireworm |
| Horistonotus uhlerii | Sand wireworm |
| Sphenophorus maidis | Maize billbug |
| Sphenophorus zeae | Timothy bilibug |
| Sphenophorus parvulus | Bluegrass billbug |
| Sphenophorus callosus | Southern corn billbug |
| Phyllophaga spp. | White grubs |
| Chaetocnema pulicaria | Corn flea beetle |
| Popillia japonica | Japanese beetle |
| Epilachna varivestis | Mexican bean beetle |
| Cerotoma trifurcate | Bean leaf beetle |
| Epicauta pestifera Epicauta lemniscata | Blister beetles |
| Homoptera Rhopalosiphum maidis | Corn leaf aphid |
| Anuraphis maidiradicis | Corn root aphid |
| Myzus persicae | Green peach aphid |
| Macrosiphum euphorbiae | Potato aphid |
| Trileurodes vaporariorum | Greenhouse whitefly |
| Bemisia tabaci | Sweetpotato whitefly |
| Bemisia argentifolii | Silverleaf whitefly |
| Brevicoryne brassicae | Cabbage aphid |
| Myzus persicae | Green peach aphid |
| Empoasca fabae | Potato leafhopper |
| Paratrioza cockerelli | Potato psyllid |
| Bemisia argentifolii | Silverleaf whitefly |
| Bemisia tabaci | Sweetpotato whitefly |
| Cavariella aegopodii | Carrot aphid |
| Brevicoryne brassicae | Cabbage aphid |
| Saccharosydne saccharivora | West Indian canefly |
| Sipha flava | Yellow sugarcane aphid |
| Spissistilus festinus | Threecornered alfalfa hopper |
| Hemiptera Lygus lineolaris | Lygus hesperus |
| Lygus rugulipennis | Lygus bug |
| Acrosternum hilare | Green stink bug |
| Euschistus servus | Brown stick bug |
| Blissus leucopterus leucopterus | Chinch bug |
| Diptera Liriomyza trifolii | Leafminer |
| Liriomyza sativae | Vegetable leafminer |
| Scrobipalpula absoluta | Tomato leafminer |
| Delia platura | Seedcorn maggot |
| Delia brassicae | Cabbage maggot |
| Delia radicum | Cabbage root fly |
| Psilia rosae | Carrot rust fly |
| Tetanops myopaeformis | Sugarbeet root maggot |
| Orthoptera Melanoplus differentialis | Differential grasshopper |
| Melanoplus femurrubrum | Redlegged grasshopper |
| Melanoplus bivittatus | Twostriped grasshopper |

Nematodes

Examples of nematodes that may be targeted with the presently disclosed formulations include, but are not limited to, those provided in Table 4.

TABLE 7

| Disease | Causative Agent |
|---|---|
| Awl | Dolichoderus spp., D. heterocephalus |
| Bulb and stem (Europe) | Ditylenchus dipsaci |
| Burrowing | Radopholus similes R. similis |
| Cyst | Heterodera avenae, H. zeae, H. schachti; Globodera rostochiensis, G. |
| | pallida, and G. tabacum; Heterodera trifolii, H. medicaginis, H. |
| | ciceri, H. mediterranea, H. cyperi, H. salixophila, H. zeae, H. |
| | goettingiana, H. riparia, H. humuli, H. latipons, H. sorghi, H. fici, H. |
| | litoralis, and H. turcomanica; Punctodera chalcoensis |
| Dagger | Xiphinema spp., X. americanum, X. Mediterraneum |
| False root-knot | Nacobbus dorsalis |
| Lance, Columbia | Hoplolaimus Columbus |
| Lance | Hoplolaimus spp., H. galeatus |
| Lesion | Pratylenchus spp., P. brachyurus, P. coffeae P. crenatus, P. |
| | hexincisus, P. neglectus, P. penetrans, P. scribneri, P. magnica, P. |
| | neglectus, P. thornei, P. vulnus, P. zeae |
| Needle | Longidorus spp., L. breviannulatus |
| Ring | Criconemella spp., C. ornata |
| Root-knot | Meloidogyne spp., M. arenaria, M. chitwoodi, M. artiellia, M. fallax, |
| | M. hapla, M. javanica, M. incognita, M. microtyla, M. partityla, M. |
| | panyuensis, M, paranaensis |
| Spiral | Helicotylenchus spp. |
| Sting | Belonolaimus spp., B. longicaudatus |
| Stubby-root | Paratrichodorus spp., P. christiei, P. minor, Quinisulcius acutus, |
| | Trichodorus spp. |
| Stunt | Tylenchorhynchus dubius |
| Others | Hirschmanniella species, Pratylenchoid magnicauda |

Fungi

Examples of fungi that may be targeted with the presently disclosed formulations include, but are not limited to, those provided in Table 5.

TABLE 8

| Disease | Causative Agent |
|---|---|
| Brown stripe downy mildew | Sclerophthora rayssiae var. zeae |
| Crazy top downy mildew | Sclerophthora macrospora = S. macrospora |
| Green ear downy mildew | Sclerospora graminicola |
| Java downy mildew | Peronosclerospora maydis = Sclerospora maydis |
| Philippine downy mildew | Peronosclerospora philippinensis = Sclerospora philippinensis |
| Sorghum downy mildew | Peronosclerospora sorghi = Sclerospora sorghi |
| Spontaneum downy mildew | Peronosclerospora spontanea = Sclerospora spontanea |
| Sugarcane downy mildew | Peronosclerospora sacchari = Sclerospora sacchari |
| Dry ear rot (cob, kernel and | Nigrospora oryzae (teleomorph: Khuskia oryzae) |
| stalk rot) | |
| Ear rots, minor | Aspergillus glaucus, A. niger, Aspergillus spp., Cunninghamella |
| | sp., Curvularia pallescens, Doratomyces stemonitis = |
| | Cephalotrichum stemonitis, Fusarium culmorum, Gonatobotrys |
| | simplex, Pithomyces maydicus, Rhizopus microsporus, R. stolonifer = |
| | R. nigricans, Scopulariopsis brumptii |
| Ergot (horse's tooth, diente | Claviceps gigantea (anamorph: Sphacelia sp.) |
| del caballo) | |
| Eyespot | Aureobasidium zeae = Kabatiella zeae |
| Fusarium ear and stalk rot | Fusarium subglutinans = F. moniliforme var. subglutinans |
| Fusarium kernel, root and | Fusarium moniliforme (teleomorph: Gibberella fujikuroi) |
| stalk rot, seed rot and | |
| seedling blight | |
| Fusarium stalk rot, seedling | Fusarium avenaceum (teleomorph: Gibberella avenacea) |
| root rot | |
| Gibberella ear and stalk rot | Gibberella zeae (anamorph: Fusarium graminearum) |
| Gray ear rot | Botryosphaeria zeae = Physalospora zeae (anamorph: |
| | Macrophoma zeae) |
| Gray leaf spot (Cercospora | Cercospora sorghi = C. sorghi var. maydis, C. zeae-maydis |
| leaf spot) | |
| Helminthosporium root rot | Exserohilum pedicellatum = Helminthosporium pedicellatum |
| | (teleomorph: Setosphaeria) |
| Hormodendrum ear rot | Cladosporium cladosporioides = Hormodendrum cladosporioides, |
| (Cladosporium rot) | C. herbarum (teleomorph: Mycosphaerella tassiana) |
| Hyalothyridium leaf spot | Hyalothyridium maydis |
| Late wilt | Cephalosporium maydis |
| Leaf spots, minor | Alternaria alternata, Ascochyta maydis, A. tritici, A. zeicola, |
| | Bipolaris victoriae = Helminthosporium victoriae (teleomorph: |
| | Cochliobolus victoriae), C. sativus (anamorph: Bipolaris |
| | sorokiniana = H. sorokinianum = H. sativum), Epicoccum nigrum, |
| | Exserohilum prolatum = Drechslera prolata (teleomorph: |
| | Setosphaeria prolata) Graphium penicillioides, Leptosphaeria |
| | maydis, Leptothyrium zeae, Ophiosphaerella herpotricha, |
| | (anamorph: Scolecosporiella sp.), Paraphaeosphaeria michotii, |
| | Phoma sp., Septoria zeae, S. zeicola, S. zeina |
| Northern corn leaf blight | Exaerohilum turcicum = Helminthosporium turcicum, Setosphaeria |
| | turcica |
| Northern corn leaf spot | Cochliobolus carbonum |
| Helminthosporium ear rot | Bipolaris zeicola = Helminthosporium carbonum |
| (race 1) | |
| Penicillium ear rot (blue | Penicillium spp., P. chrysogenum, P. expansum, P. oxalicum |
| eye, blue mold) | |
| Phaeocytostroma stalk rot | Phaeocytostroma ambiguum, Phaeocytosporella zeae |
| and root rot | |
| Phaeosphaeria leaf spot | Phaeosphaeria maydis, Sphaerulina maydis |
| Physalospora ear rot | Botryosphaeria Botryosphaeria festucae = Physalospora zeicola, |
| | (anamorph: Diplodia frumenti) |
| Purple leaf sheath | Hemiparasitic bacteria and fungi |
| Pyrenochaeta stalk rot and | Phoma terrestris, Pyrenochaeta terrestris |
| root rot | |
| Pythium root rot | Pythium spp., P. arrhenomanes, P. graminicola |
| Pythium stalk rot | Pythium aphanidermatum = P. butleri L. |
| Red kernel disease (ear | Epicoccum nigrum |
| mold, leaf and seed rot) | |
| Rhizoctonia ear rot | Rhizoctonia zeae (teleomorph: Waitea circinata) |
| Rhizoctonia root rot and | Rhizoctonia solani, Rhizoctonia zeae |
| stalk rot | |
| Root rots, minor | Alternaria alternata, Cercospora sorghi, Dictochaeta fertilis, |
| | Fusarium acuminatum (teleomorph: Gibberella acuminate), F. |
| | equiseti (teleomorph: G. intricans), F. oxysporum, F. |
| | pallidoroseum, F. poae, F. roseum, F. cyanogena, (anamorph: F. |
| | sulphureum), Microdochium bolleyi, Mucor sp., Periconia |
| | circinata, Phytophthora cactorum, P. drechsleri, P. nicotianae var. |
| | parasitica, Rhizopus arrhizus |
| Rostratum leaf spot (leaf | Setosphaeria rostrata, Helminthosporium (anamorph: Exserohilum |
| disease, ear and, stalk rot) | rostratum = Helminthosporium rostratum) |
| Rust, common corn | Puccinia sorghi |
| Rust, southern corn | Puccinia polysora |
| Rust, tropical corn | Physopella pallescens, P. zeae = Angiospora zeae |
| Sclerotium ear rot (southern | Sclerotium rolfsii (teleomorph: Athelia rolfsii) |
| blight) | |
| Seed rot-seedling blight | Bipolaris sorokiniana, B. zeicola = Helminthosporium carbonum, |
| | Diplodia maydis, Exserohilum pedicellatum, Exserohilum turcicum = |
| | Helminthosporium turcicum, Fusarium avenaceum, F. culmorum, |
| | F. moniliforme, Gibberella zeae (anamorph: F. graminearum), |
| | Macrophomina phaseolina, Penicillium spp., Phomopsis sp., |
| | Pythium spp., Rhizoctonia solani, R. zeae, Sclerotium rolfsii, |
| | Spicaria sp. |
| Selenophoma leaf spot | Selenophoma sp. |
| Sheath rot | Gaeumannomyces graminis |
| Shuck rot | Myrothecium gramineum |
| Silage mold | Monascus purpureus, M. rubber |
| Smut, common | Ustilago zeae = U. maydis |
| Smut, false | Ustilaginoidea virens |
| Smut, head | Sphacelotheca reiliana = Sporisorium holci-sorghi |
| Southern corn leaf blight | Cochliobolus heterostrophus (anamorph: Bipolaris maydis = |
| and stalk rot | Helminthosporium maydis) |
| Southern leaf spot | Stenocarpella macrospora = Diplodia macrospora |
| Stalk rots, minor | Cercospora sorghi, Fusarium episphaeria, F. merismoides, F. |
| | oxysportum, F. poae, F. roseum, F. solani (teleomorph: Nectria |
| | haematococca), F. tricinctum, Mariannaea elegans, Mucor sp., |
| | Rhopographus zeae, Spicaria sp. |
| Storage rots | Aspergillus spp., Penicillium spp. and other fungi |
| Tar spot | Phyllachora maydis |
| Trichoderma ear rot and | Trichoderma viride = T. lignorum (teleomorph: Hypocrea sp.) |
| root rot | |
| White ear rot, root and stalk | Stenocarpella maydis = Diplodia zeae |
| rot | |
| Yellow leaf blight | Ascochyta ischaemi, Phyllosticta maydis (teleomorph: |
| | Mycosphaerella zeae-maydis) |
| Zonate leaf spot | Gloeocercospora sorghi |
| Anthracnose leaf blight and | Colletotrichum graminicola anthracnose (teleomorph: |
| stalk rot | Glomerella graminicola), Glomerella tucumanensis (anamorph: |
| | Glomerella falcatum) |
| Aspergillus ear and kernel | Aspergillus flavus |
| rot | |
| Banded leaf and sheath spot | Rhizoctonia solani = Rhizoctonia microsclerotia (teleomorph: |
| | Thanatephorus cucumeris) |
| Black bundle disease | Acremonium strictum = Cephalosporium acremonium |
| Black kernel rot | Lasiodiplodia theobromae = Botryodiplodia theobromae |
| Borde blanco | Marasmiellus sp. |
| Brown spot (black spot, | Physoderma maydis |
| stalk rot) | |
| Cephalosporium kernel rot | Acremonium strictum = Cephalosporium acremonium |
| Charcoal rot | Macrophomina phaseolina |
| Corticium ear rot | Thanatephorus cucumeris = Corticium sasakii |
| Curvularia leaf spot | Curvularia clavata, C. eragrostidis, = C. maculans (teleomorph: |
| | Cochliobolus eragrostidis), Curvularia inaequalis, C. intermedia |
| | (teleomorph: Cochliobolus intermedius), Curvularia lunata |
| | (teleomorph: Cochliobolus lunatus), Curvularia pallescens |
| | (teleomorph: Cochliobolus pallescens), Curvularia senegalensis, C. |
| | tuberculata (teleomorph: Cochliobolus tuberculatus) |
| Didymella leaf spot | Didymella exitialis |
| Diplodia ear rot and stalk | Diplodia frumenti (teleomorph: Botryosphaeria festucae) |
| rot | |
| Diplodia ear rot, stalk rot, | Diplodia maydis = Stenocarpella maydis |
| seed rot and seedling blight | |
| Diplodia leaf spot or leaf | Stenocarpella macrospora = Diplodia macrospore |
| streak | |
| Corn common rust | Puccinia sorghi |
| Corn southern rust | Puccinia polysora |
| Corn tropical rust | Physopella pallescens, P. zeae = Angiospora zeae |
| Oat crown rust | Puccinia coronata |
| Oat stem Rust | Puccinia graminis |
| Stem rust | Puccinia graminis = P. graminis f. sp. secalis |
| Leaf (brown) rust | Puccinia recondita (anamorph: Aecidium clematitis) |
| Sugarcane common rust | Puccinia melanocephala = P. eriantha |
| Wheat leaf (brown) rust | Puccinia triticina = P. Recondita f. Sp. tritici = P. tritici-duri |
| Wheat stem (black) rust | Puccinia graminis = P. graminis f. sp. tritici |
| Wheat stripe (yellow) rust | Puccinia striiformis (anamorph: P. uredoglumarum) |
| Bean rust | Uromyces appendiculatus |
| Cotton rust | Puccinia schedonnardi |
| Cotton southwestern rust | Puccinia cacabata |
| Cotton tropical rust | Phakopsora gossypii |
| Peanut rust | Puccinia arachidis |
| Potato common rust | Puccinia pittierianap |
| Potato deforming rust | Aecidium cantensis |
| Soybean rust | Phakopsora pachyrhizi |

Bacteria

Examples of bacteria that may be targeted with the presently disclosed formulations include, but are not limited to, those shown in Table 6.

TABLE 9

| Disease | Causative Agent |
|---|---|
| Bacterial leaf blight and stalk rot | Pseudomonas avenae subsp. avenae |
| Bacterial leaf spot | Xanthomonas campestris pv. holcicola |
| Bacterial stalk rot | Enterobacter dissolvens = Erwinia dissolvens |
| Bacterial stalk and top rot | Erwinia carotovora subsp. carotovora, |
| | Erwinia chrysanthemi pv. Zeae |
| Bacterial stripe | Pseudomonas andropogonis |
| Chocolate spot | Pseudomonas syringae pv. Coronafaciens |
| Goss's bacterial wilt blight | Clavibacter michiganensis subsp. |
| (leaf freckles and wilt) | nebraskensis = Cornebacterium michiganense |
| | pv. Nebraskense |
| Holcus spot | Pseudomonas syringae pv. Syringae |
| Purple leaf sheath | Hemiparasitic bacteria |
| Seed rot-seedling blight | Bacillus subtilis |
| Stewart's disease (bacterial wilt) | Pantoea stewartii = Erwinia stewartii |
| Corn stunt (Mesa Central or | Achapparramiento, stunt, Spiroplasma |
| Rio Grande stunt) | kunkelii |

Viruses

Examples of plant viruses that may be targeted with the presently disclosed formulations include, but are not limited to, those shown in the Table 7.

TABLE 10

| | |
|---|---|
| Alfamoviruses: | Alfalfa mosaic alfamovirus |
| Bromoviridae | |
| Alphacryptoviruses: | Alfalfa 1 alphacryptovirus, Beet 1 alphacryptovirus, Beet 2 |
| Partitiviridae | alphacryptovirus, Beet 3 alphacryptovirus, Carnation 1 |
| | alphacryptovirus, Carrot temperate 1 alphacryptovirus, Carrot |
| | temperate 3 alphacryptovirus, Carrot temperate 4 alphacryptovirus, |
| | Cocksfoot alphacryptovirus, Hop trefoil 1 alphacryptovirus, Hop |
| | trefoil 3 alphacryptovirus, Radish yellow edge alphacryptovirus, |
| | Ryegrass alphacryptovirus, Spinach temperate alphacryptovirus, |
| | Vicia alphacryptovirus, White clover 1 alphacryptovirus, White |
| | clover 3 alphacryptovirus |
| Badnaviruses | Banana streak badnavirus, Cacao swollen shoot badnavirus, Canna |
| | yellow mottle badnavirus, Commelina yellow mottle badnavirus, |
| | Dioscorea bacilliform badnavirus, Kalanchoe top-spotting |
| | badnavirus, Rice tungro bacilliform badnavirus, Schefflera ringspot |
| | badnavirus, Sugarcane bacilliform badnavirus |
| Betacryptoviruses: | Carrot temperate 2 betacryptovirus, Hop trefoil 2 betacryptovirus, |
| Partitiviridae | Red clover 2 betacryptovirus, White clover 2 betacryptovirus |
| Bigeminiviruses: | Abutilon mosaic bigeminivirus, Ageratum yellow vein |
| Geminiviridae | bigeminivirus, Bean calico mosaic bigeminivirus, Bean golden |
| | mosaic bigeminivirus, Bhendi yellow vein mosaic bigeminivirus, |
| | Cassava African mosaic bigeminivirus, Cassava Indian mosaic |
| | bigeminivirus, Chino del tomate bigeminivirus, Cotton leaf crumple |
| | bigeminivirus, Cotton leaf curl bigeminivirus, Croton yellow vein |
| | mosaic bigeminivirus, Dolichos yellow mosaic bigeminivirus, |
| | Euphorbia mosaic bigeminivirus, Horsegram yellow mosaic |
| | bigeminivirus, Jatropha mosaic bigeminivirus, Lima bean golden |
| | mosaic bigeminivirus, Melon leaf curl bigeminivirus, Mung bean |
| | yellow mosaic bigeminivirus, Okra leaf-curl bigeminivirus, Pepper |
| | hausteco bigeminivirus, Pepper Texas bigeminivirus, Potato yellow |
| | mosaic bigeminivirus, Rhynchosia mosaic bigeminivirus, Serrano |
| | golden mosaic bigeminivirus, Squash leaf curl bigeminivirus, |
| | Tobacco leaf curl bigeminivirus, Tomato Australian leafcurl |
| | bigeminivirus, Tomato golden mosaic bigeminivirus, Tomato |
| | Indian leafcurl bigeminivirus, Tomato leaf crumple bigeminivirus, |
| | Tomato mottle bigeminivirus, Tomato yellow leaf curl |
| | bigeminivirus, Tomato yellow mosaic bigeminivirus, Watermelon |
| | chlorotic stunt bigeminivirus, Watermelon curly mottle |
| | bigeminivirus |
| Bromoviruses: | Broad bean mottle bromovirus, Brome mosaic bromovirus, Cassia |
| Bromoviridae | yellow blotch bromovirus, Cowpea chlorotic mottle bromovirus, |
| | Melandrium yellow fleck bromovirus, Spring beauty latent |
| | bromovirus |
| Bymoviruses: | Barley mild mosaic bymovirus, Barley yellow mosaic bymovirus, |
| Potyviridae | Oat mosaic bymovirus, Rice necrosis mosaic bymovirus, Wheat |
| | spindle streak mosaic bymovirus, Wheat yellow mosaic bymovirus |
| Capilloviruses | Apple stem grooving capillovirus, Cherry A capillovirus, Citrus |
| | tatter leaf capillovirus, Lilac chlorotic leafspot capillovirus |
| Carlaviruses | Blueberry scorch carlavirus, Cactus 2 carlavirus, Caper latent |
| | carlavirus, Carnation latent carlavirus, Chrysanthemum B |
| | carlavirus, Dandelion latent carlavirus, Elderberry carlavirus, Fig S |
| | carlavirus, Helenium S carlavirus, Honeysuckle latent carlavirus, |
| | Hop American latent carlavirus, Hop latent carlavirus, Hop mosaic |
| | carlavirus, Kalanchoe latent carlavirus, Lilac mottle carlavirus, Lily |
| | symptomless carlavirus, Mulberry latent carlavirus, Muskmelon |
| | vein necrosis carlavirus, Nerine latent carlavirus, Passiflora latent |
| | carlavirus, Pea streak carlavirus, Poplar mosaic carlavirus, Potato M |
| | carlavirus, Potato S carlavirus, Red clover vein mosaic carlavirus, |
| | Shallot latent carlavirus, Strawberry pseudo mild yellow edge |
| | carlavirus |
| Carmoviruses: | Bean mild mosaic carmovirus, Cardamine chlorotic fleck |
| Tombusviridae | carmovirus, Carnation mottle carmovirus, Cucumber leaf spot |
| | carmovirus, Cucumber soil-borne carmovirus, Galinsoga mosaic |
| | carmovirus, Hibiscus chlorotic ringspot carmovirus, Melon necrotic |
| | spot carmovirus, Pelargonium flower break carmovirus, Turnip |
| | crinkle carmovirus |
| Caulimoviruses | Blueberry red ringspot caulimovirus, Carnation etched ring |
| | caulimovirus, Cauliflower mosaic caulimovirus, Dahlia mosaic |
| | caulimovirus, Figwort mosaic caulimovirus, Horseradish latent |
| | caulimovirus, Mirabilis mosaic caulimovirus, Peanut chlorotic |
| | streak caulimovirus, Soybean chlorotic mottle caulimovirus, Sweet |
| | potato caulimovirus, Thistle mottle caulimovirus |
| Closteroviruses | Beet yellow stunt closterovirus, Beet yellows closterovirus, Broad |
| | bean severe chlorosis closterovirus, Burdock yellows closterovirus, |
| | Carnation necrotic fleck closterovirus, Citrus tristeza closterovirus, |
| | Clover yellows closterovirus, Grapevine stem pitting associated |
| | closterovirus, Wheat yellow leaf closterovirus |
| Comoviruses: | Bean pod mottle comovirus, Bean rugose mosaic comovirus, Broad |
| Comoviridae | bean stain comovirus, Broad bean true mosaic comovirus, Cowpea |
| | mosaic comovirus, Cowpea severe mosaic comovirus, Glycine |
| | mosaic comovirus, Pea mild mosaic comovirus, Potato Andean |
| | mottle comovirus, Quail pea mosaic comovirus, Radish mosaic |
| | comovirus, Red clover mottle comovirus, Squash mosaic |
| | comovirus, Ullucus C comovirus |
| Cucumoviruses: | Cucumber mosaic cucumovirus, Peanut stunt cucumovirus, Tomato |
| Bromoviridae | aspermy cucumovirus |
| Cytorhabdoviruses: | Barley yellow striate mosaic cytorhabdovirus, Broad bean yellow |
| Rhabdoviridae | vein cytorhabdovirus, Broccoli necrotic yellows cytorhabdovirus, |
| | Cereal northern mosaic cytorhabdovirus, Festuca leaf streak |
| | cytorhabdovirus, Lettuce necrotic yellows cytorhabdovirus, |
| | Sonchus cytorhabdovirus, Strawberry crinkle cytorhabdovirus |
| Dianthoviruses | Carnation ringspot dianthovirus, Red clover necrotic mosaic |
| | dianthovirus, Sweet clover necrotic mosaic dianthovirus |
| Enamoviruses | Pea enation mosaic enamovirus |
| Fijiviruses: | Maize rough dwarf fijivirus, Oat sterile dwarf fijivirus, Pangola |
| Reoviridae | stunt fijivirus, Rice black-streaked dwarf fijivirus, Sugarcane Fiji |
| | disease fijivirus |
| Furoviruses | Beet necrotic yellow vein furovirus, Beet soil-borne furovirus, |
| | Broad bean necrosis furovirus, Oat golden stripe furovirus, Peanut |
| | clump furovirus, Potato mop-top furovirus, Sorghum chlorotic spot |
| | furovirus, Wheat soil-borne mosaic furovirus |
| Hordeiviruses | Anthoxanthum latent blanching hordeivirus, Barley stripe mosaic |
| | hordeivirus, Lychnis ringspot hordeivirus, Poa semilatent |
| | hordeivirus |
| Hybrigeminiviruses: | Beet curly top hybrigeminivirus, Tomato pseudo curly top |
| Geminiviridae | hybrigeminivirus |
| Idaeoviruses | Raspberry bushy dwarf idaeovirus |
| Ilarviruses: | Apple mosaic ilarvirus, Asparagus 2 ilarvirus, Blueberry necrotic |
| Bromoviridae | shock ilarvirus, Citrus leaf rugose ilarvirus, Citrus variegation |
| | ilarvirus, Elm mottle ilarvirus, Humulus japonicus ilarvirus, |
| | Hydrangea mosaic ilarvirus, Lilac ring mottle ilarvirus, Parietaria |
| | mottle ilarvirus, Plum American line pattern ilarvirus, Prune dwarf |
| | ilarvirus, Prunus necrotic ringspot ilarvirus, Spinach latent ilarvirus, |
| | Tobacco streak ilarvirus, Tulare apple mosaic ilarvirus |
| Ipomoviruses: | Sweet potato mild mottle ipomovirus, Sweet potato yellow dwarf |
| Potyviridae | ipomovirus |
| Luteoviruses | Barley yellow dwarf luteovirus, Bean leaf roll luteovirus, Beet mild |
| | yellowing luteovirus, Beet western yellows luteovirus, Carrot red |
| | leaf luteovirus, Groundnut rosette assistor luteovirus, Potato leafroll |
| | luteovirus, Solanum yellows luteovirus, Soybean dwarf luteovirus, |
| | Soybean Indonesian dwarf luteovirus, Strawberry mild yellow edge |
| | luteovirus, Subterranean clover red leaf luteovirus, Tobacco |
| | necrotic dwarf luteovirus |
| Machlomoviruses | Maize chlorotic mottle machlomovirus |
| Macluraviruses | Maclura mosaic macluravirus, Narcissus latent macluravirus |
| Marafiviruses | Bermuda grass etched-line marafivirus, Maize rayado fino |
| | marafivirus, Oat blue dwarf marafivirus |
| Monogeminiviruses: | Chloris striate mosaic monogeminivirus, Digitaria striate mosaic |
| Geminiviridae | monogeminivirus, Digitaria streak monogeminivirus, Maize streak |
| | monogeminivirus, Miscanthus streak monogeminivirus, Panicum |
| | streak monogeminivirus, Paspalum striate mosaic |
| | monogeminivirus, Sugarcane streak monogeminivirus, Tobacco |
| | yellow dwarf monogeminivirus, Wheat dwarf monogeminivirus |
| Nanaviruses | Banana bunchy top nanavirus, Coconut foliar decay nanavirus, |
| | Faba bean necrotic yellows nanavirus, Milk vetch dwarf nanavirus, |
| | Subterranean clover stunt nanavirus |
| Necroviruses | Tobacco necrosis necrovirus, Carnation yellow stripe necrovirus, |
| | Lisianthus necrosis necrovirus |
| Nepoviruses: | Arabis mosaic nepovirus, Arracacha A nepovirus, Artichoke Italian |
| Comoviridae | latent nepovirus, Artichoke yellow ringspot nepovirus, Blueberry |
| | leaf mottle nepovirus, Cacao necrosis nepovirus, Cassava green |
| | mottle nepovirus, Cherry leaf roll nepovirus, Cherry rasp leaf |
| | nepovirus, Chicory yellow mottle nepovirus, Crimson clover latent |
| | nepovirus, Cycas necrotic stunt nepovirus, Grapevine Bulgarian |
| | latent nepovirus, Grapevine chrome mosaic nepovirus, Grapevine |
| | fanleaf nepovirus, Hibiscus latent ringspot nepovirus, Lucerne |
| | Australian latent nepovirus, Mulberry ringspot nepovirus, |
| | Myrobalan latent ringspot nepovirus, Olive latent ringspot |
| | nepovirus, Peach rosette mosaic nepovirus, Potato black ringspot |
| | nepovirus, Potato U nepovirus, Raspberry ringspot nepovirus, |
| | Tobacco ringspot nepovirus, Tomato black ring nepovirus, Tomato |
| | ringspot nepovirus |
| Nucleorhabdoviruses: | Carrot latent nucleorhabdovirus, Coriander feathery red vein |
| Rhabdoviridae | nucleorhabdovirus, Cow parsnip mosaic nucleorhabdovirus, |
| | Cynodon chlorotic streak nucleorhabdovirus, Datura yellow vein |
| | nucleorhabdovirus, Eggplant mottled dwarf nucleorhabdovirus, |
| | Maize mosaic nucleorhabdovirus, Pittosporum vein yellowing |
| | nucleorhabdovirus, Potato yellow dwarf nucleorhabdovirus, |
| | Sonchus yellow net nucleorhabdovirus, Sowthistle yellow vein |
| | nucleorhabdovirus, Tomato vein clearing nucleorhabdovirus, Wheat |
| | American striate mosaic nucleorhabdovirus |
| Oryzaviruses: | Echinochloa ragged stunt oryzavirus, Rice ragged stunt oryzavirus |
| Reoviridae | |
| Ourmiaviruses | Cassava Ivorian bacilliform ourmiavirus, Epirus cherry |
| | ourmiavirus, Melon Ourmia ourmiavirus, Pelargonium zonate spot |
| | ourmiavirus |
| Phytoreoviruses: | Clover wound tumor phytoreovirus, Rice dwarf phytoreovirus, Rice |
| Reoviridae | gall dwarf phytoreovirus, Rice bunchy stunt phytoreovirus, Sweet |
| | potato phytoreovirus |
| Potexviruses | Asparagus 3 potexvirus, Cactus X potexvirus, Cassava X |
| | potexvirus, Chicory X potexvirus, Clover yellow mosaic |
| | potexvirus, Commelina X potexvirus, Cymbidium mosaic |
| | potexvirus, Daphne X potexvirus, Foxtail mosaic potexvirus, |
| | Hydrangea ringspot potexvirus, Lily X potexvirus, Narcissus |
| | mosaic potexvirus, Nerine X potexvirus, Papaya mosaic potexvirus, |
| | Pepino mosaic potexvirus, Plantago asiatica mosaic potexvirus, |
| | Plantain X potexvirus, Potato aucuba mosaic potexvirus, Potato X |
| | potexvirus, Tulip X potexvirus, Viola mottle potexvirus, White |
| | clover mosaic potexvirus |
| Potyviruses: | Alstroemeria mosaic potyvirus, Amaranthus leaf mottle potyvirus, |
| Potyviridae | Araujia mosaic potyvirus, Arracacha Y potyvirus, Artichoke latent |
| | potyvirus, Asparagus 1 potyvirus, Banana bract mosaic potyvirus, |
| | Bean common mosaic necrosis potyvirus, Bean common mosaic |
| | potyvirus, Bean yellow mosaic potyvirus, Beet mosaic potyvirus, |
| | Bidens mosaic potyvirus, Bidens mottle potyvirus, Cardamom |
| | mosaic potyvirus, Carnation vein mottle potyvirus, Carrot thin leaf |
| | potyyirus, Cassava brown streak potyvirus, Cassia yellow spot |
| | potyvirus, Celery mosaic potyvirus, Chickpea bushy dwarf |
| | potyvirus, Chickpea distortion mosaic potyvirus, Clover yellow |
| | vein potyvirus, Commelina diffusa potyvirus, Commelina mosaic |
| | potyvirus, Cowpea green vein-banding potyvirus, Cowpea |
| | Moroccan aphid-borne mosaic potyvirus, Cowpea rugose mosaic |
| | potyvirus, Crinum mosaic potyvirus, Daphne Y potyvirus, Dasheen |
| | mosaic potyvirus, Datura Colombian potyvirus, Datura distortion |
| | mosaic potyvirus, Datura necrosis potyvirus, Datura shoestring |
| | potyvirus, Dendrobium mosaic potyvirus, Desmodium mosaic |
| | potyvirus, Dioscorea alata potyvirus, Dioscorea green banding |
| | mosaic potyvirus, Eggplant green mosaic potyvirus, Euphorbia |
| | ringspot potyvirus, Freesia mosaic potyvirus, Groundnut eyespot |
| | potyvirus, Guar symptomless potyvirus, Guinea grass mosaic |
| | potyvirus, Helenium Y potyvirus, Henbane mosaic potyvirus, |
| | Hippeastrum mosaic potyvirus, Hyacinth mosaic potyvirus, Iris |
| | fulva mosaic potyvirus, Iris mild mosaic potyvirus, Iris severe |
| | mosaic potyvirus, Johnsongrass mosaic potyvirus, Kennedya Y |
| | potyvirus, Leek yellow stripe potyvirus, Lettuce mosaic potyvirus, |
| | Lily mottle potyvirus, Maize dwarf mosaic potyvirus, Malva vein |
| | clearing potyvirus, Marigold mottle potyvirus, Narcissus yellow |
| | stripe potyvirus, Nerine potyvirus, Onion yellow dwarf potyvirus, |
| | Ornithogalum mosaic potyvirus, Papaya ringspot potyvirus, Parsnip |
| | mosaic potyvirus, Passiflora ringspot potyvirus, Passiflora South |
| | African potyvirus, Passionfruit woodiness potyvirus, Patchouli |
| | mosaic potyvirus, Pea mosaic potyvirus, Pea seed-borne mosaic |
| | potyvirus, Peanut green mosaic potyvirus, Peanut mottle potyvirus, |
| | Pepper Indian mottle potyvirus, Pepper mottle potyvirus, Pepper |
| | severe mosaic potyvirus, Pepper veinal mottle potyvirus, Plum pox |
| | potyvirus, Pokeweed mosaic potyvirus, Potato A potyvirus, Potato |
| | V potyvirus, Potato Y potyvirus, Primula mosaic potyvirus, |
| | Ranunculus mottle potyvirus, Sorghum mosaic potyvirus, Soybean |
| | mosaic potyvirus, Statice Y potyvirus, Sugarcane mosaic potyvirus, |
| | Sweet potato feathery mottle potyvirus, Sweet potato G potyvirus, |
| | Swordbean distortion mosaic potyvirus, Tamarillo mosaic |
| | potyvirus, Telfairia mosaic potyvirus, Tobacco etch potyvirus, |
| | Tobacco vein-banding mosaic potyvirus, Tobacco vein mottling |
| | potyvirus, Tobacco wilt potyvirus, Tomato Peru potyvirus, |
| | Tradescantia-Zebrina potyvirus, Tropaeolum 1 potyvirus, |
| | Tropaeolum 2 potyvirus, Tuberose potyvirus, Tulip band-breaking |
| | potyvirus, Tulip breaking potyvirus, Tulip chlorotic blotch |
| | potyvirus, Turnip mosaic potyvirus, Ullucus mosaic potyvirus, |
| | Vallota mosaic potyvirus, Vanilla mosaic potyvirus, Vanilla |
| | necrosis potyvirus, Voandzeia distortion mosaic potyvirus, |
| | Watermelon mosaic 1 potyvirus, Watermelon mosaic 2 potyvirus, |
| | Wild potato mosaic potyvirus, Wisteria vein mosaic potyvirus, Yam |
| | mosaic potyvirus, Zucchini yellow fleck potyvirus, Zucchini yellow |
| | mosaic potyvirus |
| Rymoviruses: | Hordeum mosaic rymovirus, Oat necrotic mottle |
| Potyviridae | |
| Agropyron mosaic | |
| rymovirus | rymovirus, Ryegrass mosaic rymovirus, Wheat streak mosaic |
| | rymovirus |
| Satellite RNAs | Arabis mosaic satellite RNA, Chicory yellow mottle satellite RNA, |
| | Cucumber mosaic satellite RNA, Grapevine fanleaf satellite RNA, |
| | Strawberry latent ringspot satellite RNA, Tobacco ringspot satellite |
| | RNA, Tomato black ring satellite RNA, Velvet tobacco mottle |
| | satellite RNA |
| Satelliviruses | Maize white line mosaic satellivirus, Panicum mosaic satellivirus, |
| | Tobacco mosaic satellivirus, Tobacco necrosis satellivirus |
| Sequiviruses: | Dandelion yellow mosaic sequivirus, Parsnip yellow fleck |
| Sequiviridae | sequivirus |
| Sobemoviruses | Bean southern mosaic sobemovirus, Blueberry shoestring |
| | sobemovirus, Cocksfoot mottle sobemovirus, Lucerne transient |
| | streak sobemovirus, Rice yellow mottle sobemovirus, Rottboellia |
| | yellow mottle sobemovirus, Solanum nodiflorum mottle |
| | sobemovirus, Sowbane mosaic sobemovirus, Subterranean clover |
| | mottle sobemovirus, Turnip rosette sobemovirus, Velvet tobacco |
| | mottle, sobemovirus |
| Tenuiviruses | Maize stripe tenuivirus, Rice grassy stunt tenuivirus, Rice hoja |
| | blanca tenuivirus, Rice stripe tenuivirus |
| Tobamoviruses | Cucumber green mottle mosaic tobamovirus, Frangipani mosaic |
| | tobamovirus, Kyuri green mottle mosaic tobamovirus, |
| | Odontoglossum ringspot tobamovirus, Paprika mild mottle |
| | tobamovirus, Pepper mild mottle tobamovirus, Ribgrass mosaic |
| | tobamovirus, Opuntia Sammons' tobamovirus, Sunn-hemp mosaic |
| | tobamovirus, Tobacco mild green mosaic tobamovirus, Tobacco |
| | mosaic tobamovirus, Tomato mosaic tobamovirus, Ullucus mild |
| | mottle tobamovirus |
| Tobraviruses | Pea early browning tobravirus, Pepper ringspot tobravirus, Tobacco |
| | rattle tobravirus |
| Tombusviruses: | Artichoke mottled crinkle tombusvirus, Carnation Italian ringspot |
| Tombusviridae | tombusvirus, Cucumber necrosis tombusvirus, Cymbidium ringspot |
| | tombusvirus, Eggplant mottled crinkle tombusvirus, Grapevine |
| | Algerian latent tombusvirus, Lato River tombusvirus, Neckar River |
| | tombusvirus, Pelargonium leaf curl tombusvirus, Pepper Moroccan |
| | tombusvirus, Petunia asteroid mosaic tombusvirus, Tomato bushy |
| | stunt tombusvirus |
| Tospoviruses: | Impatiens necrotic spot tospovirus, Peanut yellow spot tospovirus, |
| Bunyaviridae | Tomato spotted wilt tospovirus |
| Trichoviruses | Apple chlorotic leaf spot trichovirus, Heracleum latent trichovirus, |
| | Potato T trichovirus |
| Tymoviruses | Abelia latent tymovirus, Belladonna mottle tymovirus, Cacao |
| | yellow mosaic tymovirus, Clitoria yellow vein tymovirus, |
| | Desmodium yellow mottle tymovirus, Dulcamara mottle tymovirus, |
| | Eggplant mosaic tymovirus, Erysimum latent tymovirus, Kennedya |
| | yellow mosaic tymovirus, Melon rugose mosaic tymovirus, Okra |
| | mosaic tymovirus, Ononis yellow mosaic tymovirus, Passionfruit |
| | yellow mosaic tymovirus, Physalis mosaic tymovirus, Plantago |
| | mottle tymovirus, Potato Andean latent tymovirus, Scrophularia |
| | mottle tymovirus, Turnip yellow mosaic, tymovirus, Voandzeia |
| | necrotic mosaic tymovirus, Wild cucumber mosaic tymovirus |
| Umbraviruses | Bean yellow vein banding umbravirus, Carrot mottle mimic |
| | umbravirus, Carrot mottle umbravirus, Carrot mottle mimic |
| | umbravirus, Groundnut rosette umbravirus, Lettuce speckles mottle |
| | umbravirus, Tobacco mottle umbravirus |
| Varicosaviruses | Freesia leaf necrosis varicosavirus, Lettuce big-vein varicosavirus, |
| | Tobacco stunt varicosavirus |
| Waikaviruses: | Anthriscus yellows waikavirus, Maize chlorotic dwarf waikavirus, |
| Sequiviridae | Rice tungro spherical waikavirus |
| Putative | Alsike clover vein mosaic virus, Alstroemeria streak potyvirus, |
| Ungrouped | Amaranthus mosaic potyvirus, Amazon lily mosaic potyvirus, |
| Viruses | Anthoxanthum mosaic potyvirus, Apple stem pitting virus, |
| | Aquilegia potyvirus, Asclepias rhabdovirus, Atropa belladonna |
| | rhabdovirus, Barley mosaic virus, Barley yellow streak mosaic |
| | virus, Beet distortion mosaic virus, Beet leaf curl rhabdovirus, Beet |
| | western yellows ST9-associated RNA virus, Black raspberry |
| | necrosis virus, Bramble yellow mosaic potyvirus, Brinjal mild |
| | mosaic potyvirus, Broad bean B virus, Broad bean V potyvirus, |
| | Broad bean yellow ringspot virus, Bryonia mottle potyvirus, |
| | Burdock mosaic virus, Burdock mottle virus, Callistephus chinensis |
| | chlorosis rhabdovirus, Canary reed mosaic potyvirus, Canavalia |
| | maritima mosaic potyvirus, Carnation rhabdovirus, Carrot mosaic |
| | potyvirus, Cassava symptomless rhabdovirus, Cassia mosaic virus, |
| | Cassia ringspot virus, Celery yellow mosaic potyvirus, Celery |
| | yellow net virus, Cereal flame chlorosis virus, Chickpea filiform |
| | potyvirus, Chilli veinal mottle potyvirus, Chrysanthemum spot |
| | potyvirus, Chrysanthemum vein chlorosis rhabdovirus, Citrus |
| | leprosis rhabdovirus, Citrus ringspot virus, Clover mild mosaic |
| | virus, Cocksfoot streak potyvirus, Colocasia bobone disease |
| | rhabdovirus, Cucumber toad-skin rhabdovirus, Cucumber vein |
| | yellowing virus, Cypripedium calceolus potyvirus, Datura innoxia |
| | Hungarian mosaic potyvirus, Dioscorea trifida potyvirus, Dock |
| | mottling mosaic potyvirus, Dodonaea yellows-associated virus, |
| | Eggplant severe mottle potyvirus, Euonymus fasciation |
| | rhabdovirus, Euonymus rhabdovirus, Fern potyvirus, Fig potyvirus, |
| | Gerbera symptomless rhabdovirus, Grapevine fleck virus, |
| | Grapevine stunt virus, Guar top necrosis virus, Habenaria mosaic |
| | potyvirus, Holcus lanatus yellowing rhabdovirus, Holcus streak |
| | potyvirus, Iris germanica leaf stripe rhabdovirus, Iris Japanese |
| | necrotic ring virus, Isachne mosaic potyvirus, Kalanchoe isometric |
| | virus, Kenaf vein-clearing rhabdovirus, Launaea mosaic potyvirus, |
| | Lupin yellow vein rhabdovirus, Maize eyespot virus, Maize line |
| | virus, Maize mottle/chlorotic stunt virus, Maize white line mosaic |
| | virus, Malvastrum mottle virus, Melilotus mosaic potyvirus, Melon |
| | vein-banding mosaic potyvirus, Melothria mottle potyvirus, |
| | Mimosa mosaic virus, Mung bean mottle potyvirus, Narcissus |
| | degeneration potyvirus, Narcissus late season yellows potyvirus, |
| | Nerine Y potyvirus, Nothoscordum mosaic potyvirus, Oak ringspot |
| | virus, Orchid fleck rhabdovirus, Palm mosaic potyvirus, Parsley |
| | green mottle potyvirus, Parsley rhabdovirus, Parsnip leafcurl virus, |
| | Passionfruit Sri Lankan mottle potyvirus, Passionfruit vein-clearing |
| | rhabdovirus, Patchouli mottle rhabdovirus, Pea stem necrosis virus, |
| | Peanut top paralysis potyvirus, Peanut veinal chlorosis rhabdovirus, |
| | Pecteilis mosaic potyvirus, Pepper mild mosaic potyvirus, Perilla |
| | mottle potyvirus, Pigeonpea proliferation rhabdovirus, Pigeonpea |
| | sterility mosaic virus, Plantain 7 potyvirus, Plantain mottle |
| | rhabdovirus, Pleioblastus chino potyvirus, Poplar decline potyvirus, |
| | Primula mottle potyvirus, Purple granadilla mosaic virus, |
| | Ranunculus repens symptomless rhabdovirus, Rice yellow stunt |
| | virus, Saintpaulia leaf necrosis rhabdovirus, Sambucus vein |
| | clearing rhabdovirus, Sarracenia purpurea rhabdovirus, Shamrock |
| | chlorotic ringspot potyvirus, Soybean mild mosaic virus, Soybean |
| | rhabdovirus, Soybean spherical virus, Soybean yellow vein virus, |
| | Soybean Z potyvirus, Strawberry latent C rhabdovirus, Strawberry |
| | mottle virus, Strawberry pallidosis virus, Sunflower mosaic |
| | potyvirus, Sweet potato latent potyvirus, Teasel mosaic potyvirus, |
| | Thimbleberry ringspot virus, Tomato mild mottle potyvirus, |
| | Trichosanthes mottle potyvirus, Tulip halo necrosis virus, Tulip |
| | mosaic virus, Turnip vein-clearing virus, Urd bean leaf crinkle |
| | virus, Vigna sinensis mosaic rhabdovirus, Watercress yellow spot |
| | virus, Watermelon Moroccan mosaic potyvirus, Wheat chlorotic |
| | spot rhabdovirus, White bryony potyvirus, Wineberry latent virus, |
| | Zinnia mild mottle potyvirus, Zoysia mosaic potyvirus |

Weeds

In certain embodiments, the target organism is a weed. As used herein, the term “weed” refers to any unwanted plant. The weed to be controlled may include monocotyledonous species, such as species of the genus Agrostis, Alopecurus, Avena, Bromus, Cyperus, Digitaria, Echinochloa, Lolium, Monochoria, Rottboellia, Sagittaria, Scirpus, Setaria, Sida or Sorghum, and dicotyledonous species, for example species of the genus Abutilon, Amaranthus, Chenopodium, Chrysanthemum, Conyza, Galium, Ipomoea, Nasturtium, Sinapis, Solanum, Stellaria, Veronica, Viola or Xanthium. Weeds can also include plants which may be considered crop plants but which are growing outside a crop area (escapes), or which grow from seed left over from a previous planting of a different crop (volunteers). Such volunteers or escapes may be tolerant to certain other herbicides.

It has been demonstrated that several agriculturally relevant traits in plants can be modified via the introduction of transgenes that target the silencing of specific genes, including soybean oil composition and corn kernel protein composition. dsRNAs targeting specific genes in specific species can be applied topically to alter plant traits as well, and in some cases, offers the farmer more flexibility with regard to timing and endurance of application. In certain embodiments, the presently disclosed formulations may be used to enhance a yield-related trait in a plant. Yield-related traits that may be enhanced by the presently disclosed formulations include, but are not limited to, total seed germination, rate of seed germination, plant biomass, disease tolerance, insect tolerance, drought tolerance, heat tolerance, cold tolerance, salinity tolerance, tolerance to heavy metals, total yield, seed yield, root growth, early vigor, plant biomass, plant size, total plant dry weight, above-ground dry weight, above-ground fresh weight, leaf area, stem volume, plant height, rosette diameter, leaf length, root length, root mass, tiller number, and leaf number.

Crop Plants

In certain embodiments, the target organism is a crop plant. Examples of crop plants that may be target organisms include, but are not limited to, monocotyledonous and dicotyledonous plants including but not limited to fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. [canola, oilseed rape, turnip rape]), Camellia sinensis, Canna indica, Cannabis saliva, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morus nigra, Musa spp., Nicotiana spp., Olea spp., Oryza spp. (e.g., Oryza sativa, Oryza latifolia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solanum integrifolium or Solanum lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. Especially preferred are rice, oilseed rape, canola, soybean, corn (maize), cotton, sugarcane, alfalfa, sorghum, and wheat.

Non-Target Organisms

In certain embodiments, the presently disclosed formulations may be applied to an organism that is different from the target organism. For example, in certain embodiments the target organism is an insect, and the composition is applied to a non-target organism, such as a plant, that is a host for the insect. As used herein, a “non-target organism” is any organism other than the target organism. Where the target organism and host organism differ, a non-target organism can comprise a host organism and organisms that consume the host organism or otherwise contact polynucleotides (e.g., siRNAs or antisense polynucleotides) or proteins expressed in a host organism. The target-specific design of polynucleotides such as RNAi and antisense polynucleotides, as described herein, provides that such polynucleotides have little or no gene silencing activity in non-target organisms.

In certain embodiments, non-target organisms include crop plants that may be infected with a target organism, such as a plant pathogen or insect. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. [canola, oilseed rape, turnip rape]), Camellia sinensis, Canna indica, Cannabis saliva, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morus nigra, Musa spp., Nicotiana spp., Olea spp., Oryza spp. (e.g., Oryza sativa, Oryza latifolia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solanum integrifolium or Solanum lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat.

Application of the Formulations

In certain embodiments, the presently disclosed formulations can be applied as a spray or powder to the plant, plant part, seed, a pest, or an area of cultivation. The presently disclosed formulations may also be applied as concentrated emulsions, dusts, emulsifiable concentrates, fumigants, gels, granules, seed treatments, suspension concentrates, suspoemulsions, tablets, water soluble liquids, water dispersible granules or dry flowables, wettable powders, and ultra-low volume solutions. For further information on formulation types see “Catalogue of Pesticide Formulation Types and International Coding System” Technical Monograph No. 2, 5th Edition by CropLife International (2002), which is incorporated herein by reference in its entirety. Agricultural formulations are also described, for example, in U.S. Pat. No. 8,815,271, which is incorporated herein by reference in its entirety.

For example, the presently disclosed formulations may be applied as aqueous suspensions or emulsions prepared from concentrated formulations. Such water-soluble, water-suspendable, or emulsifiable formulations can either be solids, usually known as wettable powders, or water dispersible granules, or liquids usually known as emulsifiable concentrates, or aqueous suspensions. Wettable powders, which may be compacted to form water dispersible granules, comprise an intimate mixture of the composition, a carrier, and surfactants. The carrier may be selected from attapulgite clays, montmorillonite clays, diatomaceous earths, and purified silicates. Effective surfactants, comprising from about 0.5% to about 10% of the wettable powder, include sulfonated lignins, condensed naphthalenesulfonates, naphthalenesulfonates, alkylbenzenesulfonates, alkyl sulfates, and non-ionic surfactants such as ethylene oxide adducts of alkyl phenols.

Emulsifiable concentrates can comprise a suitable concentration of the presently disclosed formulation, such as from about 50 to about 500 grams per liter of liquid dissolved in a carrier that is either a water-miscible solvent or a mixture of water-immiscible organic solvent and emulsifiers. Suitable organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates can be selected from conventional anionic and non-ionic surfactants.

Aqueous suspensions comprise suspensions of water-insoluble forms of the presently disclosed formulations dispersed in an aqueous carrier at a concentration in the range from about 5% to about 50% by weight. Ingredients, such as inorganic salts and synthetic or natural gums, may also be added to increase the density and viscosity of the aqueous carrier.

The presently disclosed formulations may also be applied as granular formulations, for example, for applications to the soil. Granular formulations may contain from about 0.5% to about 10% by weight of the composition, dispersed in a carrier that comprises clay or a similar substance. Such formulations may be prepared by dissolving the formulation in a suitable solvent and applying it to a granular carrier which has been pre-formed to a suitable particle size, for example, in the range of from about 0.5 to about 3 mm. Such formulations may also be prepared by making a dough or paste of the carrier and compound and crushing and drying to obtain the desired granular particle size.

Dusts comprising the presently disclosed formulations may be prepared by intimately mixing the formulation in powdered form with a suitable dusty agricultural carrier, such as kaolin clay, ground volcanic rock, and the like. Dusts may contain from about 1% to about 10% by weight of the formulation. They may be applied as a seed dressing or as a foliage application with a dust blower machine.

The presently disclosed formulations may also be applied in the form of a solution in an appropriate organic solvent (e.g., petroleum oil) such as the spray oils, which are widely used in agricultural chemistry.

The presently disclosed formulations may also be applied in the form of an aerosol composition. The formulation can be dissolved or dispersed in a carrier, which is a pressure-generating propellant mixture. The aerosol composition is packaged in a container from which the mixture is dispensed through an atomizing valve.

The presently disclosed formulations may be applied to the crop area or plant to be treated, simultaneously or in succession with further compounds. These further compounds can be, for example, fertilizers or micronutrient donors or other preparations, which influence the growth of plants. They can also be selective herbicides or non-selective herbicides as well as insecticides, fungicides, bactericides, nematicides, viricides, or mixtures of several of these preparations, if desired together with further carriers, surfactants or application promoting adjuvants customarily employed in the art of formulation.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Examples

### Example 1—Synthesis of Intermediate A

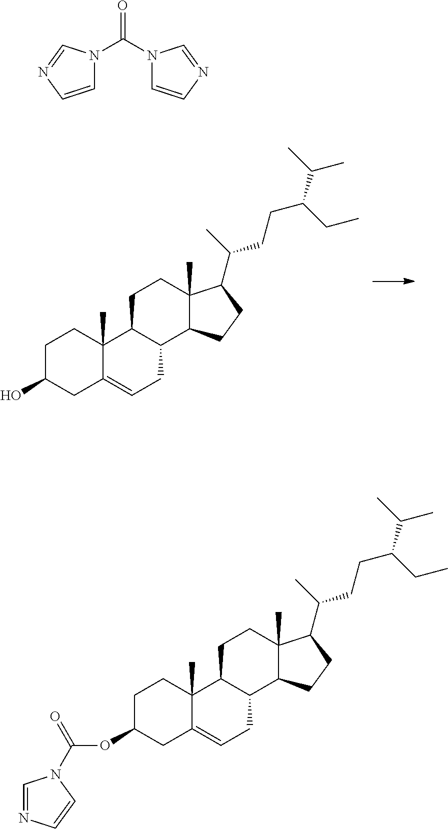 [SMILES: O=C(n1ccnc1)n1ccnc1 ; CC[C@H](CC[C@@H](C)[C@H]1CC[C@H]2[C@@H]3CC=C4C[C@@H](O)CC[C@]4(C)[C@H]3CC[C@]12C)C(C)C ; CC[C@H](CC[C@@H](C)[C@H]1CC[C@H]2[C@@H]3CC=C4C[C@@H](OC(=O)n5ccnc5)CC[C@]4(C)[C@H]3CC[C@]12C)C(C)C]

β-Sitosterol [TCI America, cat#: S0040, 40%, primary component depicted] (3.00 g, 7.23 mmol) was dissolved in DCM (14.00 mL) and treated with 1,1′-carbonyldiimidazole (1.17 g, 7.23 mmol) and triethylamine (263.53 mg, 2.60 mmol, 361 μL). The reaction mixture was stirred at 35° C. for 72 h, washed with aqueous 10% HCl (3×5 mL) and water (5 mL), dried with Na₂SO₄, and the solvent was evaporated to give a white solid, Intermediate A, (3.30 g, 6.49 mmol, 89% yield).

### Example 2—Synthesis of Compound 1

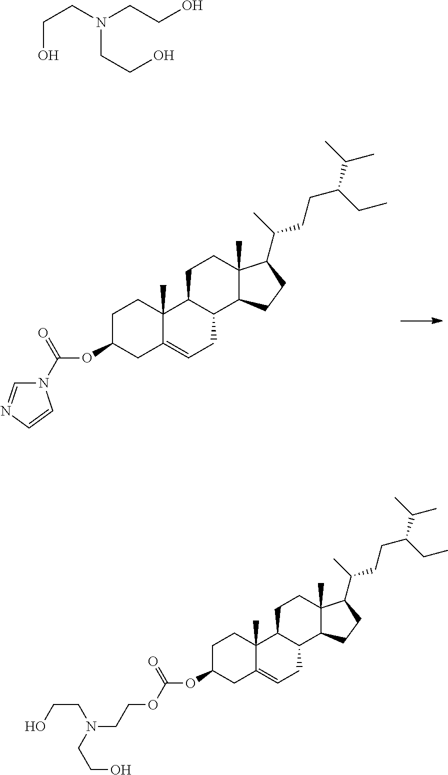 [SMILES: OCCN(CCO)CCO ; CC[C@H](CC[C@@H](C)[C@H]1CC[C@H]2[C@@H]3CC=C4C[C@@H](OC(=O)n5ccnc5)CC[C@]4(C)[C@H]3CC[C@]12C)C(C)C ; CC[C@H](CC[C@@H](C)[C@H]1CC[C@H]2[C@@H]3CC=C4C[C@@H](OC(=O)OCCN(CCO)CCO)CC[C@]4(C)[C@H]3CC[C@]12C)C(C)C]

A microwave tube was charged with Intermediate A (100 mg, 0.196 mmol) which was dissolved in 1,2-dichloroethane (980 μL) and treated with triethanolamine (59.0 mg, 0.393 mmol). The sealed reaction mixture was then stirred at 85° C. for 48 hours, until the reaction had gone to completion. The organics were then washed with 3% aqueous HCl (4 mL), water (5 mL), and 50% brine (4×5 mL). The organics were dried over Na₂SO₄, filtered, and concentrated under reduced pressure. The crude residue was then purified via flash chromatography with an eluent of methanol (with 10% aqueous ammonia)/chloroform to afford Compound 1 as a white crystalline solid (32 mg, 0.054 mmol, 28%).

Compound 2 was synthesized in an analogous manner using the corresponding diosgenin-based analog to Intermediate A.

### Example 3—Synthesis of Compound 3

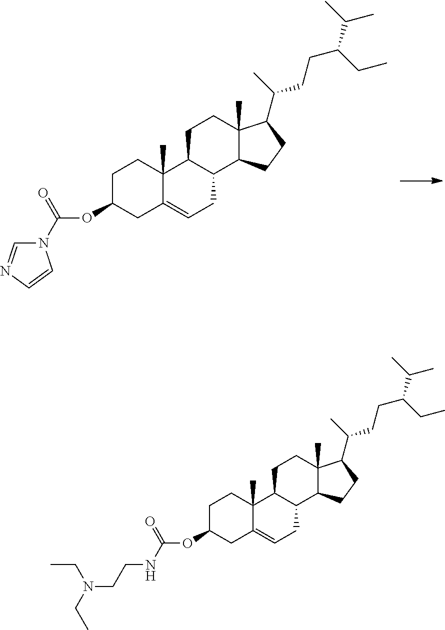

A microwave tube was charged with Intermediate A (500 mg, 0.982 mmol) which was dissolved in DCM (4.91 mL) and treated with N,N-diethylenediamine (152 mg, 1.31 mmol). The sealed reaction mixture was then stirred at 35° C. for 18 hours. The organics were then washed with 3% aqueous HCl (4 mL), water (5 mL), and 50% brine (4×5 mL). The organics were dried over Na₂SO₄, filtered, and concentrated under reduced pressure. The crude residue was then purified via flash chromatography with an eluent of methanol (with 10% aqueous ammonia)/chloroform to afford Compound 3 as a white crystalline solid (266 mg, 0.478 mmol, 49%).

Compound 5 was synthesized in an analogous manner from Intermediate A. Compounds 4 and 6 and Compounds 7 and 8 were synthesized in an analogous manner using the corresponding diosgenin- and α-tocopherol-based analogs of Intermediate A.

### Example 4—Synthesis of Intermediate B

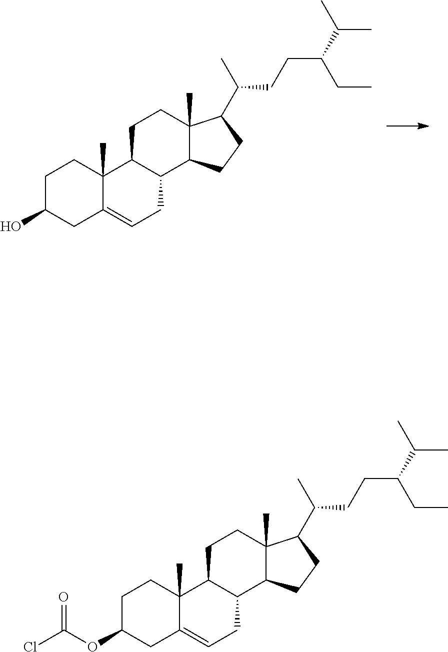 [SMILES: CC[C@H](CC[C@@H](C)[C@H]1CC[C@H]2[C@@H]3CC=C4C[C@@H](O)CC[C@]4(C)[C@H]3CC[C@]12C)C(C)C ; CC[C@H](CC[C@@H](C)[C@H]1CC[C@H]2[C@@H]3CC=C4C[C@@H](OC(=O)Cl)CC[C@]4(C)[C@H]3CC[C@]12C)C(C)C]

In dry THF (50.0 mL), triphosgene (1.43 g, 4.82 mmol) was dissolved and cooled down to 0° C. A solution of β-Sitosterol [TCI America, cat#: S0040, 40%, primary component depicted] (5.00 g, 12.1 mmol) in THF (10 mL) was added dropwise over 10 minutes. The reaction mixture was stirred at 0° C. for 2 hours before warming to room temperature. The reaction mixture was let to stir for 4 hours until the reaction completed as indicated by TLC. The reaction mixture was diluted with hexanes (300 mL) and the solid precipitates were removed via filtration. The organic solution was concentrated under reduced pressure and the crude material was directly applied to flash chromatography eluting with hexanes/DCM (4:1) to afford the desired product, Intermediate B (4.00 g, 8.40 mmol, 70%)

### Example 5—Synthesis of Compound 9

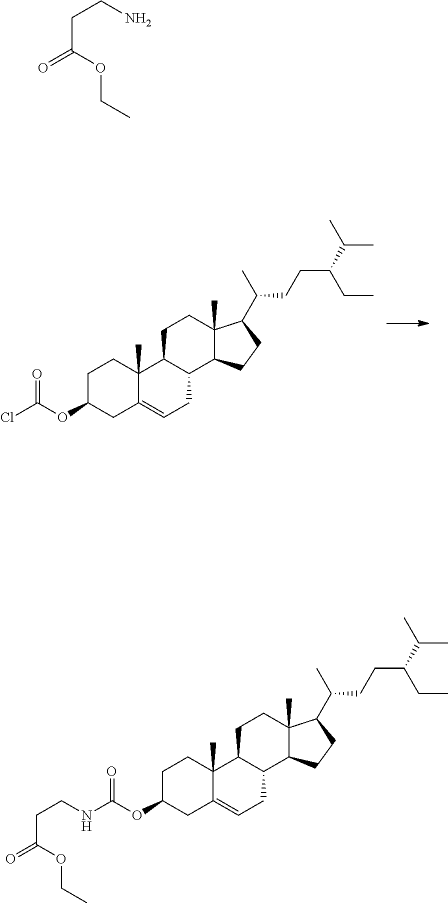

Ethyl 3-aminopropanoate (96.6 mg, 629 μmol) was dissolved in i-PrOH (10.0 mL), treated with triethylamine (63.6 mg, 629 μmol, 87.2 μL) and cooled to 0° C. Intermediate B (200 mg, 419 μmol) was dissolved in DCM (5 mL) and added dropwise over 10 minutes to the reaction mixture. The reaction mixture was stirred for 2 hours at 0° C. before being warmed to room temperature. The reaction mixture was stirred for additional 4 hours at room temperature until the reaction was completed as indicated by TLC. The reaction mixture was concentrated under reduced pressure and purified via flash chromatography with an eluent of EtOAc/hexanes (1:8) to afford Compound 9 (210 mg, 0.376 mmol, 89%).

### Example 6—Synthesis of Compound 10

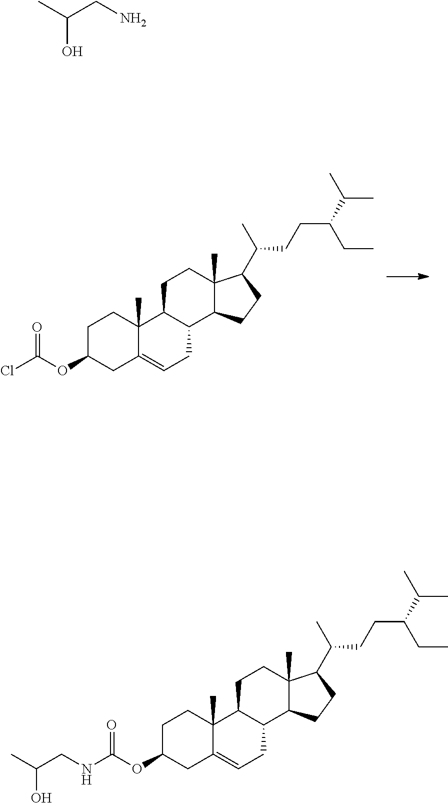

A solution of 1-aminopropan-2-ol (63.0 mg, 838 μmol) in dry iPrOH (10.0 mL) was treated with triethylamine (63.6 mg, 629 μmol, 87.2 μL) and cooled to 0° C. A solution of Intermediate B (200 mg, 419 μmol) in DCM (10 mL) was added dropwise over 10 minutes to the reaction mixture. The reaction mixture was stirred at 0° C. for 2 hours, then allowed to warm to room temperature. After stirring at room temperature for 4 hours, the reaction was complete as indicated by TLC. The reaction mixture was concentrated under reduced pressure and directly purified via flash chromatography with an eluent of EtOAc/hexanes (1:3) to afford Compound 10 (190.00 mg, 0.368 mmol, 88%).

Compounds 11-13 were synthesized in an analogous manner from Intermediate B.

### Example 7—Synthesis of Compounds 14 and 15

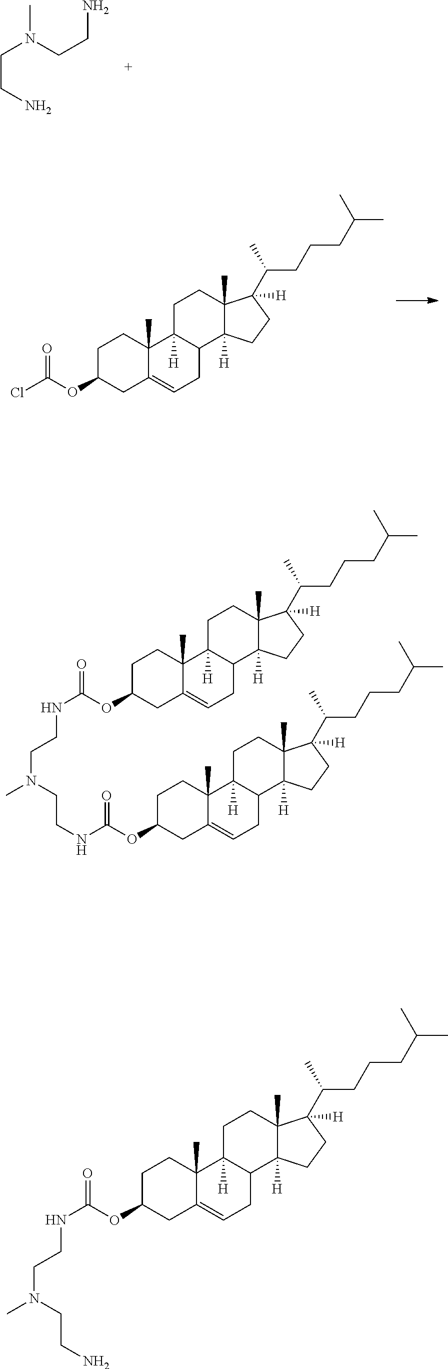

Cholesteryl chloroformate [Sigma Aldrich, C77007, 95%] (1.00 g, 2.23 mmol) was dissolved in DCM (15.00 mL) and treated with 2,2′-diamino-N-methyldiethylamine (413 mg, 3.35 mmol). The reaction mixture was stirred at room temperature in a sealed vial for 18 hours, after which a white solid precipitated was observed. TLC indicated the full consumption of the cholesteryl chloroformate starting material. The reaction mixture was concentrated under reduced pressure and purified via flash chromatography with an eluent of methanol (10% aqueous ammonia)/DCM (1:15) to yield Compound 14, (958 mg, 1.02 mmol, 46%) and Compound 15, (87.0 mg, 0.164 mmol, 7%).

### Example 8—Synthesis of Compound 16

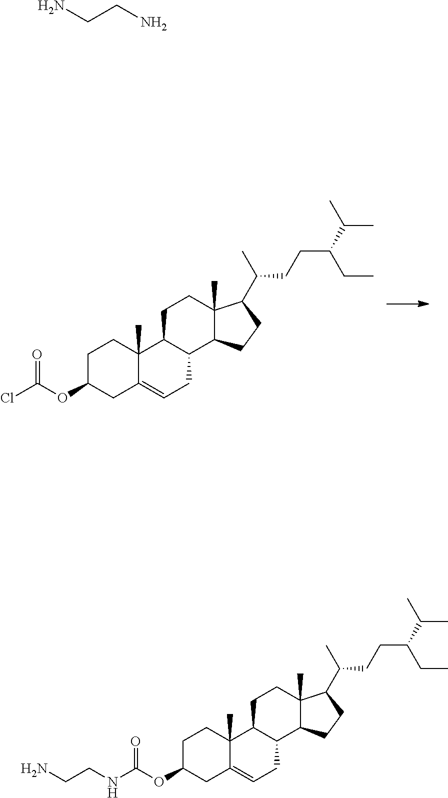

Ethane-1,2-diamine (30.2 mg, 503 μmol) was dissolved in i-PrOH (10.00 mL), treated with triethylamine (63.6 mg, 629 μmol, 87.2 μL), and cooled to 0° C. Intermediate B (200 mg, 419 μmol) was dissolved in THF (10 mL) and added in dropwise over 10 minutes to the reaction mixture. The subsequent reaction mixture was stirred for 2 hours at 0° C. before being warmed to room temperature and stirred for an additional 4 hours. Upon completion of the reaction as indicated by TLC, the reaction mixture was concentrated under reduced pressure and dried under high vacuum to afford Compound 16 as a chloride salt (212.00 mg, 0.394 mmol, 94%)

### Example 9—Synthesis of Intermediate C

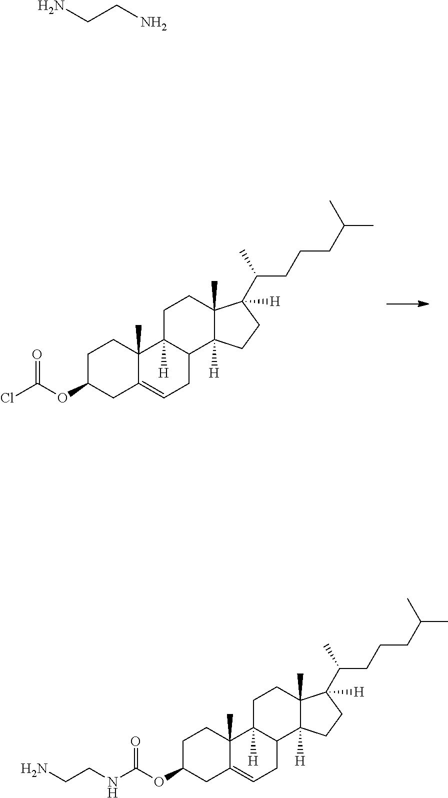

Ethane-1,2-diamine (267 mg, 4.45 mmol) was dissolved in i-PrOH/DCM 1:1 (40.00 mL), treated with triethylamine (63.6 mg, 629 μmol, 87.2 μL), and cooled to 0° C. Cholesteryl chloroformate [Sigma Aldrich, C77007, 95%] (190 mg, 419 μmol) was dissolved in THF (10 mL) and added in dropwise over 10 minutes to the reaction mixture. The subsequent reaction mixture was stirred for 2 hours at 0° C. before being warmed to room temperature and stirred for an additional 4 hours. Upon completion of the reaction as indicated by TLC, the reaction mixture was concentrated under reduced pressure and dried under high vacuum to afford the desired product, Intermediate C (2.00 g, 4.23 mmol, 95%).

### Example 10—Synthesis of Compounds 17 and 18

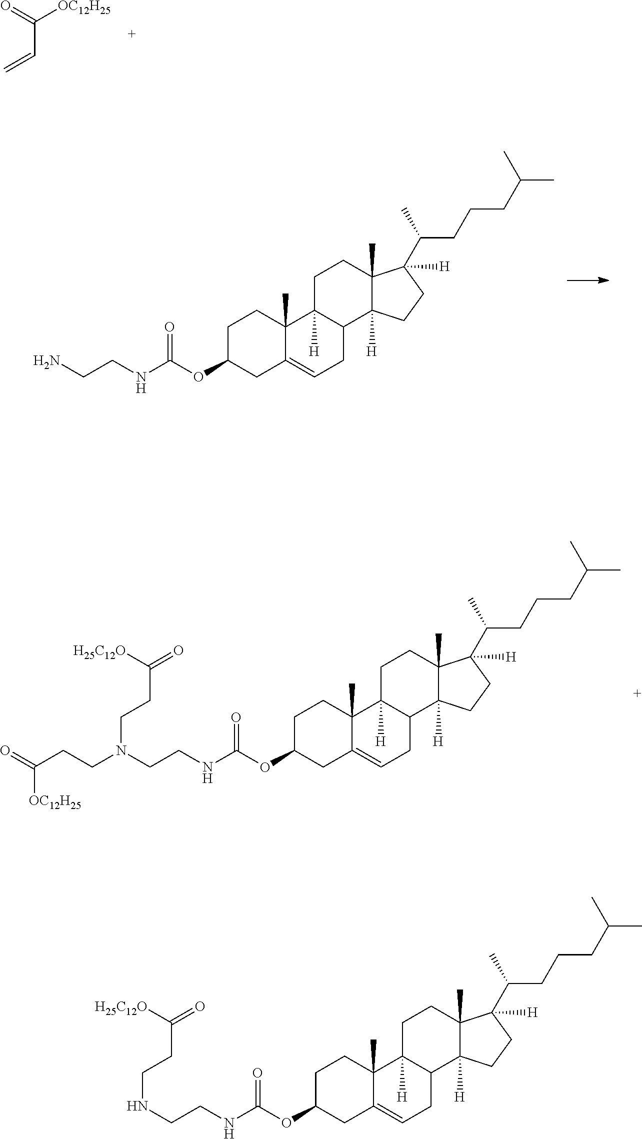

A 2 dram vial was charged with Intermediate C (283 mg, 1.18 mmol) and a 1:1 mixture of i-PrOH and DCM (10.0 mL). The vial was sealed and the reaction mixture was stirred at 75° C. for 2 days. The reaction mixture was concentrated under reduced pressure and purified via flash chromatography with an eluent of methanol (with 10% aqueous ammonia)/DCM (1:10) to afford Compound 14 (156 mg, 0.163 mmol, 42%) and Compound 15 (147.00 mg, 0.206 mmol, 52%).

### Example 11—Synthesis of Compound 19

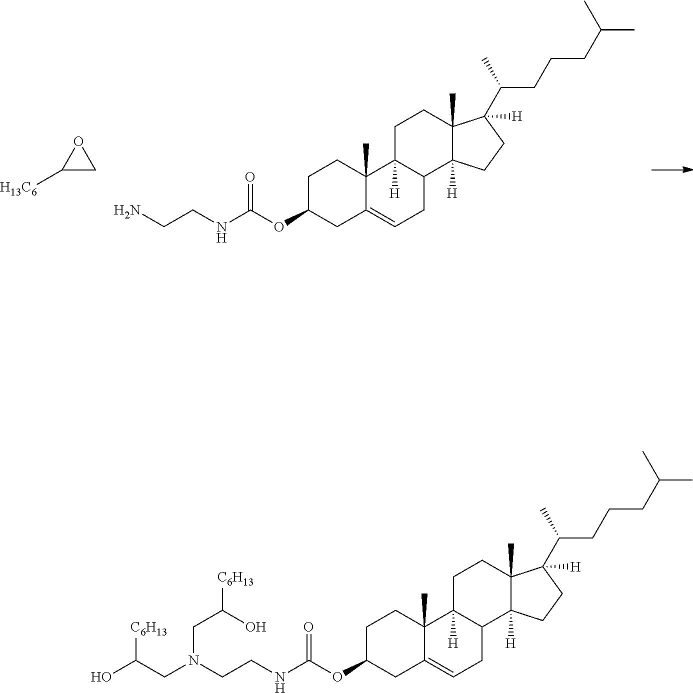

A 2 dram vial was charged with Intermediate C (300 mg, 589 μmol), 2-hexyloxirane (227 mg, 1.77 mmol), and i-PrOH (5.00 mL). The vial was sealed and the reaction mixture was stirred at 75° C. for 2 days. The reaction mixture was concentrated under reduced pressure and purified via flash chromatography with an eluent of methanol (with 10% aqueous ammonia)/DCM (1:20) to afford Compound 16 (123 mg, 0.168 mmol, 29%).

Compounds 20 and 21 were synthesized in an analogous manner from Intermediate C.

### Example 12—Synthesis of Intermediate D

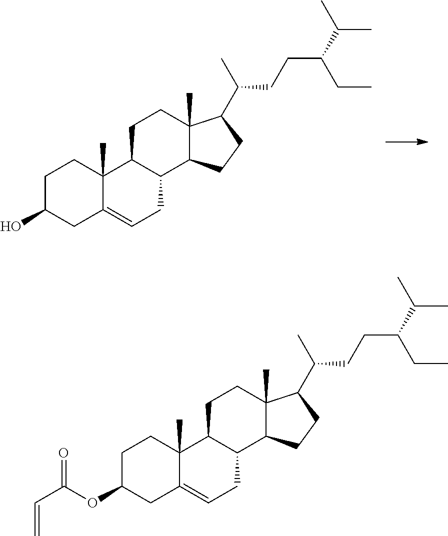 [SMILES: CC[C@H](CC[C@@H](C)[C@H]1CC[C@H]2[C@@H]3CC=C4C[C@@H](O)CC[C@]4(C)[C@H]3CC[C@]12C)C(C)C ; C=CC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1]

A solution of β-sitosterol [TCI America, cat#: S0040, 40%, primary component depicted] (3.00 g, 7.23 mmol) and DCM (50.00 mL) was treated with triethylamine (732.02 mg, 7.23 mmol, 1.00 mL). This mixture was cooled to 0° C. and treated with a solution of acryloyl chloride (655 mg, 7.23 mmol, 574 μL) in DCM (5 mL) dropwise over 10 minutes. The reaction mixture was let to stir at 0° C. for 2 hours before being warmed to room temperature and stirred for an additional 4 hours until the reaction was completed as indicated by TLC. The reaction mixture was concentrated under reduced pressure and purified via flash chromatography eluting with EtOAc/hexane (1:10) to afford the desired product, Intermediate D (3.00 g, 6.40 mmol, 66%).

### Example 13—Synthesis of Intermediate E

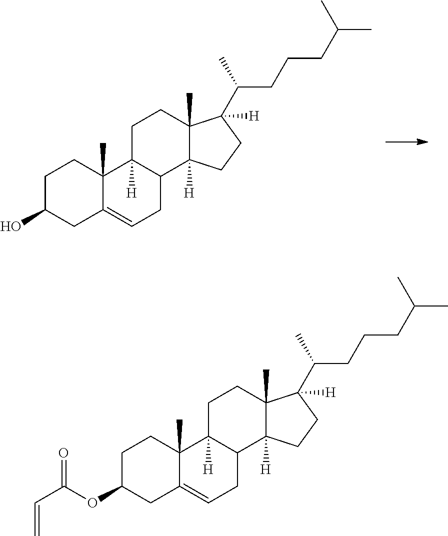

A solution of cholesterol [Sigma Aldrich, C3045, 98%] (2.70 g, 7.23 mmol) and DCM (50.0 mL) was treated with triethylamine (732.02 mg, 7.23 mmol, 1.00 mL). This mixture was cooled to 0° C. and treated with a solution of acryloyl chloride (654.76 mg, 7.23 mmol, 574.35 μL) in DCM (5 mL) dropwise over 10 minutes. The reaction mixture was let to stir at 0° C. for 2 hours before being warmed to room temperature and stirred for an additional 4 hours until the reaction was completed as indicated by TLC. The reaction mixture was concentrated under reduced pressure and purified via flash chromatography eluting with EtOAc/hexane (1:10) to afford the desired product, Intermediate E (2.70 g, 6.40 mmol, 66%).

### Example 14—Synthesis of Compounds 22 and 23

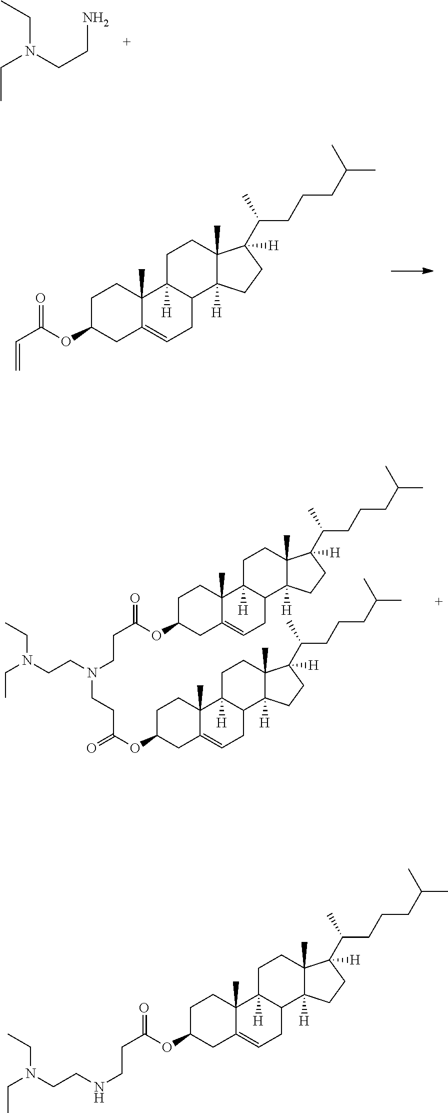

In a 2 dram vial, Intermediate E (300 mg, 681 μmol) was dissolved in anhydrous i-PrOH (5.00 mL) and charged with N,N-diethylethylenediamine (52.7 mg, 454 μmol). The reaction mixture was stirred at 80° C. for 18 hours, at which time complete consumption of starting material was observed via TLC. The reaction mixture was concentrated under reduced pressure and purified via flash chromatography with an eluent of methanol (with 10% aqueous ammonia)/DCM (1:10) to afford Compound 22, (111 mg, 0.111 mmol, 25%) and Compound 23 (61 mg, 0.11 mmol, 24%).

Compound 24 was synthesized in an analogous manner from Intermediate E. Compounds 25-27 were synthesized in an analogous manner from Intermediate D.

### Example 15—Synthesis of Compound 28

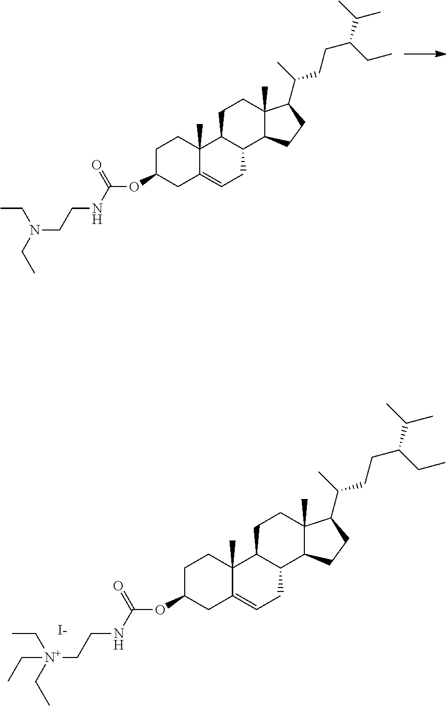

Compound 3 (100 mg, 179 μmol) was dissolved in DCM (0.90 mL) and treated with iodoethane (56 mg, 359 μmol). The reaction mixture was stirred at room temperature for 5 days before being concentrated under reduced pressure and dried under high vacuum to afford Compound 28 as deep red iodo salt (113 mg, 0.158 mmol, 88%).

Compounds 29 and 30 were synthesized in an analogous manner from Compounds 4 and 7, respectively.

### Example 16—Synthesis of Intermediate F

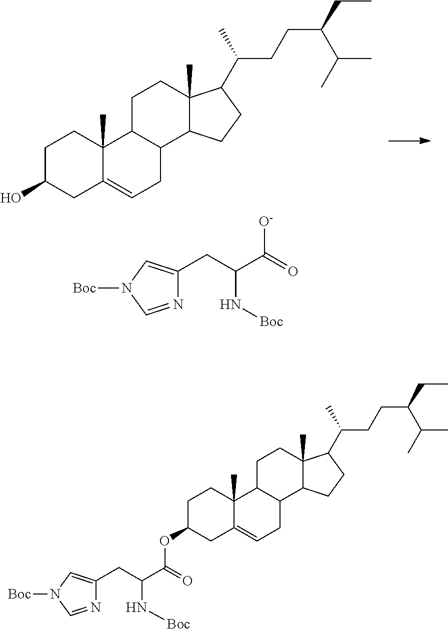 [SMILES: CC[C@H](CC[C@@H](C)C1CCC2C3CC=C4C[C@@H](O)CC[C@]4(C)C3CC[C@@]21C)C(C)C ; CC(C)(C)OC(=O)NC(Cc1cn(C(=O)OC(C)(C)C)cn1)C(=O)[O-] ; CC[C@H](CC[C@@H](C)C1CCC2C3CC=C4C[C@@H](OC(=O)C(Cc5cn(C(=O)OC(C)(C)C)cn5)NC(=O)OC(C)(C)C)CC[C@]4(C)C3CC[C@@]21C)C(C)C]

A 20 mL vial with septum cap was charged with β-Sitosterol [TCI America, cat#: S0040, 40%, primary component depicted] (200 mg, 0.482 mmol), Boc-His(Boc)-OH (206 mg, 0.579 mmol), Hunig's base (75 mg, 0.579 mmol), DMAP (12 mg, 0.0965 mmol), EDC (110 mg, 0.579 mmol), and DCM (2.0 mL). The reaction mixture was stirred at room temperature overnight, then diluted with DCM and washed with 1% aqueous HCl (3×5 mL), dried over Na₂SO₄, and the solvent evaporated. The crude residue was purified by flash chromatography with an eluent of DCM/5% Et₃N in i-PrOH to afford the desired product, Intermediate F, as a clear oil (205 mg, 0.273 mmol, 57%).

### Example 17—Synthesis of Compound 31

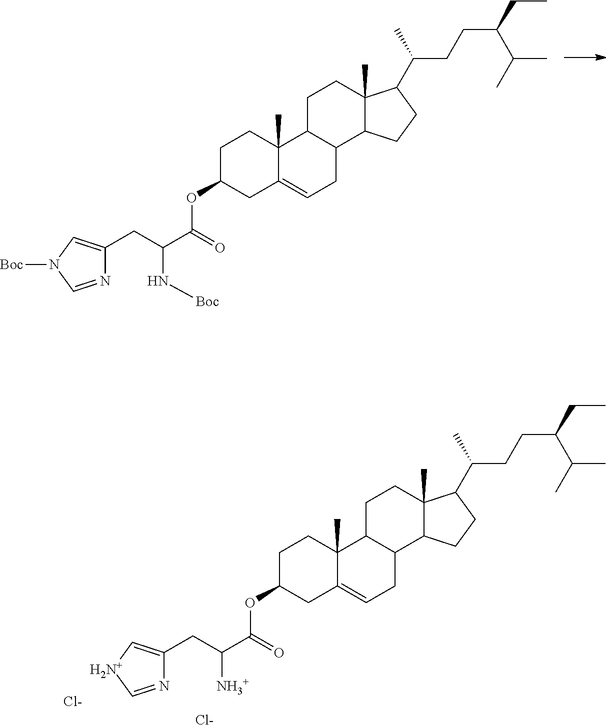 [SMILES: CC[C@H](CC[C@@H](C)C1CCC2C3CC=C4C[C@@H](OC(=O)C(Cc5cn(C(=O)OC(C)(C)C)cn5)NC(=O)OC(C)(C)C)CC[C@]4(C)C3CC[C@@]21C)C(C)C ; CC[C@H](CC[C@@H](C)C1CCC2C3CC=C4C[C@@H](OC(=O)C([NH3+])CC5=C[NH2+]C=N5)CC[C@]4(C)C3CC[C@@]21C)C(C)C ; [Cl-] ; [Cl-]]

Intermediate F (200 mg, 0.266 mmol) was dissolved in dioxane (2.0 mL) and a solution of HCl (4 M in dioxane, 1.33 mL, 5.32 mmol) was added. The reaction mixture was stirred overnight, forming a white precipitate. The solvent was decanted and the reaction mixture concentrated and dried under high vacuum to afford Compound 31 as a white solid (120 mg, 0.192 mmol, 72%).

Compounds 32 and 33 were synthesized in a manner similar to that described in Examples 16 and 17.

### Example 18—Synthesis of Compound 34

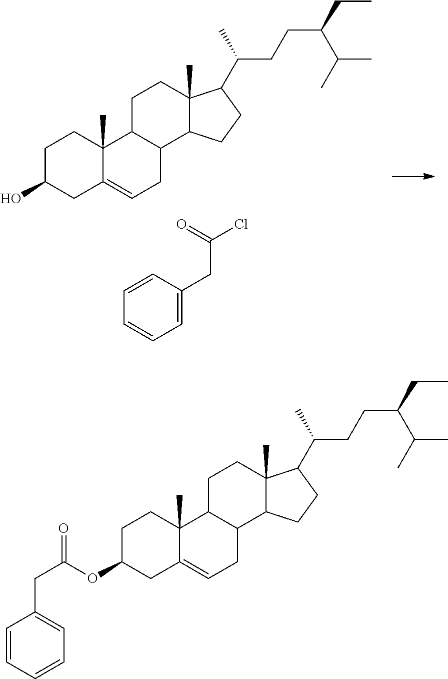 [SMILES: CC[C@H](CC[C@@H](C)C1CCC2C3CC=C4C[C@@H](O)CC[C@]4(C)C3CC[C@@]21C)C(C)C ; O=C(Cl)Cc1ccccc1 ; CC[C@H](CC[C@@H](C)C1CCC2C3CC=C4C[C@@H](OC(=O)Cc5ccccc5)CC[C@]4(C)C3CC[C@@]21C)C(C)C]

A solution of β-Sitosterol [TCI America, cat#: S0040, 40%, primary component depicted] (300 mg, 0.723 mmol) and DCM (3 mL) was treated with phenylacetyl chloride (134 mg, 0.868 mmol) followed by Hunig's base (93 mg, 0.723 mmol). The reaction mixture was stirred under N₂ for 16 hours, then diluted with DCM and washed with saturated aqueous NaHCO₃ (3×5 mL), dried over Na₂SO₄, and the solvent evaporated. The crude residue was purified by flash chromatography with an eluent of EtOAc/hexanes to afford Compound 34 as a white solid (146 mg, 0.274 mmol, 38%).

### Example 19—Synthesis of Compound 35

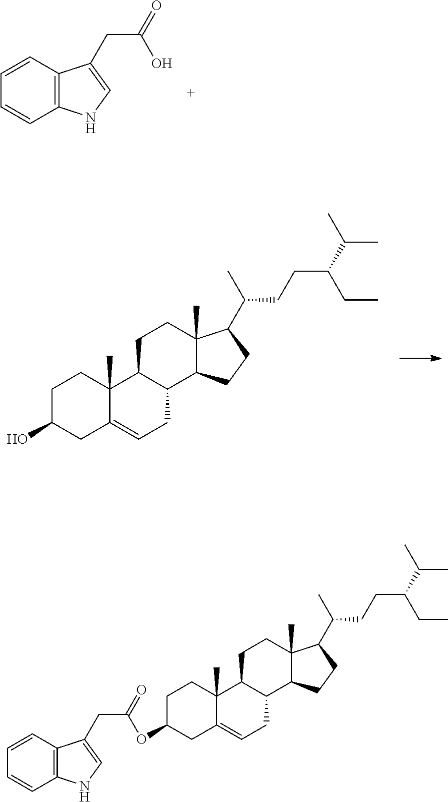 [SMILES: O=C(O)Cc1c[nH]c2ccccc12 ; CC[C@H](CC[C@@H](C)[C@H]1CC[C@H]2[C@@H]3CC=C4C[C@@H](O)CC[C@]4(C)[C@H]3CC[C@]12C)C(C)C ; CC[C@H](CC[C@@H](C)[C@H]1CC[C@H]2[C@@H]3CC=C4C[C@@H](OC(=O)Cc5c[nH]c6ccccc56)CC[C@]4(C)[C@H]3CC[C@]12C)C(C)C]

A mixture of 2-(1H-indol-3-yl)acetic acid (200 mg, 1.14 mmol), β-Sitosterol [TCI America, cat#: S0040, 40%, primary component depicted] (473 mg, 1.14 mmol), p-toluenesulfonic acid (19.6 mg, 114 μmol) and toluene (10.0 mL) under N₂ was stirred at room temperature until complete dissolution was observed. The reaction mixture was heated and stirred at 85° C. for 1 day. Upon complete consumption of starting material, as indicated via TLC, the reaction mixture was cooled to room temperature, diluted with DCM and quenched with saturated aqueous NaHCO₃. The organics were extracted, dried over Na₂SO₄, and concentrated under reduced pressure. The crude material was purified via flash chromatography with an eluent of EtOAc/hexanes (5:1) to afford Compound 35 (160.00 mg, 279.78, μmol, 25%).

### Example 20—Synthesis of Intermediate G

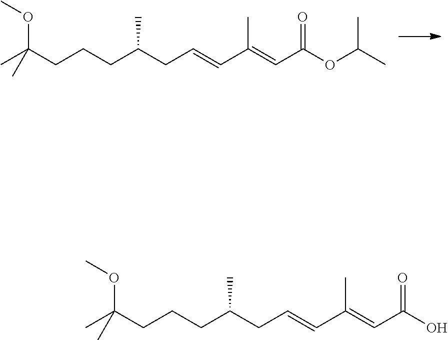

Methoprene (10.0 g, 32.2 mmol) was added to a 1:1 solution of water and methanol (40.0 mL) and treated with lithium hydroxide (1.54 g, 64.4 mmol). The reaction mixture was stirred for 2 days at 50° C. The reaction mixture's pH was then adjusted to pH 7 with 1 M HCl as indicated by pH paper and the reaction volume was concentrated down to 20 mL under reduced pressure. The reaction mixture was then diluted with DCM (500 mL), washed with brine (2×100 mL), dried over Na₂SO₄, and concentrated under reduced pressure. The crude material was purified via flash chromatography with an eluent of hexanes/EtOAc to afford Intermediate G (7.00 g, 26.1 mmol, 81%).

### Example 21—Synthesis of Intermediate H

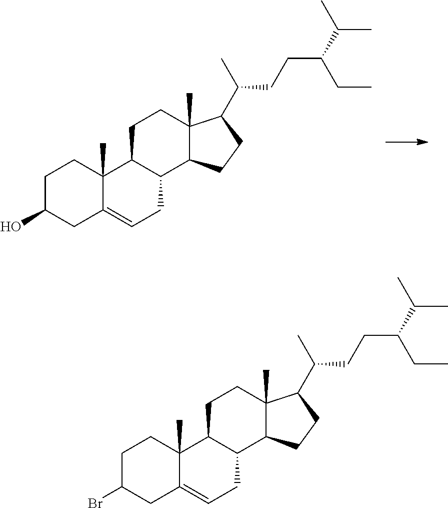 [SMILES: CC[C@H](CC[C@@H](C)[C@H]1CC[C@H]2[C@@H]3CC=C4C[C@@H](O)CC[C@]4(C)[C@H]3CC[C@]12C)C(C)C ; CC[C@H](CC[C@@H](C)[C@H]1CC[C@H]2[C@@H]3CC=C4CC(Br)CC[C@]4(C)[C@H]3CC[C@]12C)C(C)C]

A solution of β-Sitosterol [TCI America, cat#: S0040, 40%, primary component depicted] (2.00 g, 4.82 mmol), triphenylphosphine (2.53 g, 9.64 mmol) in anhydrous DCM (40.0 mL) under N₂ was cooled to 0° C. The reaction mixture was then treated with tetrabromomethane (2.40 g, 7.23 mmol) in DCM (20 mL) dropwise over 30 minutes. After addition, the reaction mixture was warmed to room temperature and stirred for an additional 4 hours. The reaction mixture was diluted with hexanes (400 mL) and filtered to remove the precipitates. The resulting filtrate solution was concentrated under reduced pressure and purified via flash chromatography with an eluent of 5:1 hexane to DCM to afford Intermediate H (2.1 g, 4.40 mmol, 91%).

### Example 22—Synthesis of Compound 38

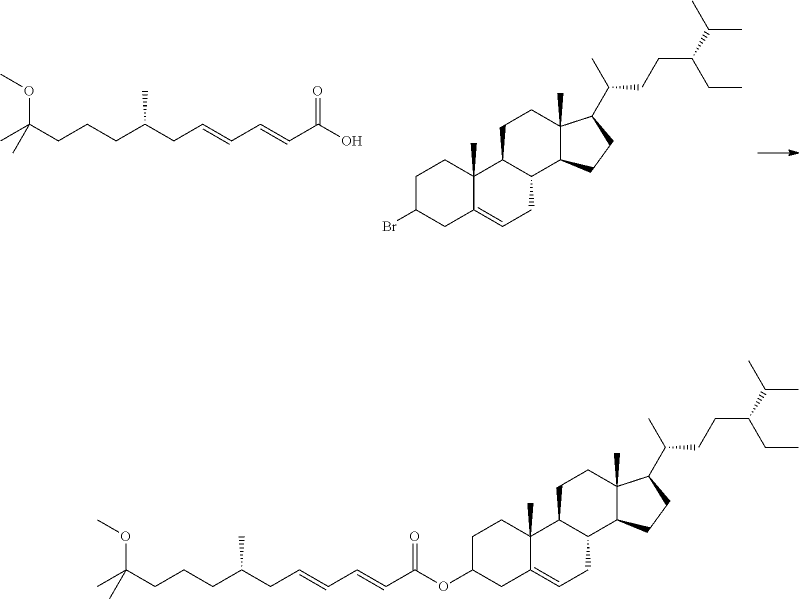 [SMILES: COC(C)(C)CCC[C@H](C)C/C=C/C=C/C(=O)O ; CC[C@H](CC[C@@H](C)[C@H]1CC[C@H]2[C@@H]3CC=C4CC(Br)CC[C@]4(C)[C@H]3CC[C@]12C)C(C)C ; CC[C@H](CC[C@@H](C)[C@H]1CC[C@H]2[C@@H]3CC=C4CC(OC(=O)/C=C/C=C/C[C@@H](C)CCCC(C)(C)OC)CC[C@]4(C)[C@H]3CC[C@]12C)C(C)C]

Intermediate G (155 mg, 576 μmol) and Intermediate H (250 mg, 524 γmol) were mixed with potassium carbonate (145 mg, 1.05 mmol) and tetrabutylammonium iodide (8.72 mg, 26.2 umol) in DMF (5.00 mL). The reaction mixture was heated to 70° C. overnight and then cooled to room temperature and diluted with ether (200 mL), washed with aqueous HCl (1 M), saturated aqueous NaHCO₃, brine, dried over Na₂SO₄, and concentrated under reduced pressure. The crude material was purified via flash chromatography with an eluent of EtOAc/hexanes (1:20) to Compound 38 (160 mg, 241 μmol, 46%).

### Example 23—Synthesis of Intermediate I

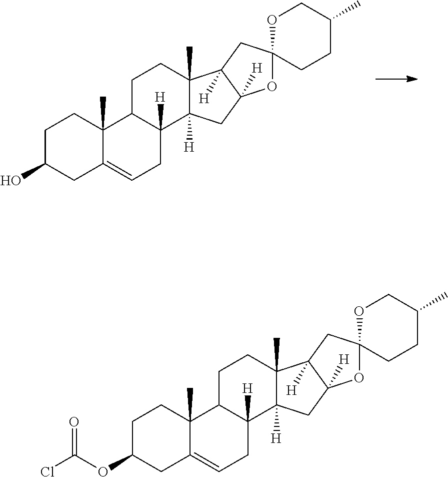

In dry THF (50.0 mL), triphosgene (1.43 g, 4.82 mmol) was dissolved and cooled down to 0° C. A solution of diosgenin [TCI America, cat#: D1474, 95%] (5.00 g, 121 mmol) in THF (10 mL) was added dropwise over 10 minutes. The reaction mixture was stirred at 0° C. for 2 hours before warming to room temperature. The reaction mixture was let to stir for 4 hours until the reaction was complete, as indicated by TLC. The reaction mixture was diluted with hexanes (300 mL) and the solid precipitates were removed via filtration. The organic solution was concentrated under reduced pressure and the crude material was purified via flash chromatography with an eluent of hexanes/DCM (4:1) to give the desired product, Intermediate I (4.00 g, 8.40 mmol, 70%).

### Example 24—Synthesis of Compound 39 (25% Loading of 2000 D PEI)

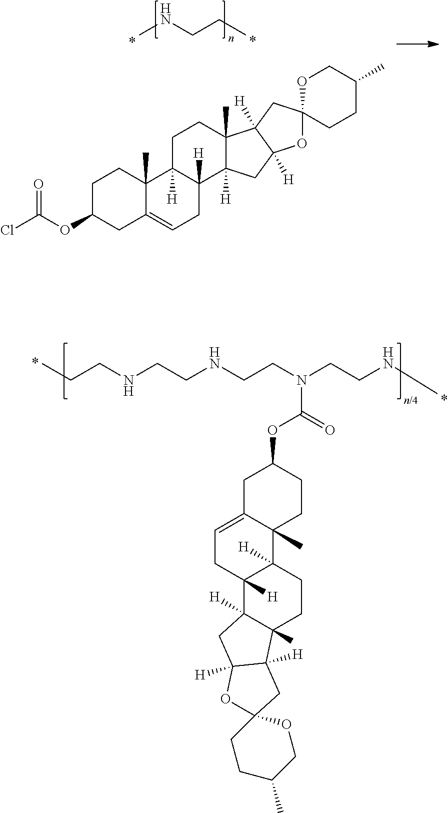

Polyethylenimine [Polysciences LLC, cat#: 24313] (2000 Dalton, Linear) (54.17 mg, 1.26 mmol) was dissolved in DCM (4.00 mL) and mixed with triethylamine (63.6 mg, 629 μmol, 87.2 μL). Intermediate I (150 mg, 314 μmol) in DCM (2 mL) was added to the reaction mixture dropwise over 5 minutes. The reaction mixture was stirred at room temperature in a sealed vial for 18 hours, forming a white precipitate. TLC indicated completion of the reaction. The reaction mixture was diluted with water (10 mL), washed with DCM (3×30 mL), and the combined organic phases were concentrated under reduced pressure and vacuumed to dryness to afford Compound 39 (160 mg, 78%).

Compound 40 was synthesized in an analogous manner from Intermediate B.

### Example 25—Synthesis of Compound 41 (10% Loading of 2000 D PEI)

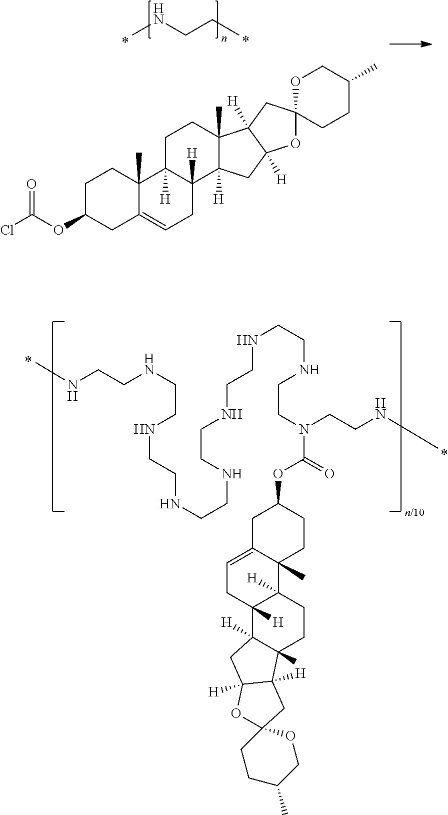

Polyethylenimine [Polysciences LLC, cat#: 24313] (2000 Dalton, Linear) (135 mg, 3.14 mmol) was dissolved in DCM (4.00 mL) and mixed with triethylamine (63.63 mg, 628.84 μmol, 87.16 μL). Intermediate I (150.00 mg, 314.42 μmol) in DCM (2 mL) was added to the reaction mixture dropwise over 5 minutes. The reaction mixture was stirred at room temperature in a sealed vial for 18 hours, forming a white precipitate. TLC indicated completion of the reaction. The reaction mixture was diluted with water (10 mL), washed with DCM (3×30 mL), and the combined organic phases were concentrated under reduced pressure and vacuumed to dryness to afford Compound 41 (240 mg, 53%).

Compound 42 was synthesized in an analogous manner from Intermediate B.

### Example 26—Synthesis of Compound 43

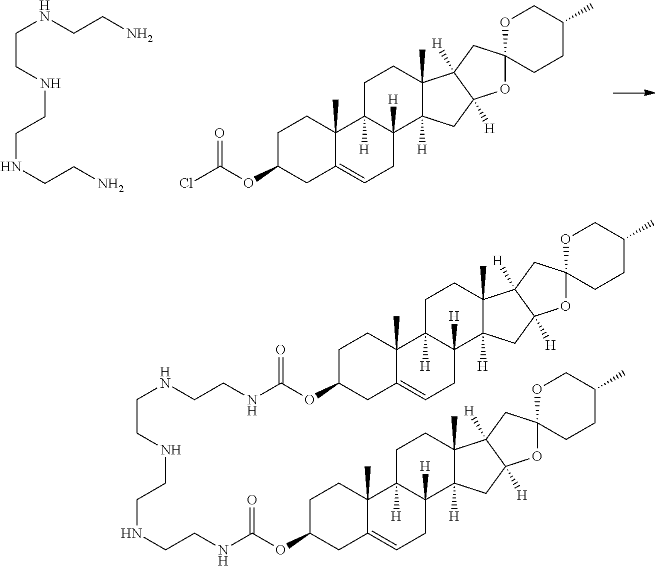

N¹-(2-aminoethyl)-N²-(2-((2-aminoethyl)amino)ethyl)ethane-1,2-diamine (1M, 314.42 μL) was dissolved in DCM (4.00 mL) and mixed with triethylamine (63.63 mg, 628.84 μmol, 87.17 μL). Intermediate I (300 mg, 629 μmol) in DCM (2 mL) was added to the reaction mixture dropwise over 5 minutes. The reaction mixture was stirred at room temperature in a sealed vial for 18 hours, forming a white precipitate. TLC indicated completion of the reaction. The reaction mixture was diluted with water (10 mL), washed with DCM (3×30 mL), and the combined organic phases were concentrated under reduced pressure and purified via flash chromatography eluting with methanol (10% ammonia)/DCM 1:4 to afford Compound 43 (15.00 mg, 0.014 mmol, 4%).

### Example 27—Synthesis of Compound 45

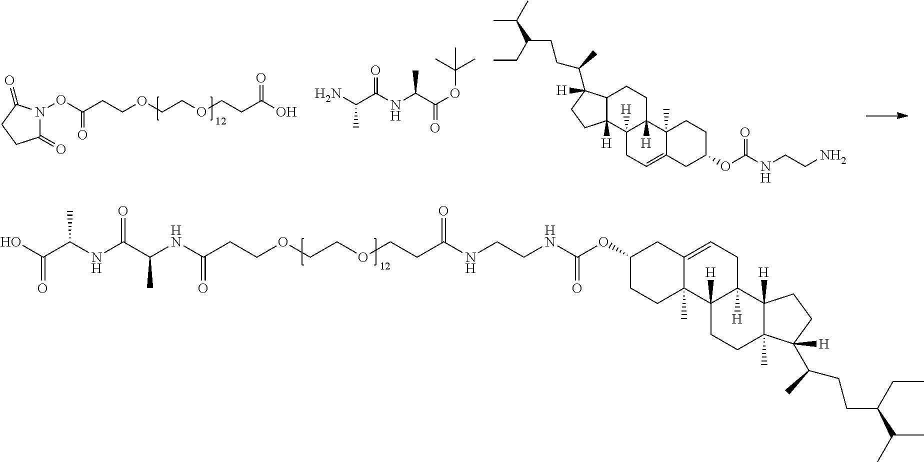

A mixture of 3-[2-[2-[2-[2-[2-[2-[2-[2-[2-[2-[2-[2-[3-(2,5-dioxopyrrolidin-1-yl)oxy-3-oxo propoxy]ethoxy]ethoxy]ethoxy]ethoxy]ethoxy]ethoxy]ethoxy]ethoxy]ethoxy]-ethoxy]ethoxy]ethoxy]propanoic acid (28.5 mg, 36.2 μmol), tert-butyl (2S)-2-[[(2S)-2-aminopropanoyl]amino]propanoate (8.6 mg, 40 μmol), and Hunig's base (9.35 mg, 72.4 μmol, 12.6 μL) was dissolved in DCM (300 μL) and stirred at room temperature for 18 hours, then 1-(3-dimethylaminopropyl)-3-ethylcarbodiimide hydrochloride (13.8 mg, 72.0 μmol) was added followed by Compound 16 (36.0 mg, 71.9 μmol) and stirred at room temperature for 18 hours. The reaction mixture was diluted with DCM, washed with a solution of brine and aqueous 3% HCl (pH 2) (2×2 mL), dried with Na₂SO₄ and concentrated under reduced pressure to afford tert-butyl (2S)-2-[[(2S)-2-[3-[2-[2-[2-[2-[2-[2-[2-[2-[2-[2-[2-[2-[3-[2- [[(3S,8S,9S,10R,13R,-14S,17R)-17-[(1R,4R)-4-ethyl-1,5-dimethyl-hexyl]-10,13-dimethyl-2,3,4,7,8,9,11,12,14,15,-16,17-dodecahydro-1H-cyclopenta[a]phenanthren-3-yl]oxycarbonylamino]ethylamino]-3-oxo-propoxy]ethoxy]ethoxy]ethoxy]ethoxy]ethoxy]ethoxy]ethoxy]ethoxy]ethoxy]ethoxy]ethoxy]etho xy]propanoylamino]propanoyl]amino]propanoate as a yellow oil which was carried on to the next step without further purification.

To a suspension of tert-butyl (2S)-2-[[(2S)-2-[3-[2-[2-[2-[2-[2-[2-[2-[2-[2-[2-[2-[2-[3-[2-[[(3S,8S,9S,10R,13R,-14S,17R)-17-[(1R,4R)-4-ethyl-1,5-dimethyl-hexyl]-10,13-dimethyl-2,3,4,7,8,9,11,12,14,15,-16,17-dodecahydro-1H-cyclopenta[a]phenanthren-3-yl]oxycarbonyl-amino]ethylamino]-3-oxo-propoxy]ethoxy]ethoxy]ethoxy]ethoxy]ethoxy]-ethoxy]ethoxy]ethoxy]ethoxy]ethoxy]ethoxy]ethoxy]propanoylamino]propanoyl]amino]propanoate (45.0 mg, 32.8 μmol) in dioxane (400 μL) was added HCl (4 M solution in dioxane, 246 μL, 0.984 mmol) and the suspension stirred at room temperature for 72 hours. The reaction mixture was concentrated under reduced pressure and purified via flash chromatography with an eluent of methanol/DCM (1:2) to afford Product 44 as a clear oil (8.0 mg, 6.1 μmol, 19%).

Synthesis of Intermediate J

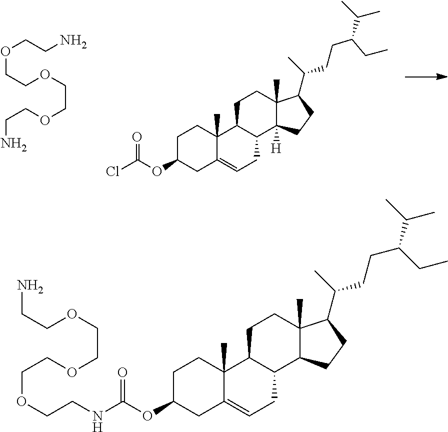 [SMILES: NCCOCCOCCOCCN ; CC[C@H](CC[C@@H](C)[C@H]1CC[C@H]2[C@@H]3CC=C4C[C@@H](OC(=O)Cl)CC[C@]4(C)[C@H]3CC[C@]12C)C(C)C ; CC[C@H](CC[C@@H](C)[C@H]1CC[C@H]2[C@@H]3CC=C4C[C@@H](OC(=O)NCCOCCOCCOCCN)CC[C@]4(C)[C@H]3CC[C@]12C)C(C)C]

To a solution of 2-[2-[2-(2-aminoethoxy)ethoxy]ethoxy]ethanamine (646 mg, 3.36 mmol) in anhydrous i-PrOH (10.0 mL) was added triethylamine (255 mg, 2.52 mmol, 349 μL) and the reaction mixture cooled to 0° C. A solution of [(3S,8S,9S,10R,13R,14S,17R)-17-[(1R,4R)-4-ethyl-1,5-dimethyl-hexyl]-10,13-dimethyl-2,3,4,7,8,9,11,12,14,15,16,17-dodecahydro-1H-cyclopenta[a]phenanthren-3-yl] carbonochloridate (800 mg, 1.68 mmol) in DCM (10 mL) was added dropwise to the reaction mixture over 10 minutes. The resultant solution was stirred at 0° C. for 2 hours then warmed to room temperature and stirred another 4 hours until completion of the reaction was indicated by TLC. The reaction mixture was concentrated under reduced pressure and purified via flash chromatography with an eluent of methanol/DCM (1:4) to afford the product [(3S,8S,9S,10R,13R,14S,17R)-17-[(1R,4R)-4-ethyl-1,5-dimethyl-hexyl]-10,13-dimethyl-2,3,4,7,8,9,11,12,14,15,16,17-dodecahydro-1H-cyclopenta[a]phenanthren-3-yl] N-[2-[2-[2-(2-aminoethoxy)ethoxy]ethoxy]ethyl]carbamate (823 mg, 1.30 mmol, 77.4% yield).

### Example 27—Synthesis of Compound 46

To a suspension of Intermediate J (358 mg, 566 μmol) and 3-[2-[2-[2-[2-[2-[2-[2-[2-[2-[2-[2-[2-[3-(2,5-dioxopyrrolidin-1-yl)oxy-3-oxo-propoxy]ethoxy]ethoxy]ethoxy]ethoxy]-ethoxy]ethoxy]ethoxy]ethoxy]ethoxy]ethoxy]ethoxy]ethoxy]propanoic acid (446 mg, 566 μmol) in dry DCM (2.50 mL) was added Hunig's base (110 mg, 848 μmol, 148 μL). The reaction mixture was stirred at room temperature for 18 hours then diluted with DCM (2 mL), added a solution of brine and aqueous 3% HCl (pH 2) (2 mL), separated phases and washed aq. with DCM, combined organics and washed with a solution of brine and aqueous 3% HCl (pH 2) (2×2 mL), dried (Na₂SO₄) and solvent evaporated to afford 3-[2-[2-[2-[2-[2-[2-[2-[2-[2-[2-[2-[2-[3-[2-[2-[2-[2-[[(3S,8S,9S,10R,13R,14S)-17-[(1R,4R)-4-ethyl-1,5-dimethyl-hexyl]-10,13-dimethyl-2,3,4,7,8,9,11,12,14,15,16,17-dodecahydro-1H-cyclopenta[a]phenanthren-3-yl]oxycarbonylamino]ethoxy]ethoxy]ethoxy]ethylamino]-3-oxo-propoxy]ethoxy]ethoxy]-ethoxy]ethoxy]ethoxy]ethoxy]ethoxy]ethoxy]ethoxy]ethoxy]ethoxy]ethoxy]propanoic acid as a white foamy resin which was carried on to the next step without further purification.

A mixture of 3-[2-[2-[2-[2-[2-[2-[2-[2-[2-[2-[2-[2-[3-[2-[2-[2-[2-[[(3S,8S,9S,10R,13R,14S)-17-[(1R,4R)-4-ethyl-1,5-dimethyl-hexyl]-10,13-dimethyl-2,3,4,7,8,9,11,12,14,15,16,17-dodecahydro-1H-cyclopenta[a]phenanthren-3-yl]oxycarbonylamino]ethoxy]ethoxy]ethoxy]-ethylamino]-3-oxo-propoxy]ethoxy]ethoxy]-ethoxy]ethoxy]ethoxy]ethoxy]ethoxy]ethoxy]-ethoxy]ethoxy]ethoxy]ethoxy]propanoic acid (300 mg, 230 μmol), dimethylaminopyridine (14.0 mg, 115 μmol, 19.2 μL), 1-(3-dimethylaminopropyl)-3-ethylcarbodiimide hydrochloride (39.6 mg, 207 μmol) and Hunig's base (59 mg, 46 μmol, 80 μL) was dissolved in dry DMF (2.00 mL), then 1-hydroxypyrrolidine-2,5-dione (29.1 mg, 253 μmol) was added. The reaction mixture was stirred at room temperature for 8 hours. An aliquot of this solution (0.40 mL, 46 μmol) was transferred to a vial with septum cap and a solution of 2-[(2R,5R,8S,11S)-8-(4-aminobutyl)-5-benzyl-11-(3-guanidinopropyl)-3,6,9,12,15-pentaoxo-1,4,7,10,13-pentazacyclopentadec-2-yl]acetic acid (12.4 mg, 20.5 μmol) in DMF (0.15 mL) was added. The reaction mixture was stirred at room temperature for 72 hours, then diluted with a solution of brine and aqueous HCl (pH 2, 2 mL) and extracted with DCM (2×2 mL), the organic phases combined and washed with brine, dried (Na₂SO₄) and concentrated under reduced pressure. Purification was accomplished by high performance liquid chromatography on a Sunfire PREP C8 column using a gradient of 5-95% solvent B over 15 minutes. Solvent A=0.1% Formic acid, B=5% IPA/MeCN/0.1% Formic acid. Column: Sunfire PREP C8 OBD 5μ19×100 mm to afford Product 45 (12.5 mg, 6.61 μmol, 14%) of a clear oil.

### Example 28—Synthesis of Intermediate K

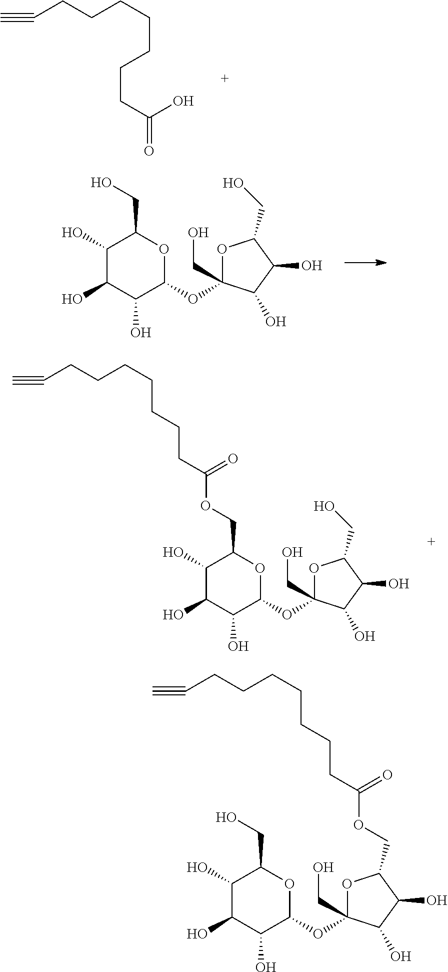

Under N₂ protection, undec-10-ynoic acid (500.00 mg, 2.74 mmol) and (2R,3R,4S,5S,6R)-2-[(2S,3S,4S,5R)-3,4-dihydroxy-2,5-bis(hydroxymethyl)tetrahydrofuran-2-yl]oxy-6-(hydroxymethyl)tetrahydropyran-3,4,5-triol (1.88 g, 5.48 mmol) and diisopropyl azodicarboxylate (1.66 g, 8.22 mmol, 1.61 mL) and DMF (20.00 mL) was added to a dry 2-dram vial. The mixture was stirred until solids dissolved and then cooled to 0° C. Triphenylphosphine (2.16 g, 8.22 mmol) in DCM (2 ml) was added dropwise to the solution. The reaction mixture was cooled for 30 minutes. The reaction mixture was allowed to warm to room temperature and stirred for 12 hours. Solvent was removed in vacuo and the crude product was purified by reverse phase HPLC to yield the regioisomer [(2R,3S,4S,5R,6R)-6-[(2S,3S,4S,5R)-3,4-dihydroxy-2,5-bis(hydroxymethyl)tetrahydrofuran-2-yl]oxy-3,4,5-trihydroxy-tetrahydropyran-2-yl]methyl undec-10-ynoate (500.00 mg, 987.09 μmol, 36.03% yield).

### Example 29—Synthesis of Intermediate L

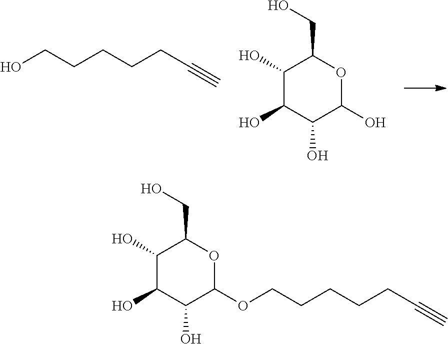

Hept-6-yn-1-ol (623.67 mg, 5.56 mmol, 692.97 μL) and (3S,4S,5S,6R)-6-(hydroxymethyl)tetrahydropyran-2,3,4,5-tetrol (500.00 mg, 2.78 mmol) were dissolved in DMF (10.00 mL), after which HCl (4 M, 695.00 μL) was added to the solution. The reaction mixture was stirred for 24 hours at 60° C. Solvent was removed in vacuo at room temperature to yield the crude product. The crude product was purified by reverse phase HPLC to yield (3S,4S,5S,6R)-2-hept-6-ynoxy-6-(hydroxymethyl)tetrahydropyran-3,4,5-triol (200.00 mg, 729.10 umol, 26.23% yield).

### Example 30—Synthesis of Intermediate M

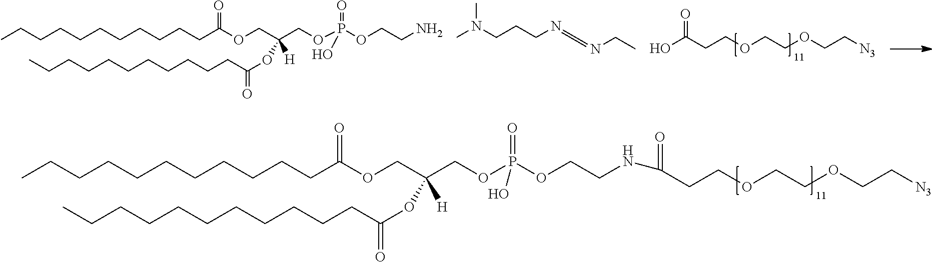

Under N₂ protection, [(2R)-3-[2-aminoethoxy(hydroxy)phosphoryl]oxy-2-dodecanoyloxy-propyl] dodecanoate (90.06 mg, 155.35 μmol) and 3-[2-[2-[2-[2-[2-[2-[2-[2-[2-[2-[2-[2-[2-[2-[2-[2-[2-[2-[2-[2-[2-(2-azidoethoxy)ethoxy]ethoxy]ethoxy]ethoxy]ethoxy]-ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]propanoic acid (100.00 mg, 155.35 μmol) was dissolved in dry DCM (3.00 mL) and dry DMF (3.00 mL) and cooled to 0° C. 3-(ethyliminomethyleneamino)-N,N-dimethyl-propan-1-amine (89.34 mg, 466.05 μmol) and DMAP (3.80 mg, 31.07 μmol) were then added in one portion. TEA (31.44 mg, 310.70 umol, 43.07 uL) was then added dropwise to the reaction mixture over 5 minutes. The reaction yielded [(2R)-3-[2-[3-[2-[2-[2-[2-[2-[2-[2-[2-[2-[2-[2-[2-[2-[2-[2-[2-[2-[2-[2-[2-[2-(2-azidoethoxy)ethoxy]ethoxy]ethoxy]ethoxy]ethoxy]-ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]ethoxy]propanoylamino]ethoxy-hydroxy-phosphoryl]oxy-2-dodecanoyloxy-propyl]dodecanoate (187.00 mg, 155.13 umol, 99.86% yield), which was used without further purification.

### Example 31—Synthesis of Compound 48

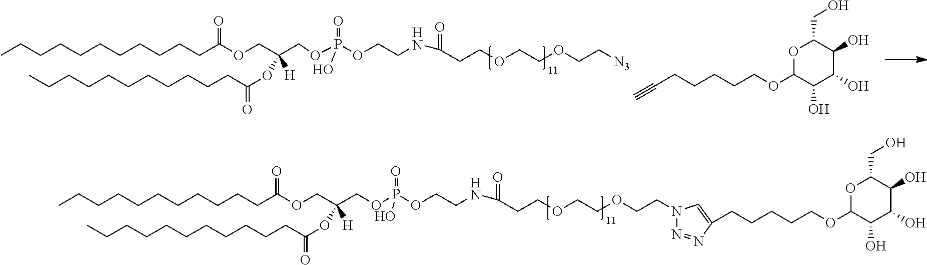

Intermediate M (30.00 mg, 24.89 umol) and Intermediate L were dissolved in MeOH (2.00 mL). CuSO₄ (6.22 mg, 24.89 μmol) and sodium ascorbate (7.39 mg, 37.34 μmol) were dissolved in water (2.00 mL) and added to the MeOH solution dropwise. The reaction mixture was stirred for 24 hours, after which it was filtered and the solvent removed in vacuo. The crude product was purified by reverse phase HPLC to yield the desired product (25.00 mg, 16.89 umol, 67.86% yield).

### Example 32—Nanoparticle Formulation of RNA with Compound 3

Compound 3 (3.04 μmol), 1,2-dioleoyl-sn-glycero-3-phosphoethanolamine (i.e., DOPE) (1.83 μmol), cholesterol (1.13 μmol), and 1,2-dimyristoyl-sn-glycero-3-phosphoethanolamine-N-[methoxy(polyethylene glycol)-2000] (i.e., C, 14-PEG) (0.09 μmol) were dissolved in absolute ethanol (1.02 mL) in a molar ratio of 50:30:18.5:1.5 (compound 3:DOPE:cholesterol:C14-PEG). Citrate buffer was added to this ethanolic solution to yield a final aqueous volume of 10% v/v. This buffered solution was added dropwise under stirring to a solution of RNA (0.568 μmol) dissolved in citrate buffer (1.02 mL) at pH 5.0. The resulting nanoparticle formulation was purified by passing it through a previously equilibrated gel filtration column (Sephadex G-25, GE Healthcare) to remove unformulated excipients. The formulation contained a final RNA concentration of 0.126 M. The particle size range as measured by dynamic light scattering (DLS) on a Wyatt DynaPro Plate Reader was 127.7 nm.

### Example 33—Nanoparticle Formulation of RNA with Compound 4

An RNA nanoparticle formulation was prepared using compound 4 in accordance with the procedure described in Example 27.

### Example 34—Nanoparticle Formulation of RNA with Compound 7

An RNA nanoparticle formulation was prepared using compound 7 in accordance with the procedure described in Example 27.

### Example 35—Nanoparticle Formulation of RNA with Compound 28

An RNA nanoparticle formulation was prepared using compound 28 in accordance with the procedure described in Example 27.

### Example 36—Nanoparticle Formulation of RNA with Compound 29

An RNA nanoparticle formulation was prepared using compound 29 in accordance with the procedure described in Example 27.

### Example 37—Nanoparticle Formulation of RNA with Compound 30

An RNA nanoparticle formulation was prepared using compound 30 in accordance with the procedure described in Example 27.

### Example 38—Nanoparticle Formulation of RNA with Compound 5

An RNA nanoparticle formulation was prepared using compound 5 in accordance with the procedure described in Example 27.

### Example 39—Nanoparticle Formulation of RNA with Compound 6

An RNA nanoparticle formulation was prepared using compound 6 in accordance with the procedure described in Example 27.

### Example 40—Nanoparticle Formulation of RNA with Compound 8

An RNA nanoparticle formulation was prepared using compound 8 in accordance with the procedure described in Example 27.

### Example 41—Nanoparticle Formulation of RNA with Compound 44

An RNA nanoparticle formulation was prepared using compound 44 in accordance with the procedure described in Example 27.

### Example 42—Nanoparticle Formulation of RNA with Compound 43

An RNA nanoparticle formulation was prepared using compound 43 in accordance with the procedure described in Example 27.

### Example 43—Nanoparticle Formulation of RNA with Compound 41

An RNA nanoparticle formulation was prepared using compound 41 in accordance with the procedure described in Example 27.

### Example 44—Nanoparticle Formulation of RNA with Compound 1

An RNA nanoparticle formulation was prepared using compound 1 in accordance with the procedure described in Example 27.

### Example 45—Nanoparticle Formulation of RNA with Compound 2

An RNA nanoparticle formulation was prepared using compound 2 in accordance with the procedure described in Example 27.

General Procedure for Evaluating Formulations is Insect Feeding Assays

The presently disclosed formulations can be evaluated in insect feeding assays to determine their efficacy in RNA delivery to an insect cell. Two model insects are used: western tarnished plant bug (WTPB, Lygus hesperus) and tarnished plant bug (TPB, Lygus lineolaris). Each formulation to be evaluated is prepared according to the general procedure described above in Examples 27-40 using as active agents an siRNA that targets an essential gene in TPB and an siRNA that targets an essential gene in WTPB. The feeding assay employed is based on a 96 well format and a sachet system as described by Habibi et al. (2002, Archives of Insect Biochem. and Phys. 50: 62-74) and U.S. Pat. No. 8,609,936, each of which is incorporated herein by reference in their entireties. The insect artificial diet is commercially available from Bio-Serv™ (Bio-Serv™ Diet F9644B, Frenchtown, N.J.).

Autoclaved boiling water is combined with Bio-Serv® Diet F9644B in a surface sterilized blender. Four surface sterilized chicken eggs are broken and the contents are added to the blender containing the diet mix. The mixture is blended until smooth and adjusted to one liter of volume and allowed to cool. Feeding samples are prepared by mixing the siRNA formulations described above in the desired concentration with an equivalent volume of the blended diet.

A sheet of Parafilm® (Pechiney Plastic Packing, Chicago, Ill.) is placed over a 96-well format vacuum manifold with a vacuum of approximately −20 millimeters mercury, which is sufficient to cause extrusion of the Parafilm® into the wells. Forty microliters of test sample are added to the Parafilm® wells. A sheet of Mylar film (Clear Lam Packaging, Inc., Elk Grove Village, Ill.) is then placed over the Parafilm® and sealed gently with a tacking iron (Bienfang Sealector II, Hunt Corporation, Philadelphia, Pa.). The Parafilm® sachets are then placed over a flat-bottom 96-well plate containing the Lygus eggs suspended in agarose. Upon hatching, Lygus nymphs will feed by piercing the sachet that is presented above them. Insect diet sachets are replaced on days two and four. Stunting and mortality scores are determined on day 5 and compared to the untreated controls. Those formulations that significantly increase stunting and mortality relative to the untreated controls demonstrate that the formulations are effective in delivering the siRNAs to the insect cells.

1. A formulation comprising:
(1) at least one formulation transport agent;
(2) at least one complexing agent; and
(3) a first active agent that modulates a trait of a target organism;
wherein the target organism is an insect, a plant, or a plant pathogen.

2-6. (canceled)

7. The formulation of claim 1, wherein the at least one formulation transport agent is a compound of formula (I):
A-B-C (I)
wherein
A is a group that facilitates transport of the formulation to, into, and within a cell of the target organism and/or decomplexation of the formulation;
B is a linker; and
C is a group that is non-covalently associated to the at least one complexing agent;
wherein the linker B is at least in part formed from a moiety of A and a moiety of C.

8-9. (canceled)

10. The formulation of claim 7, wherein:
A is group derived from glucose, sucrose, maltose, kanamycin, arginine, lysine, or histidine, or a group selected from the group consisting of formulae (II), (III), (IV), (V), (VI), (VII), (VIII), (IX), and (X): 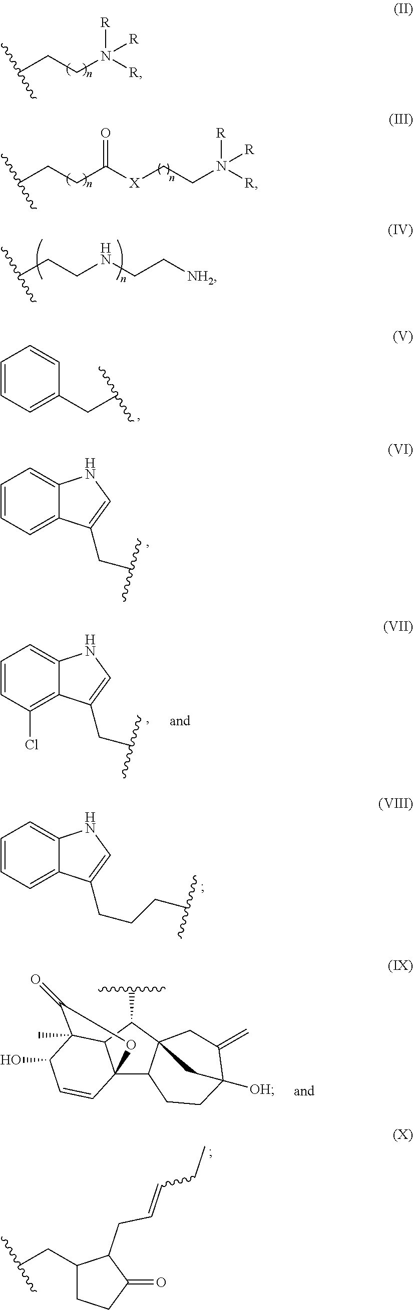
wherein
X is O or NH;
R is —H, —CH₃, —CH₂CH₃, or —CH₂CH₂OH; and
n is 0, 1, or 2.

11. The formulation of claim 7, wherein:
B is a covalent bond or a group selected from the group consisting of formulae (XI), (XII), (XIII), (XIV), (XV), (XVI), (XVII), and (XVIII): 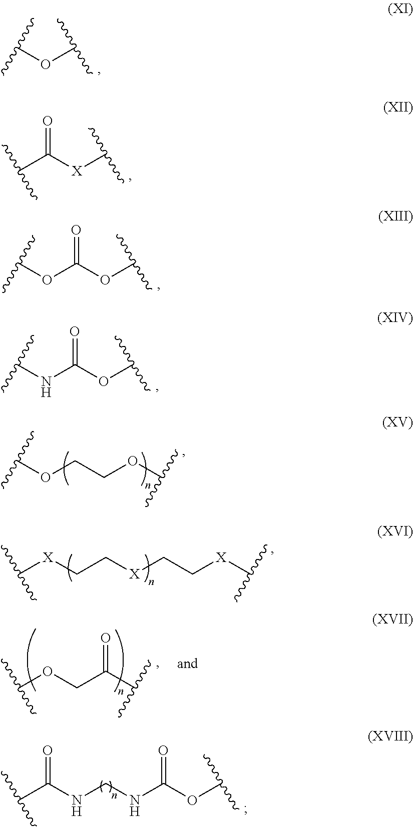
wherein
X is, independently, O or NH; and
n and integer in the range of from 1 to 10.

12. The formulation of claim 7, wherein:
C is a group selected from the group consisting of formulae (XIX), (XX), (XXI), (XXII), (XXIII), (XXIV), (XXV), and (XXVI):

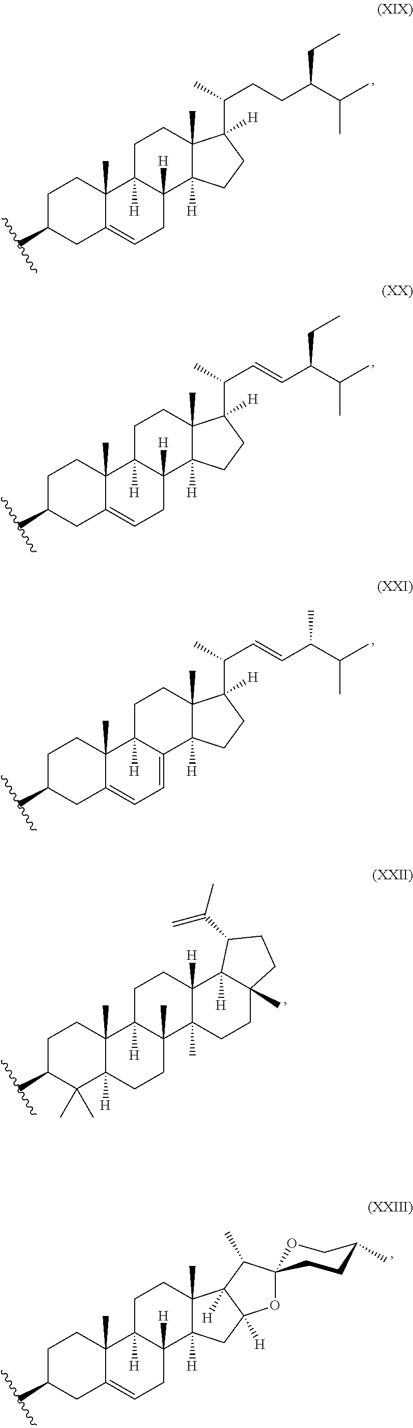

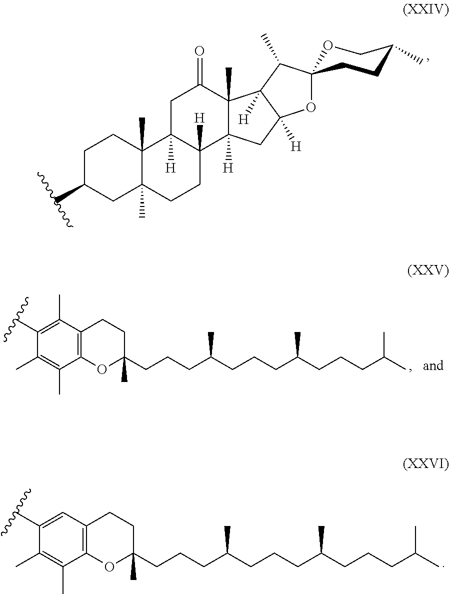

13. The formulation of claim 10, wherein:
B is a covalent bond or a group selected from the group consisting of formulae (XI), (XII), (XIII), (XIV), (XV), (XVI), (XVII), and (XVIII): 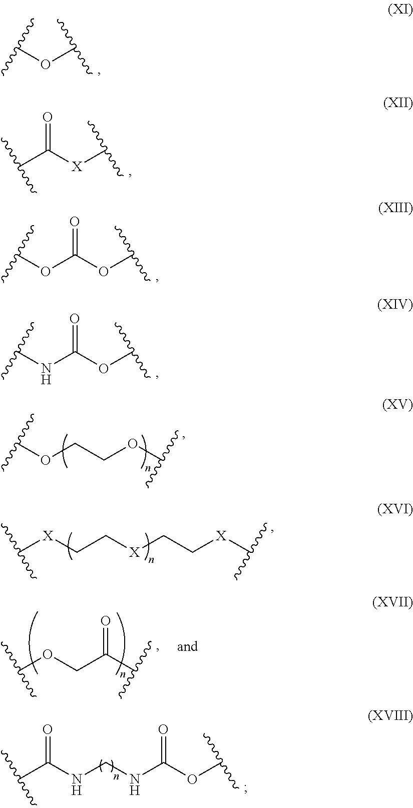
wherein
X is, independently, O or NH; and
n and integer in the range of from 1 to 10.

14. The formulation of claim 13, wherein:
C is a group selected from the group consisting of formulae (XIX), (XX), (XXI), (XXII), (XXIII), (XXIV), (XXV), and (XXVI):

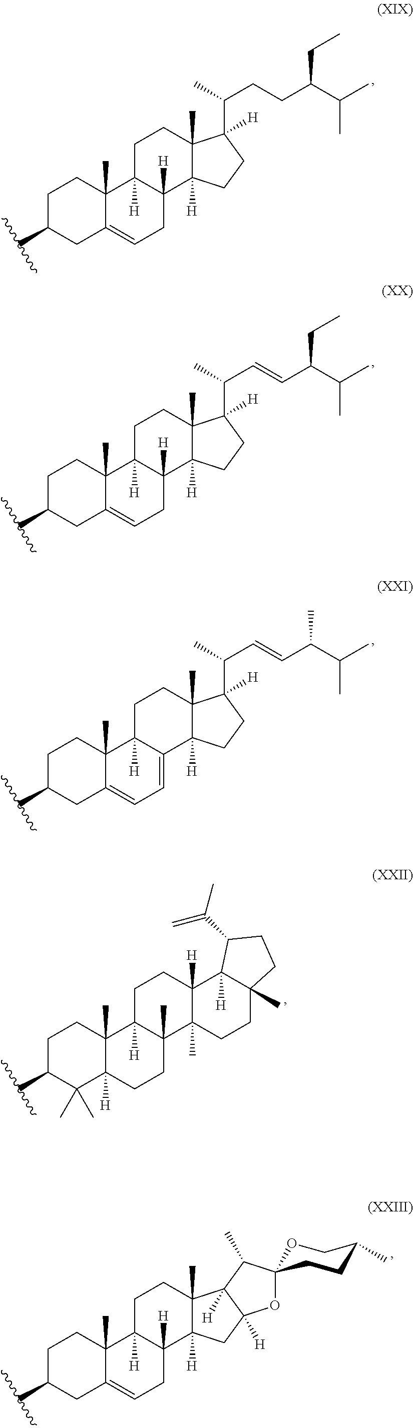

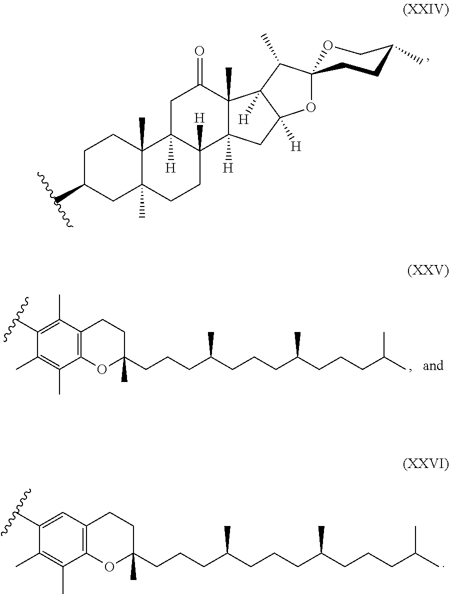

15. The formulation of claim 7, wherein the compound of formula (I) is a compound selected from the group consisting of compounds (1) through (50):

| Com- | |
|---|---|
| pound | |
| Number | Structure |
| 1 | 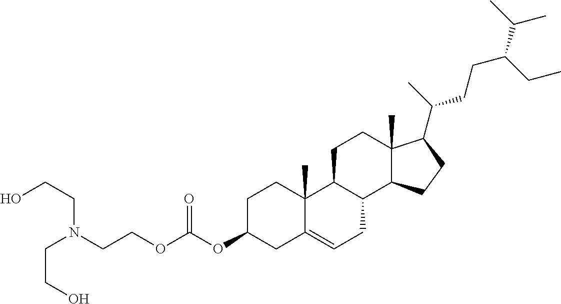 [SMILES: CC[C@H](CC[C@@H](C)[C@H]1CC[C@H]2[C@@H]3CC=C4C[C@@H](OC(=O)OCCN(CCO)CCO)CC[C@]4(C)[C@H]3CC[C@]12C)C(C)C] |
| 2 | 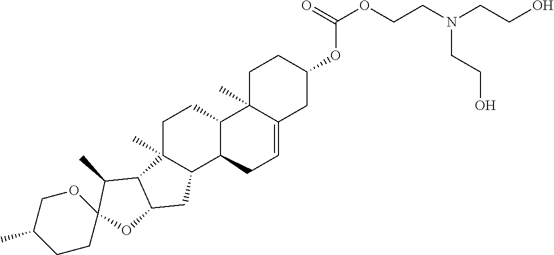 [SMILES: C[C@H]1CC[C@]2(OC1)O[C@H]1C[C@H]3[C@@H]4CC=C5C[C@@H](OC(=O)OCCN(CCO)CCO)CC[C@]5(C)[C@H]4CC[C@]3(C)[C@H]1[C@@H]2C] |
| 3 | 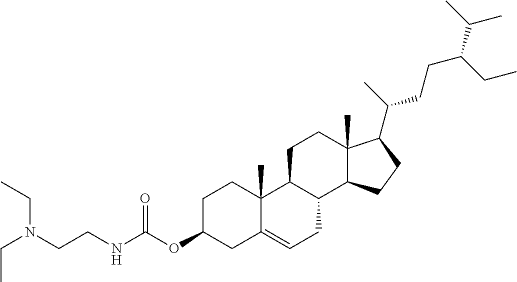 [SMILES: CC[C@H](CC[C@@H](C)[C@H]1CC[C@H]2[C@@H]3CC=C4C[C@@H](OC(=O)NCCN(CC)CC)CC[C@]4(C)[C@H]3CC[C@]12C)C(C)C] |
| 4 | 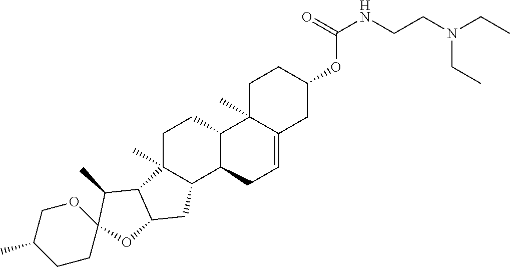 [SMILES: CCN(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1] |
| 5 | 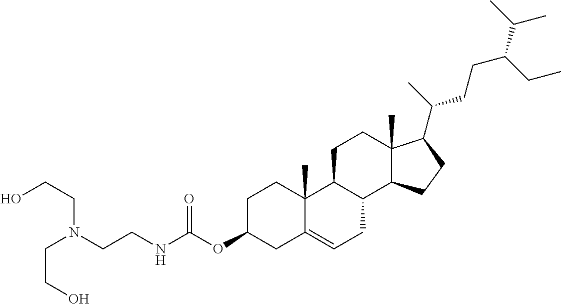 [SMILES: CC[C@H](CC[C@@H](C)[C@H]1CC[C@H]2[C@@H]3CC=C4C[C@@H](OC(=O)NCCN(CCO)CCO)CC[C@]4(C)[C@H]3CC[C@]12C)C(C)C] |
| 6 | 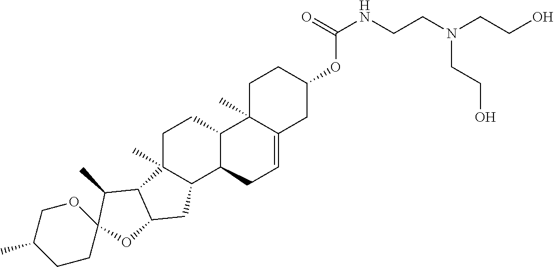 [SMILES: C[C@H]1CC[C@]2(OC1)O[C@H]1C[C@H]3[C@@H]4CC=C5C[C@@H](OC(=O)NCCN(CCO)CCO)CC[C@]5(C)[C@H]4CC[C@]3(C)[C@H]1[C@@H]2C] |
| 7 | 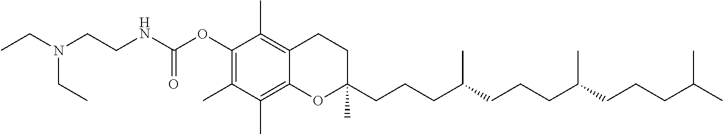 [SMILES: CCN(CC)CCNC(=O)Oc1c(C)c(C)c2c(c1C)CC[C@@](C)(CCC[C@H](C)CCC[C@H](C)CCCC(C)C)O2] |
| 8 | 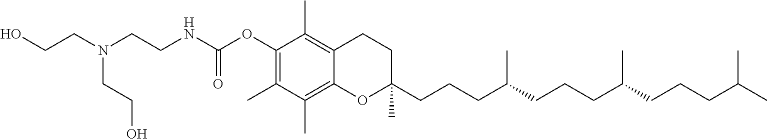 [SMILES: Cc1c(C)c2c(c(C)c1OC(=O)NCCN(CCO)CCO)CC[C@@](C)(CCC[C@H](C)CCC[C@H](C)CCCC(C)C)O2] |
| 9 | 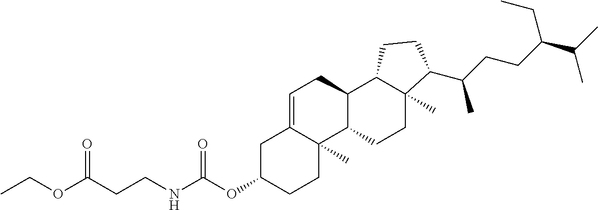 [SMILES: CCOC(=O)CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 10 | 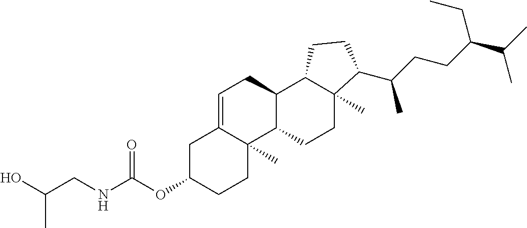 [SMILES: CC[C@H](CC[C@@H](C)[C@H]1CC[C@H]2[C@@H]3CC=C4C[C@@H](OC(=O)NCC(C)O)CC[C@]4(C)[C@H]3CC[C@]12C)C(C)C] |
| 11 | 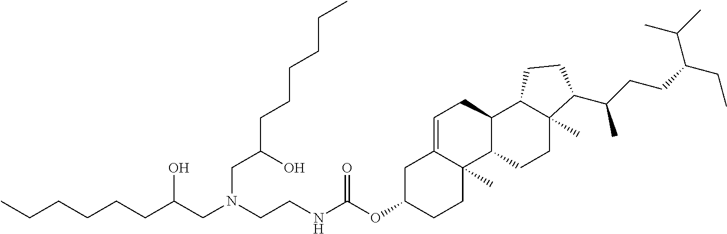 [SMILES: CCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCC] |
| 12 | 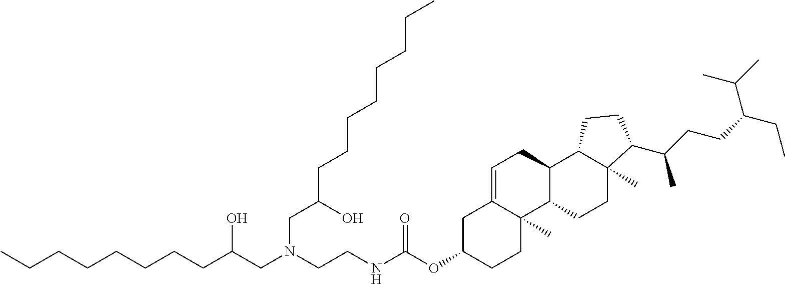 [SMILES: CCCCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCCCC] |
| 13 | 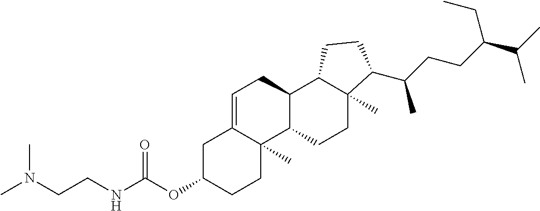 [SMILES: CC[C@H](CC[C@@H](C)[C@H]1CC[C@H]2[C@@H]3CC=C4C[C@@H](OC(=O)NCCN(C)C)CC[C@]4(C)[C@H]3CC[C@]12C)C(C)C] |
| 14 | 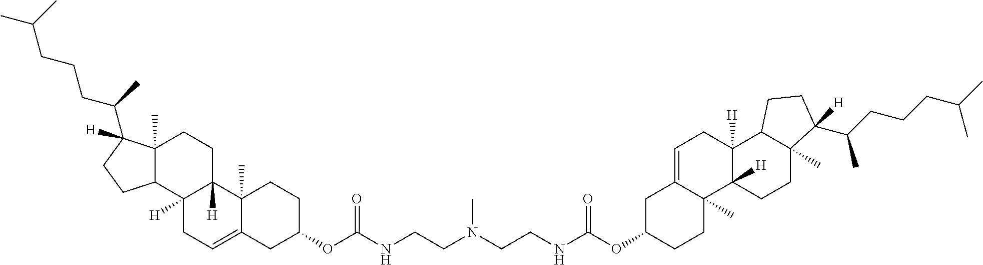 |
| 15 | 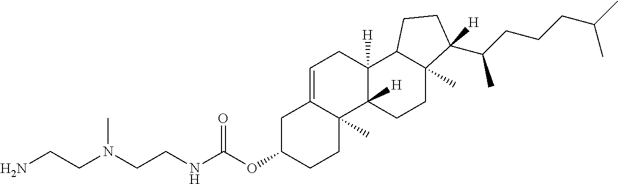 |
| 16 | 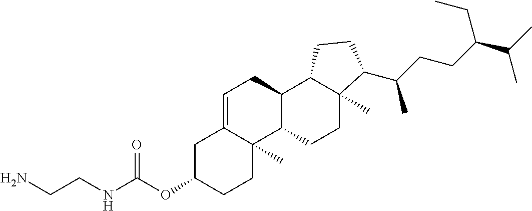 [SMILES: CC[C@H](CC[C@@H](C)[C@H]1CC[C@H]2[C@@H]3CC=C4C[C@@H](OC(=O)NCCN)CC[C@]4(C)[C@H]3CC[C@]12C)C(C)C] |
| 17 | 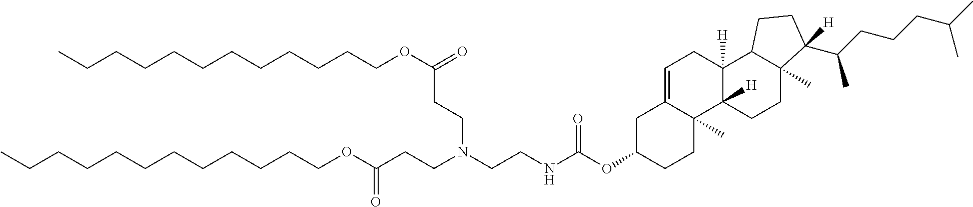 |
| 18 | 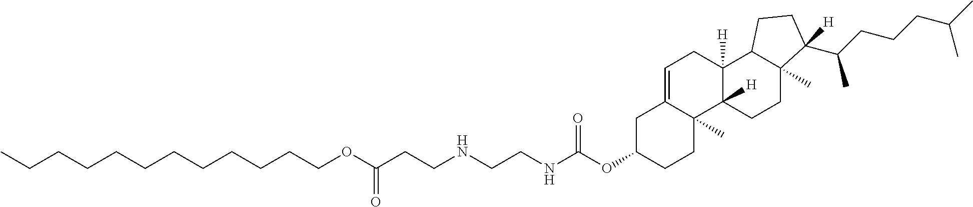 |
| 19 | 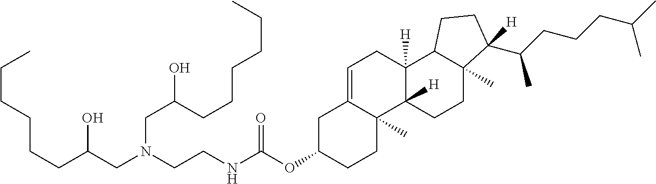 |
| 20 | 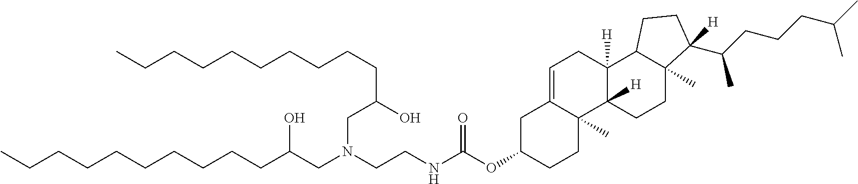 |
| 21 | 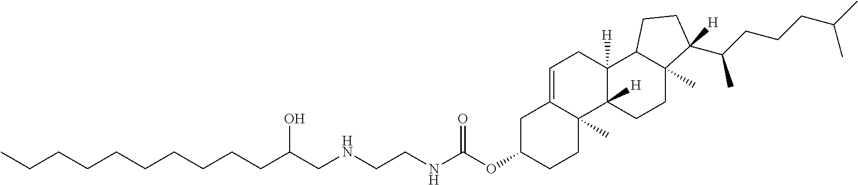 |
| 22 | 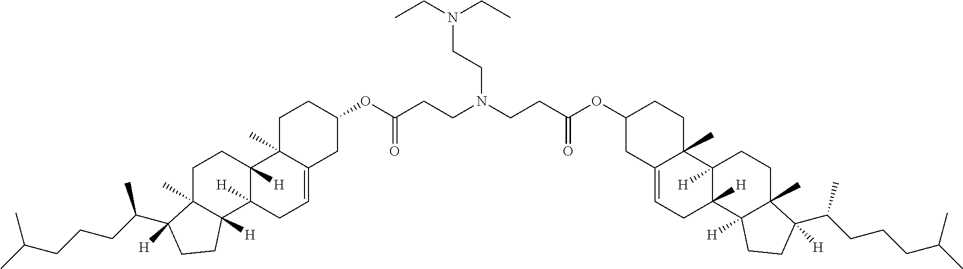 |
| 23 | 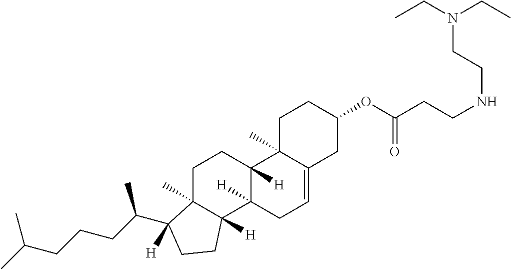 |
| 24 | 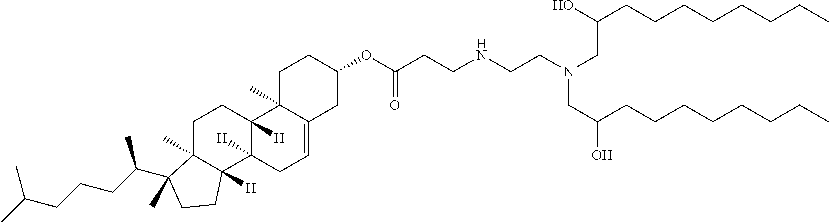 |
| 25 | 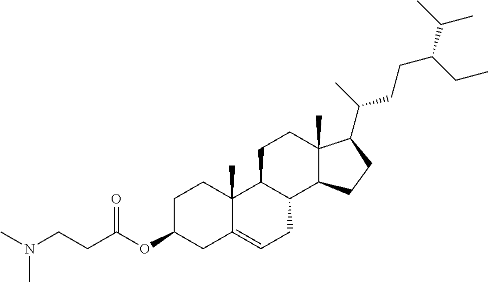 [SMILES: CC[C@H](CC[C@@H](C)[C@H]1CC[C@H]2[C@@H]3CC=C4C[C@@H](OC(=O)CCN(C)C)CC[C@]4(C)[C@H]3CC[C@]12C)C(C)C] |
| 26 | 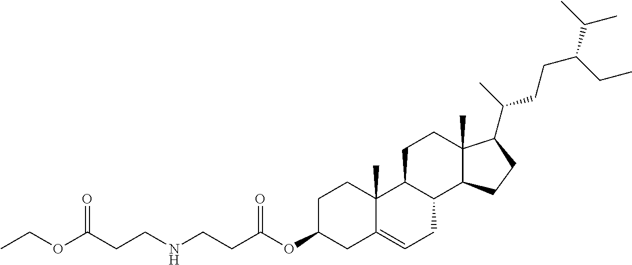 [SMILES: CCOC(=O)CCNCCC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 27 | 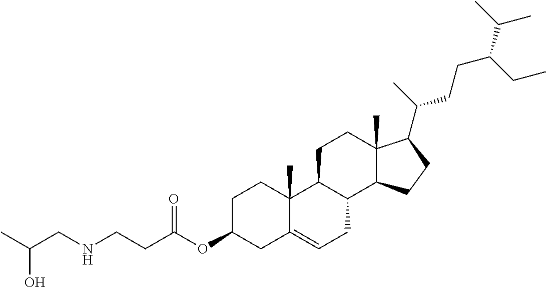 [SMILES: CC[C@H](CC[C@@H](C)[C@H]1CC[C@H]2[C@@H]3CC=C4C[C@@H](OC(=O)CCNCC(C)O)CC[C@]4(C)[C@H]3CC[C@]12C)C(C)C] |
| 28 | 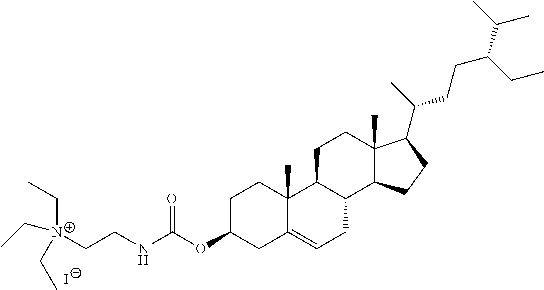 [SMILES: CC[C@H](CC[C@@H](C)[C@H]1CC[C@H]2[C@@H]3CC=C4C[C@@H](OC(=O)NCC[N+](CC)(CC)CC)CC[C@]4(C)[C@H]3CC[C@]12C)C(C)C ; [I-]] |
| 29 | 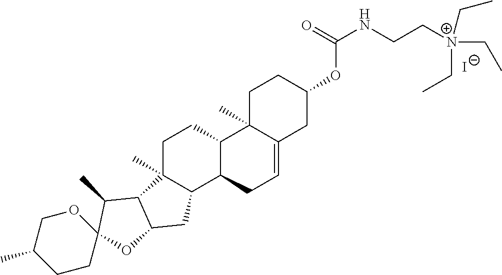 [SMILES: CC[N+](CC)(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1 ; [I-]] |
| 30 | 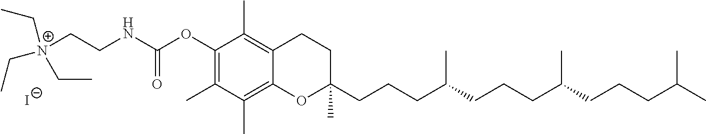 [SMILES: CC[N+](CC)(CC)CCNC(=O)Oc1c(C)c(C)c2c(c1C)CC[C@@](C)(CCC[C@H](C)CCC[C@H](C)CCCC(C)C)O2 ; [I-]] |
| 31 | 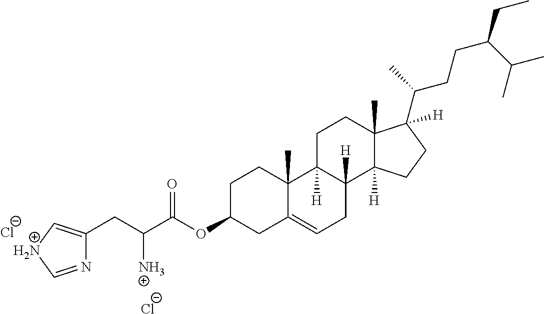 |
| 32 | 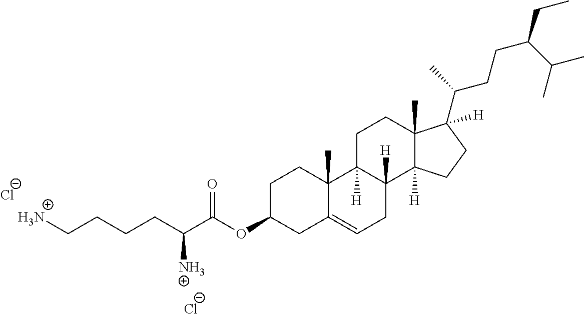 |
| 33 | 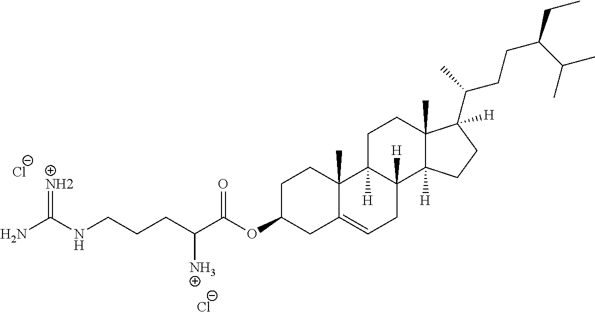 |
| 34 | 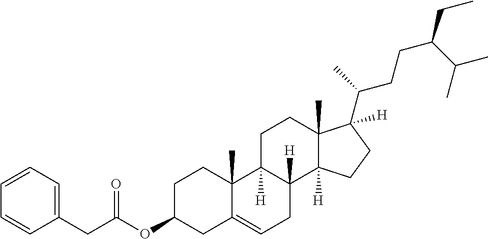 |
| 35 | 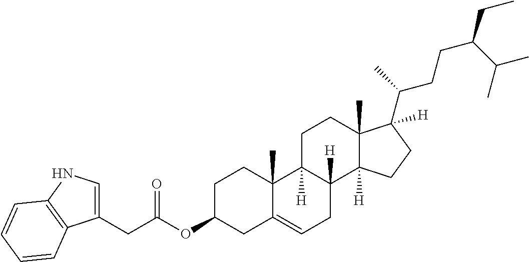 |
| 36 | 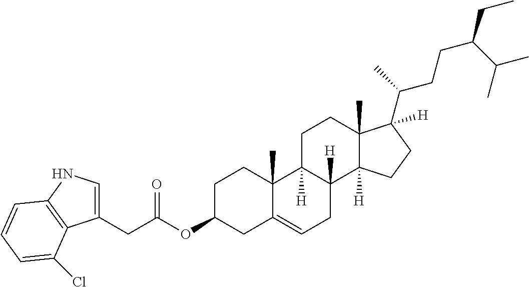 |
| 37 | 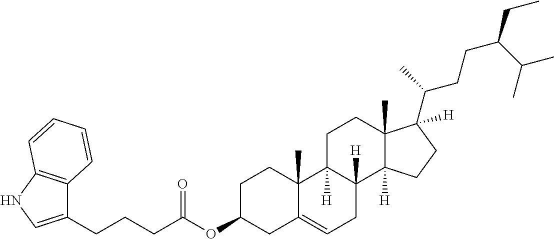 |
| 38 | 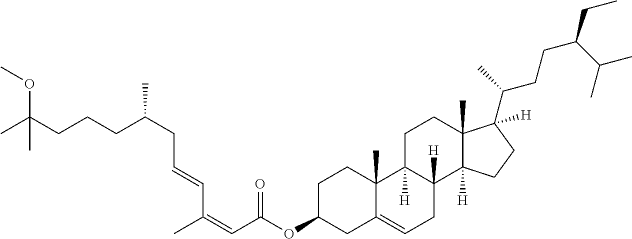 |
| 39 | 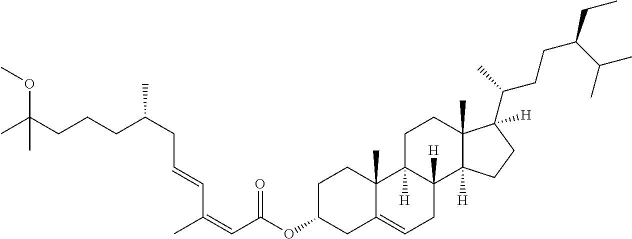 |
| 40 | 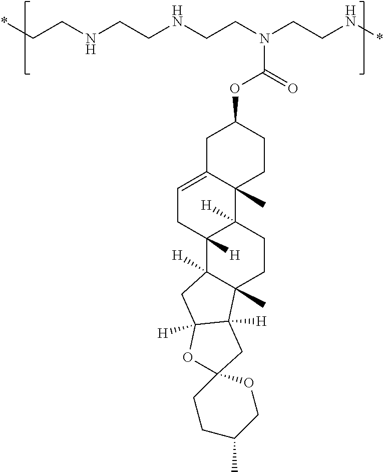 |
| 41 | 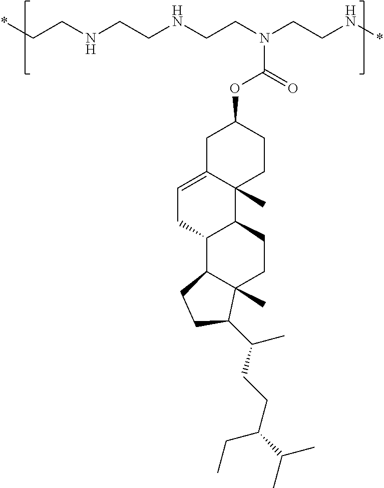 |
| 42 | 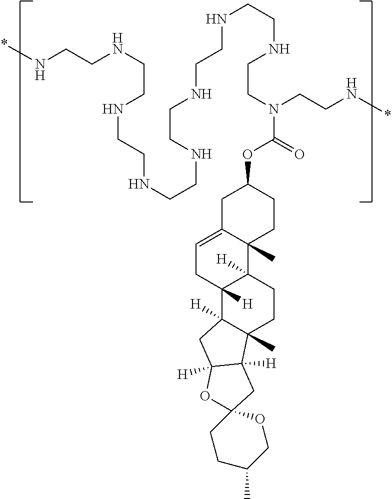 |
| 43 | 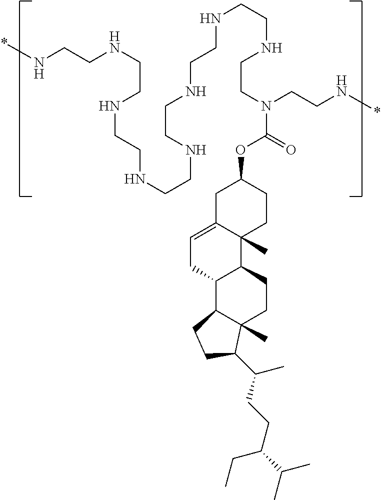 |
| 44 | 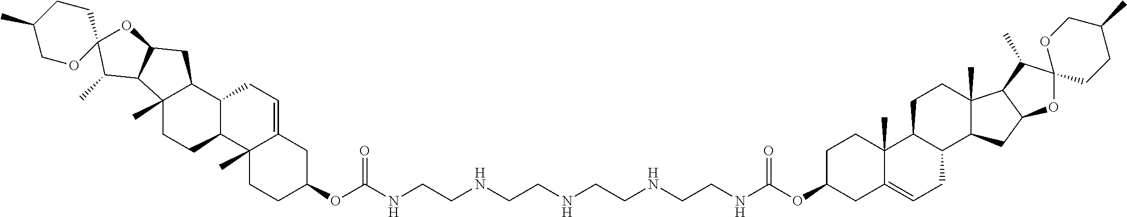 |
| 45 | 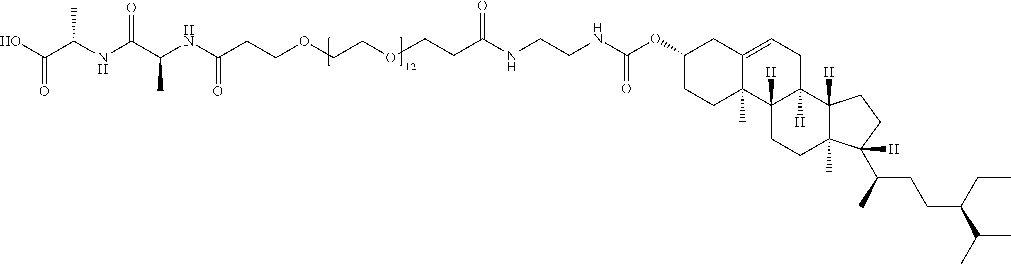 |
| 46 | 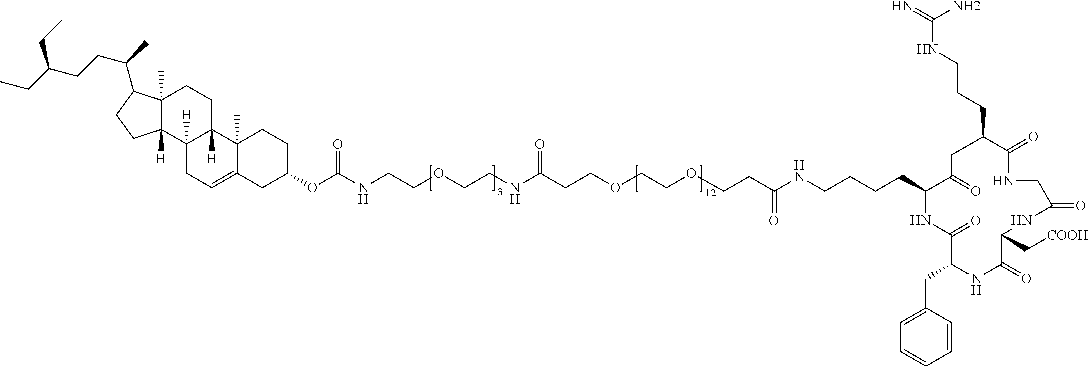 |
| 47 | 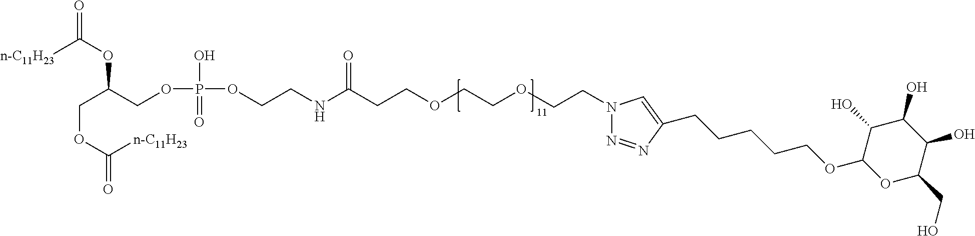 |
| 48 | 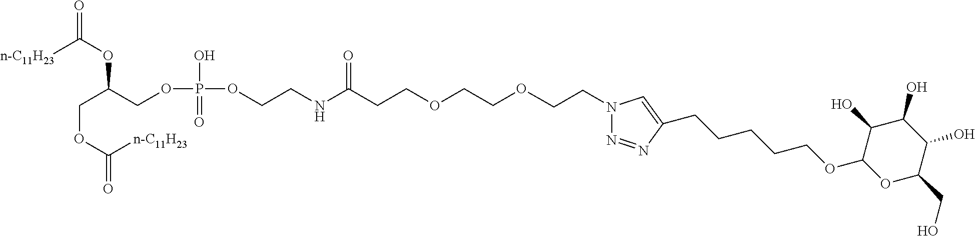 |
| 49 | 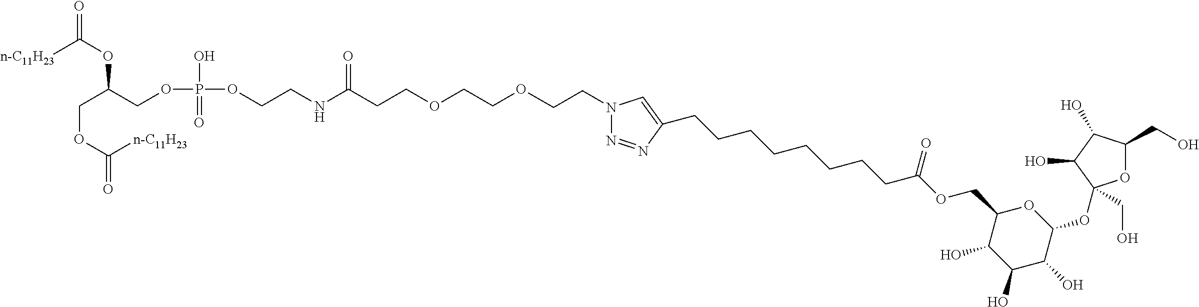 |
| 50 | 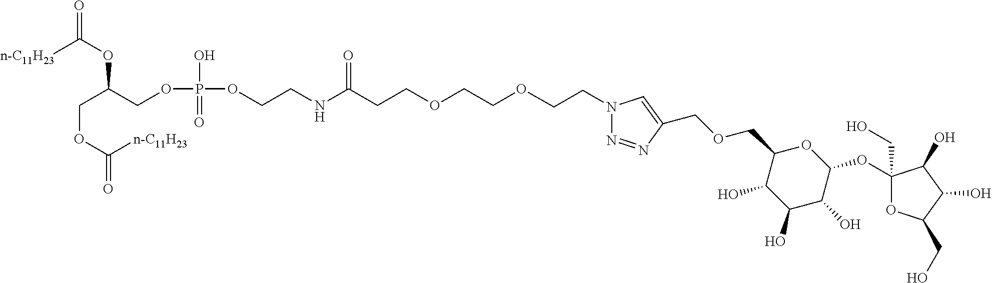 |

16. The formulation of claim 7, wherein the compound of formula (I) is a gibberellic acid derivative of formula (XXVII): 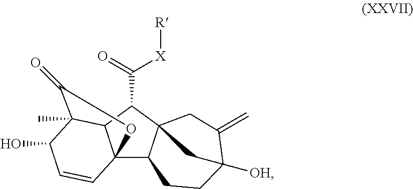
wherein
X is O or NH; and
R′ is an alkyl group or the residue of a insect-, plant-, or plant pathogen-derived steroid, tocopherol, endogenous auxin, or carbohydrate.

17. The formulation of claim 16, wherein R′ is a C₁ to C₂₀ alkyl group.

18. The formulation of claim 16, wherein:
X is O and R′ is a C₁₂ alkyl group; or
X is O or NH and R′ is a group of formula (XXVIII):

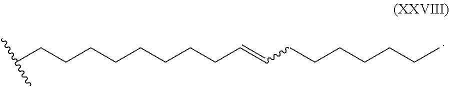 [SMILES: *CCCCCCCCC=CCCCCCCC]

19. The formulation of claim 16, wherein X is O and R′ is a group selected from the group consisting of formulae (V), (VI), (VII), (VIII), (XIX), (XX), (XXI), (XXII), (XXIII), (XXIV), (XXV), and (XXVI):

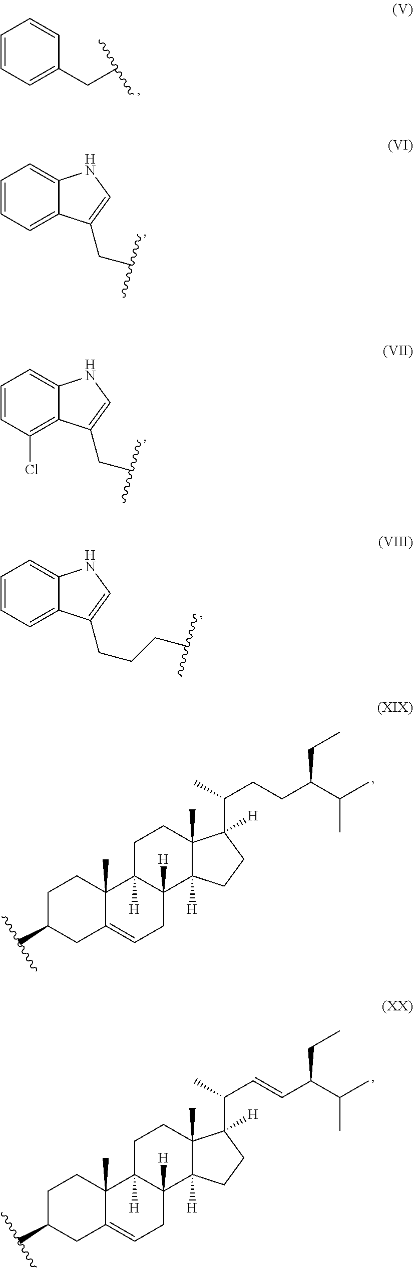

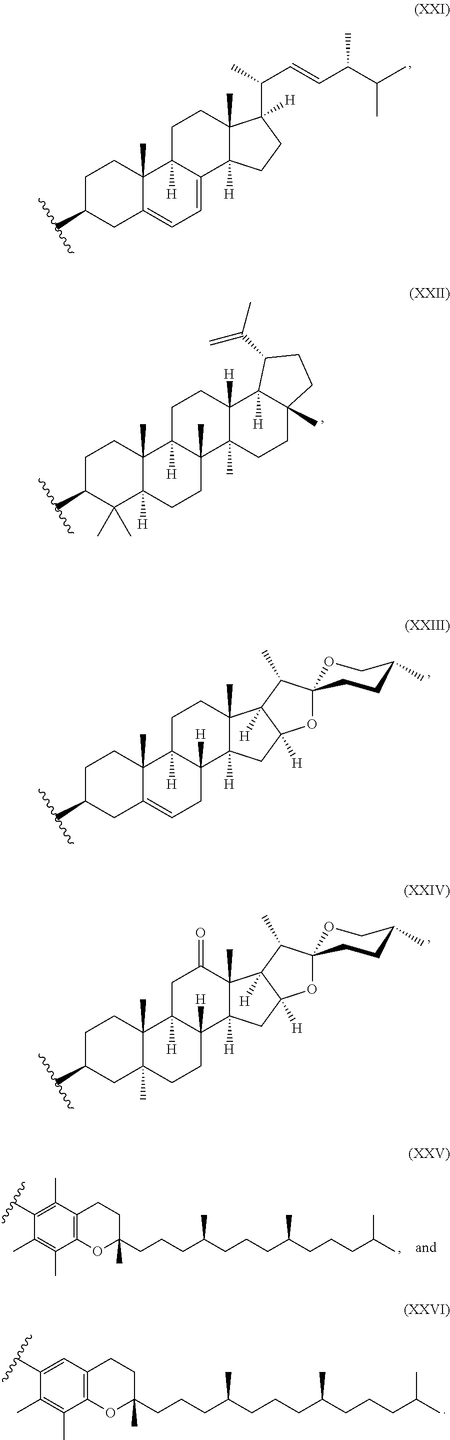

20. The formulation of claim 16, wherein X is O and R′ is a group derived from glucose, sucrose, maltose, or kanamycin.

21. (canceled)

22. The formulation of claim 1, further comprising at least one additional active agent to be delivered.

23-28. (canceled)

29. The formulation of claim 1, wherein the first active agent is an oligonucleotide or a polynucleotide.

30-39. (canceled)

40. The formulation of claim 1, wherein the first active agent is an RNA.

41-44. (canceled)

45. A method of regulating expression of a gene in a target organism, comprising applying the formulation of any one of claims 1-44 to the target organism.

46. A method of modulating a trait of a plant, comprising delivering to the plant an effective amount of the formulation of claim 29.

47-53. (canceled)

54. A method of modulating a trait of an insect, comprising delivering an effective amount of the formulation of claim 29 to the insect, to a plant infested with the insect, or to a plant prior to infestation with the insect.

55. (canceled)

56. A method of modulating the pathogenicity of a plant pathogen, comprising applying the formulation of claim 29 to the plant pathogen, to a plant infected with the plant pathogen, or to a plant prior to infection with the plant pathogen.

57. A plant cell, insect cell, fungal cell, nematodic cell, or bacterial cell comprising the formulation of claim 1.

58. A compound of formula (I):
A-B-C (I)
wherein
A is a group that can facilitate transport of a formulation to, into, and within a cell of a target organism and/or decomplexation of the formulation within the target organism;
B is a linker; and
C is a group that is non-covalently associated to at least one complexing agent of the formulation;
wherein
the linker B is at least in part formed from a moiety of A and a moiety of C;
the formulation comprises a first active agent that modulates a trait of a target organism and at least one complexing agent; and
the target organism is an insect, a plant, or a plant pathogen.

59-60. (canceled)

61. The compound of claim 58, wherein:
A is a group derived from glucose, sucrose, maltose, kanamycin, arginine, lysine, or histidine or a group selected from the group consisting of formulae (II), (III), (IV), (V), (VI), (VII), (VIII), (IX), and (X): 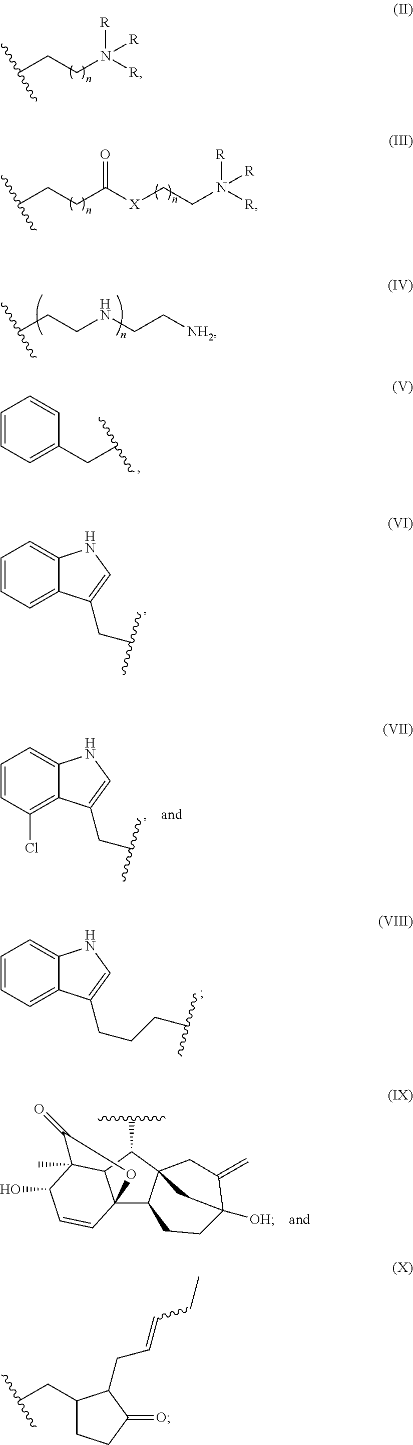
wherein
X is O or NH;
R is —H, —CH₃, —CH₂CH₃, or —CH₂CH₂OH; and
n is 0, 1, or 2.

62. The compound of claim 58, wherein:
B is a covalent bond or a group selected from the group consisting of formulae (XI), (XII), (XIII), (XIV), (XV), (XVI), (XVII), and (XVIII): 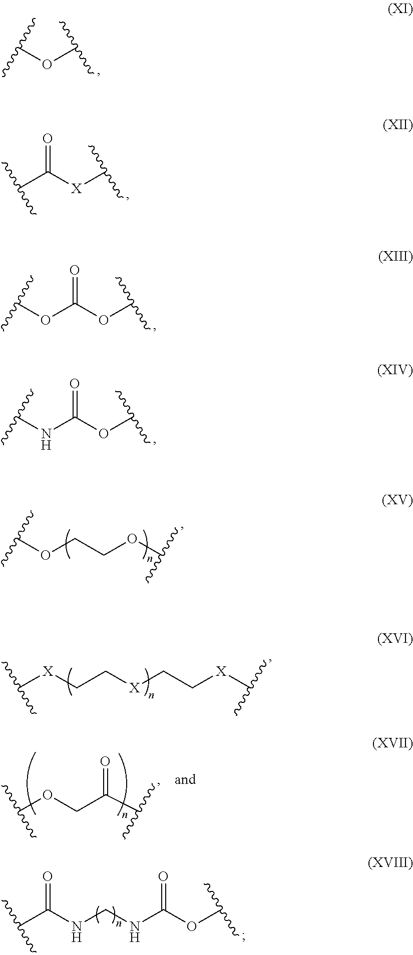
wherein
X is, independently, O or NH; and
n and integer in the range of from 1 to 10.

63. The compound of claim 58, wherein:
C is a group selected from the group consisting of formulae (XIX), (XX), (XXI), (XXII), (XXIII), (XXIV), (XXV), and (XXVI):

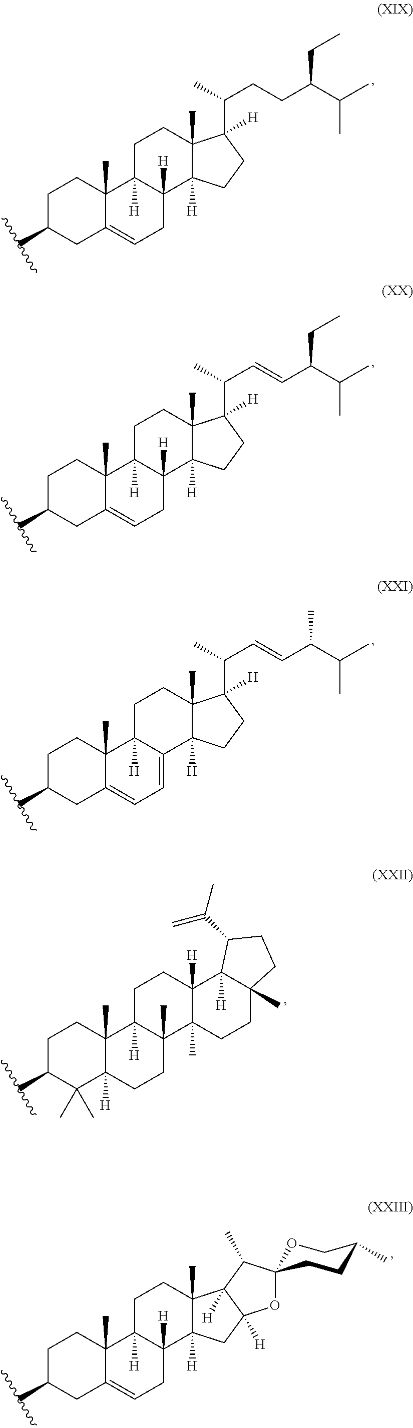

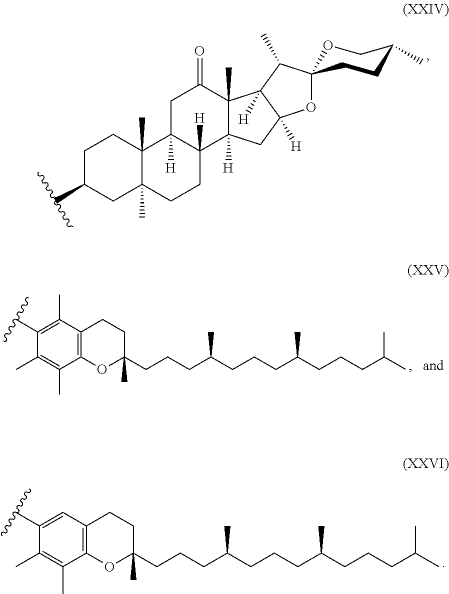

64. The compound of claim 61, wherein:
B is a covalent bond or a group selected from the group consisting of formulae (XI), (XII), (XIII), (XIV), (XV), (XVI), (XVII), and (XVIII): 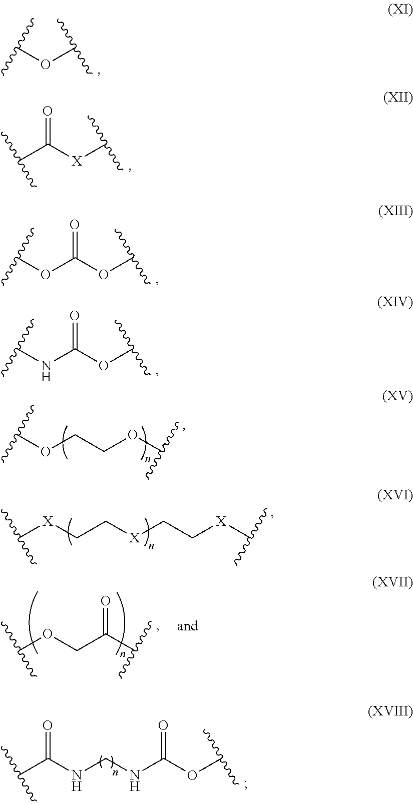
wherein
X is, independently, O or NH; and
n and integer in the range of from 1 to 10.

65. The compound of claim 64, wherein:
C is a group selected from the group consisting of formulae (XIX), (XX), (XXI), (XXII), (XXIII), (XXIV), (XXV), and (XXVI):

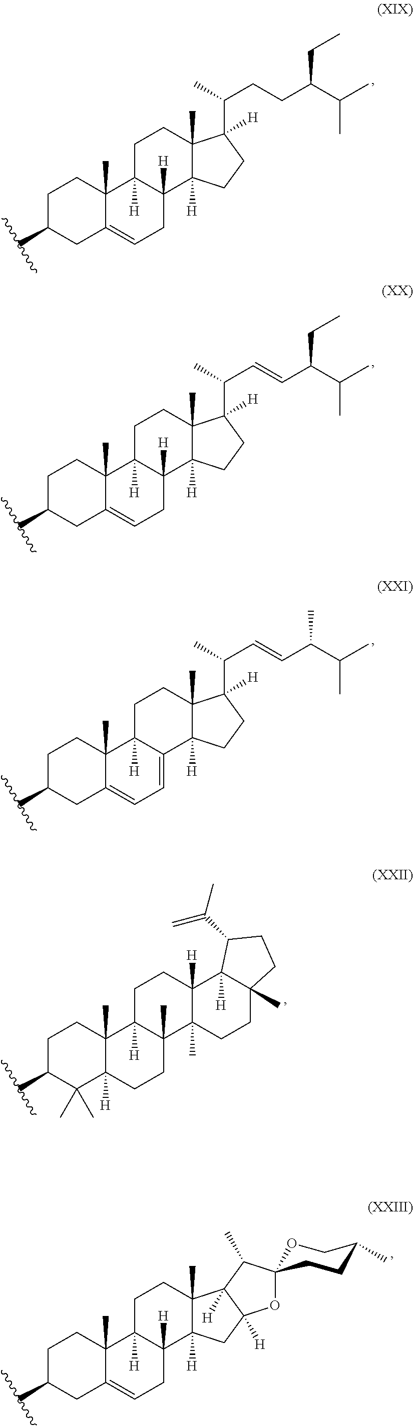

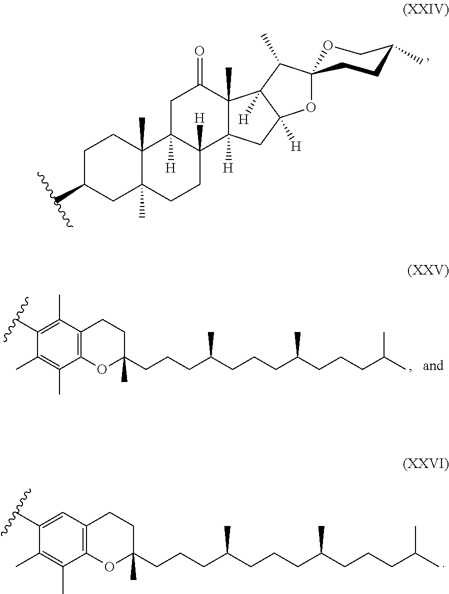

66. The compound of claim 58, wherein the compound of formula (I) is a compound selected from the group consisting of compounds (1) through (50):

| Compound | |
|---|---|
| Number | Structure |
| 1 | 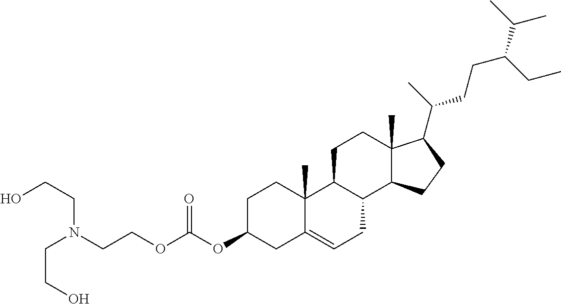 [SMILES: CC[C@H](CC[C@@H](C)[C@H]1CC[C@H]2[C@@H]3CC=C4C[C@@H](OC(=O)OCCN(CCO)CCO)CC[C@]4(C)[C@H]3CC[C@]12C)C(C)C] |
| 2 | 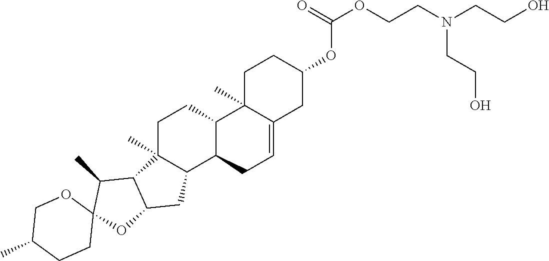 [SMILES: C[C@H]1CC[C@]2(OC1)O[C@H]1C[C@H]3[C@@H]4CC=C5C[C@@H](OC(=O)OCCN(CCO)CCO)CC[C@]5(C)[C@H]4CC[C@]3(C)[C@H]1[C@@H]2C] |
| 3 | 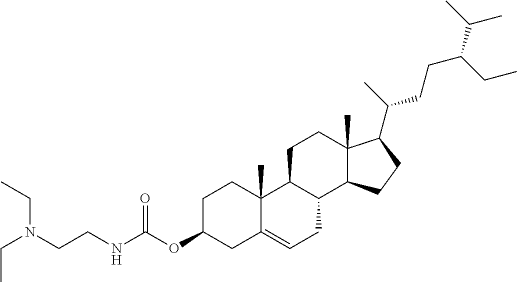 [SMILES: CC[C@H](CC[C@@H](C)[C@H]1CC[C@H]2[C@@H]3CC=C4C[C@@H](OC(=O)NCCN(CC)CC)CC[C@]4(C)[C@H]3CC[C@]12C)C(C)C] |
| 4 | 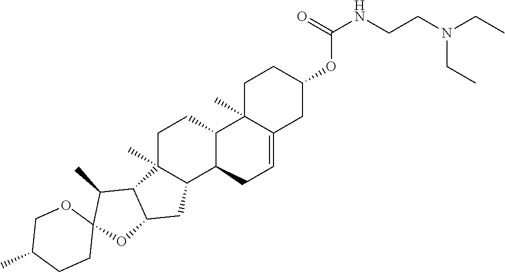 [SMILES: CCN(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1] |
| 5 | 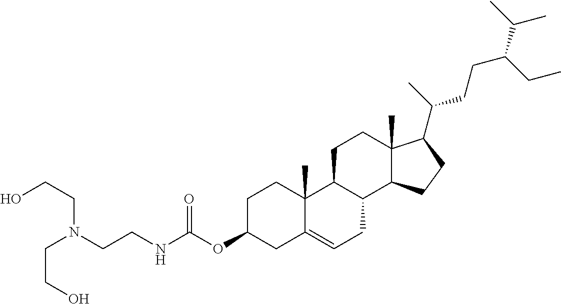 [SMILES: CC[C@H](CC[C@@H](C)[C@H]1CC[C@H]2[C@@H]3CC=C4C[C@@H](OC(=O)NCCN(CCO)CCO)CC[C@]4(C)[C@H]3CC[C@]12C)C(C)C] |
| 6 | 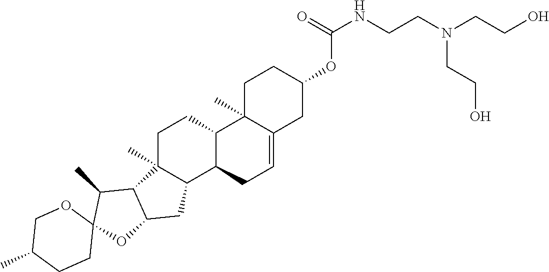 [SMILES: C[C@H]1CC[C@]2(OC1)O[C@H]1C[C@H]3[C@@H]4CC=C5C[C@@H](OC(=O)NCCN(CCO)CCO)CC[C@]5(C)[C@H]4CC[C@]3(C)[C@H]1[C@@H]2C] |
| 7 | 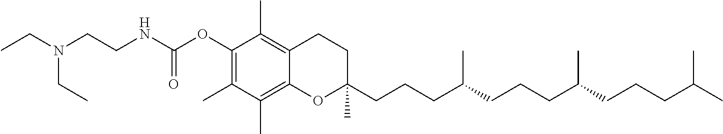 [SMILES: CCN(CC)CCNC(=O)Oc1c(C)c(C)c2c(c1C)CC[C@@](C)(CCC[C@H](C)CCC[C@H](C)CCCC(C)C)O2] |
| 8 | 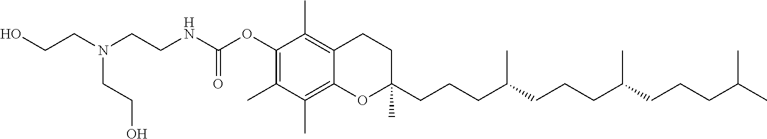 [SMILES: Cc1c(C)c2c(c(C)c1OC(=O)NCCN(CCO)CCO)CC[C@@](C)(CCC[C@H](C)CCC[C@H](C)CCCC(C)C)O2] |
| 9 | 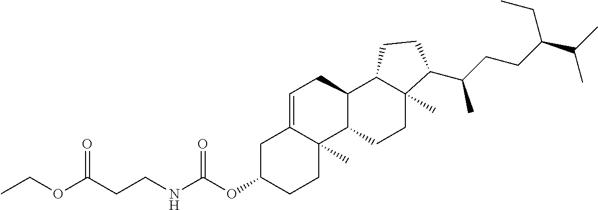 [SMILES: CCOC(=O)CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 10 | 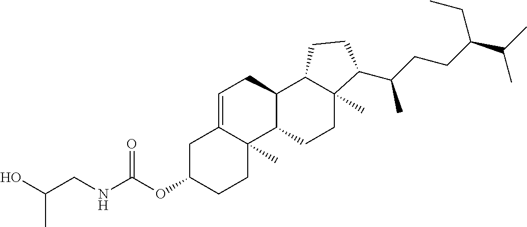 [SMILES: CC[C@H](CC[C@@H](C)[C@H]1CC[C@H]2[C@@H]3CC=C4C[C@@H](OC(=O)NCC(C)O)CC[C@]4(C)[C@H]3CC[C@]12C)C(C)C] |
| 11 | 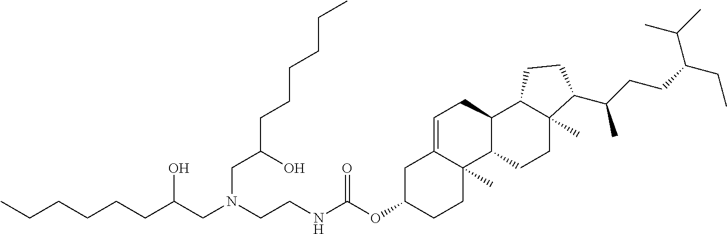 [SMILES: CCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCC] |
| 12 | 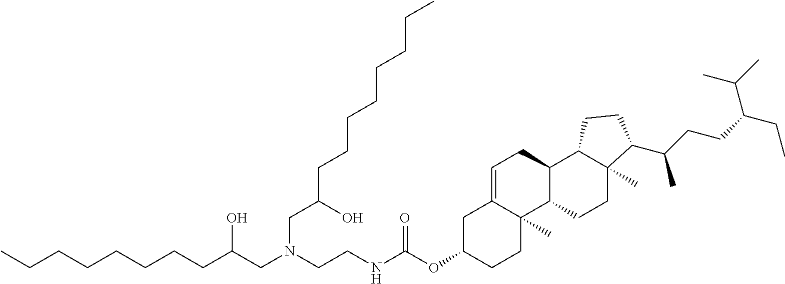 [SMILES: CCCCCCCCC(O)CN(CCNC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1)CC(O)CCCCCCCC] |
| 13 | 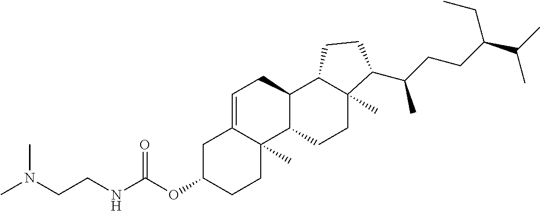 [SMILES: CC[C@H](CC[C@@H](C)[C@H]1CC[C@H]2[C@@H]3CC=C4C[C@@H](OC(=O)NCCN(C)C)CC[C@]4(C)[C@H]3CC[C@]12C)C(C)C] |
| 14 | 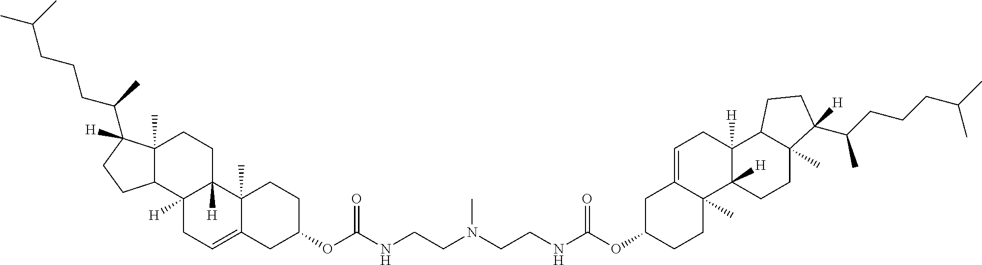 |
| 15 | 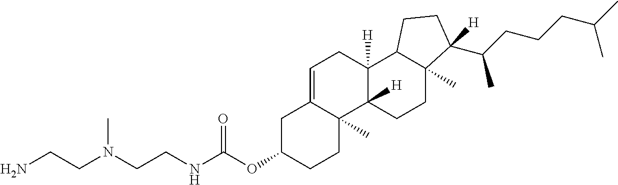 |
| 16 | 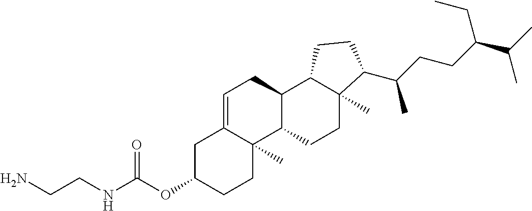 [SMILES: CC[C@H](CC[C@@H](C)[C@H]1CC[C@H]2[C@@H]3CC=C4C[C@@H](OC(=O)NCCN)CC[C@]4(C)[C@H]3CC[C@]12C)C(C)C] |
| 17 | 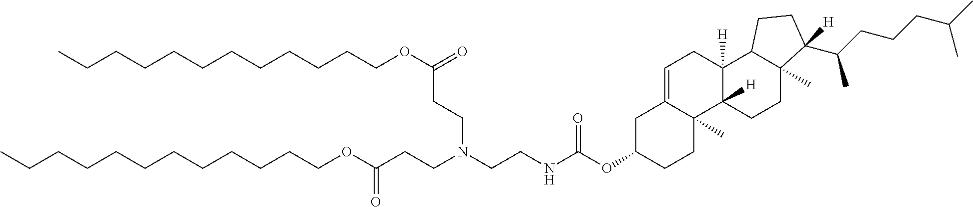 |
| 18 | 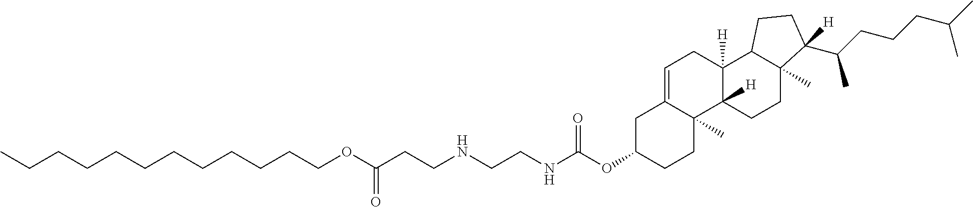 |
| 19 | 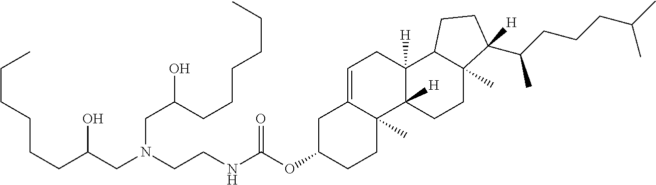 |
| 20 | 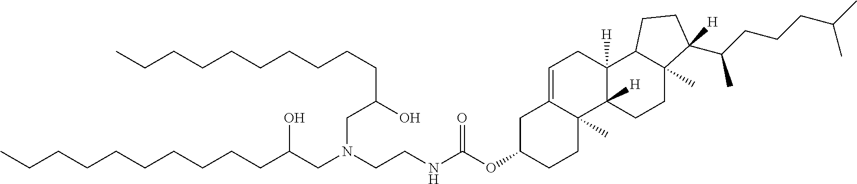 |
| 21 | 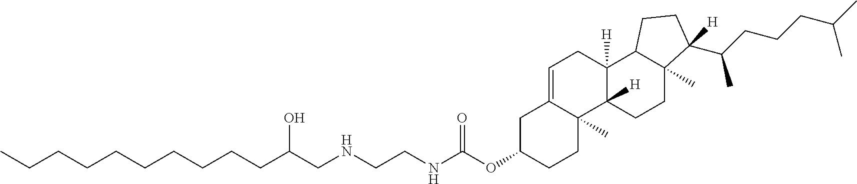 |
| 22 | 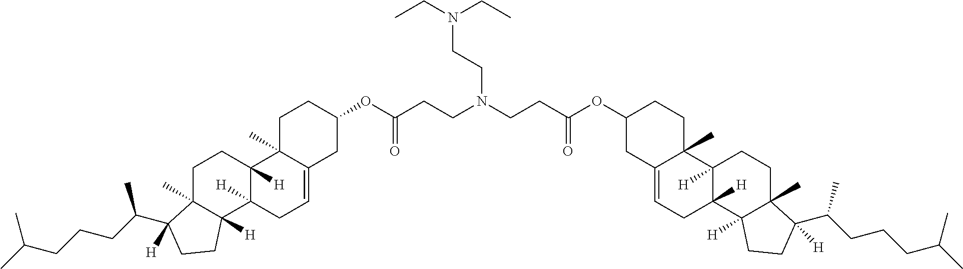 |
| 23 | 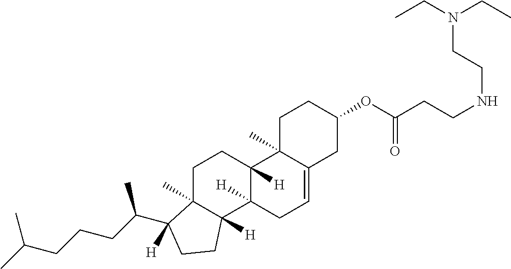 |
| 24 | 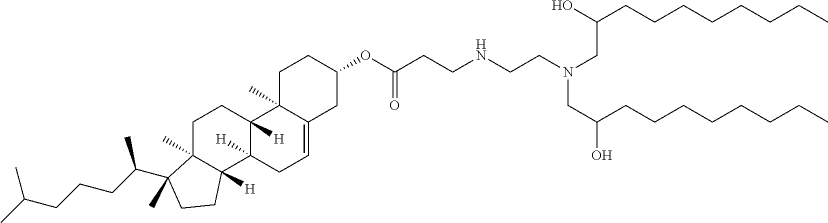 |
| 25 | 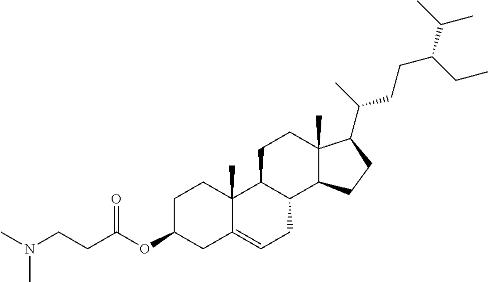 [SMILES: CC[C@H](CC[C@@H](C)[C@H]1CC[C@H]2[C@@H]3CC=C4C[C@@H](OC(=O)CCN(C)C)CC[C@]4(C)[C@H]3CC[C@]12C)C(C)C] |
| 26 | 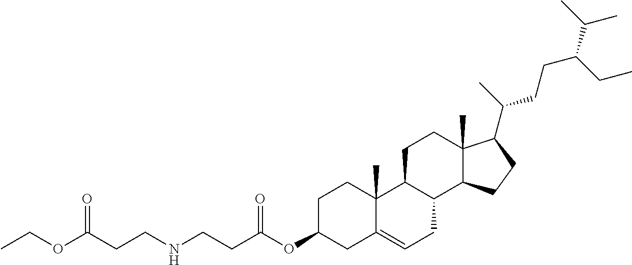 [SMILES: CCOC(=O)CCNCCC(=O)O[C@H]1CC[C@@]2(C)C(=CC[C@H]3[C@@H]4CC[C@H]([C@H](C)CC[C@@H](CC)C(C)C)[C@@]4(C)CC[C@@H]32)C1] |
| 27 | 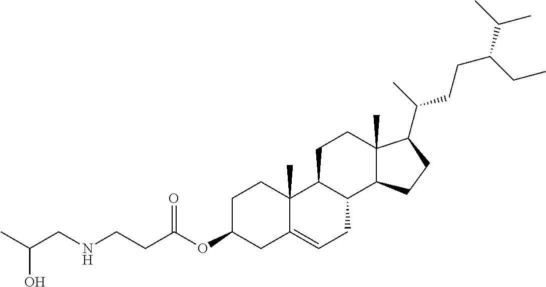 [SMILES: CC[C@H](CC[C@@H](C)[C@H]1CC[C@H]2[C@@H]3CC=C4C[C@@H](OC(=O)CCNCC(C)O)CC[C@]4(C)[C@H]3CC[C@]12C)C(C)C] |
| 28 | 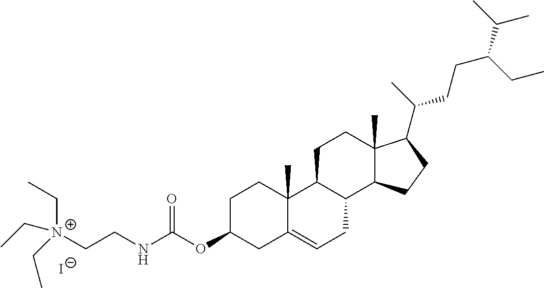 [SMILES: CC[C@H](CC[C@@H](C)[C@H]1CC[C@H]2[C@@H]3CC=C4C[C@@H](OC(=O)NCC[N+](CC)(CC)CC)CC[C@]4(C)[C@H]3CC[C@]12C)C(C)C ; [I-]] |
| 29 | 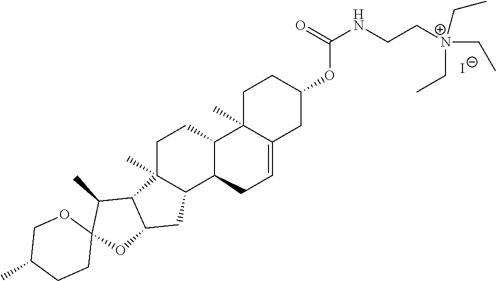 [SMILES: CC[N+](CC)(CC)CCNC(=O)O[C@H]1CC[C@@]2(C)C(=CC[C@H]3[C@@H]4C[C@@H]5O[C@@]6(CC[C@H](C)CO6)[C@@H](C)[C@@H]5[C@@]4(C)CC[C@@H]32)C1 ; [I-]] |
| 30 | 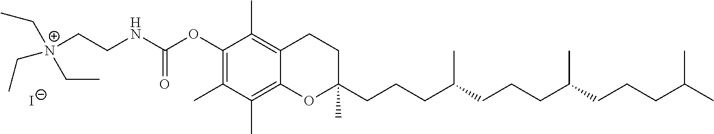 [SMILES: CC[N+](CC)(CC)CCNC(=O)Oc1c(C)c(C)c2c(c1C)CC[C@@](C)(CCC[C@H](C)CCC[C@H](C)CCCC(C)C)O2 ; [I-]] |
| 31 | 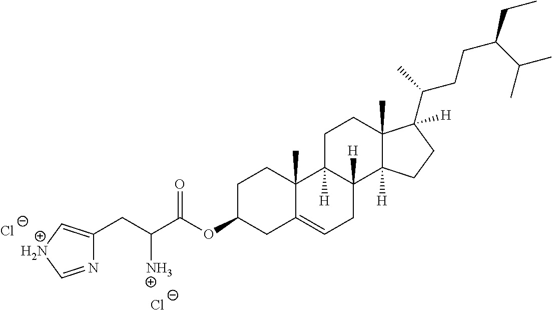 |
| 32 | 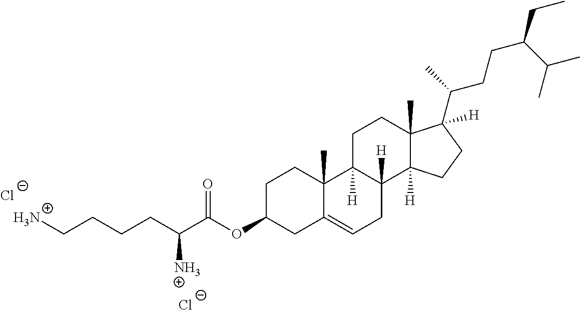 |
| 33 | 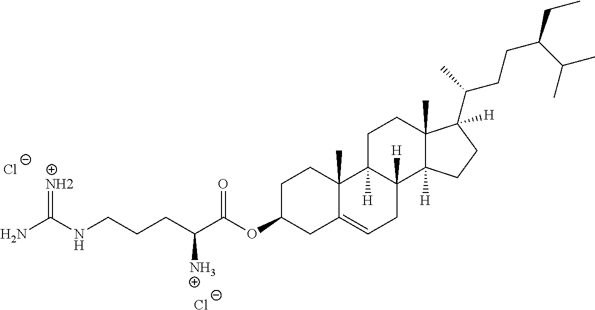 |
| 34 | 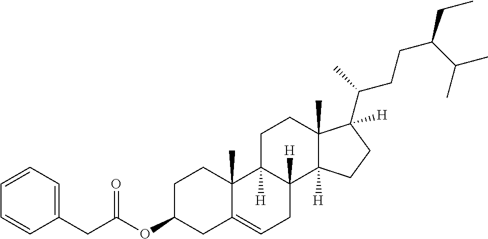 |
| 35 | 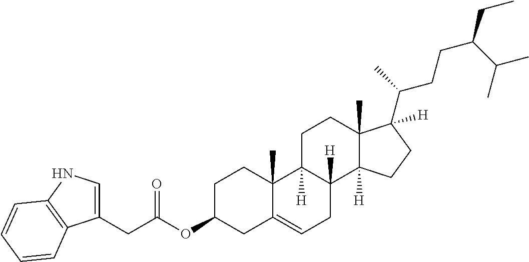 |
| 36 | 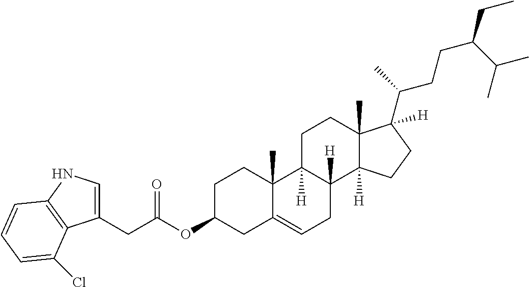 |
| 37 | 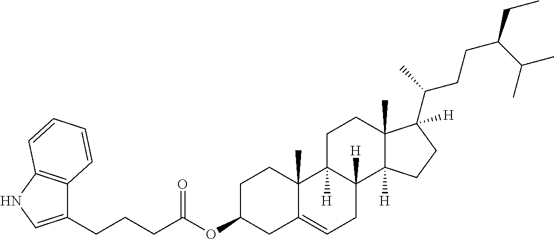 |
| 38 | 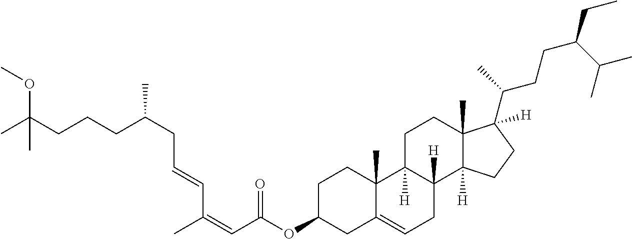 |
| 39 | 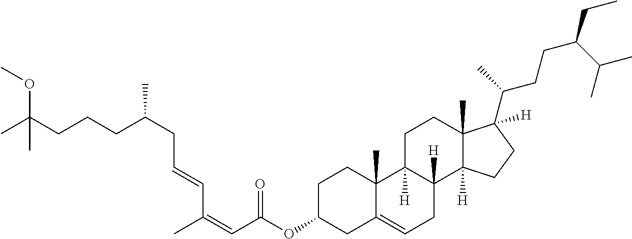 |
| 40 | 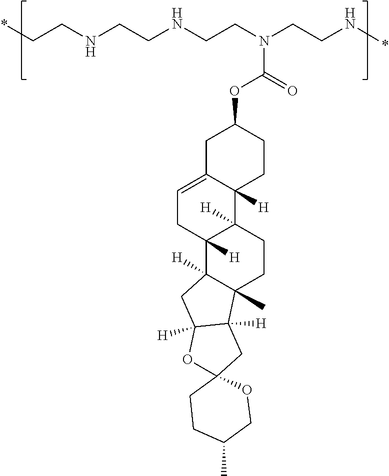 |
| 41 | 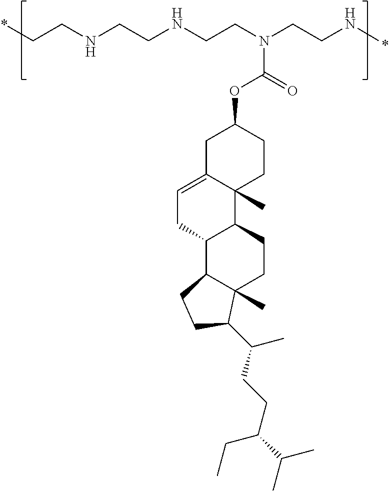 |
| 42 | 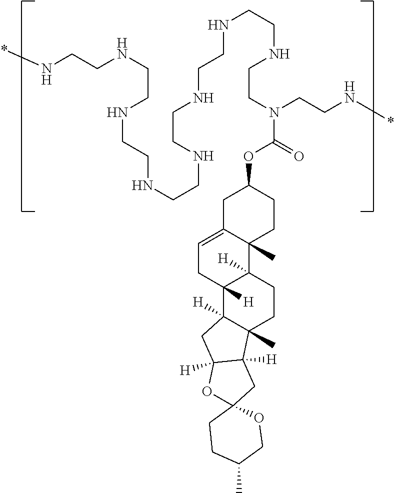 |
| 43 | 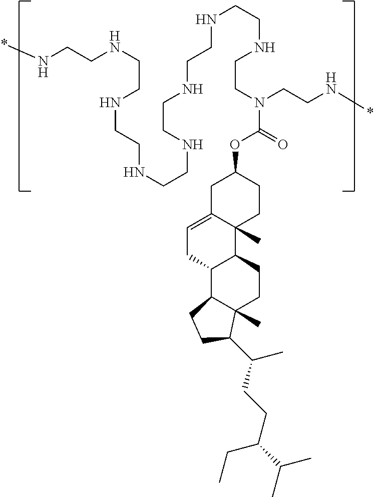 |
| 44 | 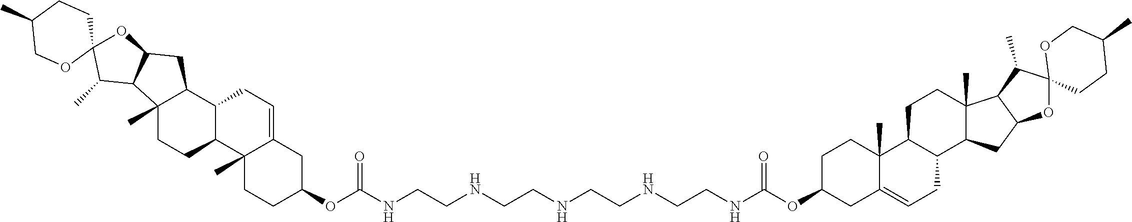 |
| 45 | 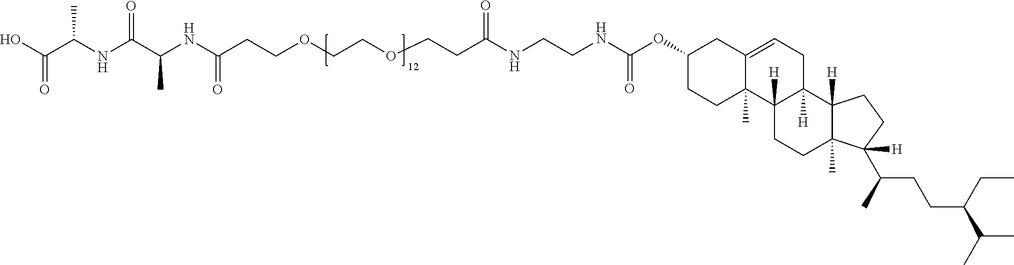 |
| 46 | 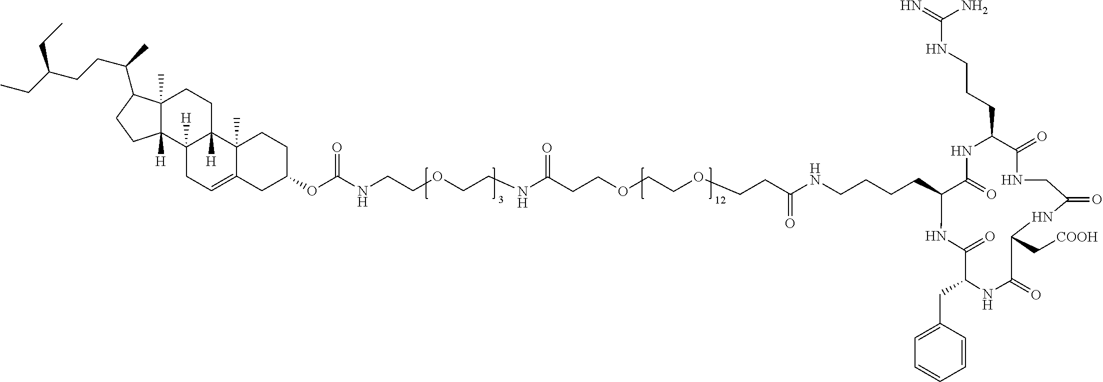 |
| 47 | 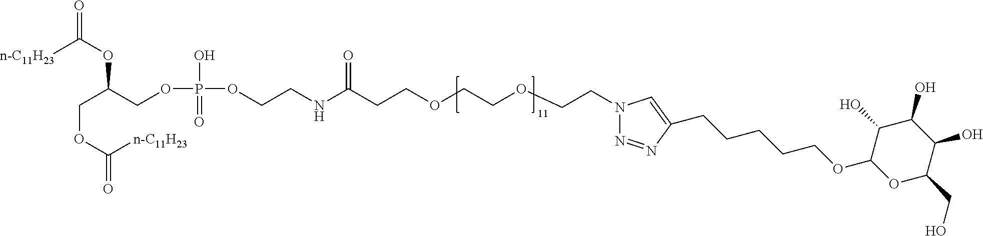 |
| 48 | 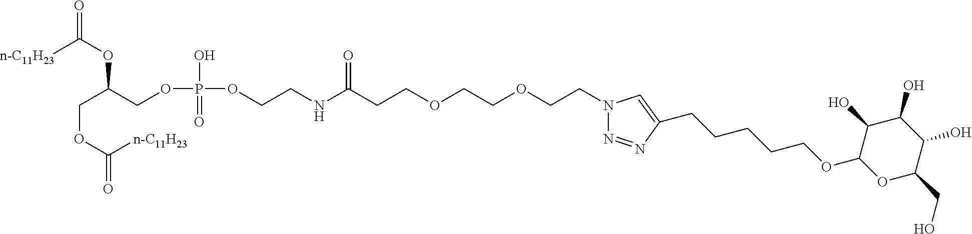 |
| 49 | 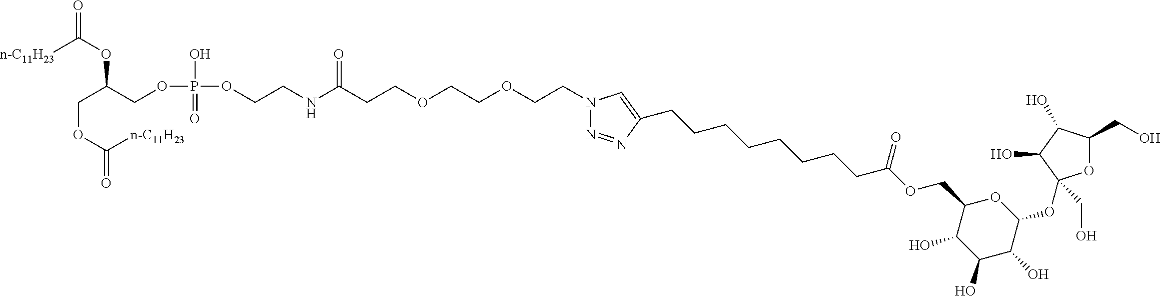 |
| 50 | 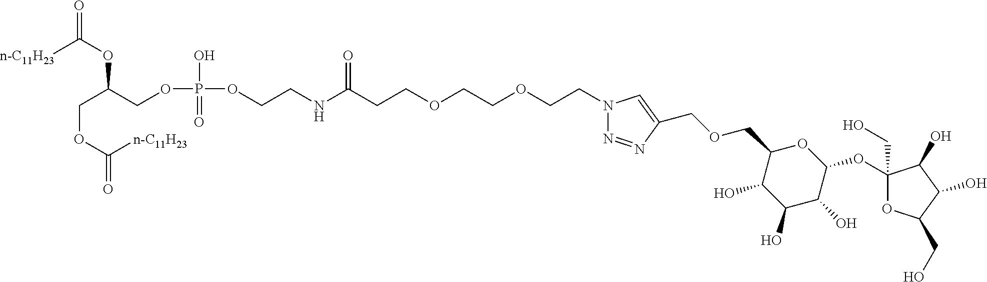 |

67. A compound of formula (XXVII): 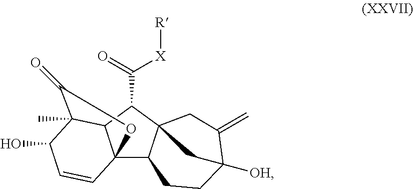
wherein
X is O or NH; and
R′ is an alkyl group or the residue of a insect-, plant-, or plant pathogen-derived steroid, tocopherol, endogenous auxin, or carbohydrate.

68. The compound of claim 67, wherein R′ is a C₁ to C₂₀ alkyl group.

69. The compound of claim 67, wherein:
X is O and R′ is a C₁₂ alkyl group; or
X is O or NH and R′ is a group of formula (XXVIII):

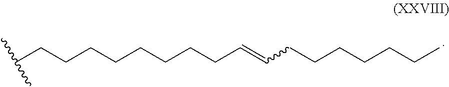 [SMILES: *CCCCCCCCC=CCCCCCCC]

70. The compound of claim 66, wherein X is O and R′ is a group selected from the group consisting of formulae (V), (VI), (VII), (VIII), (XIX), (XX), (XXI), (XXII), (XXIII), (XXIV), (XXV), and (XXVI): 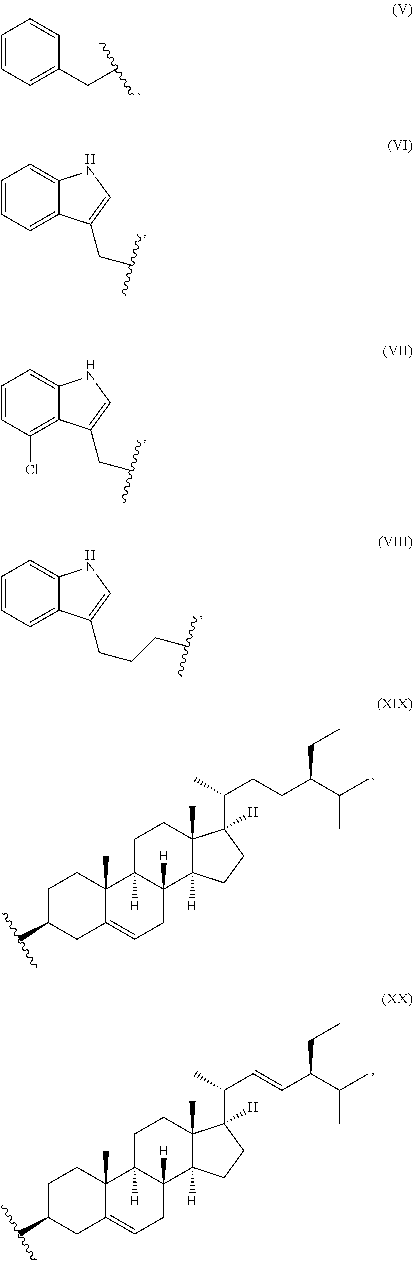 [chemical structure]
